(19) United States
(12) Patent Application Publication
(10) Pub. No.: US 2010/0029689 A1
(43) Pub. Date: Feb. 4, 2010

(54) PHOSPHODIESTERASE 4 INHIBITORS

(75) Inventors: Allen T. HOPPER, Katonah, NY (US); Joan Marie CAROON, Mountain View, CA (US); Elbert CHIN, San Jose, CA (US); Robert F. DUNN, Towaco, NJ (US); Sharada Shenvi LABADIE, Sunnyvale, CA (US); Jim LI, San Francisco, CA (US); Richard Allen SCHUMACHER, Monroe, NY (US); Francisco Xavier TALAMAS, Mountain View, CA (US)

Correspondence Address: MILLEN, WHITE, ZELANO & BRANIGAN, P.C., 2200 CLARENDON BLVD., SUITE 1400, ARLINGTON, VA, 22201, US

(73) Assignees: MEMORY PHARMACEUTICALS CORPORATION, Montvale, NJ (US); ROCHE PALO ALTO LLC, Palo Alto, CA (US)

(21) Appl. No.: 12/497,168

(22) Filed: Jul. 2, 2009

Related U.S. Application Data
(60) Provisional application No. 61/077,625, filed on Jul. 2, 2008.

(51) Int. Cl. A61K 31/505; A61K 31/50; A61K 31/445; A61K 31/4462; A61K 31/444; A61K 31/4406; A61K 31/426; A61K 31/341; C07D 237/08; C07D 239/26; C07D 241/12; C07D 213/55; C07D 277/28; C07D 263/32; A61P 29/00; A61P 25/00; A61P 37/08

(52) U.S. Cl. 514/256; 514/247; 514/319; 514/331; 514/332; 514/357; 514/365; 514/471; 544/224; 544/335; 544/336; 546/335; 548/204; 548/205; 548/236

(57) ABSTRACT

PDE4 inhibition is achieved by novel compounds, e.g., N-substituted aniline and diphenylamine analogs. The compounds of the present invention are of Formula I:

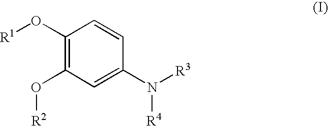

wherein R¹, R², R³, and R⁴ are as defined herein.

This application claims the benefit of U.S. Patent Application 61/077,625, filed Jul. 2, 2008.

This application is related to U.S. Pat. No. 6,699,890, U.S. Pat. No. 7,205,320 (divisional of U.S. Pat. No. 6,699,890), and copending application Ser. No. 11/602,283, filed Nov. 21, 2007 (divisional of U.S. Pat. No. 7,205,320), the disclosures of each of which are hereby incorporated by reference in their entirety.

This application is also related to copending U.S. application Ser. No. 10/622,833, filed Jul. 21, 2003, the disclosure of which is hereby incorporated by reference in their entirety.

This application is also related to U.S. Pat. No. 7,087,625, and copending U.S. application Ser. No. 11/378,615, filed Mar. 20, 2006 (continuation of U.S. Pat. No. 7,087,625), the disclosures of each of which are hereby incorporated by reference in their entirety.

This application is also related to copending U.S. application Ser. No. 11/008,775, filed Dec. 10, 2004, the disclosure of which is hereby incorporated by reference in their entirety.

This application is also related to copending U.S. application Ser. No. 11/449,868, filed Jun. 9, 2006, the disclosure of which is hereby incorporated by reference in their entirety.

### BACKGROUND OF THE INVENTION

The cyclic nucleotide specific phosphodiesterases (PDEs) represent a family of enzymes that catalyze the hydrolysis of various cyclic nucleoside monophosphates (including cAMP and cGMP). These cyclic nucleotides act as second messengers within cells, and as messengers, carry impulses from cell surface receptors having bound various hormones and neurotransmitters. PDEs act to regulate the level of cyclic nucleotides within cells and maintain cyclic nucleotide homeostasis by degrading such cyclic mononucleotides resulting in termination of their messenger role.

PDE enzymes can be grouped into eleven families according to their specificity toward hydrolysis of cAMP or cGMP, their sensitivity to regulation by calcium, calmodulin or cGMP, and their selective inhibition by various compounds. For example, PDE 1 is stimulated by Ca²⁺/calmodulin. PDE 2 is cGMP-dependent, and is found in the heart and adrenals. PDE 3 is cGMP-dependent, and inhibition of this enzyme creates positive inotropic activity. PDE is cAMP specific, and its inhibition causes airway relaxation, anti-inflammatory and antidepressant activity. PDE 5 appears to be important in regulating cGMP content in vascular smooth muscle, and therefore PDE 5 inhibitors may have cardiovascular activity. Since the PDEs possess distinct biochemical properties, it is likely that they are subject to a variety of different forms of regulation.

PDE4 is distinguished by various kinetic properties including low Michaelis constant for cAMP and sensitivity to certain drugs. The PDE4 enzyme family consists of four genes, which produce 4 isoforms of the PDE4 enzyme designated PDE4A, PDE4B, PDE4C, and PDE4D [See: Wang et al., Expression, Purification, and Characterization of human cAMP-Specific Phosphodiesterase (PDE4) Subtypes A, B, C, and D, Biochem. Biophys. Res. Comm., 234, 320-324 (1997)]. In addition, various splice variants of each PDE4 isoform have been identified.

PDE4 isoenzymes are localized in the cytosol of cells and are unassociated with any known membranous structures. PDE4 isoenzymes specifically inactivate cAMP by catalyzing its hydrolysis to adenosine 5′-monophosphate (AMP). Regulation of cAMP activity is important in many biological processes, including inflammation and memory. Inhibitors of PDE4 isoenzymes such as rolipram, piclamilast, CDP-840 and ariflo are powerful anti-inflammatory agents and therefore may be useful in treating diseases where inflammation is problematic such as asthma or arthritis. Further, rolipram improves the cognitive performance of rats and mice in learning paradigms.

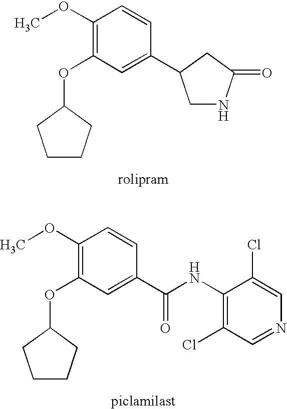

In addition to such compounds as rolipram, and xanthine derivatives such as pentoxifylline, denbufylline, and theophylline, inhibit PDE4 and have received considerable attention for their cognition enhancing effects. cAMP and cGMP are second messengers that mediate cellular responses to many different hormones and neurotransmitters. Thus, therapeutically significant effects may result from PDE inhibition and the resulting increase in intracellular cAMP or cGMP in key cells, such as those located in the nervous system and elsewhere in the body.

Rolipram, previously in development as an anti-depressant, selectively inhibits the PDE4 enzyme and has become a standard agent in the classification of PDE enzyme subtypes. Early work in the PDE4 field focused on depression and inflammation, and has subsequently been extended to include indications such as dementia. [see “The PDE IV Family Of Calcium-Phosphodiesterases Enzymes,” John A. Lowe, III, et al., Drugs of the Future, 1992, 17(9):799-807 for a general review]. Further clinical developments of rolipram and other first-generation PDE4 inhibitors were terminated due to the side effect profile of these compounds. The primary side effect in primates is emesis, while the primary side effects in rodents are testicular degranulation, weakening of vascular smooth muscle, psychotrophic effects, increased gastric acid secretion and stomach erosion.

### SUMMARY OF THE INVENTION

The present invention relates generally to the field of phosphodiesterase 4 (PDE4) enzyme inhibition. More specifically the present invention relates to novel compounds, e.g., novel N-substituted aniline and diphenylamine compounds, that inhibit PDE4 enzymes, and especially have improved side effect profiles, e.g., are relatively non-emetic, (e.g., as compared to the previously discussed prior art compounds). Preferably, the compounds selectively inhibit PDE4 enzymes. The compounds of this invention at the same time facilitate entry into cells, especially cells of the nervous system.

Still further, the present invention provides methods for synthesizing compounds with such activity and selectivity as well as methods of (and corresponding pharmaceutical compositions for) treating a patient, e.g., mammals, including humans, requiring PDE inhibition, especially PDE4 inhibition, for a disease state that involves elevated intracellular PDE 4 levels or decreased cAMP levels, e.g., involving neurological syndromes, especially those states associated with memory impairment, most especially long term memory impairment, as where such memory impairment is due in part to catabolism of intracellular cAMP levels by PDE 4 enzymes, or where such memory impairment may be improved by effectively inhibiting PDE4 enzyme activity.

In a preferred aspect, the compounds of the invention improve such diseases by inhibiting PDE4 enzymes at doses which do not induce emesis.

The present invention includes compounds that fall within the scope of the genus defined by Formula I:

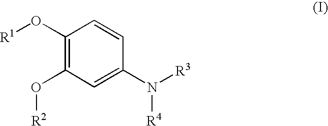

wherein
R¹ is C₁₋₄-alkyl, which is branched or unbranched, and which is unsubstituted or substituted one or more times by halogen (e.g., CH₃, C₂H₅, CHF₂, CF₃, CH₂CF₃, etc.), or
   C₃₋₁₀-cycloalkyl, preferably C₃₋₆-cycloalkyl, which is unsubstituted or substituted one or more times by halogen, hydroxy, oxo, cyano, C₁₋₄-alkyl, C₁₋₄-alkoxy, or combinations thereof (e.g., cyclopropyl, cyclobutyl, cyclopentyl);
R² is H, C₁₋₁₂-alkyl, preferably C₁₋₈-alkyl, which is branched or unbranched, and which is unsubstituted or substituted one or more times by halogen, hydroxy, cyano, C₁₋₄-alkoxy, oxo or combinations thereof, and wherein optionally one or more —CH₂CH₂— groups is replaced in each case by —CH═CH— or —C≡C— (e.g., methyl, ethyl, isopropyl, butyl, CHF₂, CF₃, CH₂CF₃, etc.),
   C₃₋₁₀-cycloalkyl, preferably C₃₋₈-cycloalkyl, which is unsubstituted or substituted one or more times by halogen, hydroxy, oxo, cyano, C₁₋₄-alkyl, C₁₋₄-alkoxy, or combinations thereof (e.g., cyclopropyl, cyclobutyl, cyclopentyl),
   C₄₋₁₆-cycloalkylalkyl, preferably C₄₋₁₂-cycloalkylalkyl, which is unsubstituted or substituted in the cycloalkyl portion and/or the alkyl portion one or more times by halogen, oxo, cyano, hydroxy, C₁₋₄-alkyl, C₁₋₄-alkoxy or combinations thereof (e.g., cyclopentylmethyl, cyclopropylmethyl, etc.),
   C₆₋₁₄-aryl which is unsubstituted or substituted one or more times by halogen, CF₃, OCF₃, C₁₋₈-alkyl, hydroxy, C₁₋₈-alkoxy, nitro, methylenedioxy, ethylenedioxy, cyano, or combinations thereof (e.g., methylphenyl, methoxyphenyl, chlorophenyl, etc.),
   C₆₋₁₄-aryl-C₁₋₅-alkyl, in which the alkyl portion is branched or unbranched, which is unsubstituted or is substituted in the aryl portion one or more times by halogen, CF₃, OCF₃, C₁₋₈-alkyl, hydroxy, C₁₋₈-alkoxy, nitro, cyano, methylenedioxy, ethylenedioxy, or combinations thereof, and wherein in the alkyl portion one or more —CH₂CH₂— groups are each optionally replaced by —CH═CH— or —C≡C—, and one or more —CH₂— groups are each optionally replaced by —O— or —NH— and/or the alkyl portion is optionally substituted by halogen, oxo, hydroxy, cyano, or combinations thereof (e.g., benzyl, phenylethyl, phenylpropyl, phenylbutyl, methoxyphenylethyl, methoxyphenylpropyl, chlorophenylethyl, chlorophenylpropyl, phenylethenyl, phenoxyethyl, phenoxybutyl, chlorophenoxyethyl, chlorophenylaminoethyl, etc., especially benzyl),
   a partially unsaturated C₅₋₁₄-carbocyclic group, which is unsubstituted or substituted one or more times by halogen, C₁₋₈-alkyl, C₁₋₈-alkoxy, hydroxy, nitro, cyano, oxo, or combinations thereof (e.g., cyclohexenyl, cyclohexadienyl, indanyl, tetrahydronaphthenyl, etc.),
   a heterocyclic group, which is saturated, partially saturated or unsaturated, having 5 to 10 ring atoms in which at least 1 ring atom is a N, O or S atom, which is unsubstituted or substituted one or more times by halogen, hydroxy, C₆₋₁₄-aryl, C₁₋₈-alkyl, C₁₋₈-alkoxy, cyano, trifluoromethyl, nitro, oxo, or combinations thereof (e.g., 3-pyrrolyl, 3-thienyl, and particularly tetrahydrofuranyl such as tetrahydrofuran-3-yl, etc.), or
   a heterocycle-alkyl group, wherein the heterocyclic portion is saturated, partially saturated or unsaturated, and has 5 to 10 ring atoms in which at least 1 ring atom is a N, O or S atom, and the alkyl portion is branched or unbranched, the heterocycle-alkyl group is unsubstituted or substituted one or more times in the heterocyclic portion by halogen, OCF₃, hydroxy, C₆₋₁₄-aryl, C₁₋₈-alkyl, C₁₋₈-alkoxy, cyano, trifluoromethyl, nitro, oxo, or combinations thereof, wherein in the alkyl portion one or more —CH₂CH₂— groups are each optionally replaced by —CH═CH— or —C≡C—, and one or more —CH₂— groups are each optionally replaced by —O— or —NH— and/or the alkyl portion is optionally substituted by halogen, oxo, hydroxy, cyano, or combinations thereof (e.g., pyridylethyl, pydridylpropyl, methylpiperazinylethyl, etc.);
R³ is H,
   C₁₋₈-alkyl, preferably C₁₋₄-alkyl, which is branched or unbranched, and which is unsubstituted or substituted one or more times with halogen, hydroxyl, cyano, C₁₋₄-alkoxy, or combinations thereof (e.g., methyl, ethyl, propyl, 2-hydroxyethyl, 2-hydroxy-2-methyl-propyl, 2-hydroxypropyl, 3-hydroxypropyl, etc.),
   a partially unsaturated C₅₋₁₄-carbocycle-C₁₋₅-alkyl group wherein the alkyl portion which is branched or unbranched, and which is unsubstituted or substituted in the carbocyclic portion one or more times by halogen, C₁₋₈-alkyl, C₁₋₈-alkoxy, nitro, cyano, oxo, or combinations thereof, and the alkyl portion is optionally substituted by halogen, C₁₋₄-alkoxy, cyano or combinations thereof (e.g., cyclohexenylmethyl, etc.),
   C₇₋₁₉-arylalkyl having 7 to 19 carbon atoms, wherein the aryl portion has 6 to 14 carbon atoms and the alkyl portion, which is branched or unbranched, has 1 to 5 carbon atoms, and which is unsubstituted or substituted, in the aryl portion, one or more times by halogen, trifluoromethyl, CF₃O, nitro, amino, C₁₋₈-alkyl, C₁₋₈-alkoxy, C₁₋₈-alkylamino, di-(C₁₋₈-alkyl)amino, and/or substituted in the alkyl portion by halogen, cyano, or methyl (e.g., benzyl, phenethyl, phenpropyl, methylbenzyl, methoxybenzyl, trifluoromethyl, benzyl, methylenedioxobenzyl, etc., particularly benzyl), or
   a heterocycle-C₁₋₅-alkyl group, wherein the heterocyclic portion is saturated, partially saturated or unsaturated (e.g., heteroarylalkyl), wherein the heterocyclic portion has 5 to 10 ring atoms in which at least 1 ring atom is a N, O or S atom, and the alkyl portion is branched or unbranched, the heterocycle-C₁₋₅-alkyl group is unsubstituted or substituted one or more times in the heterocyclic portion by halogen, C₁₋₈-alkyl, C₁₋₈-alkoxy, cyano, trifluoromethyl, CF₃O, nitro, oxo, amino, C₁₋₈-alkylamino, di-(C₁₋₈-alkyl)amino, aminocarbonyl (i.e., carbamoyl, NH₂—CO—), C₆₋₁₄-aryl which is unsubstituted or substituted (e.g., aryl substituted one or more times by C₁₋₄-alkyl, C₁₋₄-alkoxy, halogenated C₁₋₄-alkyl, and/or halogenated C₁₋₄-alkoxy, such as 4-difluoromethoxy-3-ethoxyphenyl), or combinations thereof, and/or substituted in the alkyl portion by halogen, cyano, or methyl or combinations thereof (e.g., pyrazinylmethyl, pyridylmethyl, 1-pyridylethyl, pyridylpropyl, methylpyridylmethyl, chloropyridylmethyl, dichloropyridylmethyl, thienylmethyl, pyrazolylmethyl, methylpyrazolylmethyl, oxazolylmethyl, thiazolylmethyl, quinolinylmethyl, isoquinolinylmethyl, piperidinylmethyl, tetrahydrofuranylmethyl, furanylmethyl, imidazolylmethyl, methylimidazolylmethyl, pyrrolylmethyl, pyrimidinylmethyl, pyradizinylmethyl, thiadiazolylmethyl, etc.);
R⁴ is H,
   C₁₋₈-alkyl, preferably C₁₋₄-alkyl, which is branched or unbranched and which is unsubstituted or substituted one or more times with halogen, carboxy, cyano, C₁₋₄-alkoxy, or combinations thereof (e.g., methyl, ethyl, propyl, 3-carboxypropyl, etc.),
   C₃₋₁₀-cycloalkyl, preferably C₃₋₈-cycloalkyl, which is unsubstituted or substituted one or more times by halogen, hydroxy, oxo, carboxy, cyano, C₁₋₄-alkyl, C₁₋₄-alkoxy, or combinations thereof (e.g., cyclopentyl, cyclohexyl, carboxycyclohexyl),
   C₆₋₁₄-aryl and which is unsubstituted or substituted one or more times by halogen, C₁₋₈-alkyl, C₂₋₈-alkenyl, C₂₋₈-alkynyl, hydroxy, C₁₋₈-alkoxy, C₁₋₈-alkoxy-C₁₋₈-alkoxy, nitro, methylenedioxy, ethylenedioxy, trifluoromethyl, OCF₃, amino, amino-C₁₋₈-alkyl (e.g., NH₂—CH₂—), amino-C₂₋₈-alkoxy (e.g., NH₂—CH₂CH₂O—), C₁₋₈-alkylamino (e.g., CH₃—NH—), di-(C₁₋₈-alkyl)amino (e.g., (CH₃)₂NH—), C₁₋₈-hydroxyalkyl (e.g., hydroxymethyl, hydroxypropyl, —C(CH₃)₂—OH), hydroxamic acid (—C(O)—NHOH), pyrrolyl, tetrazole-5-yl, 2(-heterocycle)tetrazole-5-yl (e.g., 2-(2-tetrahydropyranyl)tetrazole-5-yl), C₁₋₈-hydroxyalkoxy (e.g., HO—C(CH₃)₂—CH₂—O—), C₁₋₈-dihydroxyalkoxy (e.g., OCH₂CH(OH)CH₂OH), carboxy, C₁₋₈-alkoxy-carbonyl (e.g., tert-butoxycarbonyl, ethoxycarbonyl), cyano, acyl, C₁₋₈-alkylthio (e.g., methylthio, ethylthio), C₁₋₈-alkylsulfinyl, C₁₋₈-alkylsulfonyl (e.g., methylsulfonyl, ethylsulfonyl), phenoxy, tri-C₁₋₈-alkylsilyloxy (e.g. tert-butyldimethylsilyloxy), R⁵-L-, or combinations thereof (e.g., substituted or unsubstituted phenyl, naphthyl, and biphenyl, such as phenyl, methylphenyl, chlorophenyl, fluorophenyl, vinylphenyl, cyanophenyl, methylenedioxophenyl, ethylphenyl, dichlorophenyl, carboxyphenyl, ethoxycarbonylphenyl, dimethylphenyl, hydroxymethylphenyl, nitrophenyl, aminophenyl, etc.), or
   a heterocyclic group, which is fully saturated, partially saturated or unsaturated (e.g., heteroaryl), having 5 to 10 ring atoms in which at least 1 ring atom is a heteroatom, which is unsubstituted or substituted one or more times by halogen, C₁₋₈-alkyl, hydroxy, C₁₋₈-alkoxyl, oxo, C₁₋₈-alkoxy-C₁₋₈-alkoxy, nitro, methylenedioxy, ethylenedioxy, trifluoromethyl, amino, amino-C₁₋₈-alkyl, C₁₋₈-alkylamino, amino-C₂₋₈-alkoxy, di-C₁₋₈-alkylamino, C₁₋₈-hydroxyalkyl (e.g., hydroxymethyl), hydroxamic acid (—C(O)—NHOH), tetrazole-5-yl, C₁₋₈-hydroxyalkoxy, carboxy, C₁₋₈-alkoxy-carbonyl (e.g., tert-butyloxycarbonyl, ethoxycarbonyl), cyano, acyl, C₁₋₈-alkylthio (e.g., methylthio, ethylthio), C₁₋₈-alkylsulfinyl, C₁₋₈-alkylsulfonyl (e.g., methylsulfonyl, ethylsulfonyl), phenoxy, tri-C₁₋₈-alkylsilyloxy (e.g. tert-butyldimethylsilyloxy), R⁵-L-, or combinations thereof (e.g., pyridyl, thienyl, pyrazinyl, quinolinyl, isoquinolinyl, pyrimidinyl, imidazolyl, thiazolyl, benzisoxazolyl, etc.);
R⁵ is H,
   C₁₋₈-alkyl, preferably C₁₋₄-alkyl, which is unsubstituted or substituted one or more times with halogen, C₁₋₄-alkyl, C₁₋₄-alkoxy, oxo, hydroxy, or combinations thereof (e.g., methyl, ethyl, propyl, etc.),
   C₁₋₈-alkylamino or di-C₁₋₈-alkylamino, preferably C₁₋₄-alkylamino or di-C₁₋₄-alkylamino (e.g., dimethylamino, etc.),
   a partially unsaturated carbocycle-alkyl group wherein the carbocyclic portion has 5 to 14 carbon atoms and the alkyl portion has 1 to 5 carbon atoms, which is unsubstituted or substituted, preferably in the carbocyclic portion, one or more times by halogen, alkyl, alkoxy, nitro, cyano, oxo, or combinations thereof (e.g., cyclohexenylmethyl, etc.),
   C₃₋₁₀-cycloalkyl, preferably C₃₋₈-cycloalkyl, which is unsubstituted or substituted one or more times by halogen, hydroxy, oxo, cyano, C₁₋₄-alkoxy, C₁₋₄-alkyl, or combinations thereof (e.g., cyclopentyl),
   C₄₋₁₆-cycloalkylalkyl, preferably C₄₋₁₂-cycloalkylalkyl, which is unsubstituted or substituted in the cycloalkyl portion and/or the alkyl portion one or more times by halogen, oxo, cyano, hydroxy, alkyl, alkoxy or combinations thereof (e.g., cyclopentylmethyl, cyclopropylmethyl, etc.),
   C₆₋₁₄-aryl which is unsubstituted or substituted one or more times by halogen, C₁₋₈-alkyl, hydroxy, C₁₋₈-alkoxy, C₁₋₈-alkoxy-C₁₋₈-alkoxy, nitro, methylenedioxy, ethylenedioxy, trifluoromethyl, amino, amino-C₁₋₈-alkyl, amino-C₂₋₈-alkoxy, C₁₋₈-alkylamino, di-C₁₋₈-alkylamino, C₁₋₈-hydroxyalkyl (e.g., hydroxymethyl), hydroxamic acid (—C(O)—NHOH), tetrazole-5-yl, C₁₋₈-hydroxyalkoxy, carboxy, C₁₋₈-alkoxy-carbonyl (e.g., tert-butyloxycarbonyl, ethoxycarbonyl), cyano, acyl, C₁₋₈-alkylthio (e.g., methylthio, ethylthio), C₁₋₈-alkylsulfinyl, C₁₋₈-alkylsulfonyl (e.g., methylsulfonyl, ethylsulfonyl), phenoxy, C₃₋₁₀-cycloalkyl, C₆₋₁₄-aryl, heteroaryl or combinations thereof (e.g., substituted or unsubstituted phenyl and naphthyl, methylphenyl, chlorophenyl, fluorophenyl, vinylphenyl, cyanophenyl, methylenedioxophenyl, ethylphenyl, dichlorophenyl, carboxyphenyl, ethoxycarbonylphenyl, dimethylphenyl, hydroxymethylphenyl, nitrophenyl, aminophenyl, etc.),
   C₇₋₁₉-arylalkyl wherein the aryl portion has 6 to 14 carbon atoms and the alkyl portion, which is branched or unbranched, has 1 to 5 carbon atoms, and which is unsubstituted or substituted, in the aryl portion, one or more times by halogen, trifluoromethyl, CF₃O, nitro, amino, C₁₋₈-alkyl, C₁₋₈-alkoxy, amino, C₁₋₈-alkylamino, or di-C₁₋₈-alkylamino, and/or substituted in the alkyl portion by halogen, cyano, or methyl (e.g., benzyl, phenethyl, phenpropyl, methylbenzyl, methoxybenzyl, trifluoromethyl, benzyl, methylenedioxobenzyl, etc.),
   a heterocyclic group, which is saturated, partially saturated or unsaturated, having 5 to 10 ring atoms in which at least 1 ring atom is a N, O or S atom, which is unsubstituted or substituted one or more times by halogen, C₁₋₈-alkyl, hydroxy, C₁₋₈-alkoxy, C₁₋₈-alkoxy-C₁₋₈-alkoxy, oxo, nitro, methylenedioxy, ethylenedioxy, trifluoromethyl, amino, amino-C₁₋₈-alkyl, amino-C₂₋₈-alkoxy, C₁₋₈-alkylamino, di-C₁₋₈-alkylamino, C₁₋₈-hydroxyalkyl (e.g., hydroxymethyl), hydroxamic acid (—C(O)—NHOH), tetrazole-5-yl, C₁₋₈-hydroxyalkoxy, carboxy, C₁₋₈-alkoxy-carbonyl (e.g., tert-butyloxycarbonyl, ethoxycarbonyl), cyano, acyl, C₁₋₈-alkylthio, C₁₋₈-alkylsulfinyl, C₁₋₈-alkylsulfonyl, phenoxy, C₃₋₁₀-cycloalkyl, C₆₋₁₄-aryl, heteroaryl or combinations thereof (e.g., pyrrolidinyl, 1,3-oxazoldiny, 1,3-dioxanyl, pyrazolyl, pyranyl, 1,2,4-triazolyl, imidazolidinyl, pyridyl, thienyl, pyrazinyl, quinolinyl, isoquinolinyl, pyrimidinyl, imidazolyl, thiazolyl, etc.), or
   a heterocycle-alkyl group, wherein the heterocyclic portion is saturated, partially saturated or unsaturated, and has 5 to 10 ring atoms in which at least 1 ring atom is a N, O or S atom, and the alkyl portion which is branched or unbranched and has 1 to 5 carbon atoms, the heterocycle-alkyl group is unsubstituted or substituted one or more times in the heterocyclic portion by halogen, C₁₋₈-alkyl, C₁₋₈-alkoxy, cyano, trifluoromethyl, CF₃O, nitro, oxo, amino, C₁₋₈-alkylamino, di-C₁₋₈-alkylamino, or combinations thereof and/or substituted in the alkyl portion by halogen, cyano, or methyl or combinations thereof (e.g., pyridylmethyl, pyridylpropyl, methylpridylmethyl, morpholinylethyl, pyrrolidinylmethyl, etc.);
L is a single bond or a divalent aliphatic radical having 1 to 8 carbon atoms wherein one or more —CH₂— groups are each optionally replaced by —O—, —S—, —SO—, —SO₂—, —NR⁶—, —SO₂NH—, —NHSO₂—, —SO₂NR⁶—, —NRSO₂—, —CHOH—, —CO—, —NR⁶CO—, —CONR⁶—, —NHCONH—, —OCONH, —NHCOO—, —SCONH—, —SCSNH—, or —NHCSNH— (e.g., —O—, CH₂—, —CO—, —CO—O—, —O—CO—, —CH₂—CO—O—, —CH₂CH₂—CO—O—, —CH₂CH₂CH₂—CO—O—, —CO—NH—, —NH—CO—, —CO—NH—O—, —NCH₃—CH₂—CO—, —CH₂CH₂CH₂—NH—CO—, —CH₂—CH₂—O—, —SO₂—NH—CH₂CH₂—O—, —O—CH₂CH₂—O—, —O—CH₂—CO—O—, —CH₂—NH—CO—, —CO—NH—CH₂—, —SO₂—NH—, —NH—SO₂—, —CH₂—NH—SO₂—, —CH₂CH₂CH₂—SO₂—NH—, —NH—CH₂CH₂—SO₂—, —SO₂—, —CO—NH—SO₂—, etc.); and
R⁶ is H,
   C₁₋₈-alkyl, preferably C₁₋₄-alkyl, which is branched or unbranched and which is unsubstituted or substituted one or more times with halogen, C₁₋₄-alkyl, C₁₋₄-alkoxy, oxo, or combinations thereof (e.g., methyl, ethyl, propyl, etc.);
   C₇₋₁₉-arylalkyl wherein the aryl portion has 6 to 14 carbon atoms and the alkyl portion, which is branched or unbranched, has 1 to 5 carbon atoms, and which is unsubstituted or substituted, in the aryl portion, one or more times by halogen, trifluoromethyl, CF₃O, nitro, amino, C₁₋₈-alkyl, C₁₋₈-alkoxy, C₁₋₈-alkylamino, or di-C₁₋₈-alkylamino, and/or substituted in the alkyl portion by halogen, cyano, or methyl (e.g., benzyl, phenethyl, phenpropyl, methylbenzyl, methoxybenzyl, trifluoromethyl, benzyl, methylenedioxobenzyl, etc.);
   C₆₋₁₄-aryl which is unsubstituted or substituted one or more times by halogen, C₁₋₈-alkyl, hydroxy, C₁₋₈-alkoxy, C₁₋₈-alkoxy-C₁₋₈-alkoxy, nitro, methylenedioxy, ethylenedioxy, trifluoromethyl, amino, amino-C₁₋₈-alkyl, amino-C₂₋₈-alkoxy, C₁₋₈-alkylamino, di-C₁₋₈-alkylamino, C₁₋₈-hydroxyalkyl (e.g., hydroxymethyl), hydroxamic acid (—C(O)—NHOH), tetrazole-5-yl, C₁₋₈-hydroxyalkoxy, carboxy, C₁₋₈-alkoxy-carbonyl (e.g., tert-butyloxycarbonyl, ethoxycarbonyl), cyano, acyl, C₁₋₈-alkylthio, C₁₋₈-alkylsulfinyl, or C₁₋₈-alkylsulfonyl, (e.g., substituted or unsubstituted phenyl and naphthyl, methylphenyl, chlorophenyl, fluorophenyl, vinylphenyl, cyanophenyl, methylenedioxophenyl, ethylphenyl, dichlorophenyl, carboxyphenyl, ethoxycarbonylphenyl, dimethylphenyl, hydroxymethylphenyl, nitrophenyl, aminophenyl, etc.),
   and pharmaceutically acceptable salts or solvates (e.g., hydrates) or N-oxides thereof, or solvates of pharmaceutically acceptable salts or N-oxides thereof.
   wherein at least one of R³ and R⁴ is other than H.

Some compounds encompassed by the genus of Formula I are disclosed in U.S. Pat. No. 6,699,890, U.S. Pat. No. 7,205,320, copending application Ser. No. 11/602,283, copending U.S. application Ser. No. 10/622,833, U.S. Pat. No. 7,087,625, copending U.S. application Ser. No. 11/378,615, filed Mar. 20, 2006, copending U.S. application Ser. No. 11/008,775, filed Dec. 10, 2004, and copending U.S. application Ser. No. 11/449,868, filed Jun. 9, 2006.

According to a further aspect of the invention, the compounds are of Formula (I) in which:
R¹ is cycloalkyl having 3 to 10, preferably 3 to 6 carbon atoms, which is unsubstituted or substituted one or more times by halogen, hydroxy, oxo, cyano, C₁₋₄-alkyl, C₁₋₄-alkoxy, or combinations thereof, e.g., cyclopropyl, cyclobutyl, cyclopentyl (such as 3-[[4-(cyclopropyloxy)-3-ethoxyphenyl](pyrimidin-5-ylmethyl)amino]benzoic acid, compound 272); and/or
R³ is alkyl having 1 to 8, preferably 1 to 4 carbon atoms, which is branched or unbranched and which is substituted one or more times with hydroxyl, e.g., 2-hydroxyethyl, 2-hydroxy-2-methyl-propyl, 2-hydroxypropyl, 3-hydroxypropyl (such as 2-[[3,4bis(difluoromethoxy)phenyl](phenyl)amino]-ethanol, compound 266; 1-[[3,4-bis(difluoromethoxy)phenyl](3-chlorophenyl)amino]-2-methylpropan-2-ol, compound 267; 1-[[3,4-bis(difluoromethoxy)phenyl](3-chlorophenyl)amino]propan-2-ol, compound 268; and 3-[[3,4-bis(difluoromethoxy)phenyl](3-chlorophenyl)amino]propan-1-ol, compound 269), or
   a heterocycle-alkyl group, wherein the heterocyclic portion is saturated, partially saturated or unsaturated (e.g., heteroarylalkyl) and has 5 to 10 ring atoms in which at least 1 ring atom is a N, O or S atom, and the alkyl portion, which is branched or unbranched, has 1 to 5 carbon atoms, the heterocycle-alkyl group is substituted one or more times in the heterocycle portion by at least aminocarbonyl (i.e., carbamoyl, NH₂—CO—), unsubstituted aryl having 6 to 14 carbon atoms, aryl having 6 to 14 carbon atoms and which is substituted (e.g., aryl substituted one or more times by C₁₋₄-alkyl, C₁₋₄-alkoxy, halogenated C₁₋₄-alkyl, and/or halogenated C₁₋₄-alkoxy), or combinations thereof, and is optionally substituted in the alkyl portion by halogen, cyano, or methyl or combinations thereof (e.g., 4-difluoromethoxy-3-ethoxyphenyl), etc.) (such as 3-{{[1-(aminocarbonyl)-piperidin-3-yl]methyl}[3-(cyclopentyloxy)-4-methoxyphenyl]amino}benzoic acid, compound 234; and 4-[[4-(difluoromethoxy)-3-ethoxyphenyl]({2-[4-(difluoromethoxy)-3-ethoxyphenyl]-1,3-oxazol-5-yl}methyl)amino]benzoic acid, compound 273); and/or
R⁴ is alkyl having 1 to 8, preferably 1 to 4 carbon atoms, which is branched or unbranched and which is unsubstituted or substituted one or more times with halogen, carboxy, cyano, C₁₋₄-alkoxy, or combinations thereof (e.g., methyl, ethyl, propyl, 3-carboxypropyl, etc.) (such as 4-[[3,4-bis(difluoromethoxy)phenyl](1,3-thiazol-5-ylmethyl)amino]butanoic acid, compound 270), or
   cycloalkyl having 3 to 10, preferably 3 to 8 carbon atoms, which is substituted one or more times by carboxy, e.g., carboxycyclohexyl (such as cis-4-[[3,4-bis(difluoromethoxy)phenyl](1,3-thiazol-5-ylmethyl)amino]cyclohexane-carboxylic acid, compound 271), or
   aryl having 6 to 14 carbon atoms and which is substituted one or more times by at least (substituted-C₁₋₈-alkoxy)-C₁₋₈-alkoxy (see R⁵-L) (e.g., (alkoxy substituted one or more times by halogen, hydroxy, carboxy, and/or cyano)-alkoxy such as hydroxyalkoxy)-alkoxy (e.g., OCH₂CH(OH)CH₂OCH₃), dihydroxyalkoxy (e.g., OCH₂CH(OH)CH₂OH), or combinations thereof (such as (2R)-3-{4-[[3-(cyclopentyloxy)-4-methoxyphenyl](pyridin-3-ylmethyl)amino]-phenoxy}propane-1,2-diol, compound 233; (2R)-3-{3-[[3-(cyclopentyloxy)-4-methoxyphenyl](pyridin-3-ylmethyl)amino]phenoxy}propane-1,2-diol, compound 238; (2R)-3-{4-[[3,4-bis(difluoromethoxy)phenyl](pyridin-3-ylmethyl)amino]phenoxy}propane-1,2-diol, compound 240; (2R)-3-{4-[[3-(cyclopentyloxy)-4-methoxyphenyl](1,3-thiazol-5-ylmethyl)amino]-phenoxy}propane-1,2-diol, compound 241; (2R)-3-{4-[(3,4-dimethoxyphenyl)(pyridin-3-ylmethyl)amino]phenoxy}propane-1,2-diol, compound 244; 5-[[3,4-bis(difluoromethoxy)phenyl](pyridin-3-ylmethyl)amino]-2-{[(2R)-2,3-dihydroxypropyl]oxy}benzoic acid, compound 246; (2R)-3-{3-[[3,4-bis(difluoromethoxy)phenyl](pyridin-3-ylmethyl)amino]phenoxy}propane-1,2-diol, compound 255; (2R)-3-{4-[[3,4-bis(difluoromethoxy)phenyl](1,3-thiazol-5-ylmethyl)amino]phenoxy}propane-1,2-diol, compound 258; (2R)-3-{3-[[3,4-bis(difluoromethoxy)phenyl](1,3-thiazol-5-ylmethyl)amino]-phenoxy}propane-1,2-diol, compound 259; and 1-{3-[[3,4-bis(difluoromethoxy)phenyl](1,3-thiazol-5-ylmethyl)amino]phenoxy}-3-methoxypropan-2-ol, compound 265), or
   a heterocyclic group, which is fully saturated or partially saturated, having 5 to 10 ring atoms in which at least 1 ring atom is a heteroatom, which is unsubstituted or substituted one or more times by halogen, C₁₋₈-alkyl, hydroxy, C₁₋₈-alkoxy, oxo, C₁₋₈-alkoxy-C₁₋₈-alkoxy, nitro, methylenedioxy, ethylenedioxy, trifluoromethyl, amino, amino-C₁₋₈-alkyl, amino-C₂₋₈-alkoxy, C₁₋₈-alkylamino, di-C₁₋₈-alkylamino, (substituted-C₁₋₈-alkyl)amino (see R⁵-L) (e.g., the alkyl group is substituted one or more times by halogen, hydroxy, carboxy and/or cyano, for example, —NHCH(CH₃)CH₂OH, NHCH(CH₂OH)CH₂CH₃, NHCH₂CH(OH)CH₂CH₃, NHCH₂CH₂CH(OH)CH₃, NHCH₂CH(OH)CH₃, NHCH₂C(OH)(CH₃)₂, etc.), C₁₋₈-alkyl(substituted-C₁₋₈-alkyl)amino (See R⁵-L) (e.g., alkyl(alkyl substituted one or more times by halogen, hydroxy, carboxy and/or cyano)-amino such as —N(CH₃)CH₂CH(OH)—CH₂OH), C₁₋₈-hydroxyalkyl (e.g., hydroxymethyl), hydroxamic acid (—C(O)—NHOH), tetrazole-5-yl, C₁₋₈-hydroxyalkoxy, carboxy, C₁₋₈-alkoxy-carbonyl (e.g., tert-butyloxycarbonyl, ethoxycarbonyl), cyano, acyl, C₁₋₈-alkylthio, C₁₋₈-alkylsulfinyl, C₁₋₈-alkylsulfonyl, phenoxy, tri-C₁₋₈-alkylsilyloxy (e.g. tert-butyldimethylsilyloxy), R⁵-L-, or combinations thereof (such as 5-[[3-(cyclopentyloxy)-4-methoxyphenyl](pyridin-3-ylmethyl)amino]pyridin-2(1H)-one, compound 235; 5-[[3-(cyclopentyloxy)-4-methoxyphenyl](pyridin-3-ylmethyl)amino]-2-methyl-1,2-benzisoxazol-3(2H)-one, compound 242; 5-[[3-(cyclopentyloxy)-4-methoxyphenyl](pyridin-3-ylmethyl)amino]-1,2-benzisoxazol-3(2H)-one, compound 243; 5-[[3,4-bis(difluoromethoxy)phenyl](1,3-thiazol-5-ylmethyl)amino]-1,2-benzisoxazol-3(2H)-one, compound 261; and 6-[[3,4-bis(difluoromethoxy)phenyl](1,3-thiazol-5-ylmethyl)amino]-1,2-benzisoxazol-3(2H)-one, compound 263), or
   a heterocyclic group, which is unsaturated (i.e., heteroaryl) (e.g., pyridyl, thienyl, pyrazinyl, quinolinyl, isoquinolinyl, pyrimidinyl, imidazolyl, thiazolyl, etc.), having 5 to 10 ring atoms in which at least 1 ring atom is a heteroatom, which is substituted one or more times by at least oxo, (substituted-C₁₋₈-alkyl)amino (See R⁵-L) (e.g., the alkyl group is substituted one or more times by halogen, hydroxy, carboxy and/or cyano, for example, —NHCH(CH₃)CH₂OH, NHCH(CH₂OH)CH₂CH₃, NHCH₂CH(OH)CH₂CH₃, NHCH₂CH₂CH(OH)CH₃, NHCH₂CH(OH)CH₃, NHCH₂C(OH)(CH₃)₂, etc.), or C₁₋₈-alkyl(substituted-C₁₋₈-alkyl)amino (See R⁵-L) (e.g., alkyl(alkyl substituted one or more times by halogen, hydroxy, carboxy and/or cyano)-amino such as —N(CH₃)CH₂CH(OH)CH₂OH), or combinations thereof (such as 5-[[3-(cyclopentyloxy)-4-methoxyphenyl](pyridin-3-ylmethyl)amino]pyridin-2(1H)-one, compound 235; 2-({5-[[3-(cyclopentyloxy)-4-methoxyphenyl](pyridin-3-ylmethyl)amino]pyridin-2-yl}amino)propan-1-ol, compound 236; 3-[{5-[[3-(cyclopentyloxy)-4-methoxyphenyl](pyridin-3-ylmethyl)amino]pyridin-2-yl}(methyl)amino]propane-1,2-diol, compound 237; 2-({5-[[3-(cyclopentyloxy)-4-methoxyphenyl](pyridin-3-ylmethyl)amino]pyridin-2-yl}amino)butan-1-ol, compound 247; 1-({5-[[3-(cyclopentyloxy)-4-methoxyphenyl](pyridin-3-ylmethyl)amino]pyridin-2-yl}amino)butan-2-ol, compound 248; 4-({5-[[3-(cyclopentyloxy)-4-methoxyphenyl](pyridin-3-ylmethyl)amino]pyridin-2-yl}amino)butan-2-ol, compound 249; 1-({5-[[3-(cyclopentyloxy)-4-methoxyphenyl](pyridin-3-ylmethyl)amino]pyridin-2-yl}amino)-2-methylpropan-2-ol trifluoroacetic acid salt, compound 252; 3-[{5-[[3-(cyclopentyloxy)-4-methoxyphenyl](1,3-thiazol-5-ylmethyl)amino]pyridin-2-yl}(methyl)amino]propane-1,2-diol, compound 253; and 3-[{5-[[3,4-bis(difluoromethoxy)phenyl](pyridin-3-ylmethyl)amino]pyridin-2-yl}(methyl)amino]propane-1,2-diol, compound 254); and/or
R⁵ is a heterocyclic group, which is saturated, partially saturated or unsaturated, having 5 to 10 ring atoms in which at least 1 ring atom is a N, O or S atom, which is substituted one or more times by at least oxo (such as (5S)-5-({3-[[3-(cyclopentyloxy)-4-methoxyphenyl](pyridin-3-ylmethyl)amino]phenoxy}-methyl)pyrrolidin-2-one, compound 58; 4-{3-[[3,4-bis(difluoromethoxy)phenyl]-(pyridin-3-ylmethyl)amino]phenyl}-2-methyl-1,2-dihydro-3H-pyrazol-3-one, compound 105; 5-{3-[[3-(cyclopentyloxy)-4-methoxyphenyl](pyridin-3-ylmethyl)amino]phenyl}imidazolidine-2,4-dione, compound 239; 5-{4-[[3-(cyclopentyloxy)-4-methoxyphenyl](pyridin-3-ylmethyl)amino]phenyl}-imidazolidine-2,4-dione, compound 245; 1-({5-[[3-(cyclopentyloxy)-4-methoxyphenyl](pyridin-3-ylmethyl)amino]pyridin-2-yl}amino)propan-2-ol, compound 250; 5-{3-[[3,4-bis(difluoromethoxy)phenyl](1,3-thiazol-5-ylmethyl)amino]phenyl}-1,3,5-trimethylimidazolidine-2,4-dione, compound 256; 5-{3-[[3,4-bis(difluoromethoxy)phenyl](1,3-thiazol-5-ylmethyl)amino]phenyl}imidazolidine-2,4-dione, compound 257; 4-{3-[[3,4-bis(difluoromethoxy)phenyl](pyridin-3-ylmethyl)amino]phenyl}imidazolidin-2-one, compound 260; 5-{3-[[3,4-bis(difluoromethoxy)phenyl](pyridin-3-ylmethyl)amino]phenyl}-1,3-oxazolidin-2-one, compound 262; and 4-{3-[[3,4-bis(difluoromethoxy)phenyl](pyridin-3-ylmethyl)amino]phenyl}-1,3-oxazolidin-2-one, compound 264);
   and pharmaceutically acceptable salts or solvates (e.g., hydrates) or N-oxides thereof, or solvates of pharmaceutically acceptable salts or N-oxides thereof.

According to another aspect of the invention, both R³ and R⁴ are other than H.

In the compounds of Formula I, R¹ preferably is C₁₋₄-alkyl, halogenated C₁₋₄-alkyl preferably fluorinated and/or chlorinated, or C₃₋₁₀-cycloalkyl, especially C₃₋₆-cycloalkyl, which is optionally substituted. Suitable examples of R¹ include, but are not limited to, methyl, ethyl, isopropyl, difluoromethyl, trifluoroethyl, trifluoromethyl, or cyclopropyl, in particular CH₃, C₂H₅, CHF₂, CF₃, CH₂CF₃, and cyclopropyl, especially methyl and difluoromethyl.

In the compounds of Formula I, R² is preferably (i) C₁₋₈-alkyl, especially C₁₋₄-alkyl, or halogenated C₁₋₈-alkyl, especially halogenated C₁₋₄-alkyl, preferably fluorinated and/or chlorinated, (ii) C₃₋₈-cycloalkyl, especially C₃₋₆-cycloalkyl, which is unsubstituted or substituted, (iii) C₄₋₁₂-cycloalkylalkyl which is unsubstituted or substituted, (iv) arylalkyl which is unsubstituted or substituted, especially benzyl, or (v) tetrahydrofuranyl, especially tetrahydrofuran-3-yl. Suitable examples of R² include, but are not limited to, methyl, ethyl, isopropyl, butyl, difluoromethyl, trifluoroethyl, trifluoromethyl, cyclopropyl, cyclobutyl, cyclopentyl, tetrahydrofuranyl, cyclopropylmethyl, and benzyl, in particular methyl, ethyl, cyclopentyl, and difluoromethyl.

In the compounds of Formula I, R³ is preferably

(i) C₁₋₄-alkyl which is branched or unbranched and which is unsubstituted or substituted one or more times by, for example, hydroxyl and/or carboxy (such as, but not limited to, methyl, ethyl, n-propyl, n-butyl, (CH₂)₂OH, CH₂C(CH₃)₂OH, CH₂CH(CH₃)OH, (CH₂)₃OH or (CH₂)₃COOH;

(ii) C₇₋₁₉-arylalkyl, which may be substituted or unsubstituted in the alkyl and/or the aryl portions, such as, but not limited to, substituted or unsubstituted benzyl (e.g., fluorobenzyl, difluorobenzyl, cyanobenzyl),

(iii) a heterocyclic-alkyl group, which may be substituted or unsubstituted in the alkyl and/or heterocyclic portions, wherein the heterocyclic portion is saturated or partially unsaturated. Suitable examples include, but not limited to, substituted or unsubstituted pyridinylmethyl (e.g., pyridin-3-ylmethyl, pyridin-4-ylmethyl, 5-methoxypyridin-3-ylmethyl), substituted or unsubstituted dihydropyridinylmethyl (e.g., 6-oxo-1,6-dihydropyridin-3-yl)methyl), substituted or unsubstituted oxazolylmethyl (e.g., 1,3-oxazol-5-ylmethyl, 4-methyl-1,3-oxazol-5-ylmethyl, 2-[4-(difluoromethoxy)-3-ethoxyphenyl]-1,3-oxazol-5-yl}methyl), substituted or unsubstituted pyrazolylmethyl (e.g., 1H-pyrazol-4-ylmethyl, 1-methyl-1H-pyrazol-4-yl)methyl), substituted or unsubstituted thiazolylmethyl (e.g., 1,3-thiazol-5-ylmethyl), substituted or unsubstituted pyrimidinylmethyl (e.g., pyrimidin-5-ylmethyl, 2-(dimethylamino)pyrimidin-5-ylmethyl), substituted or unsubstituted pyridazinylmethyl (e.g., pyridazin-4-ylmethyl), and substituted or unsubstituted pyrazinylmethyl (e.g., pyrazin-2-ylmethyl);

(iv) a heterocyclic-alkyl group, which may be substituted or unsubstituted, wherein the heterocyclic portion is fully saturated. Suitable examples include, but not limited to, substituted or unsubstituted piperidinylmethyl (e.g., piperidin-3-ylmethyl, 1-aminocarbonyl piperidin-3-ylmethyl, and piperidin-4-ylmethyl).

In the compounds of Formula I, examples of further preferred R³ groups include, but are not limited to, benzyl, pyrazolylmethyl, pyridinylmethyl, oxazolylmethyl, thiazolylmethyl, pyrimidinylmethyl, pyridizinylmethyl, pyrazinylmethyl, and thiadiazolylmethyl, which in each case is unsubstituted or substituted (e.g., substituted one or more times by halogen). Examples of such R³ groups are pyridin-3-ylmethyl, 1,3-oxazol-5-ylmethyl, 1,3-thiazol-5-ylmethyl, and pyrimidin-5-ylmethyl.

In the compounds of Formula I, R⁴ is preferably aryl or heteroaryl, especially phenyl, pyridinyl, or benzisoxazole, more preferably phenyl and pyridinyl, which in each case is unsubstituted or is substituted one or more times. Preferred substituents include, but are not limited to, OH, F, Cl, CF₃, alkyl (such as methyl or ethyl), oxo, alkoxy (such as methoxy and ethoxy), hydroxyalkoxy, (dihydroxy)alkoxy, NHCOCH₃, (substituted-alkyl)amino, alkyl(substituted-alkyl)amino, CN, CONHOH, CONHCH₂CH₂OH, COOH, CH₂COOH, (CH₂)₂COOH, COOalkyl, and combinations thereof.

Thus, for example, R⁴ can be phenyl or pyridinyl (especially phenyl) which is substituted one or more times and at least one of the substituents is a R⁵-L-group selected from the following: —COOH, —CO—NH₂, —CO—NH—C₁₋₄-alkyl, —CO—N(C₁₋₄-alkyl)₂, —CO—NH—SO₂—C₁₋₄-alkyl, —CO—N(C₁₋₄-alkyl)-SO₂—C₁₋₄-alkyl, —NH—SO₂—C₁₋₄-alkyl, —N(C₁₋₄-alkyl)-SO₂—C₁₋₄-alkyl, —SO₂—NH₂, —SO₂—NH—C₁₋₄-alkyl, —SO₂—N(C₁₋₄-alkyl)₂, —O—C₁₋₄-alkylene-COO—C₁₋₄-alkyl (e.g., —O—CH₂—COO—C₁₋₄-alkyl), —O—CH₂—CHOH—CH₂—O—CH₃, —O—C₁₋₄-alkylene-OH (e.g., —O—CH₂CH₂—OH), —O—C₁₋₄-alkylene-heterocycle (e.g., —O—CH₂-heterocycle), —NH—CH₂-aryl, —N(C₁₋₄-alkyl)-CH₂-aryl, —C₁₋₄-alkylene-OH, —C₁₋₄-alkylene-COOH, —NH-heterocycle, —NH—C₁₋₄-alkylene-heterocycle, —S—C₁₋₄-alkyl, —SO₂—C₁₋₄-alkyl, —NH—C₁₋₄-alkylene-SO₂—C₁₋₄-alkyl, —N(C₁₋₄-alkyl)-C₁₋₄-alkylene-CO—N(C₁₋₄-alkyl)₂, —CO—NH—OH, —CO—C₁₋₄-alkyl, —O—C₁₋₄-alkylene-C(OH)(CH₃)₂, —NH—C₁₋₄-alkylene-OH, —N(C₁₋₄-alkyl)-C₁₋₄-alkylene-OH, —N(C₁₋₄-alkyl)-CH₂—CHOH—CH₂—CHOH, —O—CH₂—CHOH—CH₂—CHOH, and —NH—C₁₋₄-alkylene-CHOH—C₁₋₄-alkyl.

R⁴ can also preferably be substituted or unsubstituted cycloalkyl, e.g., carboxycyclohexyl.

In addition, when R⁴ is aryl, especially, phenyl, preferred substituents include R⁵-L-, e.g., R⁵—, R⁵—O—, R⁵—CO—, R⁵—NH—CO—, R⁵—SO₂—NH—, R⁵—SO₂—NR⁶—, R⁵—NHR⁶—SO₂—, R⁵—SO₂—NH-alkylene-O—, NH₂-alkyl-NH—CO—, R⁵-alkylene-NH—CO—, alkyl-CO—NH-alkyl, as well as methyl, ethyl, Cl, F, CN, OCH₃, CF₃, amino, nitro, HOCH₂ and COOH.

In R⁵—SO₂—NR⁶—, R⁵—NHR⁶—SO₂—, R⁶ is preferably aryl or arylalkyl, e.g., substituted or unsubstituted phenyl or benzyl.

When R⁴ is aryl substituted by R⁵—SO₂—NH— it is preferably a substituted phenyl group and R⁵ is preferably methyl, ethyl, propyl or phenyl.

When R⁴ is aryl substituted by R⁵—SO₂—NH-alkylene-O— it is preferably a substituted phenyl. In such cases, R⁵ is preferably methyl, ethyl, propyl or phenyl and alkylene is preferably —CH₂—, —CH₂CH₂— or —CH₂CH₂CH₂—.

When R⁴ is aryl substituted by R⁵-L- it is preferably substituted phenyl. In such cases, preferred R⁵ groups include benzyl, tetrazolyl, oxazinyl, piperazinyl, methylpiperazinyl, pyridyl, methylpyridyl, pyrrolinyl, methylpyrrolinyl, piperadinyl, or methylpiperadinyl, and L is preferably a single bond, —O—, —CO—, —CH₂—, —CH₂CH₂—, —CH₂CH₂CH₂—, —CH₂—O—, —CH₂CH₂—O—, —CH₂CH₂CH₂—O—, —CH₂—NH—CH₂CH₂—O—, —CO—NH—, —NH—CO—, —SO₂—NR⁶—, —NHR⁶—SO₂—, —SO₂—, or —CONHSO₂—.

According to a further aspect of the invention, the compounds of Formula I are selected from:
1) 4-[[3,4-bis(difluoromethoxy)phenyl](pyridin-3-ylmethyl)amino]-N-(methylsulfonyl)benzamide
2) 4-[[3,4-bis(difluoromethoxy)phenyl](pyridin-3-ylmethyl)amino]-N-(ethylsulfonyl)benzamide
3) 3-{(3-fluorobenzyl)[4-methoxy-3-(2,2,2-trifluoroethoxy)phenyl]amino}benzoic acid
4) 3-[(3,4-dimethoxyphenyl)(3-fluorobenzyl)amino]benzoic acid
5) 3-[(3-ethoxy-4-methoxyphenyl)(3-fluorobenzyl)amino]benzoic acid
6) 3-[[3-(cyclobutyloxy)-4-methoxyphenyl](3-fluorobenzyl)amino]benzoic acid
7) 3-[[3-(cyclopentyloxy)-4-methoxyphenyl](3-fluorobenzyl)amino]benzoic acid
8) 3-[(2,6-difluorobenzyl)(3,4-dimethoxyphenyl)amino]benzoic acid
9) 3-[(2,6-difluorobenzyl)(3-ethoxy-4-methoxyphenyl)amino]benzoic acid
10) 3-[(2,6-difluorobenzyl)(3-isopropoxy-4-methoxyphenyl)amino]benzoic acid
11) 3-[[3-(cyclobutyloxy)-4-methoxyphenyl](2,6-difluorobenzyl)amino]benzoic acid
12) 3-((2,6-difluorobenzyl) {4-methoxy-3-[(3R)-tetrahydrofuran-3-yloxy]phenyl}amino)benzoic acid
13) 3-[[4-(difluoromethoxy)-3-ethoxyphenyl](pyridin-3-ylmethyl)amino]benzoic acid
14) 4-[[4-(difluoromethoxy)-3-ethoxyphenyl](pyridin-3-ylmethyl)amino]benzoic acid
15) 3-[[3,4-bis(difluoromethoxy)phenyl](2,6-difluorobenzyl)amino]benzoic acid
16) 3-[[3,4-bis(difluoromethoxy)phenyl](3-fluorobenzyl)amino]benzoic acid
17) 3-{(2,6-difluorobenzyl) [4-(difluoromethoxy)-3-methoxyphenyl]amino}benzoic acid
18) 3-[[4-(difluoromethoxy)-3-methoxyphenyl](pyridin-3-ylmethyl)amino]benzoic acid
19) 3-[[4-(difluoromethoxy)-3-methoxyphenyl](3-fluorobenzyl)amino]benzoic acid
20) 3-[[3,4-bis(difluoromethoxy)phenyl](2-fluorobenzyl)amino]benzoic acid
21) 3-[[3,4-bis(difluoromethoxy)phenyl](4-fluorobenzyl)amino]benzoic acid
22) 3-((3-fluorobenzyl) {4-methoxy-3-[(3R)-tetrahydrofuran-3-yloxy]phenyl}amino)benzonitrile
23) N-(3-fluorobenzyl)-4-methoxy-N-[4-(methylsulfonyl)phenyl]-3-[(3R)-tetrahydrofuran-3-yloxy]aniline
24) N-ethyl-N-[4-((3-fluorobenzyl) {4-methoxy-3-[(3R)-tetrahydrofuran-3-yloxy]phenyl}amino)phenyl]ethanesulfonamide
25) 3-{(3-cyanobenzyl) [3-(cyclopentyloxy)-4-methoxyphenyl]amino}benzoic acid
26) 3-{[3-(cyclopentyloxy)-4-methoxyphenyl][(6-oxo-1,6-dihydropyridin-3-yl)methyl]amino}benzoic acid
27) 5-bromo-N-[3-(cyclopentyloxy)-4-methoxyphenyl]-N-(pyridin-3-ylmethyl)pyridin-2-amine
28) 3-[[3-(cyclopentyloxy)-4-methoxyphenyl](piperidin-3-ylmethyl)amino]benzoic acid
29) 4-{[3-(cyclopentyloxy)-4-methoxyphenyl][(6-oxo-1,6-dihydropyridin-3-yl)methyl]amino}benzoic acid
30) Ethyl {4-[[3-(cyclopentyloxy)-4-methoxyphenyl](pyridin-3-ylmethyl)amino]phenoxy}acetate
31) 2-{4-[[3-(cyclopentyloxy)-4-methoxyphenyl](pyridin-3-ylmethyl)amino]phenoxy}ethanol
32) 3-(cyclopentyloxy)-N-(4-{[(4S)-2,2-dimethyl-1,3-dioxolan-4-yl]methoxy}phenyl)-4-methoxy-N-(pyridin-3-ylmethyl)aniline
33) N(2)-benzyl-N(5)-[3-(cyclopentyloxy)-4-methoxyphenyl]-N(2)-methyl-N(5)-(pyridin-3-ylmethyl)pyridine-2,5-diamine
34) 3-[[3-(cyclopentyloxy)-4-methoxyphenyl](1H-pyrazol-4-ylmethyl)amino]benzoic acid
35) 3-[[3-(cyclopentyloxy)-4-methoxyphenyl](1,3-thiazol-5-ylmethyl)amino]benzoic acid
36) {3-[[3-(cyclopentyloxy)-4-methoxyphenyl](pyridin-3-ylmethyl)amino]phenyl}acetic acid
37) 5-[[3-(cyclopentyloxy)-4-methoxyphenyl](pyridin-3-ylmethyl)amino]-2-ethylbenzoic acid
38) 3-[[3-(cyclopentyloxy)-4-methoxyphenyl](pyridin-3-ylmethyl)amino]-4-methylbenzoic acid
39) 4-[[3-(cyclopentyloxy)-4-methoxyphenyl](1H-pyrazol-4-ylmethyl)amino]benzoic acid
40) 4-[[3-(cyclopentyloxy)-4-methoxyphenyl](1,3-thiazol-5-ylmethyl)amino]benzoic acid
41) N-[3-(cyclopentyloxy)-4-methoxyphenyl]-N-(pyridin-3-ylmethyl)-6-pyrrolidin-1-ylpyridin-3-amine
42) N(5)-[3-(cyclopentyloxy)-4-methoxyphenyl]-N(2)-(2-morpholin-4-ylethyl)-N(5)-(pyridin-3-ylmethyl)pyridine-2,5-diamine
43) 4-[[3-(cyclopentyloxy)-4-methoxyphenyl](pyrimidin-5-ylmethyl)amino]benzoic acid
44) N(2)-benzyl-N(5)-[3-(cyclopentyloxy)-4-methoxyphenyl]-N(5)-(pyridin-3-ylmethyl)pyridine-2,5-diamine
45) 3-[[3-(cyclopentyloxy)-4-methoxyphenyl](1,3-thiazol-4-ylmethyl)amino]benzoic acid
46) 3-[[3-(cyclopentyloxy)-4-methoxyphenyl](pyrimidin-5-ylmethyl)amino]benzoic acid
47) 4-[[3-(cyclopentyloxy)-4-methoxyphenyl](pyridazin-3-ylmethyl)amino]benzoic acid
48) 3-[[3-(cyclopentyloxy)-4-methoxyphenyl](pyridazin-3-ylmethyl)amino]benzoic acid
49) {4-[[3-(cyclopentyloxy)-4-methoxyphenyl](pyridin-3-ylmethyl)amino]phenyl}acetic acid
50) 3-[[3-(cyclopentyloxy)-4-methoxyphenyl](piperidin-4-ylmethyl)amino]benzoic acid
51) 4-([3-(cyclopentyloxy)-4-methoxyphenyl]{[2-(dimethylamino)pyrimidin-5-yl]methyl}amino)benzoic acid
52) 3-([3-(cyclopentyloxy)-4-methoxyphenyl]{[2-(dimethylamino)pyrimidin-5-yl]methyl}amino)benzoic acid
53) 3-{4-[[3-(cyclopentyloxy)-4-methoxyphenyl](pyridin-3-ylmethyl)amino]phenyl}propan-1-ol
54) 5-[[3-(cyclopentyloxy)-4-methoxyphenyl](pyridin-3-ylmethyl)amino]nicotinic acid
55) 3-{4-[[3-(cyclopentyloxy)-4-methoxyphenyl](pyridin-3-ylmethyl)amino]phenyl}propanoic acid
56) 3-(cyclopentyloxy)-4-methoxy-N-[3-(2-methyl-1,3-thiazol-4-yl)phenyl]-N-(pyridin-3-ylmethyl)aniline
57) {3-[[3,4-bis(difluoromethoxy)phenyl](pyridin-3-ylmethyl)amino]phenyl}acetic acid
58) (5S)-5-({3-[[3-(cyclopentyloxy)-4-methoxyphenyl](pyridin-3-ylmethyl)amino]phenoxy}methyl)pyrrolidin-2-one
59) 4-[[3,4-bis(difluoromethoxy)phenyl](1,3-thiazol-5-ylmethyl)amino]benzoic acid
60) 3-[[3,4-bis(difluoromethoxy)phenyl](1,3-thiazol-5-ylmethyl)amino]benzoic acid
61) N(5)-[3-(cyclopentyloxy)-4-methoxyphenyl]-N(2)-[2-(methylsulfonyl)ethyl]-N(5)-(pyridin-3-ylmethyl)pyridine-2,5-diamine
62) 3,4-bis(difluoromethoxy)-N-[3-(methylthio)phenyl]-N-(pyridin-3-ylmethyl)aniline
63) 3,4-bis(difluoromethoxy)-N-[4-(methylsulfonyl)phenyl]-N-(pyridin-3-ylmethyl)aniline
64) 2-[{5-[[3-(cyclopentyloxy)-4-methoxyphenyl](pyridin-3-ylmethyl)amino]pyridin-2-yl}(methyl)amino]-N,N-dimethylacetamide
65) N(5)-[3-(cyclopentyloxy)-4-methoxyphenyl]-N(5)-(pyridin-3-ylmethyl)-N(2)-(tetrahydro-2H-pyran-4-yl)pyridine-2,5-diamine
66) 3,4-bis(difluoromethoxy)-N-[3-(methylsulfonyl)phenyl]-N-(pyridin-3-ylmethyl)aniline
67) 5-[[3-(cyclopentyloxy)-4-methoxyphenyl](pyridin-3-ylmethyl)amino]-N,2-dihydroxybenzamide
68) 4-[[3-(cyclopentyloxy)-4-methoxyphenyl](pyridin-3-ylmethyl)amino]-3-methylbenzoic acid
69) {4-[[3,4-bis(difluoromethoxy)phenyl](pyridin-3-ylmethyl)amino]phenyl}acetic acid
70) 3-[{4-methoxy-3-[(3R)-tetrahydrofuran-3-yloxy]phenyl}(1,3-thiazol-5-ylmethyl)amino]benzoic acid
71) 4-[{4-methoxy-3-[(3R)-tetrahydrofuran-3-yloxy]phenyl}(1,3-thiazol-5-ylmethyl)amino]benzoic acid
72) 4-[[3-(cyclopropylmethoxy)-4-methoxyphenyl](1,3-thiazol-5-ylmethyl)amino]benzoic acid
73) 3-[[3-(cyclopropylmethoxy)-4-methoxyphenyl](1,3-thiazol-5-ylmethyl)amino]benzoic acid
74) 4-[[3,4-bis(difluoromethoxy)phenyl](pyridazin-3-ylmethyl)amino]benzoic acid
75) 3-[[3,4-bis(difluoromethoxy)phenyl](pyridazin-3-ylmethyl)amino]benzoic acid
76) 3,4-bis(difluoromethoxy)-N-[4-(ethylthio)phenyl]-N-(pyridin-3-ylmethyl)aniline
77) 3,4-bis(difluoromethoxy)-N-[4-(methylsulfonyl)phenyl]-N-(1,3-thiazol-5-ylmethyl)aniline
78) 3,4-bis(difluoromethoxy)-N-[4-(ethylsulfonyl)phenyl]-N-(1,3-thiazol-5-ylmethyl)aniline
79) 3,4-bis(difluoromethoxy)-N-[3-(methylsulfonyl)phenyl]-N-(1,3-thiazol-5-ylmethyl)aniline
80) 4-[[3,4-bis(difluoromethoxy)phenyl](pyrimidin-5-ylmethyl)amino]benzoic acid
81) 5-[[3-(cyclopropylmethoxy)-4-methoxyphenyl](1,3-thiazol-5-ylmethyl)amino]nicotinic acid
82) 3-[[3,4-bis(difluoromethoxy)phenyl](pyrimidin-5-ylmethyl)amino]benzoic acid
83) 5-[[3,4-bis(difluoromethoxy)phenyl](pyridin-3-ylmethyl)amino]nicotinic acid
84) 1-{4-[[3,4-bis(difluoromethoxy)phenyl](pyridin-3-ylmethyl)amino]-2-hydroxyphenyl}ethanone
85) 1-{4-[[3,4-bis(difluoromethoxy)phenyl](pyridin-3-ylmethyl)amino]phenyl}-2,2,2-trifluoroethanone
86) 1-{4-[[3,4-bis(difluoromethoxy)phenyl](pyridin-3-ylmethyl)amino]phenyl}ethanone
87) 4-[[3,4-bis(difluoromethoxy)phenyl](pyridin-3-ylmethyl)amino]phenol
88) 1-{3-[[3,4-bis(difluoromethoxy)phenyl](pyridin-3-ylmethyl)amino]phenyl}ethanone
89) 2-{3-[[3,4-bis(difluoromethoxy)phenyl](pyridin-3-ylmethyl)amino]phenyl}propan-2-ol
90) 5-[[3,4-bis(difluoromethoxy)phenyl](pyridin-3-ylmethyl)amino]-2,3-difluorophenol
91) 5-[[3,4-bis(difluoromethoxy)phenyl](1,3-thiazol-5-ylmethyl)amino]-2-hydroxybenzoic acid
92) 4-[[3,4-bis(difluoromethoxy)phenyl](1,3-thiazol-5-ylmethyl)amino]-2-hydroxybenzoic acid
93) 1-{5-[[3,4-bis(difluoromethoxy)phenyl](1,3-thiazol-5-ylmethyl)amino]pyridin-3-yl}ethanone
94) 1-{5-[[3,4-bis(difluoromethoxy)phenyl](pyridin-3-ylmethyl)amino]pyridin-3-yl}ethanone
95) N-[3,4-bis(difluoromethoxy)phenyl]-5-(2-methyl-1,3-dioxolan-2-yl)-N-(pyridin-3-ylmethyl)pyridin-3-amine
96) 3,4-bis(difluoromethoxy)-N-[3-(1H-pyrazol-3-yl)phenyl]-N-(pyridin-3-ylmethyl)aniline
97) 3-[[4-(difluoromethoxy)-3-ethoxyphenyl](1,3-thiazol-5-ylmethyl)amino]benzoic acid
98) 5-[[3,4-bis(difluoromethoxy)phenyl](1,3-thiazol-5-ylmethyl)amino]nicotinic acid
99) 4-[[4-(difluoromethoxy)-3-ethoxyphenyl](1,3-thiazol-5-ylmethyl)amino]benzoic acid
100) {4-[[3,4-bis(difluoromethoxy)phenyl](1,3-thiazol-5-ylmethyl)amino]phenyl}acetic acid
101) 3,4-bis(difluoromethoxy)-N-(pyridin-3-ylmethyl)-N-[3-(1H-1,2,4-triazol-5-yl)phenyl]aniline
102) 4-{3-[[3-(cyclopentyloxy)-4-methoxyphenyl](pyridin-3-ylmethyl)amino]phenyl}-1-methyl-1H-pyrazol-5-ol
103) {3-[[3,4-bis(difluoromethoxy)phenyl](1,3-thiazol-5-ylmethyl)amino]phenyl}acetic acid
104) 4-[[3-(cyclopentyloxy)-4-(difluoromethoxy)phenyl](1,3-thiazol-5-ylmethyl)amino]benzoic acid
105) 4-{3-[[3,4-bis(difluoromethoxy)phenyl](pyridin-3-ylmethyl)amino]phenyl}-2-methyl-1,2-dihydro-3H-pyrazol-3-one
106) 4-[[4-(difluoromethoxy)-3-isopropoxyphenyl](1,3-thiazol-5-ylmethyl)amino]benzoic acid
107) 4-[[4-(difluoromethoxy)-3-isopropoxyphenyl](pyridin-3-ylmethyl)amino]benzoic acid
108) 3-[[4-(difluoromethoxy)-3-isopropoxyphenyl](pyridin-3-ylmethyl)amino]benzoic acid
109) 3-[[4-(difluoromethoxy)-3-isopropoxyphenyl](1,3-thiazol-5-ylmethyl)amino]benzoic acid
110) 3-[[3-(cyclopentyloxy)-4-(difluoromethoxy)phenyl](1,3-thiazol-5-ylmethyl)amino]benzoic acid
111) 4-methoxy-N-[4-(methylsulfonyl)phenyl]-3-[(3R)-tetrahydrofuran-3-yloxy]-N-(1,3-thiazol-5-ylmethyl)aniline
112) 1-{4-[[3,4-bis(difluoromethoxy)phenyl](1,3-thiazol-5-ylmethyl)amino]phenyl}ethanone
113) 3,4-bis(difluoromethoxy)-N-[3-(methylsulfonyl)phenyl]-N-(pyrimidin-5-ylmethyl)aniline
114) 3,4-bis(difluoromethoxy)-N-[4-(methylsulfonyl)phenyl]-N-(pyrimidin-5-ylmethyl)aniline
115) 4-methoxy-N-[3-(methylsulfonyl)phenyl]-3-[(3R)-tetrahydrofuran-3-yloxy]-N-(1,3-thiazol-5-ylmethyl)aniline
116) 3-[[4-(difluoromethoxy)-3-methoxyphenyl](1,3-thiazol-5-ylmethyl)amino]benzoic acid
117) 1-{4-[[3,4-bis(difluoromethoxy)phenyl](pyrimidin-5-ylmethyl)amino]phenyl}ethanone
118) 3,4-bis(difluoromethoxy)-N-[4-(methylsulfonyl)phenyl]-N-(pyrazin-2-ylmethyl)aniline
119) 1-{4-[[3,4-bis(difluoromethoxy)phenyl](pyrazin-2-ylmethyl)amino]phenyl}ethanone
120) 4-[[4-(difluoromethoxy)-3-methoxyphenyl](1,3-thiazol-5-ylmethyl)amino]benzoic acid
121) 4-[[3-(cyclopropylmethoxy)-4-(difluoromethoxy)phenyl](1,3-thiazol-5-ylmethyl)amino]benzoic acid
122) 1-{3-[[3,4-bis(difluoromethoxy)phenyl](pyridin-3-ylmethyl)amino]phenoxy}-2-methylpropan-2-ol
123) 1-{3-[[3,4-bis(difluoromethoxy)phenyl](1,3-thiazol-5-ylmethyl)amino]phenoxy}-2-methylpropan-2-ol
124) 4-[[4-(difluoromethoxy)-3-methoxyphenyl](pyridin-3-ylmethyl)amino]benzoic acid
125) 3-[[3-butoxy-4-(difluoromethoxy)phenyl](1,3-thiazol-5-ylmethyl)amino]benzoic acid
126) 3-[[3-butoxy-4-(difluoromethoxy)phenyl](pyridin-3-ylmethyl)amino]benzoic acid
127) 3-{[3,4-bis(difluoromethoxy)phenyl][(1-methyl-1H-pyrazol-4-yl)methyl]amino}benzoic acid
128) 4-[[3-butoxy-4-(difluoromethoxy)phenyl](pyridin-3-ylmethyl)amino]benzoic acid
129) 4-[[3-butoxy-4-(difluoromethoxy)phenyl](1,3-thiazol-5-ylmethyl)amino]benzoic acid
130) 4-[(3-ethoxy-4-methoxyphenyl)(1,3-thiazol-5-ylmethyl)amino]benzoic acid
131) 4-[(3-isopropoxy-4-methoxyphenyl)(1,3-thiazol-5-ylmethyl)amino]benzoic acid
132) 3-(cyclopropylmethoxy)-4-methoxy-N-[4-(methylsulfonyl)phenyl]-N-(1,3-thiazol-5-ylmethyl)aniline
133) 1-{4-[[3-(cyclopropylmethoxy)-4-methoxyphenyl](1,3-thiazol-5-ylmethyl)amino]phenyl}ethanone
134) 3,4-bis(difluoromethoxy)-N-methyl-N-[4-(methylsulfonyl)phenyl]aniline
135) 4-[[3-(cyclopropylmethoxy)-4-(difluoromethoxy)phenyl](pyridin-3-ylmethyl)amino]benzoic acid
136) 3,4-bis(difluoromethoxy)-N-[4-methyl-3-(methylsulfonyl)phenyl]-N-(1,3-thiazol-5-ylmethyl)aniline
137) {4-[[3,4-bis(difluoromethoxy)phenyl](pyrimidin-5-ylmethyl)amino]phenyl}acetic acid
138) 4-[(3,4-dimethoxyphenyl)(1,3-thiazol-5-ylmethyl)amino]benzoic acid
139) {3-[[3,4-bis(difluoromethoxy)phenyl](pyrimidin-5-ylmethyl)amino]phenyl}acetic acid
140) {4-[[3,4-bis(difluoromethoxy)phenyl](pyrazin-2-ylmethyl)amino]phenyl}acetic acid
141) 4-[[3,4-bis(difluoromethoxy)phenyl](1,3-oxazol-5-ylmethyl)amino]benzoic acid
142) 4-[[3,4-bis(difluoromethoxy)phenyl](pyrazin-2-ylmethyl)amino]benzoic acid
143) 4-[[4-(difluoromethoxy)-3-ethoxyphenyl](pyrimidin-5-ylmethyl)amino]benzoic acid
144) 4-[[4-(difluoromethoxy)-3-ethoxyphenyl](1,3-oxazol-5-ylmethyl)amino]benzoic acid
145) 4-[[3,4-bis(difluoromethoxy)phenyl](1,3-thiazol-5-ylmethyl)amino]-N,N-dimethylbenzenesulfonamide
146) 4-[[3,4-bis(difluoromethoxy)phenyl](1,3-thiazol-5-ylmethyl)amino]benzenesulfonamide
147) 4-[[4-(difluoromethoxy)-3-ethoxyphenyl](pyrazin-2-ylmethyl)amino]benzoic acid
148) 3-[[3,4-bis(difluoromethoxy)phenyl](pyrazin-2-ylmethyl)amino]benzoic acid
149) 3-[[3-(difluoromethoxy)-4-methoxyphenyl](1,3-thiazol-5-ylmethyl)amino]benzoic acid
150) 4-[[3-(difluoromethoxy)-4-methoxyphenyl](1,3-thiazol-5-ylmethyl)amino]benzoic acid
151) 4-[[3,4-bis(difluoromethoxy)phenyl](1,3-thiazol-5-ylmethyl)amino]-N-methylbenzamide
152) 4-[[3,4-bis(difluoromethoxy)phenyl](1,3-thiazol-5-ylmethyl)amino]benzamide
153) 4-[[4-(difluoromethoxy)-3-methoxyphenyl](pyrimidin-5-ylmethyl)amino]benzoic acid
154) 4-[[4-(difluoromethoxy)-3-methoxyphenyl](pyrazin-2-ylmethyl)amino]benzoic acid
155) 4-[[4-(difluoromethoxy)-3-isopropoxyphenyl](pyrazin-2-ylmethyl)amino]benzoic acid
156) 3-[[4-(difluoromethoxy)-3-isopropoxyphenyl](pyrazin-2-ylmethyl)amino]benzoic acid
157) 4-[[3-(cyclopropylmethoxy)-4-(difluoromethoxy)phenyl](pyrazin-2-ylmethyl)amino]benzoic acid
158) 4-[[3,4-bis(difluoromethoxy)phenyl](1,3-thiazol-5-ylmethyl)amino]-N,N-dimethylbenzamide
159) 3-[[3-(cyclopropylmethoxy)-4-(difluoromethoxy)phenyl](pyrazin-2-ylmethyl)amino]benzoic acid
160) 5-[[4-(difluoromethoxy)-3-isopropoxyphenyl](1,3-thiazol-5-ylmethyl)amino]nicotinic acid
161) 4-[[3-(difluoromethoxy)-4-methoxyphenyl](pyrazin-2-ylmethyl)amino]benzoic acid
162) N-{5-[[3,4-bis(difluoromethoxy)phenyl](1,3-thiazol-5-ylmethyl)amino]-2-methylphenyl}methane sulfonamide
163) N-{3-[[3,4-bis(difluoromethoxy)phenyl](1,3-thiazol-5-ylmethyl)amino]phenyl}methane sulfonamide
164) N-{3-[[3,4-bis(difluoromethoxy)phenyl](1,3-thiazol-5-ylmethyl)amino]phenyl}ethanesulfonamide
165) 4-[[3,4-bis(difluoromethoxy)phenyl](1,3-thiazol-5-ylmethyl)amino]-N-methylbenzenesulfonamide
166) 4-[[4-(difluoromethoxy)-3-isopropoxyphenyl](1,3-oxazol-5-ylmethyl)amino]benzoic acid
167) 4-[[3-(cyclopropylmethoxy)-4-(difluoromethoxy)phenyl](1,3-oxazol-5-ylmethyl)amino]benzoic acid
168) 4-[[3-(cyclopropylmethoxy)-4-(difluoromethoxy)phenyl](pyrimidin-5-ylmethyl)amino]benzoic acid
169) 4-[[4-(difluoromethoxy)-3-isopropoxyphenyl](pyrimidin-5-ylmethyl)amino]benzoic acid
170) 4-[(3,4-diethoxyphenyl)(1,3-thiazol-5-ylmethyl)amino]benzoic acid
171) 3-[[3-(cyclopentyloxy)-4-methoxyphenyl](1-pyridin-3-ylethyl)amino]benzoic acid
172) 3-[(3,4-dimethoxyphenyl)(1-pyridin-3-ylethyl)amino]benzoic acid
173) 4-[(4-ethoxy-3-methoxyphenyl)(1,3-thiazol-5-ylmethyl)amino]benzoic acid
174) 4-(difluoromethoxy)-3-ethoxy-N-[4-(methylsulfonyl)phenyl]-N-(1,3-thiazol-5-ylmethyl)aniline
175) {3-[[4-(difluoromethoxy)-3-ethoxyphenyl](1,3-oxazol-5-ylmethyl)amino]phenyl}acetic acid
176) 4-(difluoromethoxy)-3-ethoxy-N-[4-(methylsulfonyl)phenyl]-N-(1,3-oxazol-5-ylmethyl)aniline
177) {4-[[4-(difluoromethoxy)-3-ethoxyphenyl](1,3-oxazol-5-ylmethyl)amino]phenyl}acetic acid
178) {3-[[4-(difluoromethoxy)-3-ethoxyphenyl](1,3-thiazol-5-ylmethyl)amino]phenyl}acetic acid
179) 4-[[4-(difluoromethoxy)-3-methoxyphenyl](1,3-oxazol-5-ylmethyl)amino]benzoic acid
180) 4-[(3-isopropoxy-4-methoxyphenyl)(1,3-oxazol-5-ylmethyl)amino]benzoic acid
181) 4-[(3-ethoxy-4-methoxyphenyl)(1,3-oxazol-5-ylmethyl)amino]benzoic acid
182) 4-[[3-(cyclobutyloxy)-4-(difluoromethoxy)phenyl](1,3-oxazol-5-ylmethyl)amino]benzoic acid
183) 4-[[3-(cyclopropylmethoxy)-4-methoxyphenyl](1,3-oxazol-5-ylmethyl)amino]benzoic acid
184) 4-[[3-(cyclobutyloxy)-4-methoxyphenyl](1,3-oxazol-5-ylmethyl)amino]benzoic acid
185) 4-[[3-methoxy-4-(2,2,2-trifluoroethoxy)phenyl](1,3-oxazol-5-ylmethyl)amino]benzoic acid
186) 4-[[3-methoxy-4-(2,2,2-trifluoroethoxy)phenyl](pyrimidin-5-ylmethyl)amino]benzoic acid
187) 4-{[4-(difluoromethoxy)-3-ethoxyphenyl][(5-methoxypyridin-3-yl)methyl]amino}benzoic acid
188) 4-{[4-(difluoromethoxy)-3-ethoxyphenyl][(4-methyl-1,3-oxazol-5-yl)methyl]amino}benzoic acid
189) 4-[[3-(cyclopropyloxy)-4-methoxyphenyl](1,3-oxazol-5-ylmethyl)amino]benzoic acid
190) 4-[[4-(difluoromethoxy)-3-ethoxyphenyl](1,3-oxazol-4-ylmethyl)amino]benzoic acid
191) 4-[(3,4-diethoxyphenyl)(pyrimidin-5-ylmethyl)amino]benzoic acid
192) 4-[(3-isopropoxy-4-methoxyphenyl)(pyrimidin-5-ylmethyl)amino]benzoic acid
193) 4-[[3-(cyclobutyloxy)-4-methoxyphenyl](pyrimidin-5-ylmethyl)amino]benzoic acid
194) 4-[[3-(cyclopropyloxy)-4-methoxyphenyl](pyrimidin-5-ylmethyl)amino]benzoic acid
195) 4-[[3-(cyclopropylmethoxy)-4-methoxyphenyl](pyrimidin-5-ylmethyl)amino]benzoic acid
196) 4-[(3-ethoxy-4-methoxyphenyl)(pyrimidin-5-ylmethyl)amino]benzoic acid
197) 3-[[4-(difluoromethoxy)-3-isopropoxyphenyl](1,3-oxazol-5-ylmethyl)amino]benzoic acid
198) 3-[[3-(cyclopropylmethoxy)-4-(difluoromethoxy)phenyl](1,3-oxazol-5-ylmethyl)amino]benzoic acid
199) 3-[[4-(difluoromethoxy)-3-ethoxyphenyl](pyrimidin-5-ylmethyl)amino]benzoic acid
200) 3-[(3-ethoxy-4-methoxyphenyl)(1,3-oxazol-5-ylmethyl)amino]benzoic acid
201) 3-[[3-(cyclobutyloxy)-4-(difluoromethoxy)phenyl](1,3-oxazol-5-ylmethyl)amino]benzoic acid
202) 4-[(4-ethoxy-3-isopropoxyphenyl)(pyrimidin-5-ylmethyl)amino]benzoic acid
203) 3-[(3-isopropoxy-4-methoxyphenyl)(1,3-oxazol-5-ylmethyl)amino]benzoic acid
204) 3-[[4-(difluoromethoxy)-3-ethoxyphenyl](1,3-oxazol-5-ylmethyl)amino]benzoic acid
205) 4-[[3-(cyclobutyloxy)-4-(difluoromethoxy)phenyl](pyrimidin-5-ylmethyl)amino]benzoic acid
206) 3-[[4-(difluoromethoxy)-3-methoxyphenyl](1,3-oxazol-5-ylmethyl)amino]benzoic acid
207) 4-[(3-ethoxy-4-isopropoxyphenyl)(1,3-thiazol-5-ylmethyl)amino]benzoic acid
208) 4-[(4-ethoxy-3-isopropoxyphenyl)(1,3-oxazol-5-ylmethyl)amino]benzoic acid
209) 4-[(4-ethoxy-3-methoxyphenyl)(pyrimidin-5-ylmethyl)amino]benzoic acid
210) 3-[(3-ethoxy-4-methoxyphenyl)(pyrimidin-5-ylmethyl)amino]benzoic acid
211) 3-[[3-(cyclopropylmethoxy)-4-(difluoromethoxy)phenyl](pyrimidin-5-ylmethyl)amino]benzoic acid
212) 3-[[3-(cyclopropylmethoxy)-4-methoxyphenyl](pyrimidin-5-ylmethyl)amino]benzoic acid
213) 3-[(3-isopropoxy-4-methoxyphenyl)(pyrimidin-5-ylmethyl)amino]benzoic acid
214) 4-[[3-(benzyloxy)-4-(difluoromethoxy)phenyl](pyrimidin-5-ylmethyl)amino]benzoic acid
215) 3-[[3-(cyclopropylmethoxy)-4-methoxyphenyl](1,3-oxazol-5-ylmethyl)amino]benzoic acid
216) 3-[[3-(cyclobutyloxy)-4-methoxyphenyl](1,3-oxazol-5-ylmethyl)amino]benzoic acid
217) 4-[[3-ethoxy-4-(trifluoromethoxy)phenyl](1,3-oxazol-5-ylmethyl)amino]benzoic acid
218) 3-[[3-(cyclopropyloxy)-4-methoxyphenyl](pyrimidin-5-ylmethyl)amino]benzoic acid
219) 4-[(4-ethoxy-3-methoxyphenyl)(1,3-oxazol-5-ylmethyl)amino]benzoic acid
220) 4-[(3,4-diethoxyphenyl)(1,3-oxazol-5-ylmethyl)amino]benzoic acid
221) 4-[(4-ethoxy-3-methoxyphenyl)(pyridin-3-ylmethyl)amino]benzoic acid
222) 3-[(4-ethoxy-3-methoxyphenyl)(pyridin-3-ylmethyl)amino]benzoic acid
223) 3-[[4-(difluoromethoxy)-3-isopropoxyphenyl](pyrimidin-5-ylmethyl)amino]benzoic acid
224) 3-[(4-ethoxy-3-methoxyphenyl)(1,3-oxazol-5-ylmethyl)amino]benzoic acid
225) 4-{[4-(difluoromethoxy)-3-ethoxyphenyl][(2-methyl-1,3-thiazol-5-yl)methyl]amino}benzoic acid
226) 3-[(4-ethoxy-3-methoxyphenyl)(1,3-thiazol-5-ylmethyl)amino]benzoic acid
227) 3-[(3,4-diethoxyphenyl)(1,3-thiazol-5-ylmethyl)amino]benzoic acid
228) 3-[[3-(cyclobutyloxy)-4-methoxyphenyl](pyrimidin-5-ylmethyl)amino]benzoic acid
229) 4-[[4-(difluoromethoxy)-3-ethoxyphenyl](tetrahydrofuran-3-ylmethyl)amino]benzoic acid
230) 4-[[3-(cyclopropyloxy)-4-(difluoromethoxy)phenyl](pyrimidin-5-ylmethyl)amino]benzoic acid
231) 4-[[3-(cyclopropyloxy)-4-(difluoromethoxy)phenyl](1,3-oxazol-5-ylmethyl)amino]benzoic acid,
232) 4-[[4-methoxy-3-(trifluoromethoxy)phenyl](1,3-oxazol-5-ylmethyl)amino]benzoic acid,
233) (2R)-3-{4-[[3-(cyclopentyloxy)-4-methoxyphenyl](pyridin-3-ylmethyl)amino]phenoxy}propane-1,2-diol
234) 3-{{[1-(aminocarbonyl)piperidin-3-yl]methyl}[3-(cyclopentyloxy)-4-methoxyphenyl]amino}benzoic acid
235) 5-[[3-(cyclopentyloxy)-4-methoxyphenyl](pyridin-3-ylmethyl)amino]pyridin-2(1H)-one
236) 2-({5-[[3-(cyclopentyloxy)-4-methoxyphenyl](pyridin-3-ylmethyl)amino]pyridin-2-yl}amino)propan-1-ol
237) 3-[{5-[[3-(cyclopentyloxy)-4-methoxyphenyl](pyridin-3-ylmethyl)amino]pyridin-2-yl}(methyl)amino]propane-1,2-diol
238) (2R)-3-{3-[[3-(cyclopentyloxy)-4-methoxyphenyl](pyridin-3-ylmethyl)amino]phenoxy}propane-1,2-diol
239) 5-{3-[[3-(cyclopentyloxy)-4-methoxyphenyl](pyridin-3-ylmethyl)amino]phenyl}imidazolidine-2,4-dione
240) (2R)-3-{4-[[3,4-bis(difluoromethoxy)phenyl](pyridin-3-ylmethyl)amino]phenoxy}propane-1,2-diol
241) (2R)-3-{4-[[3-(cyclopentyloxy)-4-methoxyphenyl](1,3-thiazol-5-ylmethyl)amino]phenoxy}propane-1,2-diol
242) 5-[[3-(cyclopentyloxy)-4-methoxyphenyl](pyridin-3-ylmethyl)amino]-2-methyl-1,2-benzisoxazol-3(2H)-one
243) 5-[[3-(cyclopentyloxy)-4-methoxyphenyl](pyridin-3-ylmethyl)amino]-1,2-benzisoxazol-3(2H)-one
244) (2R)-3-{4-[(3,4-dimethoxyphenyl)(pyridin-3-ylmethyl)amino]phenoxy}propane-1,2-diol
245) 5-{4-[[3-(cyclopentyloxy)-4-methoxyphenyl](pyridin-3-ylmethyl)amino]phenyl}imidazolidine-2,4-dione
246) 5-[[3,4-bis(difluoromethoxy)phenyl](pyridin-3-ylmethyl)amino]-2-{[(2R)-2,3-dihydroxypropyl]oxy}benzoic acid
247) 2-({5-[[3-(cyclopentyloxy)-4-methoxyphenyl](pyridin-3-ylmethyl)amino]pyridin-2-yl}amino)butan-1-ol
248) 1-({5-[[3-(cyclopentyloxy)-4-methoxyphenyl](pyridin-3-ylmethyl)amino]pyridin-2-yl}amino)butan-2-ol
249) 4-({5-[[3-(cyclopentyloxy)-4-methoxyphenyl](pyridin-3-ylmethyl)amino]pyridin-2-yl}amino)butan-2-ol
250) 1-({5-[[3-(cyclopentyloxy)-4-methoxyphenyl](pyridin-3-ylmethyl)amino]pyridin-2-yl}amino)propan-2-ol
251) 5-{3-[[3,4-bis(difluoromethoxy)phenyl](pyridin-3-ylmethyl)amino]phenyl}imidazolidine-2,4-dione
252) 1-({5-[[3-(cyclopentyloxy)-4-methoxyphenyl](pyridin-3-ylmethyl)amino]pyridin-2-yl}amino)-2-methylpropan-2-ol trifluoroacetic acid salt
253) 3-[{5-[[3-(cyclopentyloxy)-4-methoxyphenyl](1,3-thiazol-5-ylmethyl)amino]pyridin-2-yl}(methyl)amino]propane-1,2-diol
254) 3-[{5-[[3,4-bis(difluoromethoxy)phenyl](pyridin-3-ylmethyl)amino]pyridin-2-yl}(methyl)amino]propane-1,2-diol
255) (2R)-3-{3-[[3,4-bis(difluoromethoxy)phenyl](pyridin-3-ylmethyl)amino]phenoxy}propane-1,2-diol
256) 5-{3-[[3,4-bis(difluoromethoxy)phenyl](1,3-thiazol-5-ylmethyl)amino]phenyl}-1,3,5-trimethylimidazolidine-2,4-dione
257) 5-{3-[[3,4-bis(difluoromethoxy)phenyl](1,3-thiazol-5-ylmethyl)amino]phenyl}imidazolidine-2,4-dione
258) (2R)-3-{4-[[3,4-bis(difluoromethoxy)phenyl](1,3-thiazol-5-ylmethyl)amino]phenoxy}propane-1,2-diol
259) (2R)-3-{3-[[3,4-bis(difluoromethoxy)phenyl](1,3-thiazol-5-ylmethyl)amino]phenoxy}propane-1,2-diol
260) 4-{3-[[3,4-bis(difluoromethoxy)phenyl](pyridin-3-ylmethyl)amino]phenyl}imidazolidin-2-one
261) 5-[[3,4-bis(difluoromethoxy)phenyl](1,3-thiazol-5-ylmethyl)amino]-1,2-benzisoxazol-3(2H)-one
262) 5-{3-[[3,4-bis(difluoromethoxy)phenyl](pyridin-3-ylmethyl)amino]phenyl}-1,3-oxazolidin-2-one
263) 6-[[3,4-bis(difluoromethoxy)phenyl](1,3-thiazol-5-ylmethyl)amino]-1,2-benzisoxazol-3(2H)-one
264) 4-{3-[[3,4-bis(difluoromethoxy)phenyl](pyridin-3-ylmethyl)amino]phenyl}-1,3-oxazolidin-2-one
265) 1-{3-[[3,4-bis(difluoromethoxy)phenyl](1,3-thiazol-5-ylmethyl)amino]phenoxy}-3-methoxypropan-2-ol
266) 2-[[3,4-bis(difluoromethoxy)phenyl](phenyl)amino]ethanol
267) 1-[[3,4-bis(difluoromethoxy)phenyl](3-chlorophenyl)amino]-2-methylpropan-2-ol
268) 1-[[3,4-bis(difluoromethoxy)phenyl](3-chlorophenyl)amino]propan-2-ol
269) 3-[[3,4-bis(difluoromethoxy)phenyl](3-chlorophenyl)amino]propan-1-ol
270) 4-[[3,4-bis(difluoromethoxy)phenyl](1,3-thiazol-5-ylmethyl)amino]butanoic acid
271) cis-4-[[3,4-bis(difluoromethoxy)phenyl](1,3-thiazol-5-ylmethyl)amino]cyclohexanecarboxylic acid
272) 3-[[4-(cyclopropyloxy)-3-ethoxyphenyl](pyrimidin-5-ylmethyl)amino]benzoic acid,
273) 4-[[4-(difluoromethoxy)-3-ethoxyphenyl]({2-[4-(difluoromethoxy)-3-ethoxyphenyl]-1,3-oxazol-5-yl}methyl)amino]benzoic acid
279) 3-[[3-(difluoromethoxy)-4-methoxyphenyl](1,3-oxazol-5-ylmethyl)amino]benzoic acid,
280) 4-[[3-(difluoromethoxy)-4-methoxyphenyl](pyridin-3-ylmethyl)amino]benzoic acid,
281) 4-[[4-(difluoromethoxy)-3-ethoxyphenyl](1,2,3-thiadiazol-5-ylmethyl)amino]benzoic acid,
282) 4-[[3-hydroxy-4-methoxyphenyl](1,3-oxazol-5-ylmethyl)amino]benzoic acid,
283) 3-[[4-ethoxy-3-methoxyphenyl](pyrimidin-5-ylmethyl)amino]benzoic acid,
284) 3-[[3,4-diethoxyphenyl](pyrimidin-5-ylmethyl)amino]benzoic acid,
285) 3-[[3,4-diethoxyphenyl](1,3-oxazol-5-ylmethyl)amino]benzoic acid,
286) 3-[[3-cyclopropyloxy-4-methoxyphenyl](1,3-oxazol-5-ylmethyl)amino]benzoic acid,
287) 3-[[3-cyclobutyloxy-4-difluoromethoxyphenyl](pyrimidin-5-ylmethyl)amino]benzoic acid, and
288) 3-[[3-cyclopropyloxy-4-difluoromethoxyphenyl](1,3-oxazol-5-ylmethyl)amino]benzoic acid,

wherein a compound listed above (in either a free base form or in the form of a pharmaceutically acceptable salt) can also be in the form of a solvate (such as a hydrate),

wherein a compound listed above (in either a free base form or in the form of a pharmaceutically acceptable salt) can also be in the form of an N-oxide,

wherein a compound listed above (in a free base form or solvate or N-oxide thereof, or in the form of a pharmaceutically acceptable salt or solvate thereof,) can also be in the form of a polymorph, and

wherein if the compound exhibits chirality it can be in the form of a mixture of enantiomers such as a racemate or a mixture of diastereomers, or can be in the form of a single enantiomer or a single diastereomer.

According to further aspect of the invention, the compounds of Formula I are selected from:
233) (2R)-3-{4-[[3-(cyclopentyloxy)-4-methoxyphenyl](pyridin-3-ylmethyl)amino]phenoxy}propane-1,2-diol
234) 3-{{[1-(aminocarbonyl)piperidin-3-yl]methyl}[3-(cyclopentyloxy)-4-methoxyphenyl]amino}benzoic acid
235) 5-[[3-(cyclopentyloxy)-4-methoxyphenyl](pyridin-3-ylmethyl)amino]pyridin-2(1H)-one
236) 2-({5-[[3-(cyclopentyloxy)-4-methoxyphenyl](pyridin-3-ylmethyl)amino]pyridin-2-yl}amino)propan-1-ol
237) 3-[{5-[[3-(cyclopentyloxy)-4-methoxyphenyl](pyridin-3-ylmethyl)amino]pyridin-2-yl}(methyl)amino]propane-1,2-diol
238) (2R)-3-{3-[[3-(cyclopentyloxy)-4-methoxyphenyl](pyridin-3-ylmethyl)amino]phenoxy}propane-1,2-diol
239) 5-{3-[[3-(cyclopentyloxy)-4-methoxyphenyl](pyridin-3-ylmethyl)amino]phenyl}imidazolidine-2,4-dione
240) (2R)-3-{4-[[3,4-bis(difluoromethoxy)phenyl](pyridin-3-ylmethyl)amino]phenoxy}propane-1,2-diol
241) (2R)-3-{4-[[3-(cyclopentyloxy)-4-methoxyphenyl](1,3-thiazol-5-ylmethyl)amino]phenoxy}propane-1,2-diol
242) 5-[[3-(cyclopentyloxy)-4-methoxyphenyl](pyridin-3-ylmethyl)amino]-2-methyl-1,2-benzisoxazol-3(2H)-one
243) 5-[[3-(cyclopentyloxy)-4-methoxyphenyl](pyridin-3-ylmethyl)amino]-1,2-benzisoxazol-3(2H)-one
244) (2R)-3-{4-[(3,4-dimethoxyphenyl)(pyridin-3-ylmethyl)amino]phenoxy}propane-1,2-diol
245) 5-{4-[[3-(cyclopentyloxy)-4-methoxyphenyl](pyridin-3-ylmethyl)amino]phenyl}imidazolidine-2,4-dione
246) 5-[[3,4-bis(difluoromethoxy)phenyl](pyridin-3-ylmethyl)amino]-2-{[(2R)-2,3-dihydroxypropyl]oxy}benzoic acid
247) 2-({5-[[3-(cyclopentyloxy)-4-methoxyphenyl](pyridin-3-ylmethyl)amino]pyridin-2-yl}amino)butan-1-ol
248) 1-({5-[[3-(cyclopentyloxy)-4-methoxyphenyl](pyridin-3-ylmethyl)amino]pyridin-2-yl}amino)butan-2-ol
249) 4-({5-[[3-(cyclopentyloxy)-4-methoxyphenyl](pyridin-3-ylmethyl)amino]pyridin-2-yl}amino)butan-2-ol
250) 1-({5-[[3-(cyclopentyloxy)-4-methoxyphenyl](pyridin-3-ylmethyl)amino]pyridin-2-yl}amino)propan-2-ol
251) 5-{3-[[3,4-bis(difluoromethoxy)phenyl](pyridin-3-ylmethyl)amino]phenyl}imidazolidine-2,4-dione
252) 1-({5-[[3-(cyclopentyloxy)-4-methoxyphenyl](pyridin-3-ylmethyl)amino]pyridin-2-yl}amino)-2-methylpropan-2-ol trifluoroacetic acid salt
253) 3-[{5-[[3-(cyclopentyloxy)-4-methoxyphenyl](1,3-thiazol-5-ylmethyl)amino]pyridin-2-yl}(methyl)amino]propane-1,2-diol
254) 3-[{5-[[3,4-bis(difluoromethoxy)phenyl](pyridin-3-ylmethyl)amino]pyridin-2-yl}(methyl)amino]propane-1,2-diol
255) (2R)-3-{3-[[3,4-bis(difluoromethoxy)phenyl](pyridin-3-ylmethyl)amino]phenoxy}propane-1,2-diol
256) 5-{3-[[3,4-bis(difluoromethoxy)phenyl](1,3-thiazol-5-ylmethyl)amino]phenyl}-1,3,5-trimethylimidazolidine-2,4-dione
257) 5-{3-[[3,4-bis(difluoromethoxy)phenyl](1,3-thiazol-5-ylmethyl)amino]phenyl}imidazolidine-2,4-dione
258) (2R)-3-{4-[[3,4-bis(difluoromethoxy)phenyl](1,3-thiazol-5-ylmethyl)amino]phenoxy}propane-1,2-diol
259) (2R)-3-{3-[[3,4-bis(difluoromethoxy)phenyl](1,3-thiazol-5-ylmethyl)amino]phenoxy}propane-1,2-diol
260) 4-{3-[[3,4-bis(difluoromethoxy)phenyl](pyridin-3-ylmethyl)amino]phenyl}imidazolidin-2-one
261) 5-[[3,4-bis(difluoromethoxy)phenyl](1,3-thiazol-5-ylmethyl)amino]-1,2-benzisoxazol-3(2H)-one
262) 5-{3-[[3,4-bis(difluoromethoxy)phenyl](pyridin-3-ylmethyl)amino]phenyl}-1,3-oxazolidin-2-one
263) 6-[[3,4-bis(difluoromethoxy)phenyl](1,3-thiazol-5-ylmethyl)amino]-1,2-benzisoxazol-3(2H)-one
264) 4-{3-[[3,4-bis(difluoromethoxy)phenyl](pyridin-3-ylmethyl)amino]phenyl}-1,3-oxazolidin-2-one
265) 1-{3-[[3,4-bis(difluoromethoxy)phenyl](1,3-thiazol-5-ylmethyl)amino]phenoxy}-3-methoxypropan-2-ol
266) 2-[[3,4-bis(difluoromethoxy)phenyl](phenyl)amino]ethanol
267) 1-[[3,4-bis(difluoromethoxy)phenyl](3-chlorophenyl)amino]-2-methylpropan-2-ol
268) 1-[[3,4-bis(difluoromethoxy)phenyl](3-chlorophenyl)amino]propan-2-ol
269) 3-[[3,4-bis(difluoromethoxy)phenyl](3-chlorophenyl)amino]propan-1-ol
270) 4-[[3,4-bis(difluoromethoxy)phenyl](1,3-thiazol-5-ylmethyl)amino]butanoic acid
271) cis-4-[[3,4-bis(difluoromethoxy)phenyl](1,3-thiazol-5-ylmethyl)amino]cyclohexanecarboxylic acid
272) 3-[[4-(cyclopropyloxy)-3-ethoxyphenyl](pyrimidin-5-ylmethyl)amino]benzoic acid, and
273) 4-[[4-(difluoromethoxy)-3-ethoxyphenyl]({2-[4-(difluoromethoxy)-3-ethoxyphenyl]-1,3-oxazol-5-yl}methyl)amino]benzoic acid,

wherein a compound listed above (in either a free base form or in the form of a pharmaceutically acceptable salt) can also be in the form of a solvate (such as a hydrate),

wherein a compound listed above (in either a free base form or in the form of a pharmaceutically acceptable salt) can also be in the form of an N-oxide,

wherein a compound listed above (in a free base form or solvate or N-oxide thereof, or in the form of a pharmaceutically acceptable salt or solvate thereof,) can also be in the form of a polymorph, and

wherein if the compound exhibits chirality it can be in the form of a mixture of enantiomers such as a racemate or a mixture of diastereomers, or can be in the form of a single enantiomer or a single diastereomer.

According to further aspect of the invention, the compounds of Formula I are selected from:
3) 3-{(3-fluorobenzyl)[4-methoxy-3-(2,2,2-trifluoroethoxy)phenyl]amino}benzoic acid,
4) 3-[(3,4-dimethoxyphenyl)(3-fluorobenzyl)amino]benzoic acid,
5) 3-[(3-ethoxy-4-methoxyphenyl)(3-fluorobenzyl)amino]benzoic acid,
6) 3-[[3-(cyclobutyloxy)-4-methoxyphenyl](3-fluorobenzyl)amino]benzoic acid,
7) 3-[[3-(cyclopentyloxy)-4-methoxyphenyl](3-fluorobenzyl)amino]benzoic acid,
8) 3-[(2,6-difluorobenzyl)(3,4-dimethoxyphenyl)amino]benzoic acid,
9) 3-[(2,6-difluorobenzyl)(3-ethoxy-4-methoxyphenyl)amino]benzoic acid,
10) 3-[(2,6-difluorobenzyl)(3-isopropoxy-4-methoxyphenyl)amino]benzoic acid,
11) 3-[[3-(cyclobutyloxy)-4-methoxyphenyl](2,6-difluorobenzyl)amino]benzoic acid,
12) 3-((2,6-difluorobenzyl) {4-methoxy-3-[(3R)-tetrahydrofuran-3-yloxy]phenyl}amino)benzoic acid,
15) 3-[[3,4-bis(difluoromethoxy)phenyl](2,6-difluorobenzyl)amino]benzoic acid,
16) 3-[[3,4-bis(difluoromethoxy)phenyl](3-fluorobenzyl)amino]benzoic acid,
17) 3-{(2,6-difluorobenzyl) [4-(difluoromethoxy)-3-methoxyphenyl]amino}benzoic acid,
19) 3-[[4-(difluoromethoxy)-3-methoxyphenyl](3-fluorobenzyl)amino]benzoic acid,
20) 3-[[3,4-bis(difluoromethoxy)phenyl](2-fluorobenzyl)amino]benzoic acid,
21) 3-[[3,4-bis(difluoromethoxy)phenyl](4-fluorobenzyl)amino]benzoic acid,
22) 3-((3-fluorobenzyl){4-methoxy-3-[(3R)-tetrahydrofuran-3-yloxy]phenyl}amino)benzonitrile,
23) N-(3-fluorobenzyl)-4-methoxy-N-[4-(methylsulfonyl)phenyl]-3-[(3R)-tetrahydrofuran-3-yloxy]aniline,
24) N-ethyl-N-[4-((3-fluorobenzyl) {4-methoxy-3-[(3R)-tetrahydrofuran-3-yloxy]phenyl}amino)phenyl]ethanesulfonamide, and
25) 3-{(3-cyanobenzyl) [3-(cyclopentyloxy)-4-methoxyphenyl]amino}benzoic acid,

wherein a compound listed above (in either a free base form or in the form of a pharmaceutically acceptable salt) can also be in the form of a solvate (such as a hydrate),

wherein a compound listed above (in either a free base form or in the form of a pharmaceutically acceptable salt) can also be in the form of an N-oxide,

wherein a compound listed above (in a free base form or solvate or N-oxide thereof, or in the form of a pharmaceutically acceptable salt or solvate thereof,) can also be in the form of a polymorph, and

wherein if the compound exhibits chirality it can be in the form of a mixture of enantiomers such as a racemate or a mixture of diastereomers, or can be in the form of a single enantiomer or a single diastereomer.

In addition, preferred compounds of formula I are those of the subformulae II and III:

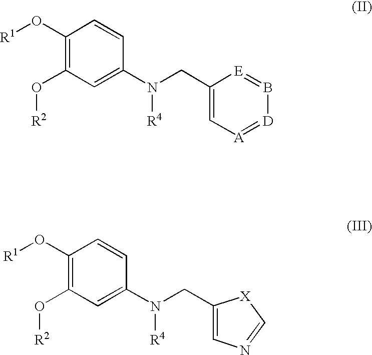

wherein R¹, R², and R⁴ are as defined in Formula I, one or two of A, B, D and E are N, and the others are CH, and X is O or S; and

pharmaceutically acceptable salts or solvates (e.g., hydrates) or N-oxides thereof, or solvates of pharmaceutically acceptable salts or N-oxides thereof.

In subformulas II and III, preferably, A is N, and B, D and E are CH; or

A and B are both N, and D and E are CH; or

A and E are both N, and D and B are CH; or

A and D are both N, and B and E are CH.

In subformulas II and III, R⁴ is preferably aryl, more preferably phenyl, which is substituted or unsubstituted. Preferred substituents when R⁴ is substituted phenyl include carboxy (e.g., 4-carboxyphenyl).

In subformulas II and III, preferably R¹ is halogenated alkyl (e.g., such as CF₃, or CHF₂, especially CHF₂), and R² is alkyl (e.g., such as methyl, ethyl) or halogenated alkyl (e.g., such as CF₃, CHF₂, especially CHF₂).

In particular, in subformulas II and III, R¹ is preferably CHF₂, R² is preferably methyl, ethyl, or CHF₂, A is N, B is CH or N, D is CH, X is O or S, and R⁴ is substituted phenyl (especially carboxy substituted phenyl, e.g., 4-carboxyphenyl).

Suitable compounds of subformulas II and III include:
14) 4-[[4-(difluoromethoxy)-3-ethoxyphenyl](pyridin-3-ylmethyl)amino]benzoic acid,
59) 4-[[3,4-bis(difluoromethoxy)phenyl](1,3-thiazol-5-ylmethyl)amino]benzoic acid,
120) 4-[[4-(difluoromethoxy)-3-methoxyphenyl](1,3-thiazol-5-ylmethyl)amino]benzoic acid,
124) 4-[[4-(difluoromethoxy)-3-methoxyphenyl](pyridin-3-ylmethyl)amino]benzoic acid,
143) 4-[[4-(difluoromethoxy)-3-ethoxyphenyl](pyrimidin-5-ylmethyl)amino]benzoic acid, and
144) 4-[[4-(difluoromethoxy)-3-ethoxyphenyl](1,3-oxazol-5-ylmethyl)amino]benzoic acid,

wherein a compound listed above (in either a free base form or in the form of a pharmaceutically acceptable salt) can also be in the form of a solvate (such as a hydrate),

wherein a compound listed above (in either a free base form or in the form of a pharmaceutically acceptable salt) can also be in the form of an N-oxide,

wherein a compound listed above (in a free base form or solvate or N-oxide thereof, or in the form of a pharmaceutically acceptable salt or solvate thereof,) can also be in the form of a polymorph, and

wherein if the compound exhibits chirality it can be in the form of a mixture of enantiomers such as a racemate or a mixture of diastereomers, or can be in the form of a single enantiomer or a single diastereomer.

According to a further aspect of the invention, the compounds of Formula II are selected from the following compounds wherein only one of A, B, D, and E is N:
1) 4-[[3,4-bis(difluoromethoxy)phenyl](pyridin-3-ylmethyl)amino]-N-(methylsulfonyl)benzamide
2) 4-[[3,4-bis(difluoromethoxy)phenyl](pyridin-3-ylmethyl)amino]-N-(ethylsulfonyl)benzamide
13) 3-[[4-(difluoromethoxy)-3-ethoxyphenyl](pyridin-3-ylmethyl)amino]benzoic acid
14) 4-[[4-(difluoromethoxy)-3-ethoxyphenyl](pyridin-3-ylmethyl)amino]benzoic acid
18) 3-[[4-(difluoromethoxy)-3-methoxyphenyl](pyridin-3-ylmethyl)amino]benzoic acid
26) 3-{[3-(cyclopentyloxy)-4-methoxyphenyl][(6-oxo-1,6-dihydropyridin-3-yl)methyl]amino}benzoic acid
27) 5-bromo-N-[3-(cyclopentyloxy)-4-methoxyphenyl]-N-(pyridin-3-ylmethyl)pyridin-2-amine
30) Ethyl {4-[[3-(cyclopentyloxy)-4-methoxyphenyl](pyridin-3-ylmethyl)amino]phenoxy}acetate
31) 2-{4-[[3-(cyclopentyloxy)-4-methoxyphenyl](pyridin-3-ylmethyl)amino]phenoxy}ethanol
32) 3-(cyclopentyloxy)-N-(4-{[(4S)-2,2-dimethyl-1,3-dioxolan-4-yl]methoxy}phenyl)-4-methoxy-N-(pyridin-3-ylmethyl)aniline
33) N(2)-benzyl-N(5)-[3-(cyclopentyloxy)-4-methoxyphenyl]-N(2)-methyl-N(5)-(pyridin-3-ylmethyl)pyridine-2,5-diamine
36) {3-[[3-(cyclopentyloxy)-4-methoxyphenyl](pyridin-3-ylmethyl)amino]phenyl}acetic acid
37) 5-[[3-(cyclopentyloxy)-4-methoxyphenyl](pyridin-3-ylmethyl)amino]-2-ethylbenzoic acid
38) 3-[[3-(cyclopentyloxy)-4-methoxyphenyl](pyridin-3-ylmethyl)amino]-4-methylbenzoic acid
41) N-[3-(cyclopentyloxy)-4-methoxyphenyl]-N-(pyridin-3-ylmethyl)-6-pyrrolidin-1-ylpyridin-3-amine
42) N(5)-[3-(cyclopentyloxy)-4-methoxyphenyl]-N(2)-(2-morpholin-4-ylethyl)-N(5)-(pyridin-3-ylmethyl)pyridine-2,5-diamine
44) N(2)-benzyl-N(5)-[3-(cyclopentyloxy)-4-methoxyphenyl]-N(5)-(pyridin-3-ylmethyl)pyridine-2,5-diamine
49) {4-[[3-(cyclopentyloxy)-4-methoxyphenyl](pyridin-3-ylmethyl)amino]phenyl}acetic acid
53) 3-{4-[[3-(cyclopentyloxy)-4-methoxyphenyl](pyridin-3-ylmethyl)amino]phenyl}propan-1-ol
54) 5-[[3-(cyclopentyloxy)-4-methoxyphenyl](pyridin-3-ylmethyl)amino]nicotinic acid
55) 3-{4-[[3-(cyclopentyloxy)-4-methoxyphenyl](pyridin-3-ylmethyl)amino]phenyl}propanoic acid
56) 3-(cyclopentyloxy)-4-methoxy-N-[3-(2-methyl-1,3-thiazol-4-yl)phenyl]-N-(pyridin-3-ylmethyl)aniline
57) {3-[[3,4-bis(difluoromethoxy)phenyl](pyridin-3-ylmethyl)amino]phenyl}acetic acid
58) (5S)-5-({3-[[3-(cyclopentyloxy)-4-methoxyphenyl](pyridin-3-ylmethyl)amino]phenoxy}methyl)pyrrolidin-2-one
61) N(5)-[3-(cyclopentyloxy)-4-methoxyphenyl]-N(2)-[2-(methylsulfonyl)ethyl]-N(5)-(pyridin-3-ylmethyl)pyridine-2,5-diamine
62) 3,4-bis(difluoromethoxy)-N-[3-(methylthio)phenyl]-N-(pyridin-3-ylmethyl)aniline
63) 3,4-bis(difluoromethoxy)-N-[4-(methylsulfonyl)phenyl]-N-(pyridin-3-ylmethyl)aniline
64) 2-[{5-[[3-(cyclopentyloxy)-4-methoxyphenyl](pyridin-3-ylmethyl)amino]pyridin-2-yl}(methyl)amino]-N,N-dimethylacetamide
65) N(5)-[3-(cyclopentyloxy)-4-methoxyphenyl]-N(5)-(pyridin-3-ylmethyl)-N(2)-(tetrahydro-2H-pyran-4-yl)pyridine-2,5-diamine
66) 3,4-bis(difluoromethoxy)-N-[3-(methylsulfonyl)phenyl]-N-(pyridin-3-ylmethyl)aniline
67) 5-[[3-(cyclopentyloxy)-4-methoxyphenyl](pyridin-3-ylmethyl)amino]-N,2-dihydroxybenzamide
68) 4-[[3-(cyclopentyloxy)-4-methoxyphenyl](pyridin-3-ylmethyl)amino]-3-methylbenzoic acid
69) {4-[[3,4-bis(difluoromethoxy)phenyl](pyridin-3-ylmethyl)amino]phenyl}acetic acid
76) 3,4-bis(difluoromethoxy)-N-[4-(ethylthio)phenyl]-N-(pyridin-3-ylmethyl)aniline
83) 5-[[3,4-bis(difluoromethoxy)phenyl](pyridin-3-ylmethyl)amino]nicotinic acid
84) 1-{4-[[3,4-bis(difluoromethoxy)phenyl](pyridin-3-ylmethyl)amino]-2-hydroxyphenyl}ethanone
85) 1-{4-[[3,4-bis(difluoromethoxy)phenyl](pyridin-3-ylmethyl)amino]phenyl}-2,2,2-trifluoroethanone
86) 1-{4-[[3,4-bis(difluoromethoxy)phenyl](pyridin-3-ylmethyl)amino]phenyl}ethanone
87) 4-[[3,4-bis(difluoromethoxy)phenyl](pyridin-3-ylmethyl)amino]phenol
88) 1-{3-[[3,4-bis(difluoromethoxy)phenyl](pyridin-3-ylmethyl)amino]phenyl}ethanone
89) 2-{3-[[3,4-bis(difluoromethoxy)phenyl](pyridin-3-ylmethyl)amino]phenyl}propan-2-ol
90) 5-[[3,4-bis(difluoromethoxy)phenyl](pyridin-3-ylmethyl)amino]-2,3-difluorophenol
94) 1-{5-[[3,4-bis(difluoromethoxy)phenyl](pyridin-3-ylmethyl)amino]pyridin-3-yl}ethanone
95) N-[3,4-bis(difluoromethoxy)phenyl]-5-(2-methyl-1,3-dioxolan-2-yl)-N-(pyridin-3-ylmethyl)pyridin-3-amine
96) 3,4-bis(difluoromethoxy)-N-[3-(1H-pyrazol-3-yl)phenyl]-N-(pyridin-3-ylmethyl)aniline
101) 3,4-bis(difluoromethoxy)-N-(pyridin-3-ylmethyl)-N-[3-(1H-1,2,4-triazol-5-yl)phenyl]aniline
102) 4-{3-[[3-(cyclopentyloxy)-4-methoxyphenyl](pyridin-3-ylmethyl)amino]phenyl}-1-methyl-1H-pyrazol-5-ol
105) 4-{3-[[3,4-bis(difluoromethoxy)phenyl](pyridin-3-ylmethyl)amino]phenyl}-2-methyl-1,2-dihydro-3H-pyrazol-3-one
107) 4-[[4-(difluoromethoxy)-3-isopropoxyphenyl](pyridin-3-ylmethyl)amino]benzoic acid
108) 3-[[4-(difluoromethoxy)-3-isopropoxyphenyl](pyridin-3-ylmethyl)amino]benzoic acid
122) 1-{3-[[3,4-bis(difluoromethoxy)phenyl](pyridin-3-ylmethyl)amino]phenoxy}-2-methylpropan-2-ol
124) 4-[[4-(difluoromethoxy)-3-methoxyphenyl](pyridin-3-ylmethyl)amino]benzoic acid
126) 3-[[3-butoxy-4-(difluoromethoxy)phenyl](pyridin-3-ylmethyl)amino]benzoic acid
128) 4-[[3-butoxy-4-(difluoromethoxy)phenyl](pyridin-3-ylmethyl)amino]benzoic acid
135) 4-[[3-(cyclopropylmethoxy)-4-(difluoromethoxy)phenyl](pyridin-3-ylmethyl)amino]benzoic acid
171) 3-[[3-(cyclopentyloxy)-4-methoxyphenyl](1-pyridin-3-ylethyl)amino]benzoic acid
172) 3-[(3,4-dimethoxyphenyl)(1-pyridin-3-ylethyl)amino]benzoic acid
221) 4-[(4-ethoxy-3-methoxyphenyl)(pyridin-3-ylmethyl)amino]benzoic acid
222) 3-[(4-ethoxy-3-methoxyphenyl)(pyridin-3-ylmethyl)amino]benzoic acid
233) (2R)-3-{4-[[3-(cyclopentyloxy)-4-methoxyphenyl](pyridin-3-ylmethyl)amino]phenoxy}propane-1,2-diol
235) 5-[[3-(cyclopentyloxy)-4-methoxyphenyl](pyridin-3-ylmethyl)amino]pyridin-2(1H)-one
236) 2-({5-[[3-(cyclopentyloxy)-4-methoxyphenyl](pyridin-3-ylmethyl)amino]pyridin-2-yl}amino)propan-1-ol
237) 3-[{5-[[3-(cyclopentyloxy)-4-methoxyphenyl](pyridin-3-ylmethyl)amino]pyridin-2-yl}(methyl)amino]propane-1,2-diol
238) (2R)-3-{3-[[3-(cyclopentyloxy)-4-methoxyphenyl](pyridin-3-ylmethyl)amino]phenoxy}propane-1,2-diol
239) 5-{3-[[3-(cyclopentyloxy)-4-methoxyphenyl](pyridin-3-ylmethyl)amino]phenyl}imidazolidine-2,4-dione
240) (2R)-3-{4-[[3,4-bis(difluoromethoxy)phenyl](pyridin-3-ylmethyl)amino]phenoxy}propane-1,2-diol
242) 5-[[3-(cyclopentyloxy)-4-methoxyphenyl](pyridin-3-ylmethyl)amino]-2-methyl-1,2-benzisoxazol-3(2H)-one
243) 5-[[3-(cyclopentyloxy)-4-methoxyphenyl](pyridin-3-ylmethyl)amino]-1,2-benzisoxazol-3(2H)-one
244) (2R)-3-{4-[(3,4-dimethoxyphenyl)(pyridin-3-ylmethyl)amino]phenoxy}propane-1,2-diol
245) 5-{4-[[3-(cyclopentyloxy)-4-methoxyphenyl](pyridin-3-ylmethyl)amino]phenyl}imidazolidine-2,4-dione
246) 5-[[3,4-bis(difluoromethoxy)phenyl](pyridin-3-ylmethyl)amino]-2-{[(2R)-2,3-dihydroxypropyl]oxy}benzoic acid
247) 2-({5-[[3-(cyclopentyloxy)-4-methoxyphenyl](pyridin-3-ylmethyl)amino]pyridin-2-yl}amino)butan-1-ol
248) 1-({5-[[3-(cyclopentyloxy)-4-methoxyphenyl](pyridin-3-ylmethyl)amino]pyridin-2-yl}amino)butan-2-ol
249) 4-({5-[[3-(cyclopentyloxy)-4-methoxyphenyl](pyridin-3-ylmethyl)amino]pyridin-2-yl}amino)butan-2-ol
250) 1-({5-[[3-(cyclopentyloxy)-4-methoxyphenyl](pyridin-3-ylmethyl)amino]pyridin-2-yl}amino)propan-2-ol
251) 5-{3-[[3,4-bis(difluoromethoxy)phenyl](pyridin-3-ylmethyl)amino]phenyl}imidazolidine-2,4-dione
252) 1-({5-[[3-(cyclopentyloxy)-4-methoxyphenyl](pyridin-3-ylmethyl)amino]pyridin-2-yl}amino)-2-methylpropan-2-ol trifluoroacetic acid salt
254) 3-[{5-[[3,4-bis(difluoromethoxy)phenyl](pyridin-3-ylmethyl)amino]pyridin-2-yl}(methyl)amino]propane-1,2-diol
255) (2R)-3-{3-[[3,4-bis(difluoromethoxy)phenyl](pyridin-3-ylmethyl)amino]phenoxy}propane-1,2-diol
260) 4-{3-[[3,4-bis(difluoromethoxy)phenyl](pyridin-3-ylmethyl)amino]phenyl}imidazolidin-2-one
262) 5-{3-[[3,4-bis(difluoromethoxy)phenyl](pyridin-3-ylmethyl)amino]phenyl}-1,3-oxazolidin-2-one
264) 4-{3-[[3,4-bis(difluoromethoxy)phenyl](pyridin-3-ylmethyl)amino]phenyl}-1,3-oxazolidin-2-one, and
280) 4-[[3-(difluoromethoxy)-4-methoxyphenyl](pyridin-3-ylmethyl)amino]benzoic acid,

wherein a compound listed above (in either a free base form or in the form of a pharmaceutically acceptable salt) can also be in the form of a solvate (such as a hydrate),

wherein a compound listed above (in either a free base form or in the form of a pharmaceutically acceptable salt) can also be in the form of an N-oxide,

wherein a compound listed above (in a free base form or solvate or N-oxide thereof, or in the form of a pharmaceutically acceptable salt or solvate thereof,) can also be in the form of a polymorph, and

wherein if the compound exhibits chirality it can be in the form of a mixture of enantiomers such as a racemate or a mixture of diastereomers, or can be in the form of a single enantiomer or a single diastereomer.

According to a further aspect of the invention, the compounds of Formula II are selected from the following compounds wherein A and B are N:
43) 4-[[3-(cyclopentyloxy)-4-methoxyphenyl](pyrimidin-5-ylmethyl)amino]benzoic acid
46) 3-[[3-(cyclopentyloxy)-4-methoxyphenyl](pyrimidin-5-ylmethyl)amino]benzoic acid
80) 4-[[3,4-bis(difluoromethoxy)phenyl](pyrimidin-5-ylmethyl)amino]benzoic acid
82) 3-[[3,4-bis(difluoromethoxy)phenyl](pyrimidin-5-ylmethyl)amino]benzoic acid
113) 3,4-bis(difluoromethoxy)-N-[3-(methylsulfonyl)phenyl]-N-(pyrimidin-5-ylmethyl)aniline
114) 3,4-bis(difluoromethoxy)-N-[4-(methylsulfonyl)phenyl]-N-(pyrimidin-5-ylmethyl)aniline
117) 1-{4-[[3,4-bis(difluoromethoxy)phenyl](pyrimidin-5-ylmethyl)amino]phenyl}ethanone
137) {4-[[3,4-bis(difluoromethoxy)phenyl](pyrimidin-5-ylmethyl)amino]phenyl}acetic acid
139) {3-[[3,4-bis(difluoromethoxy)phenyl](pyrimidin-5-ylmethyl)amino]phenyl}acetic acid
143) 4-[[4-(difluoromethoxy)-3-ethoxyphenyl](pyrimidin-5-ylmethyl)amino]benzoic acid
144) 4-[[4-(difluoromethoxy)-3-ethoxyphenyl](1,3-oxazol-5-ylmethyl)amino]benzoic acid
153) 4-[[4-(difluoromethoxy)-3-methoxyphenyl](pyrimidin-5-ylmethyl)amino]benzoic acid
168) 4-[[3-(cyclopropylmethoxy)-4-(difluoromethoxy)phenyl](pyrimidin-5-ylmethyl)amino]benzoic acid
169) 4-[[4-(difluoromethoxy)-3-isopropoxyphenyl](pyrimidin-5-ylmethyl)amino]benzoic acid
186) 4-[[3-methoxy-4-(2,2,2-trifluoroethoxy)phenyl](pyrimidin-5-ylmethyl)amino]benzoic acid
191) 4-[(3,4-diethoxyphenyl)(pyrimidin-5-ylmethyl)amino]benzoic acid
192) 4-[(3-isopropoxy-4-methoxyphenyl)(pyrimidin-5-ylmethyl)amino]benzoic acid
193) 4-[[3-(cyclobutyloxy)-4-methoxyphenyl](pyrimidin-5-ylmethyl)amino]benzoic acid
194) 4-[[3-(cyclopropyloxy)-4-methoxyphenyl](pyrimidin-5-ylmethyl)amino]benzoic acid
195) 4-[[3-(cyclopropylmethoxy)-4-methoxyphenyl](pyrimidin-5-ylmethyl)amino]benzoic acid
196) 4-[(3-ethoxy-4-methoxyphenyl)(pyrimidin-5-ylmethyl)amino]benzoic acid
199) 3-[[4-(difluoromethoxy)-3-ethoxyphenyl](pyrimidin-5-ylmethyl)amino]benzoic acid
202) 4-[(4-ethoxy-3-isopropoxyphenyl)(pyrimidin-5-ylmethyl)amino]benzoic acid
205) 4-[[3-(cyclobutyloxy)-4-(difluoromethoxy)phenyl](pyrimidin-5-ylmethyl)amino]benzoic acid
209) 4-[(4-ethoxy-3-methoxyphenyl)(pyrimidin-5-ylmethyl)amino]benzoic acid
210) 3-[(3-ethoxy-4-methoxyphenyl)(pyrimidin-5-ylmethyl)amino]benzoic acid
211) 3-[[3-(cyclopropylmethoxy)-4-(difluoromethoxy)phenyl](pyrimidin-5-ylmethyl)amino]benzoic acid
212) 3-[[3-(cyclopropylmethoxy)-4-methoxyphenyl](pyrimidin-5-ylmethyl)amino]benzoic acid
213) 3-[(3-isopropoxy-4-methoxyphenyl)(pyrimidin-5-ylmethyl)amino]benzoic acid
214) 4-[[3-(benzyloxy)-4-(difluoromethoxy)phenyl](pyrimidin-5-ylmethyl)amino]benzoic acid
218) 3-[[3-(cyclopropyloxy)-4-methoxyphenyl](pyrimidin-5-ylmethyl)amino]benzoic acid
223) 3-[[4-(difluoromethoxy)-3-isopropoxyphenyl](pyrimidin-5-ylmethyl)amino]benzoic acid
228) 3-[[3-(cyclobutyloxy)-4-methoxyphenyl](pyrimidin-5-ylmethyl)amino]benzoic acid
230) 4-[[3-(cyclopropyloxy)-4-(difluoromethoxy)phenyl](pyrimidin-5-ylmethyl)amino]benzoic acid
272) 3-[[4-(cyclopropyloxy)-3-ethoxyphenyl](pyrimidin-5-ylmethyl)amino]benzoic acid,
283) 3-[[4-ethoxy-3-methoxyphenyl](pyrimidin-5-ylmethyl)amino]benzoic acid,
284) 3-[[3,4-diethoxyphenyl](pyrimidin-5-ylmethyl)amino]benzoic acid,
285) 3-[[3,4-diethoxyphenyl](1,3-oxazol-5-ylmethyl)amino]benzoic acid, and
287) 3-[[3-cyclobutyloxy-4-difluoromethoxyphenyl](pyrimidin-5-ylmethyl)amino]benzoic acid,

wherein a compound listed above (in either a free base form or in the form of a pharmaceutically acceptable salt) can also be in the form of a solvate (such as a hydrate),

wherein a compound listed above (in either a free base form or in the form of a pharmaceutically acceptable salt) can also be in the form of an N-oxide,

wherein a compound listed above (in a free base form or solvate or N-oxide thereof, or in the form of a pharmaceutically acceptable salt or solvate thereof,) can also be in the form of a polymorph, and

wherein if the compound exhibits chirality it can be in the form of a mixture of enantiomers such as a racemate or a mixture of diastereomers, or can be in the form of a single enantiomer or a single diastereomer.

According to a further aspect of the invention, the compounds of Formula II are selected from the following compounds wherein E and B are N:
47) 4-[[3-(cyclopentyloxy)-4-methoxyphenyl](pyridazin-3-ylmethyl)amino]benzoic acid,
48) 3-[[3-(cyclopentyloxy)-4-methoxyphenyl](pyridazin-3-ylmethyl)amino]benzoic acid,
74) 4-[[3,4-bis(difluoromethoxy)phenyl](pyridazin-3-ylmethyl)amino]benzoic acid, and
75) 3-[[3,4-bis(difluoromethoxy)phenyl](pyridazin-3-ylmethyl)amino]benzoic acid,

wherein a compound listed above (in either a free base form or in the form of a pharmaceutically acceptable salt) can also be in the form of a solvate (such as a hydrate),

wherein a compound listed above (in either a free base form or in the form of a pharmaceutically acceptable salt) can also be in the form of an N-oxide,

wherein a compound listed above (in a free base form or solvate or N-oxide thereof, or in the form of a pharmaceutically acceptable salt or solvate thereof,) can also be in the form of a polymorph, and

wherein if the compound exhibits chirality it can be in the form of a mixture of enantiomers such as a racemate or a mixture of diastereomers, or can be in the form of a single enantiomer or a single diastereomer.

According to a further aspect of the invention, the compounds of Formula II are selected from the following compounds wherein A and E are N:
118) 3,4-bis(difluoromethoxy)-N-[4-(methylsulfonyl)phenyl]-N-(pyrazin-2-ylmethyl)aniline,
119) 1-{4-[[3,4-bis(difluoromethoxy)phenyl](pyrazin-2-ylmethyl)amino]phenyl}ethanone,
140) {4-[[3,4-bis(difluoromethoxy)phenyl](pyrazin-2-ylmethyl)amino]phenyl}acetic acid,
142) 4-[[3,4-bis(difluoromethoxy)phenyl](pyrazin-2-ylmethyl)amino]benzoic acid,
147) 4-[[4-(difluoromethoxy)-3-ethoxyphenyl](pyrazin-2-ylmethyl)amino]benzoic acid,
148) 3-[[3,4-bis(difluoromethoxy)phenyl](pyrazin-2-ylmethyl)amino]benzoic acid,
154) 4-[[4-(difluoromethoxy)-3-methoxyphenyl](pyrazin-2-ylmethyl)amino]benzoic acid,
155) 4-[[4-(difluoromethoxy)-3-isopropoxyphenyl](pyrazin-2-ylmethyl)amino]benzoic acid,
156) 3-[[4-(difluoromethoxy)-3-isopropoxyphenyl](pyrazin-2-ylmethyl)amino]benzoic acid,
157) 4-[[3-(cyclopropylmethoxy)-4-(difluoromethoxy)phenyl](pyrazin-2-ylmethyl)amino]benzoic acid,
159) 3-[[3-(cyclopropylmethoxy)-4-(difluoromethoxy)phenyl](pyrazin-2-ylmethyl)amino]benzoic acid, and
161) 4-[[3-(difluoromethoxy)-4-methoxyphenyl](pyrazin-2-ylmethyl)amino]benzoic acid

wherein a compound listed above (in either a free base form or in the form of a pharmaceutically acceptable salt) can also be in the form of a solvate (such as a hydrate),

wherein a compound listed above (in either a free base form or in the form of a pharmaceutically acceptable salt) can also be in the form of an N-oxide,

wherein a compound listed above (in a free base form or solvate or N-oxide thereof, or in the form of a pharmaceutically acceptable salt or solvate thereof,) can also be in the form of a polymorph, and

wherein if the compound exhibits chirality it can be in the form of a mixture of enantiomers such as a racemate or a mixture of diastereomers, or can be in the form of a single enantiomer or a single diastereomer.

According to a further aspect of the invention, the compounds of Formula III are selected from the following compounds wherein X is S:
35) 3-[[3-(cyclopentyloxy)-4-methoxyphenyl](1,3-thiazol-5-ylmethyl)amino]benzoic acid,
40) 4-[[3-(cyclopentyloxy)-4-methoxyphenyl](1,3-thiazol-5-ylmethyl)amino]benzoic acid,
59) 4-[[3,4-bis(difluoromethoxy)phenyl](1,3-thiazol-5-ylmethyl)amino]benzoic acid,
60) 3-[[3,4-bis(difluoromethoxy)phenyl](1,3-thiazol-5-ylmethyl)amino]benzoic acid,
70) 3-[{4-methoxy-3-[(3R)-tetrahydrofuran-3-yloxy]phenyl}(1,3-thiazol-5-ylmethyl)amino]benzoic acid,
71) 4-[{4-methoxy-3-[(3R)-tetrahydrofuran-3-yloxy]phenyl}(1,3-thiazol-5-ylmethyl)amino]benzoic acid,
72) 4-[[3-(cyclopropylmethoxy)-4-methoxyphenyl](1,3-thiazol-5-ylmethyl)amino]benzoic acid,
73) 3-[[3-(cyclopropylmethoxy)-4-methoxyphenyl](1,3-thiazol-5-ylmethyl)amino]benzoic acid,
77) 3,4-bis(difluoromethoxy)-N-[4-(methylsulfonyl)phenyl]-N-(1,3-thiazol-5-ylmethyl)aniline,
78) 3,4-bis(difluoromethoxy)-N-[4-(ethylsulfonyl)phenyl]-N-(1,3-thiazol-5-ylmethyl)aniline,
79) 3,4-bis(difluoromethoxy)-N-[3-(methylsulfonyl)phenyl]-N-(1,3-thiazol-5-ylmethyl)aniline,
81) 5-[[3-(cyclopropylmethoxy)-4-methoxyphenyl](1,3-thiazol-5-ylmethyl)amino]nicotinic acid,
91) 5-[[3,4-bis(difluoromethoxy)phenyl](1,3-thiazol-5-ylmethyl)amino]-2-hydroxybenzoic acid,
92) 4-[[3,4-bis(difluoromethoxy)phenyl](1,3-thiazol-5-ylmethyl)amino]-2-hydroxybenzoic acid,
93) 1-{5-[[3,4-bis(difluoromethoxy)phenyl](1,3-thiazol-5-ylmethyl)amino]pyridin-3-yl}ethanone,
97) 3-[[4-(difluoromethoxy)-3-ethoxyphenyl](1,3-thiazol-5-ylmethyl)amino]benzoic acid,
98) 5-[[3,4-bis(difluoromethoxy)phenyl](1,3-thiazol-5-ylmethyl)amino]nicotinic acid,
99) 4-[[4-(difluoromethoxy)-3-ethoxyphenyl](1,3-thiazol-5-ylmethyl)amino]benzoic acid,
100) {4-[[3,4-bis(difluoromethoxy)phenyl](1,3-thiazol-5-ylmethyl)amino]phenyl}acetic acid,
103) {3-[[3,4-bis(difluoromethoxy)phenyl](1,3-thiazol-5-ylmethyl)amino]phenyl}acetic acid,
104) 4-[[3-(cyclopentyloxy)-4-(difluoromethoxy)phenyl](1,3-thiazol-5-ylmethyl)amino]benzoic acid,
106) 4-[[4-(difluoromethoxy)-3-isopropoxyphenyl](1,3-thiazol-5-ylmethyl)amino]benzoic acid,
109) 3-[[4-(difluoromethoxy)-3-isopropoxyphenyl](1,3-thiazol-5-ylmethyl)amino]benzoic acid,
110) 3-[[3-(cyclopentyloxy)-4-(difluoromethoxy)phenyl](1,3-thiazol-5-ylmethyl)amino]benzoic acid,
111) 4-methoxy-N-[4-(methylsulfonyl)phenyl]-3-[(3R)-tetrahydrofuran-3-yloxy]-N-(1,3-thiazol-5-ylmethyl)aniline,
112) 1-{4-[[3,4-bis(difluoromethoxy)phenyl](1,3-thiazol-5-ylmethyl)amino]phenyl}ethanone,
115) 4-methoxy-N-[3-(methylsulfonyl)phenyl]-3-[(3R)-tetrahydrofuran-3-yloxy]-N-(1,3-thiazol-5-ylmethyl)aniline,
116) 3-[[4-(difluoromethoxy)-3-methoxyphenyl](1,3-thiazol-5-ylmethyl)amino]benzoic acid,
120) 4-[[4-(difluoromethoxy)-3-methoxyphenyl](1,3-thiazol-5-ylmethyl)amino]benzoic acid,
121) 4-[[3-(cyclopropylmethoxy)-4-(difluoromethoxy)phenyl](1,3-thiazol-5-ylmethyl)amino]benzoic acid,
123) 1-{3-[[3,4-bis(difluoromethoxy)phenyl](1,3-thiazol-5-ylmethyl)amino]phenoxy}-2-methylpropan-2-ol,
125) 3-[[3-butoxy-4-(difluoromethoxy)phenyl](1,3-thiazol-5-ylmethyl)amino]benzoic acid,
129) 4-[[3-butoxy-4-(difluoromethoxy)phenyl](1,3-thiazol-5-ylmethyl)amino]benzoic acid,
130) 4-[(3-ethoxy-4-methoxyphenyl)(1,3-thiazol-5-ylmethyl)amino]benzoic acid,
131) 4-[(3-isopropoxy-4-methoxyphenyl)(1,3-thiazol-5-ylmethyl)amino]benzoic acid,
132) 3-(cyclopropylmethoxy)-4-methoxy-N-[4-(methylsulfonyl)phenyl]-N-(1,3-thiazol-5-ylmethyl)aniline,
133) 1-{4-[[3-(cyclopropylmethoxy)-4-methoxyphenyl](1,3-thiazol-5-ylmethyl)amino]phenyl}ethanone,
136) 3,4-bis(difluoromethoxy)-N-[4-methyl-3-(methylsulfonyl)phenyl]-N-(1,3-thiazol-5-ylmethyl)aniline,
138) 4-[(3,4-dimethoxyphenyl)(1,3-thiazol-5-ylmethyl)amino]benzoic acid,
145) 4-[[3,4-bis(difluoromethoxy)phenyl](1,3-thiazol-5-ylmethyl)amino]-N,N-dimethylbenzenesulfonamide,
146) 4-[[3,4-bis(difluoromethoxy)phenyl](1,3-thiazol-5-ylmethyl)amino]benzenesulfonamide,
149) 3-[[3-(difluoromethoxy)-4-methoxyphenyl](1,3-thiazol-5-ylmethyl)amino]benzoic acid,
150) 4-[[3-(difluoromethoxy)-4-methoxyphenyl](1,3-thiazol-5-ylmethyl)amino]benzoic acid,
151) 4-[[3,4-bis(difluoromethoxy)phenyl](1,3-thiazol-5-ylmethyl)amino]-N-methylbenzamide,
152) 4-[[3,4-bis(difluoromethoxy)phenyl](1,3-thiazol-5-ylmethyl)amino]benzamide,
158) 4-[[3,4-bis(difluoromethoxy)phenyl](1,3-thiazol-5-ylmethyl)amino]-N,N-dimethylbenzamide,
160) 5-[[4-(difluoromethoxy)-3-isopropoxyphenyl](1,3-thiazol-5-ylmethyl)amino]nicotinic acid,
162) N-{5-[[3,4-bis(difluoromethoxy)phenyl](1,3-thiazol-5-ylmethyl)amino]-2-methylphenyl}methane sulfonamide,
163) N-{3-[[3,4-bis(difluoromethoxy)phenyl](1,3-thiazol-5-ylmethyl)amino]phenyl}methane sulfonamide,
164) N-{3-[[3,4-bis(difluoromethoxy)phenyl](1,3-thiazol-5-ylmethyl)amino]phenyl}ethanesulfonamide,
165) 4-[[3,4-bis(difluoromethoxy)phenyl](1,3-thiazol-5-ylmethyl)amino]-N-methylbenzenesulfonamide,
170) 4-[(3,4-diethoxyphenyl)(1,3-thiazol-5-ylmethyl)amino]benzoic acid,
173) 4-[(4-ethoxy-3-methoxyphenyl)(1,3-thiazol-5-ylmethyl)amino]benzoic acid,
174) 4-(difluoromethoxy)-3-ethoxy-N-[4-(methylsulfonyl)phenyl]-N-(1,3-thiazol-5-ylmethyl)aniline,
178) {3-[[4-(difluoromethoxy)-3-ethoxyphenyl](1,3-thiazol-5-ylmethyl)amino]phenyl}acetic acid,
207) 4-[(3-ethoxy-4-isopropoxyphenyl)(1,3-thiazol-5-ylmethyl)amino]benzoic acid,
226) 3-[(4-ethoxy-3-methoxyphenyl)(1,3-thiazol-5-ylmethyl)amino]benzoic acid,
227) 3-[(3,4-diethoxyphenyl)(1,3-thiazol-5-ylmethyl)amino]benzoic acid,
241) (2R)-3-{4-[[3-(cyclopentyloxy)-4-methoxyphenyl](1,3-thiazol-5-ylmethyl)amino]phenoxy}propane-1,2-diol,
253) 3-[{5-[[3-(cyclopentyloxy)-4-methoxyphenyl](1,3-thiazol-5-ylmethyl)amino]pyridin-2-yl}(methyl)amino]propane-1,2-diol,
256) 5-{3-[[3,4-bis(difluoromethoxy)phenyl](1,3-thiazol-5-ylmethyl)amino]phenyl}-1,3,5-trimethylimidazolidine-2,4-dione,
257) 5-{3-[[3,4-bis(difluoromethoxy)phenyl](1,3-thiazol-5-ylmethyl)amino]phenyl}imidazolidine-2,4-dione,
258) (2R)-3-{4-[[3,4-bis(difluoromethoxy)phenyl](1,3-thiazol-5-ylmethyl)amino]phenoxy}propane-1,2-diol,
259) (2R)-3-{3-[[3,4-bis(difluoromethoxy)phenyl](1,3-thiazol-5-ylmethyl)amino]phenoxy}propane-1,2-diol,
261) 5-[[3,4-bis(difluoromethoxy)phenyl](1,3-thiazol-5-ylmethyl)amino]-1,2-benzisoxazol-3(2H)-one,
263) 6-[[3,4-bis(difluoromethoxy)phenyl](1,3-thiazol-5-ylmethyl)amino]-1,2-benzisoxazol-3(2H)-one,
265) 1-{3-[[3,4-bis(difluoromethoxy)phenyl](1,3-thiazol-5-ylmethyl)amino]phenoxy}-3-methoxypropan-2-ol,
270) 4-[[3,4-bis(difluoromethoxy)phenyl](1,3-thiazol-5-ylmethyl)amino]butanoic acid, and
271) cis-4-[[3,4-bis(difluoromethoxy)phenyl](1,3-thiazol-5-ylmethyl)amino]cyclohexanecarboxylic acid

wherein a compound listed above (in either a free base form or in the form of a pharmaceutically acceptable salt) can also be in the form of a solvate (such as a hydrate),

wherein a compound listed above (in either a free base form or in the form of a pharmaceutically acceptable salt) can also be in the form of an N-oxide,

wherein a compound listed above (in a free base form or solvate or N-oxide thereof, or in the form of a pharmaceutically acceptable salt or solvate thereof,) can also be in the form of a polymorph, and

wherein if the compound exhibits chirality it can be in the form of a mixture of enantiomers such as a racemate or a mixture of diastereomers, or can be in the form of a single enantiomer or a single diastereomer.

According to a further aspect of the invention, the compounds of Formula III are selected from the following compounds wherein X is O:
141) 4-[[3,4-bis(difluoromethoxy)phenyl](1,3-oxazol-5-ylmethyl)amino]benzoic acid,
144) 4-[[4-(difluoromethoxy)-3-ethoxyphenyl](1,3-oxazol-5-ylmethyl)amino]benzoic acid,
166) 4-[[4-(difluoromethoxy)-3-isopropoxyphenyl](1,3-oxazol-5-ylmethyl)amino]benzoic acid,
167) 4-[[3-(cyclopropylmethoxy)-4-(difluoromethoxy)phenyl](1,3-oxazol-5-ylmethyl)amino]benzoic acid,
175) {3-[[4-(difluoromethoxy)-3-ethoxyphenyl](1,3-oxazol-5-ylmethyl)amino]phenyl}acetic acid,
176) 4-(difluoromethoxy)-3-ethoxy-N-[4-(methylsulfonyl)phenyl]-N-(1,3-oxazol-5-ylmethyl)aniline,
177) {4-[[4-(difluoromethoxy)-3-ethoxyphenyl](1,3-oxazol-5-ylmethyl)amino]phenyl}acetic acid,
179) 4-[[4-(difluoromethoxy)-3-methoxyphenyl](1,3-oxazol-5-ylmethyl)amino]benzoic acid,
180) 4-[(3-isopropoxy-4-methoxyphenyl)(1,3-oxazol-5-ylmethyl)amino]benzoic acid,
181) 4-[(3-ethoxy-4-methoxyphenyl)(1,3-oxazol-5-ylmethyl)amino]benzoic acid,
182) 4-[[3-(cyclobutyloxy)-4-(difluoromethoxy)phenyl](1,3-oxazol-5-ylmethyl)amino]benzoic acid,
183) 4-[[3-(cyclopropylmethoxy)-4-methoxyphenyl](1,3-oxazol-5-ylmethyl)amino]benzoic acid,
184) 4-[[3-(cyclobutyloxy)-4-methoxyphenyl](1,3-oxazol-5-ylmethyl)amino]benzoic acid,
185) 4-[[3-methoxy-4-(2,2,2-trifluoroethoxy)phenyl](1,3-oxazol-5-ylmethyl)amino]benzoic acid,
189) 4-[[3-(cyclopropyloxy)-4-methoxyphenyl](1,3-oxazol-5-ylmethyl)amino]benzoic acid,
190) 4-[[4-(difluoromethoxy)-3-ethoxyphenyl](1,3-oxazol-4-ylmethyl)amino]benzoic acid,
197) 3-[[4-(difluoromethoxy)-3-isopropoxyphenyl](1,3-oxazol-5-ylmethyl)amino]benzoic acid,
198) 3-[[3-(cyclopropylmethoxy)-4-(difluoromethoxy)phenyl](1,3-oxazol-5-ylmethyl)amino]benzoic acid,
200) 3-[(3-ethoxy-4-methoxyphenyl)(1,3-oxazol-5-ylmethyl)amino]benzoic acid,
201) 3-[[3-(cyclobutyloxy)-4-(difluoromethoxy)phenyl](1,3-oxazol-5-ylmethyl)amino]benzoic acid,
203) 3-[(3-isopropoxy-4-methoxyphenyl)(1,3-oxazol-5-ylmethyl)amino]benzoic acid,
204) 3-[[4-(difluoromethoxy)-3-ethoxyphenyl](1,3-oxazol-5-ylmethyl)amino]benzoic acid,
206) 3-[[4-(difluoromethoxy)-3-methoxyphenyl](1,3-oxazol-5-ylmethyl)amino]benzoic acid,
208) 4-[(4-ethoxy-3-isopropoxyphenyl)(1,3-oxazol-5-ylmethyl)amino]benzoic acid,
215) 3-[[3-(cyclopropylmethoxy)-4-methoxyphenyl](1,3-oxazol-5-ylmethyl)amino]benzoic acid,
216) 3-[[3-(cyclobutyloxy)-4-methoxyphenyl](1,3-oxazol-5-ylmethyl)amino]benzoic acid,
217) 4-[[3-ethoxy-4-(trifluoromethoxy)phenyl](1,3-oxazol-5-ylmethyl)amino]benzoic acid,
219) 4-[(4-ethoxy-3-methoxyphenyl)(1,3-oxazol-5-ylmethyl)amino]benzoic acid,
220) 4-[(3,4-diethoxyphenyl)(1,3-oxazol-5-ylmethyl)amino]benzoic acid,
224) 3-[(4-ethoxy-3-methoxyphenyl)(1,3-oxazol-5-ylmethyl)amino]benzoic acid,
231) 4-[[3-(cyclopropyloxy)-4-(difluoromethoxy)phenyl](1,3-oxazol-5-ylmethyl)amino]benzoic acid,
232) 4-[[4-methoxy-3-(trifluoromethoxy)phenyl](1,3-oxazol-5-ylmethyl)amino]benzoic acid,
279) 3-[[3-(difluoromethoxy)-4-methoxyphenyl](1,3-oxazol-5-ylmethyl)amino]benzoic acid,
282) 4-[[3-hydroxy-4-methoxyphenyl](1,3-oxazol-5-ylmethyl)amino]benzoic acid,
285) 3-[[3,4-diethoxyphenyl](1,3-oxazol-5-ylmethyl)amino]benzoic acid,
286) 3-[[3-cyclopropyloxy-4-methoxyphenyl](1,3-oxazol-5-ylmethyl)amino]benzoic acid, and
288) 3-[[3-cyclopropyloxy-4-difluoromethoxyphenyl](1,3-oxazol-5-ylmethyl)amino]benzoic acid,

wherein a compound listed above (in either a free base form or in the form of a pharmaceutically acceptable salt) can also be in the form of a solvate (such as a hydrate),

wherein a compound listed above (in either a free base form or in the form of a pharmaceutically acceptable salt) can also be in the form of an N-oxide,

wherein a compound listed above (in a free base form or solvate or N-oxide thereof, or in the form of a pharmaceutically acceptable salt or solvate thereof,) can also be in the form of a polymorph, and

wherein if the compound exhibits chirality it can be in the form of a mixture of enantiomers such as a racemate or a mixture of diastereomers, or can be in the form of a single enantiomer or a single diastereomer.

According to a further aspect, the compounds of the invention are of subformula IV:

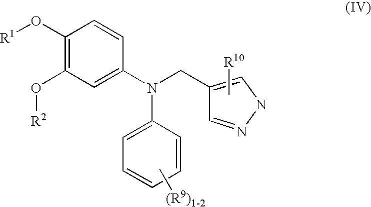

wherein

R¹ is C₁₋₄-alkyl or halogenated C₁₋₄-alkyl,

R² is C₁₋₄-alkyl, halogenated C₁₋₄-alkyl, or C-₃₋₁₀-cycloalkyl,

R⁹ is halogen, C₁₋₄-alkyl, hydroxy, C₁₋₄-alkoxy, C₁₋₄-alkoxy-C₁₋₈-alkoxy, nitro, trifluoromethyl, OCF₃, amino, C₁₋₄-alkylamino, di-(C₁₋₄-alkyl)amino, C₁₋₈-hydroxyalkyl, C₁₋₄-hydroxyalkoxy, C₁₋₄-dihydroxyalkoxy, carboxy, carboxy-C₁₋₄-alkyl, C₁₋₄-alkoxy-carbonyl, C₁₋₄-alkylsulfonyl, aminosulfonyl, aminocarbonyl, C₁₋₄-alkylaminocarbonyl, di-C₁₋₄-alkylaminocarbonyl, C₁₋₄-alkyl-SO₂—NH—, C₁₋₄-alkyl-NH—SO₂—, or cyano, and

R¹⁰ is H or C₁₋₄-alkyl,

and pharmaceutically acceptable salts or solvates (e.g., hydrates) or N-oxides thereof, or solvates of pharmaceutically acceptable salts or N-oxides thereof.

According to a further aspect of the invention, the compounds of Formula I are selected from:
34) 3-[[3-(cyclopentyloxy)-4-methoxyphenyl](1H-pyrazol-4-ylmethyl)amino]benzoic acid, and
39) 4-[[3-(cyclopentyloxy)-4-methoxyphenyl](1H-pyrazol-4-ylmethyl)amino]benzoic acid,

wherein a compound listed above (in either a free base form or in the form of a pharmaceutically acceptable salt) can also be in the form of a solvate (such as a hydrate),

wherein a compound listed above (in either a free base form or in the form of a pharmaceutically acceptable salt) can also be in the form of an N-oxide,

wherein a compound listed above (in a free base form or solvate or N-oxide thereof, or in the form of a pharmaceutically acceptable salt or solvate thereof,) can also be in the form of a polymorph, and

wherein if the compound exhibits chirality it can be in the form of a mixture of enantiomers such as a racemate or a mixture of diastereomers, or can be in the form of a single enantiomer or a single diastereomer.

According to a further aspect, the compounds of the invention are of subformulae Va or Vb:

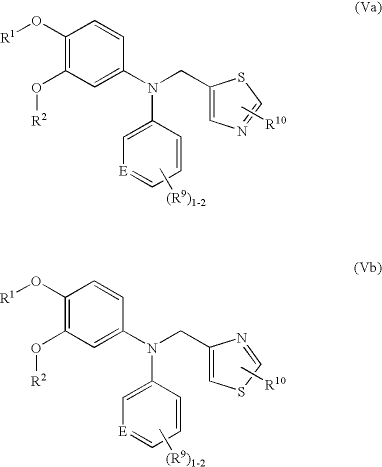

wherein

E is N or CH,

R¹ is C₁₋₄-alkyl or halogenated C₁₋₄-alkyl,

R² is C₁₋₄-alkyl, halogenated C₁₋₄-alkyl, C-₃₋₁₀-cycloalkyl, C₄₋₁₂-cycloalkylalkyl (e.g., cyclopropylmethyl), or tetrahydrofuranyl (e.g., (3R)-tetrahydrofuran-3-yl),

R⁹ is halogen, C₁₋₄-alkyl, hydroxy, C₁₋₄-alkoxy, C₁₋₄-alkoxy-C₁₋₈-alkoxy, nitro, trifluoromethyl, OCF₃, amino, C₁₋₄-alkylamino, di-(C₁₋₄-alkyl)amino, C₁₋₈-hydroxyalkyl, C₁₋₄-hydroxyalkoxy, C₁₋₄-dihydroxyalkoxy, C₁₋₄-alkoxy-C₁₋₄-hydroxyalkoxy, carboxy, carboxy-C₁₋₄-alkyl, C₁₋₄-alkoxy-carbonyl, C₁₋₄-alkylsulfonyl, aminosulfonyl, aminocarbonyl, C₁₋₄-alkylaminocarbonyl, di-C₁₋₄-alkylaminocarbonyl, C₁₋₄-alkyl-SO₂—NH—, C₁₋₄-alkyl-NH—SO₂—, cyano, trimethylimidazolidinedione, or imidazolidinedione, and

R¹⁰ is H or C₁₋₄-alkyl,

and pharmaceutically acceptable salts or solvates (e.g., hydrates) or N-oxides thereof, or solvates of pharmaceutically acceptable salts or N-oxides thereof.

According to a further aspect of the invention, the compounds of Formulas Va and Vb are selected from:
35) 3-[[3-(cyclopentyloxy)-4-methoxyphenyl](1,3-thiazol-5-ylmethyl)amino]benzoic acid,
40) 4-[[3-(cyclopentyloxy)-4-methoxyphenyl](1,3-thiazol-5-ylmethyl)amino]benzoic acid,
45) 3-[[3-(cyclopentyloxy)-4-methoxyphenyl](1,3-thiazol-4-ylmethyl)amino]benzoic acid
59) 4-[[3,4-bis(difluoromethoxy)phenyl](1,3-thiazol-5-ylmethyl)amino]benzoic acid,
60) 3-[[3,4-bis(difluoromethoxy)phenyl](1,3-thiazol-5-ylmethyl)amino]benzoic acid,
70) 3-[{4-methoxy-3-[(3R)-tetrahydrofuran-3-yloxy]phenyl}(1,3-thiazol-5-ylmethyl)amino]benzoic acid,
71) 4-[{4-methoxy-3-[(3R)-tetrahydrofuran-3-yloxy]phenyl}(1,3-thiazol-5-ylmethyl)amino]benzoic acid,
72) 4-[[3-(cyclopropylmethoxy)-4-methoxyphenyl](1,3-thiazol-5-ylmethyl)amino]benzoic acid,
73) 3-[[3-(cyclopropylmethoxy)-4-methoxyphenyl](1,3-thiazol-5-ylmethyl)amino]benzoic acid,
77) 3,4-bis(difluoromethoxy)-N-[4-(methylsulfonyl)phenyl]-N-(1,3-thiazol-5-ylmethyl)aniline,
78) 3,4-bis(difluoromethoxy)-N-[4-(ethylsulfonyl)phenyl]-N-(1,3-thiazol-5-ylmethyl)aniline,
79) 3,4-bis(difluoromethoxy)-N-[3-(methylsulfonyl)phenyl]-N-(1,3-thiazol-5-ylmethyl)aniline,
81) 5-[[3-(cyclopropylmethoxy)-4-methoxyphenyl](1,3-thiazol-5-ylmethyl)amino]nicotinic acid,
91) 5-[[3,4-bis(difluoromethoxy)phenyl](1,3-thiazol-5-ylmethyl)amino]-2-hydroxybenzoic acid,
92) 4-[[3,4-bis(difluoromethoxy)phenyl](1,3-thiazol-5-ylmethyl)amino]-2-hydroxybenzoic acid,
93) 1-{5-[[3,4-bis(difluoromethoxy)phenyl](1,3-thiazol-5-ylmethyl)amino]pyridin-3-yl}ethanone,
97) 3-[[4-(difluoromethoxy)-3-ethoxyphenyl](1,3-thiazol-5-ylmethyl)amino]benzoic acid,
98) 5-[[3,4-bis(difluoromethoxy)phenyl](1,3-thiazol-5-ylmethyl)amino]nicotinic acid,
99) 4-[[4-(difluoromethoxy)-3-ethoxyphenyl](1,3-thiazol-5-ylmethyl)amino]benzoic acid,
100) {4-[[3,4-bis(difluoromethoxy)phenyl](1,3-thiazol-5-ylmethyl)amino]phenyl}acetic acid,
103) {3-[[3,4-bis(difluoromethoxy)phenyl](1,3-thiazol-5-ylmethyl)amino]phenyl}acetic acid,
104) 4-[[3-(cyclopentyloxy)-4-(difluoromethoxy)phenyl](1,3-thiazol-5-ylmethyl)amino]benzoic acid,
106) 4-[[4-(difluoromethoxy)-3-isopropoxyphenyl](1,3-thiazol-5-ylmethyl)amino]benzoic acid,
109) 3-[[4-(difluoromethoxy)-3-isopropoxyphenyl](1,3-thiazol-5-ylmethyl)amino]benzoic acid,
110) 3-[[3-(cyclopentyloxy)-4-(difluoromethoxy)phenyl](1,3-thiazol-5-ylmethyl)amino]benzoic acid,
111) 4-methoxy-N-[4-(methylsulfonyl)phenyl]-3-[(3R)-tetrahydrofuran-3-yloxy]-N-(1,3-thiazol-5-ylmethyl)aniline,
112) 1-{4-[[3,4-bis(difluoromethoxy)phenyl](1,3-thiazol-5-ylmethyl)amino]phenyl}ethanone,
115) 4-methoxy-N-[3-(methylsulfonyl)phenyl]-3-[(3R)-tetrahydrofuran-3-yloxy]-N-(1,3-thiazol-5-ylmethyl)aniline,
116) 3-[[4-(difluoromethoxy)-3-methoxyphenyl](1,3-thiazol-5-ylmethyl)amino]benzoic acid,
120) 4-[[4-(difluoromethoxy)-3-methoxyphenyl](1,3-thiazol-5-ylmethyl)amino]benzoic acid,
121) 4-[[3-(cyclopropylmethoxy)-4-(difluoromethoxy)phenyl](1,3-thiazol-5-ylmethyl)amino]benzoic acid,
123) 1-{3-[[3,4-bis(difluoromethoxy)phenyl](1,3-thiazol-5-ylmethyl)amino]phenoxy}-2-methylpropan-2-ol,
125) 3-[[3-butoxy-4-(difluoromethoxy)phenyl](1,3-thiazol-5-ylmethyl)amino]benzoic acid,
129) 4-[[3-butoxy-4-(difluoromethoxy)phenyl](1,3-thiazol-5-ylmethyl)amino]benzoic acid,
130) 4-[(3-ethoxy-4-methoxyphenyl)(1,3-thiazol-5-ylmethyl)amino]benzoic acid,
131) 4-[(3-isopropoxy-4-methoxyphenyl)(1,3-thiazol-5-ylmethyl)amino]benzoic acid,
132) 3-(cyclopropylmethoxy)-4-methoxy-N-[4-(methylsulfonyl)phenyl]-N-(1,3-thiazol-5-ylmethyl)aniline,
133) 1-{4-[[3-(cyclopropylmethoxy)-4-methoxyphenyl](1,3-thiazol-5-ylmethyl)amino]phenyl}ethanone,
136) 3,4-bis(difluoromethoxy)-N-[4-methyl-3-(methylsulfonyl)phenyl]-N-(1,3-thiazol-5-ylmethyl)aniline,
138) 4-[(3,4-dimethoxyphenyl)(1,3-thiazol-5-ylmethyl)amino]benzoic acid,
145) 4-[[3,4-bis(difluoromethoxy)phenyl](1,3-thiazol-5-ylmethyl)amino]-N,N-dimethylbenzenesulfonamide,
146) 4-[[3,4-bis(difluoromethoxy)phenyl](1,3-thiazol-5-ylmethyl)amino]benzenesulfonamide,
149) 3-[[3-(difluoromethoxy)-4-methoxyphenyl](1,3-thiazol-5-ylmethyl)amino]benzoic acid,
150) 4-[[3-(difluoromethoxy)-4-methoxyphenyl](1,3-thiazol-5-ylmethyl)amino]benzoic acid,
151) 4-[[3,4-bis(difluoromethoxy)phenyl](1,3-thiazol-5-ylmethyl)amino]-N-methylbenzamide,
152) 4-[[3,4-bis(difluoromethoxy)phenyl](1,3-thiazol-5-ylmethyl)amino]benzamide,
158) 4-[[3,4-bis(difluoromethoxy)phenyl](1,3-thiazol-5-ylmethyl)amino]-N,N-dimethylbenzamide,
160) 5-[[4-(difluoromethoxy)-3-isopropoxyphenyl](1,3-thiazol-5-ylmethyl)amino]nicotinic acid,
162) N-{5-[[3,4-bis(difluoromethoxy)phenyl](1,3-thiazol-5-ylmethyl)amino]-2-methylphenyl}methane sulfonamide,
163) N-{3-[[3,4-bis(difluoromethoxy)phenyl](1,3-thiazol-5-ylmethyl)amino]phenyl}methane sulfonamide,
164) N-{3-[[3,4-bis(difluoromethoxy)phenyl](1,3-thiazol-5-ylmethyl)amino]phenyl}ethanesulfonamide,
165) 4-[[3,4-bis(difluoromethoxy)phenyl](1,3-thiazol-5-ylmethyl)amino]-N-methylbenzenesulfonamide,
170) 4-[(3,4-diethoxyphenyl)(1,3-thiazol-5-ylmethyl)amino]benzoic acid,
173) 4-[(4-ethoxy-3-methoxyphenyl)(1,3-thiazol-5-ylmethyl)amino]benzoic acid,
174) 4-(difluoromethoxy)-3-ethoxy-N-[4-(methylsulfonyl)phenyl]-N-(1,3-thiazol-5-ylmethyl)aniline,
178) {3-[[4-(difluoromethoxy)-3-ethoxyphenyl](1,3-thiazol-5-ylmethyl)amino]phenyl}acetic acid,
207) 4-[(3-ethoxy-4-isopropoxyphenyl)(1,3-thiazol-5-ylmethyl)amino]benzoic acid,
226) 3-[(4-ethoxy-3-methoxyphenyl)(1,3-thiazol-5-ylmethyl)amino]benzoic acid,
227) 3-[(3,4-diethoxyphenyl)(1,3-thiazol-5-ylmethyl)amino]benzoic acid,
241) (2R)-3-{4-[[3-(cyclopentyloxy)-4-methoxyphenyl](1,3-thiazol-5-ylmethyl)amino]phenoxy}propane-1,2-diol,
256) 5-{3-[[3,4-bis(difluoromethoxy)phenyl](1,3-thiazol-5-ylmethyl)amino]phenyl}-1,3,5-trimethylimidazolidine-2,4-dione,
257) 5-{3-[[3,4-bis(difluoromethoxy)phenyl](1,3-thiazol-5-ylmethyl)amino]phenyl}imidazolidine-2,4-dione,
258) (2R)-3-{4-[[3,4-bis(difluoromethoxy)phenyl](1,3-thiazol-5-ylmethyl)amino]phenoxy}propane-1,2-diol,
259) (2R)-3-{3-[[3,4-bis(difluoromethoxy)phenyl](1,3-thiazol-5-ylmethyl)amino]phenoxy}propane-1,2-diol,
261) 5-[[3,4-bis(difluoromethoxy)phenyl](1,3-thiazol-5-ylmethyl)amino]-1,2-benzisoxazol-3(2H)-one,
263) 6-[[3,4-bis(difluoromethoxy)phenyl](1,3-thiazol-5-ylmethyl)amino]-1,2-benzisoxazol-3(2H)-one,
265) 1-{3-[[3,4-bis(difluoromethoxy)phenyl](1,3-thiazol-5-ylmethyl)amino]phenoxy}-3-methoxypropan-2-ol,
270) 4-[[3,4-bis(difluoromethoxy)phenyl](1,3-thiazol-5-ylmethyl)amino]butanoic acid, and
271) cis-4-[[3,4-bis(difluoromethoxy)phenyl](1,3-thiazol-5-ylmethyl)amino]cyclohexanecarboxylic acid

wherein a compound listed above (in either a free base form or in the form of a pharmaceutically acceptable salt) can also be in the form of a solvate (such as a hydrate),

wherein a compound listed above (in either a free base form or in the form of a pharmaceutically acceptable salt) can also be in the form of an N-oxide,

wherein a compound listed above (in a free base form or solvate or N-oxide thereof, or in the form of a pharmaceutically acceptable salt or solvate thereof,) can also be in the form of a polymorph, and

wherein if the compound exhibits chirality it can be in the form of a mixture of enantiomers such as a racemate or a mixture of diastereomers, or can be in the form of a single enantiomer or a single diastereomer.

According to a further aspect, the compounds of the invention are of subformula VI:

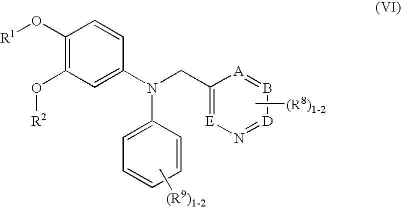

wherein

one of A, B, D and E is N and the others are CH (or CR⁸ if the carbon atom carries the R⁸ substituent),

R¹ is C₁₋₄-alkyl or halogenated C₁₋₄-alkyl,

R² is C₁₋₄-alkyl, halogenated C₁₋₄-alkyl, C-₃₋₁₀-cycloalkyl, or C₄₋₁₂-cycloalkylalkyl,

R⁸ is amino, C₁₋₄-alkylamino, or di-(C₁₋₄-alkyl)amino, and

R⁹ is halogen, C₁₋₄-alkyl, hydroxy, C₁₋₄-alkoxy, C₁₋₄-alkoxy-C₁₋₈-alkoxy, nitro, trifluoromethyl, OCF₃, amino, C₁₋₄-alkylamino, di-(C₁₋₄-alkyl)amino, C₁₋₈-hydroxyalkyl, C₁₋₄-hydroxyalkoxy, C₁₋₄-dihydroxyalkoxy, carboxy, carboxy-C₁₋₄-alkyl (HOOC—CH₂—), C₁₋₄-alkyl-carbonyl, C₁₋₄-alkoxy-carbonyl, C₁₋₄-alkylsulfonyl, aminosulfonyl, aminocarbonyl, C₁₋₄-alkylaminocarbonyl, di-C₁₋₄-alkylaminocarbonyl, C₁₋₄-alkyl-SO₂—NH—, C₁₋₄-alkyl-NH—SO₂—, or cyano,

and pharmaceutically acceptable salts or solvates (e.g., hydrates) or N-oxides thereof, or solvates of pharmaceutically acceptable salts or N-oxides thereof.

According to a further aspect, the compounds of the invention are of subformula VIa which corresponds to subformula VI wherein A is N.

According to a further aspect, the compounds of the invention are of subformula VIb which corresponds to subformula VI wherein B is N.

According to a further aspect, the compounds of the invention are of subformula VIc which corresponds to subformula VI wherein D is N.

According to a further aspect, the compounds of the invention are of subformula VId which corresponds to subformula VI wherein E is N.

According to a further aspect of the invention, the compounds of Formula VI are selected from:
51) 4-([3-(cyclopentyloxy)-4-methoxyphenyl]{[2-(dimethylamino)pyrimidin-5-yl]methyl}amino)benzoic acid, and
52) 3-([3-(cyclopentyloxy)-4-methoxyphenyl]{[2-(dimethylamino)pyrimidin-5-yl]methyl}amino)benzoic acid

wherein a compound listed above (in either a free base form or in the form of a pharmaceutically acceptable salt) can also be in the form of a solvate (such as a hydrate),

wherein a compound listed above (in either a free base form or in the form of a pharmaceutically acceptable salt) can also be in the form of an N-oxide,

wherein a compound listed above (in a free base form or solvate or N-oxide thereof, or in the form of a pharmaceutically acceptable salt or solvate thereof,) can also be in the form of a polymorph, and

wherein if the compound exhibits chirality it can be in the form of a mixture of enantiomers such as a racemate or a mixture of diastereomers, or can be in the form of a single enantiomer or a single diastereomer.

According to a further aspect, the compounds of the invention are of subformula VII:

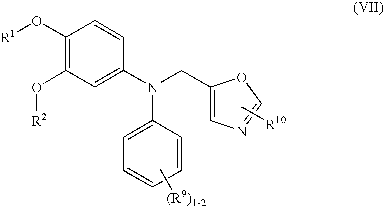

wherein

R¹ is C₁₋₄-alkyl or halogenated C₁₋₄-alkyl,

R² is H, C₁₋₄-alkyl, halogenated C₁₋₄-alkyl, C-₃₋₁₀-cycloalkyl, or C-₄₋₁₂-cycloalkylalkyl,

R⁹ is halogen, C₁₋₄-alkyl, hydroxy, C₁₋₄-alkoxy, C₁₋₄-alkoxy-C₁₋₈-alkoxy, nitro, trifluoromethyl, OCF₃, amino, C₁₋₄-alkylamino, di-(C₁₋₄-alkyl)amino, C₁₋₈-hydroxyalkyl, C₁₋₄-hydroxyalkoxy, C₁₋₄-dihydroxyalkoxy, carboxy, carboxy-C₁₋₄-alkyl, C₁₋₄-alkoxy-carbonyl, C₁₋₄-alkylsulfonyl, aminosulfonyl, aminocarbonyl, C₁₋₄-alkylaminocarbonyl, di-C₁₋₄-alkylaminocarbonyl, C₁₋₄-alkyl-SO₂—NH—, C₁₋₄-alkyl-NH—SO₂—, or cyano, and

R¹⁰ is H or C₁₋₄-alkyl,

and pharmaceutically acceptable salts or solvates (e.g., hydrates) or N-oxides thereof, or solvates of pharmaceutically acceptable salts or N-oxides thereof.

According to a further aspect of the invention, the compounds of Formula VII are selected from:
141) 4-[[3,4-bis(difluoromethoxy)phenyl](1,3-oxazol-5-ylmethyl)amino]benzoic acid,
144) 4-[[4-(difluoromethoxy)-3-ethoxyphenyl](1,3-oxazol-5-ylmethyl)amino]benzoic acid,
166) 4-[[4-(difluoromethoxy)-3-isopropoxyphenyl](1,3-oxazol-5-ylmethyl)amino]benzoic acid,
167) 4-[[3-(cyclopropylmethoxy)-4-(difluoromethoxy)phenyl](1,3-oxazol-5-ylmethyl)amino]benzoic acid,
175) {3-[[4-(difluoromethoxy)-3-ethoxyphenyl](1,3-oxazol-5-ylmethyl)amino]phenyl}acetic acid,
176) 4-(difluoromethoxy)-3-ethoxy-N-[4-(methylsulfonyl)phenyl]-N-(1,3-oxazol-5-ylmethyl)aniline,
177) {4-[[4-(difluoromethoxy)-3-ethoxyphenyl](1,3-oxazol-5-ylmethyl)amino]phenyl}acetic acid,
179) 4-[[4-(difluoromethoxy)-3-methoxyphenyl](1,3-oxazol-5-ylmethyl)amino]benzoic acid,
180) 4-[(3-isopropoxy-4-methoxyphenyl)(1,3-oxazol-5-ylmethyl)amino]benzoic acid,
181) 4-[(3-ethoxy-4-methoxyphenyl)(1,3-oxazol-5-ylmethyl)amino]benzoic acid,
182) 4-[[3-(cyclobutyloxy)-4-(difluoromethoxy)phenyl](1,3-oxazol-5-ylmethyl)amino]benzoic acid,
183) 4-[[3-(cyclopropylmethoxy)-4-methoxyphenyl](1,3-oxazol-5-ylmethyl)amino]benzoic acid,
184) 4-[[3-(cyclobutyloxy)-4-methoxyphenyl](1,3-oxazol-5-ylmethyl)amino]benzoic acid,
185) 4-[[3-methoxy-4-(2,2,2-trifluoroethoxy)phenyl](1,3-oxazol-5-ylmethyl)amino]benzoic acid,
189) 4-[[3-(cyclopropyloxy)-4-methoxyphenyl](1,3-oxazol-5-ylmethyl)amino]benzoic acid,
190) 4-[[4-(difluoromethoxy)-3-ethoxyphenyl](1,3-oxazol-4-ylmethyl)amino]benzoic acid,
197) 3-[[4-(difluoromethoxy)-3-isopropoxyphenyl](1,3-oxazol-5-ylmethyl)amino]benzoic acid,
198) 3-[[3-(cyclopropylmethoxy)-4-(difluoromethoxy)phenyl](1,3-oxazol-5-ylmethyl)amino]benzoic acid,
200) 3-[(3-ethoxy-4-methoxyphenyl)(1,3-oxazol-5-ylmethyl)amino]benzoic acid,
201) 3-[[3-(cyclobutyloxy)-4-(difluoromethoxy)phenyl](1,3-oxazol-5-ylmethyl)amino]benzoic acid,
203) 3-[(3-isopropoxy-4-methoxyphenyl)(1,3-oxazol-5-ylmethyl)amino]benzoic acid,
204) 3-[[4-(difluoromethoxy)-3-ethoxyphenyl](1,3-oxazol-5-ylmethyl)amino]benzoic acid,
206) 3-[[4-(difluoromethoxy)-3-methoxyphenyl](1,3-oxazol-5-ylmethyl)amino]benzoic acid,
208) 4-[(4-ethoxy-3-isopropoxyphenyl)(1,3-oxazol-5-ylmethyl)amino]benzoic acid,
215) 3-[[3-(cyclopropylmethoxy)-4-methoxyphenyl](1,3-oxazol-5-ylmethyl)amino]benzoic acid,
216) 3-[[3-(cyclobutyloxy)-4-methoxyphenyl](1,3-oxazol-5-ylmethyl)amino]benzoic acid,
217) 4-[[3-ethoxy-4-(trifluoromethoxy)phenyl](1,3-oxazol-5-ylmethyl)amino]benzoic acid,
219) 4-[(4-ethoxy-3-methoxyphenyl)(1,3-oxazol-5-ylmethyl)amino]benzoic acid,
220) 4-[(3,4-diethoxyphenyl)(1,3-oxazol-5-ylmethyl)amino]benzoic acid,
224) 3-[(4-ethoxy-3-methoxyphenyl)(1,3-oxazol-5-ylmethyl)amino]benzoic acid,
231) 4-[[3-(cyclopropyloxy)-4-(difluoromethoxy)phenyl](1,3-oxazol-5-ylmethyl)amino]benzoic acid,
232) 4-[[4-methoxy-3-(trifluoromethoxy)phenyl](1,3-oxazol-5-ylmethyl)amino]benzoic acid,
279) 3-[[3-(difluoromethoxy)-4-methoxyphenyl](1,3-oxazol-5-ylmethyl)amino]benzoic acid,
282) 4-[[3-hydroxy-4-methoxyphenyl](1,3-oxazol-5-ylmethyl)amino]benzoic acid,
285) 3-[[3,4-diethoxyphenyl](1,3-oxazol-5-ylmethyl)amino]benzoic acid,
286) 3-[[3-cyclopropyloxy-4-methoxyphenyl](1,3-oxazol-5-ylmethyl)amino]benzoic acid, and
288) 3-[[3-cyclopropyloxy-4-difluoromethoxyphenyl](1,3-oxazol-5-ylmethyl)amino]benzoic acid,

wherein a compound listed above (in either a free base form or in the form of a pharmaceutically acceptable salt) can also be in the form of a solvate (such as a hydrate),

wherein a compound listed above (in either a free base form or in the form of a pharmaceutically acceptable salt) can also be in the form of an N-oxide,

wherein a compound listed above (in a free base form or solvate or N-oxide thereof, or in the form of a pharmaceutically acceptable salt or solvate thereof,) can also be in the form of a polymorph, and

wherein if the compound exhibits chirality it can be in the form of a mixture of enantiomers such as a racemate or a mixture of diastereomers, or can be in the form of a single enantiomer or a single diastereomer.

According to another aspect of the invention there is provided a genus of novel compounds according to the formulas VIII and IX:

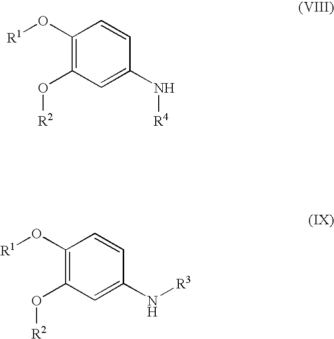

wherein R¹, R², R³, and R⁴ are as defined above. The compounds of these subgenera of formula I not only have PDE4 inhibitory activity, but may also be useful as intermediates for preparing compounds of Formula I in which R³ and R⁴ are both other than H.

Intermediate compounds according to formulas VIII and IX include:
289) 3-{[3,4-bis(difluoromethoxy)phenyl]amino}benzoic acid,
290) 2-methoxy-4-[(pyridin-3-ylmethyl)amino]benzamide,
291) 3,4-bis(difluoromethoxy)-N-(1,3-thiazol-5-ylmethyl)aniline,
292) 3,4-bis(difluoromethoxy)-N-[4-(methylsulfonyl)phenyl]aniline,
293) 4-{[4-(difluoromethoxy)-3-methoxyphenyl]amino}benzoic acid,
294) 4-{[4-(difluoromethoxy)-3-ethoxyphenyl]amino}benzoic acid, and
295) 4-{[3,4-bis(difluoromethoxy)phenyl]amino}benzoic acid.

The compounds of the present invention are effective in inhibiting, or modulating the activity of PDE4 in patients, e.g., mammals, especially humans. These compounds exhibit neurological activity, especially where such activity affects cognition, including long term memory. These compounds will also be effective in treating diseases where decreased cAMP levels are involved. This includes, but is not limited to, inflammation and inflammatory diseases. These compounds may also function as antidepressants, or be useful in treating cognitive and negative symptoms of schizophrenia.

Assays for determining PDE inhibiting activity as well as selectivity of PDE 4 inhibiting activity and selectivity of inhibiting PDE 4 isoenzymes are known within the art. See, e.g., U.S. Pat. No. 6,136,821, the disclosure of which is incorporated herein by reference.

According to a further aspect of the invention there are provided compounds useful as intermediates for the production of the PDE4 inhibitors described herein (e.g., PDE4 inhibitors of Formula I) and/or useful for the synthesis of radio-labeled analogs of the PDE4 inhibitors within this application.

Thus, there are provided intermediate compounds which correspond to compounds of Formula I, wherein R², R³, and R⁴ are as previously defined for Formula I, R¹ is R⁶, and R⁶ is H, tert-butyldimethylsilyl-, or a suitable phenolic protecting group. Suitable phenolic protecting groups are described, for example, in Greene, T. W. and Wuts, P. G. M., Protective Groups in Organic Synthesis, 3rd Edition, John Wiley & Sons, 1999, pp. 246-293. These intermediates are also useful for the synthesis of radio-labeled compounds, such as where R⁶ is 3H₃C—, 14-CH₃— or ¹¹CH₃—, for example, by removing the protecting group and reacting the resultant compound in which R⁶ is H with suitable radio-labeled reagents. Such radio-labeled compounds are useful for determining compound tissue distribution in animals, in PET imaging studies, and for in vivo, ex vivo, and in vitro binding studies.

Also provided are intermediate compounds which correspond to compounds of Formula I, wherein R¹, R³, and R⁴ are as previously defined for Formula I, R² is R⁷, and R⁷ is H, tert-butyldimethylsilyloxy-, or a suitable phenolic protecting group. Suitable phenolic protecting groups are described, for example, in Greene, T. W. and Wuts, P. G. M., Protective Groups in Organic Synthesis, 3rd Edition, John Wiley & Sons, 1999, pp. 246-293. Compounds in which R⁷ is H are useful as intermediates, for example, as scaffolds for parallel or combinatorial chemistry applications. Further, these compounds are useful for the introduction of radio-labels such as ³H, ¹⁴C, or ¹¹C.

As previously described, compounds according to Formula VIII, wherein R¹, R² and R⁴ are as previously described are useful intermediates for the production of compounds according to formula I wherein R³ is other than H.

Also, as previously described, compounds according to Formula IX, wherein R¹, R² and R³ are as previously described are useful intermediates for the production of compounds according to formula I wherein R⁴ is other than H.

Acyl refers to alkanoyl radicals having 1 to 13 carbon atoms in which the alkyl portion can be substituted by halogen, alkyl, aryl and/or alkoxy, or aroyl radicals having 7 to 15 carbon atoms in which the aryl portion can be substituted by, for example, halogen, alkyl and/or alkoxy. Suitable acyl groups include formyl, acetyl, propionyl, butanoyl and benzoyl.

Alkyl, as a group or substituent per se or as part of a group or substituent (e.g., alkylamino, trialkylsilyloxy, aminoalkyl, hydroxyalkyl), means a straight-chain or branched-chain aliphatic hydrocarbon radical having 1 to 12 carbon atoms, preferably 1 to 8 carbon atoms, especially 1 to 4 carbon atoms. Suitable alkyl groups include, but are not limited to, methyl, ethyl, propyl, isopropyl, butyl, sec-butyl, tert-butyl, pentyl, hexyl, heptyl, octyl, nonyl, decyl, undecyl, and dodecyl. Other examples of suitable alkyl groups include, but are not limited to, 1-, 2- or 3-methylbutyl, 1,1-, 1,2- or 2,2-dimethylpropyl, 1-ethylpropyl, 1-, 2-, 3- or 4-methylpentyl, 1,1-, 1,2-, 1,3-, 2,2-, 2,3- or 3,3-dimethylbutyl, 1- or 2-ethylbutyl, ethylmethylpropyl, trimethylpropyl, methylhexyl, dimethylpentyl, ethylpentyl, ethylmethylbutyl, dimethylbutyl, and the like.

Substituted alkyl groups are alkyl groups as described above which are substituted in one or more positions by, for example, halogens, oxo, hydroxyl, C₁₋₄-alkoxy, and/or cyano. Halogens are preferred substituents, especially F and Cl.

Alkenyl refers to straight-chain or branched-chain aliphatic radicals containing 2 to 12 carbon atoms in which one or more —CH₂—CH₂— structures are each replaced by —CH═CH—. Suitable alkenyl groups include, but are not limited to, ethenyl, 1-propenyl, 2-methylethenyl, 1-butene, 2-butene, 1-pentenyl, and 2-pentenyl.

Alkynyl refers to straight-chain or branched-chain aliphatic radicals containing 2 to 12 carbon atoms in which one or more —CH₂—CH₂— structures are each replaced by —C≡C—. Suitable alkynyl groups include, but are not limited to, ethynyl, propynyl, 1-butynyl, and 2-butynyl.

Alkoxy means alkyl-O— groups and alkoxyalkoxy means alkyl-O-alkyl-O— groups in which the alkyl portions are in accordance with the previous discussion. Suitable alkoxy and alkoxyalkoxy groups include, but are not limited to, methoxy, ethoxy, propoxy, butoxy, pentoxy, hexoxy, heptoxy, octoxy, methoxymethoxy, ethoxymethoxy, propoxymethoxy, and methoxyethoxy. Preferred alkoxy groups are methoxy and ethoxy. Similarly, alkoxycarbonyl means alkyl —O—CO— in which the alkyl portion is in accordance with the previous discussion. Examples include, but are not limited to, methoxycarbonyl, ethoxycarbonyl, propoxycarbonyl, and tert-butoxycarbonyl.

Aryl, as a group or substituent per se or as part of a group or substituent, refers to an aromatic carbocyclic radical containing 6 to 14 carbon atoms, preferably 6 to 12 carbon atoms, especially 6 to 10 carbon atoms. Suitable aryl groups include, but are not limited to, phenyl, naphthyl and biphenyl. Substituted aryl groups include the above-described aryl groups which are substituted one or more times by, for example, halogen, alkyl, fluorinated alkyl, hydroxy, alkoxy, fluorinated alkoxy (e.g., OCF₃, OCHF₂), nitro, methylenedioxy, ethylenedioxy, amino, alkylamino, dialkylamino, hydroxyalkyl, hydroxyalkoxy, carboxy, cyano, acyl, alkoxycarbonyl, alkylthio, alkylsulfinyl, alkylsulfonyl, and phenoxy.

Arylalkyl refers to an aryl-alkyl-radical in which the aryl and alkyl portions are in accordance with the previous descriptions. Suitable examples include, but are not limited to, benzyl, 1-phenethyl, 2-phenethyl, phenpropyl, phenbutyl, phenpentyl, and napthylmethyl.

Substituted arylalkyl refers to an aryl-alkyl-group wherein the aryl and alkyl portions are in accordance with the previous discussions.

Cycloalkyl means a monocyclic, bicyclic or tricyclic nonaromatic saturated hydrocarbon radical having 3 to 10 carbon atoms, preferably 3 to 8 carbon atoms, especially 3 to 6 carbon atoms. Suitable cycloalkyl groups include, but are not limited to, cyclopropyl, cyclobutyl, cyclopentyl, cyclohexyl, cycloheptyl, cyclooctyl, norbornyl, 1-decalin, adamant-1-yl, and adamant-2-yl. Other suitable cycloalkyl groups include, but are not limited to, spiropentyl, bicyclo[2.1.0]pentyl, bicyclo[3.1.0]hexyl, spiro[2.4]heptyl, spiro[2.5]octyl, bicyclo[5.1.0]octyl, spiro[2.6]nonyl, bicyclo[2.2.0]hexyl, spiro[3.3]heptyl, bicyclo[4.2.0]octyl, and spiro[3.5]nonyl. Preferred cycloalkyl groups are cyclopropyl, cyclobutyl, cyclopentyl and cyclohexyl. The cycloalkyl group can be substituted, for example, by one or more halogens and/or alkyl groups.

Cycloalkylalkyl refers to cycloalkyl-alkyl radicals in which the cycloalkyl and alkyl portions are in accordance with previous discussions. Suitable examples include, but are not limited to, cyclopropylmethyl and cyclopentylmethyl.

Halogen herein refers to F, Cl, Br, and I. Preferred halogens are F and Cl.

Heteroaryl refers to an aromatic heterocyclic group having one or two rings and a total number of 5 to 10 ring atoms wherein at least one of the ring atoms is a heteroatom. Preferably, the heteroaryl group contains 1 to 3, especially 1 or 2, hetero-ring atoms which are selected from N, O and S. Suitable heteroaryl groups include, but are not limited to, furyl, thienyl, pyrrolyl, pyrazolyl, imidazolyl, triazolyl, tetrazolyl, isoxazolyl, oxazolyl, thiazolyl, isothiazolyl, oxadiazolyl, oxatriazolyl, thiadiazolyl, pyridyl, pyridazinyl, pyrimidinyl, pyrazinyl, triazinyl, benzofuranyl, isobenzofuranyl, thionaphthenyl, isothionaphthenyl, indolyl, isoindolyl, indazolyl, benzisoxazolyl, benzoxazolyl, benzthiazolyl, benzisothiazolyl, purinyl, benzopyranyl, quinolinyl, isoquinolinyl, cinnolinyl, quinazolinyl, naphthyridinyl, and benzoxazinyl, e.g., 2-thienyl, 3-thienyl, 2-, 3- or 4-pyridyl, 2-, 3-, 4-, 5-, 6-, 7- or 8-quinolinyl, and 1-, 3-, 4-, 5-, 6-, 7- or 8-isoquinolinyl.

Substituted heteroaryl refers to the heteroaryl groups described above which are substituted in one or more places by, for example, halogen, aryl, alkyl, alkoxy, carboxy, methylene, cyano, trifluoromethyl, nitro, oxo, amino, alkylamino, and dialkylamino.

Heterocycles include heteroaryl groups as described above as well as non-aromatic cyclic groups (i.e., saturated and partially saturated heterocyclic groups) containing at least one hetero-ring atom, preferably selected from N, S and O, for example, but not limited to, tetrahydrofuranyl, piperidinyl, dithianyl, oxathianyl, dioxazolyl, oxathiazolyl, oxazinyl, isoxazinyl, oxathiazinyl, oxadiazinyl, and pyrrolidinyl.

Heterocycle-alkyl refers to a heterocyclic group which is attached to the remainder of the molecule via an alkyl bridge group wherein the heterocyclic and alkyl portions are in accordance with the previous discussions. Suitable examples include, but are not limited to, pyridylmethyl, thiazolylmethyl, thienylmethyl, pyrimidinylmethyl, pyrazinylmethyl, and isoquinolinylmethyl.

Partially unsaturated carbocyclic structures are non-aromatic monocyclic or bicyclic structures containing 5 to 14 carbon atoms, preferably 6 to 10 carbon atoms, wherein the ring structure(s) contains at least one C═C bond. Suitable examples include, but are not limited to, cyclopentenyl, cyclohexenyl, cyclohexadienyl, tetrahydronaphthenyl and indan-2-yl.

Substituted radicals preferably have 1 to 3 substituents, especially 1 to 2 substituents.

Additional aspects of the present invention include pharmaceutical compositions comprising a compound of Formulae I-VII and a pharmaceutically acceptable carrier and, optionally, another active agent as discussed below; a method of inhibiting a PDE4 enzyme, especially an isoenzyme, e.g., as determined by a conventional assay or one described herein, either in vitro or in vivo (in an animal, e.g., in an animal model, or in a mammal or in a human); a method of treating neurological syndrome, e.g., loss of memory, especially long-term memory, cognitive impairment or decline, memory impairment, etc. a method of treating a disease state modulated by PDE4 activity, in a mammal, e.g., a human, e.g., those mentioned herein.

The compounds of the present invention may be prepared conventionally. Some of the processes which can be used are described below. All starting materials are known or can be conventionally prepared from known starting materials.

The reaction schemes shown below are for illustrative purposes only and should not be viewed as limiting the scope of the synthetic methods available for the production of the compounds described within this application. Note that alternative methods, reagents, solvents, bases, acids etc., which are considered standard in the art, can be utilized in addition or can rep

Starting nitrophenols of the type 1 are either commercially available (e.g., R¹═CH₃) or prepared by published procedures (e.g., R¹═CHF₂ or both R¹ and R²═CHF₂, see Mueller, Klaus-Helmut. Eur. Pat. Appl. (1994), 8 pp. CODEN: EPXXDW EP 626361A1; Touma, Toshihiko; Asai, Tomoyuki. Jpn. Kokai Tokyo Koho (1999), 6 pp. CODEN: JKXXAF JP 11071319 A2; Platonov, Andrew; Seavakov, Andrew; Maiyorova, Helen; Chistokletov, Victor. Int. Symp. Wood. Pulping Chem., 1995, 8th, 3, 295-299; Christensen, Siegfried Benjamin; Dabbs, Steven; Karpinski, Joseph M. PCT Int. Appl. (1996), 12 pp. CODEN: PIXXD2 WO 9623754 A1 19960808). For example, as seen in Scheme 1, aniline intermediates 3 are produced in two steps; first, an addition reaction provides intermediate 2, followed by reduction of the nitro group. Intermediate nitro compounds 2 can be prepared by numerous published procedures, such as by Mitsunobu reactions or standard alkylation reactions. Compounds where R₂ is aryl or heteroaryl can be prepared by copper catalyzed reactions with aryl or heteroaryl iodides under Ullman conditions or by coupling aryl-, vinyl-, or heteroaryl-boronic acids with phenol 2 in the presence of a copper catalyst (e.g., Cu(OAc)₂) and base such as TEA. Mitsunobu reaction between an appropriately substituted nitrophenol and a primary or secondary alcohol using an azodicarboxylate (e.g., DEAD, DIAD), and a suitable phosphine (e.g., Ph₃P, Bu₃P) provides nitroaryl ethers of structure 2. Mitsunobu reactions are general performed in aprotic solvents such as dichloromethane or THF. Alternatively, alkylation can be achieved by the reaction between an appropriately substituted nitrophenol and an alkyl halide in the presence of a base (e.g., K₂CO₃ or NaH) in a polar aprotic solvent (e.g., DMF or CH₃CN).

Nitrocatechols 2 are subsequently reduced to the corresponding anilines 3 by methods standard in the art such as by hydrogenation using a suitable catalyst (e.g., Pd on carbon) in a polar protic solvent (e.g., MeOH or EtOH) under an atmosphere of hydrogen. Alternatively, nitrocatechols 3 can be reduced by using a hydride source (e.g., NaBH₄) and a transition metal catalyst (e.g., NiCl₂, Pd on carbon) or by using metals (e.g., Zn, Sn, Fe) in mineral acid solutions (e.g., HCl) to produce the corresponding anilines. Generally polar protic solvents such as ethanol or methanol are used in these reactions.

N-Arylalkylanilines 4 are synthesized by standard methods in the art such as by reductive amination reaction, alkylation reaction, or by reduction of corresponding amides. For example, the reductive amination reaction of an aryl or arylalkyl aldehyde with appropriately substituted anilines in the presence of a borohydride reducing agent such as NaBH₄, NaBH₃CN or Na(OAc)₃BH with an acid catalyst such as acetic acid or pTsOH provides desired N-arylalkylanilines. These reactions generally take place in polar protic solvents such as methanol, ethanol, isopropanol, n-propanol and the like, or in chlorinated solvents such as dichloroethane.

N-Arylalkylanilines 4 readily undergo N-arylation by methods standard to the art including Ullman coupling reaction, metal-catalyzed coupling, or aromatic nucleophilic substitution reaction. For example, the metal catalyzed reaction between an N-benzylaniline and an aryl halide using a palladium catalyst, (e.g., Pd₂(dba)_{z}, a bulky electron rich phosphine ligand (e.g., tributylphosphine or “X-Phos”), and suitable base (e.g., NaOtBu, NaOH, Cs₂CO₃) provides N-Arylalkyldiphenylamines. Nickel and copper catalysts have been employed as well. Solvents useful in this reaction include non-polar aprotic solvents such as toluene, benzene, xylenes, tetrahydrofuran, ether, dimethoxyethane. When synthesizing compounds of the type 5 wherein R₄ is an alkoxycarbonylphenyl, it is advantageous that amine 4 is coupled with 1.1 equivalents of tert-butyl 3-iodobenzene and that 22 mol % of (tBu)₃P, 5.5 mol % of Pd₂(dba)₃ and 1.3 equivalents of tBuONa are used.

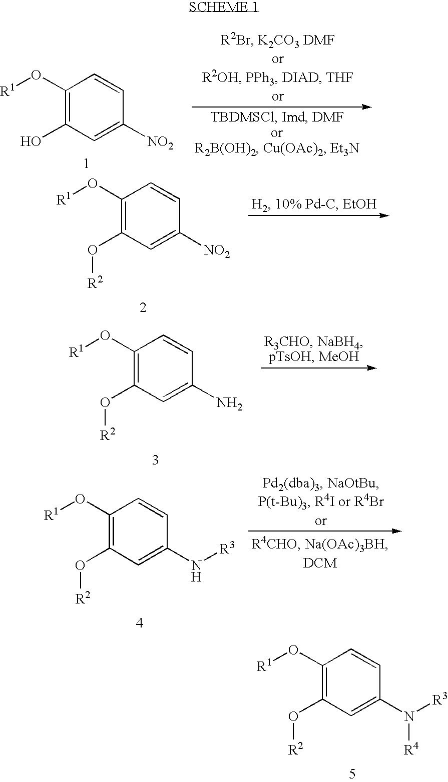

Alternatively, targets of general formula I, represented as structure 5 in the following Scheme 2A, can be prepared by N-arylation reaction between catechol ether halides of general structure 7 and N-substituted anilines of general structure 9 under conditions standard to the art (see Scheme 1). Intermediates 7 and 9 can be synthesized by numerous methods standard in the art, or as represented in Scheme 2.

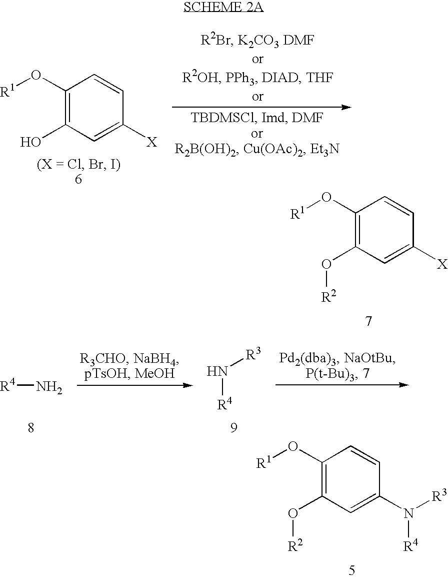

Carboxylic ester intermediates 10 can be hydrolyzed under acidic or basic conditions to give the corresponding carboxylic acids 7, as shown in Scheme 2B. For example, an ethyl ester (R⁵=Et, Me) can be hydrolyzed using a mixture of aqueous base (e.g., NaOH, KOH, LiOH) and a water miscible solvent (e.g., EtOH, THF). While t-butyl esters (R⁵=t-butyl) can be hydrolyzed using an aqueous acid (e.g., HCl, formic acid, TFA) in a water miscible organic solvent. If necessary, microwave heating can be used.

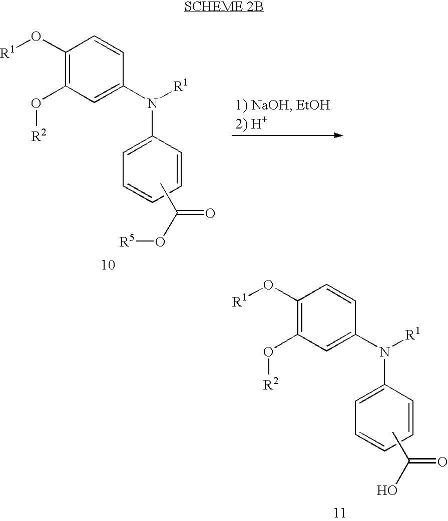

Coupling of protected tetrazole bromo or iodobenzenes (e.g., 5-(3-iodophenyl)-2-(2-tetrahydropyran)tetrazole) with N-substituted aniline derivatives 9 produce THP-protected tetrazoles 12. As shown in Scheme 3, hydrolysis of THP-protected tetrazoles 12 can be accomplished by using an aqueous acid, such as HCl in water and a miscible solvent such as THF or EtOH to provide tetrazoles 12. Further, THP tetrazoles 12 can also be oxidatively cleaved using reagents such as CAN and DDQ in halogenated hydrocarbon solvents such as dichloromethane, chloroform, dichloroethane and the like to yield tetrazoles 13.

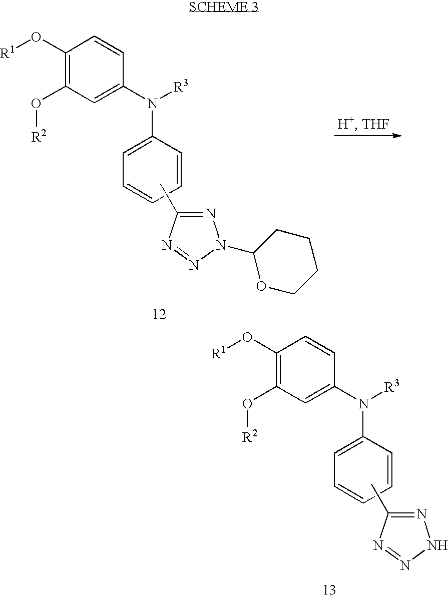

Alternatively, tetrazole analogs 13 can be prepared from the corresponding nitriles by treatment with azide ion (e.g., KN₃, NaN₃, etc.) and a proton source (e.g., NH₄Cl) in a polar aprotic solvent such as DMF. They also may be prepared by treatment with an azide ion and a Lewis acid (e.g., ZnBr₂) in water, using a water miscible co-solvent such as isopropanol if necessary. Another method of preparation is by treatment of a nitrile with tin or silicon azides (e.g., Me₃SiN3, Bu₃SnN₃) in an aprotic organic solvent such as benzene, toluene, dichloromethane, dichloroethane, ether, THF, and the like.

As illustrated in Scheme 4, diphenylamines 14 can be prepared by coupling appropriately substituted anilines 3, such as 3-cyclopentyloxy-4-methoxyaniline, with arylboronic acids in the presence of a base such as triethylamine and a copper catalyst such as copper acetate (as described by Chan et al, Tetrahedron Lett., 39, 2933-2936 (1998)). In general, halogenated solvents such as dichloromethane, chloroform, dichloroethane, and the like as well as nonpolar aprotic solvents such as benzene, toluene, or xylene are utilized. Such diphenylamines (e.g., 14) can more preferably be synthesized by metal catalyzed amination reactions. For example, reaction of an appropriately substituted aniline 3 with an arylhalide in the presence of a base (e.g., K₃PO₄, CsCO₃, or NaOtBu) and a palladium or nickel catalyst, for example Pd(dppf)Cl₂, a ligand (e.g., dppf) and a base (e.g., NaOtBu) (JACS. 1996, 118, 7217) or with Pd₂ dba₃, a bulky electron rich phosphine such as P(tBu)₃, and a base (e.g., NaOtBu) (J. Org. Chem. 1999, 64, 5575) provides the desired diphenylamines 14. Solvents most commonly utilized in this type of reaction include non-polar aprotic solvents such as benzene, toluene, tetrahydrofuran, ether, and the like.

Diphenylamines 14 can then be alkylated with various alkyl halides or arylalkyl halides such as, but not limited to iodomethane, ethylbromide, benzylchloride, 3-(chloromethyl)pyridine, 4-(chloromethyl)-2,6-dichloropyridine, and 4-(bromomethyl)-benzoic acid, or salts thereof, in the presence of a non-nucleophilic base such as sodium hydride, potassium hexamethyldisilazide or potassium diisopropylamide to provide N-substituted diphenylamines 5. Solvents useful in this reaction include aprotic solvents such as benzene, toluene, tetrahydrofuran, ether, DMF, and the like.

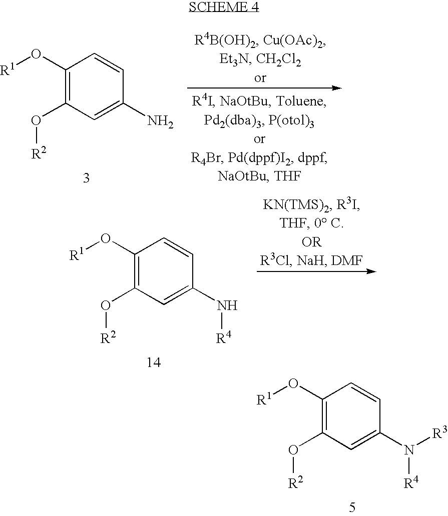

Carboxylic acids 15 can be further manipulated to form carboxamides 16 using methods standard in the art. For example, as shown in Scheme 5, a carboxylic acid can be treated with a suitable primary or secondary amine, in the presence of a suitable coupling reagent such as BOP, pyBOP or DCC, and a base such as Et₃N or DIEA to yield a carboxamide. These reactions generally take place in non-polar aprotic solvents such as dichloromethane, chloroform, or dichloroethane.

Carboxylic esters 10 or acids 15 can be reduced using methods standard in the art to give the corresponding carboxaldehyde or hydroxymethyl analogs. For example, an aryl ethyl ester (e.g., Scheme 2 structure 10, R=ethyl) can be treated with an appropriate reducing agent (e.g., LAH, DIBAL, etc.) in an aprotic solvent such as ether or THF, to produce the corresponding carboxaldehydes or hydroxymethyl analogs. Such aldehydes and alcohols can be further derivatized by methods standard in the art.

Similarly carboxamides (e.g., structure 16) and nitriles can be reduced using methods standard in the art to provide the corresponding substituted amines or aminomethyl analogs. For example, an aryl carboxamide 16 can be reduced with an appropriate reducing agent (e.g., LAH) in an aprotic solvent (e.g., benzene, toluene, ether, THF, etc.) to give the corresponding substituted aminomethyl analog. Whereas reduction of an aryl nitrile yields the corresponding primary aminomethyl analog.

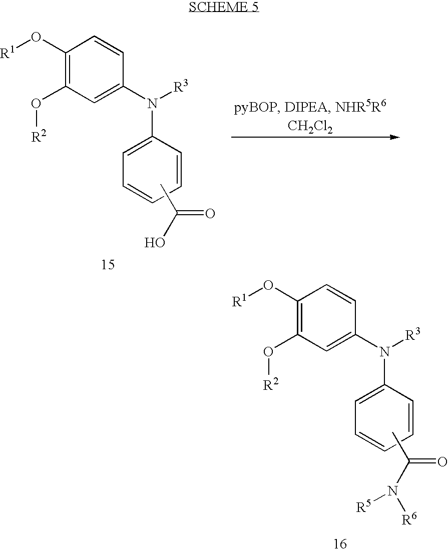

Nitrobenzene compounds 17 can be reduced to the corresponding anilines 13 by methods standard in the art such as hydrogenation using a suitable catalyst (e.g., Pd on carbon) in a polar protic solvent (e.g., EtOH, MeOH, etc.). Nitrobenzenes 17 can also be reduced using a hydride source (e.g., NaBH₄) and a transition metal catalyst (e.g., NiCl₂, Pd on carbon) in polar protic solvents such as EtOH, to produce the corresponding anilines 18, as shown in Scheme 6. These anilines can then be further substituted by methods standard in the art. For example, anilines of the type 13 can be alkylated, acylated, or sulfonylated to give the corresponding N-alkyl amines, carboxamides (e.g., structure 20) or sulfonamides (e.g., structure 19) respectively. For example, a sulfonamide can be prepared from an aniline and an appropriate sulfonyl halide or sulfonic anhydride (e.g., MeSO₂Cl, EtSO₂Cl, BnSO₂Cl, PhSO₂Cl, etc.) in the presence of a base (e.g., Et₃N, pyridine, DIEA, etc.). Suitable solvents for this reaction include non-polar aprotic solvents such as dichloromethane, chloroform, ether, and the like.

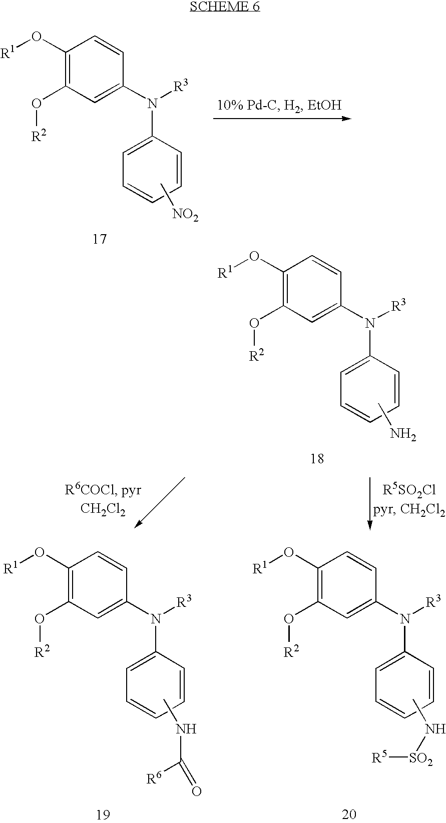

Trialkylsilylethers of the type 21 (Scheme 1, structure 5, where R²=TBDMS) are prepared in the manner shown in Scheme 1. The tert-butyldimethylsilyl protected catechol intermediates 21 are readily deprotected by numerous literature methods (see Greene, T. W. and Wuts, P. G. M., Protective Groups in Organic Synthesis, 3rd Edition, John Wiley & Sons, 1999, pp. 273-276.) such as by using a fluoride ion source (e.g., Bu₄NF) in an aprotic solvent such as ether or THF; or under acidic conditions (e.g., KF, 48% HBr, DMF), as illustrated in Scheme 7. The resultant phenol 22, which is a very useful synthetic intermediate, can then be alkylated by methods standard in the art and in a similar manner as described for the alkylation of nitrophenol 2 in Scheme 1, and further illustrated in Scheme 7. For example, by the Mitsunobu reaction, by reaction with an alkyl halide in the presence of a base, or by Ullman type aryl coupling or by reaction with vinyl-, aryl- or heteroaryl-boronic acids in the presence of a copper catalyst.

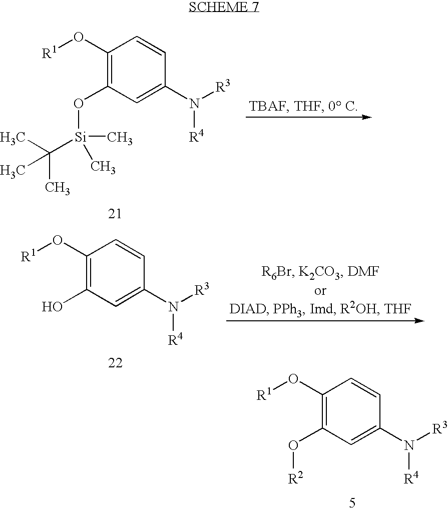

Haloalkoxy intermediates 23, prepared by alkylation of the corresponding phenol, can be alkylated by reactions with substituted amines, alcohols, or thiols in the presence of a base to provide analogs such as 24, as illustrated in Scheme 8. For example, an alkyl halide can be aminated with an appropriate primary or secondary amine and a base such as K₂CO₃, in a polar aprotic solvent such as THF, DMF, or CH₃CN.

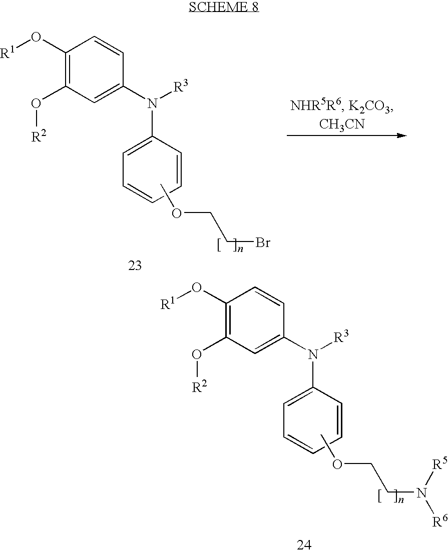

Many of these synthetic procedures are described more fully in the examples set forth below.

One of ordinary skill in the art will recognize that some of the compounds of Formulae I-IX, including the specific compounds listed above, can exist in different geometrical isomeric forms. In addition, some of the compounds of the present invention possess one or more asymmetric carbon atoms and are thus capable of existing in the form of optical isomers, as well as in the form of racemic or nonracemic mixtures thereof, and in the form of diastereomers and diastereomeric mixtures inter alia. All of these compounds, including cis isomers, trans isomers, diastereomic mixtures, racemates, nonracemic mixtures of enantiomers, and substantially pure and pure enantiomers, are within the scope of the present invention. Substantially pure enantiomers contain no more than 5% w/w of the corresponding opposite enantiomer, preferably no more than 2%, most preferably no more than 1%.

The optical isomers can be obtained by resolution of the racemic mixtures according to conventional processes, for example, by the formation of diastereoisomeric salts using an optically active acid or base or formation of covalent diastereomers. Examples of appropriate acids include tartaric, diacetyltartaric, dibenzoyltartaric, ditoluoyltartaric and camphorsulfonic acid. Mixtures of diastereoisomers can be separated into their individual diastereomers on the basis of their physical and/or chemical differences by methods known to those skilled in the art, for example, by chromatography or fractional crystallization. The optically active bases or acids are then liberated from the separated diastereomeric salts. A different process for separation of optical isomers involves the use of chiral chromatography (e.g., chiral HPLC columns), with or without conventional derivation, optimally chosen to maximize the separation of the enantiomers. Suitable chiral HPLC columns are manufactured by Diacel, e.g., Chiracel OD and Chiracel OJ among many others, all routinely selectable. Enzymatic separations, with or without derivitization, are also useful. The optically active compounds of Formulae I-IX can likewise be obtained by chiral syntheses utilizing optically active starting materials.

In addition, one of ordinary skill in the art will recognize that the compounds can be used in different enriched isotopic forms, e.g., enriched in the content of ²H, ³H, ¹¹C and/or ¹⁴C.

The present invention also relates to useful forms of the compounds as disclosed herein, such as pharmaceutically acceptable salts and prodrugs of all the compounds of the present invention. Pharmaceutically acceptable salts include those obtained by reacting the main compound, functioning as a base, with an inorganic or organic acid to form a salt, for example, salts of hydrochloric acid, sulfuric acid, phosphoric acid, methane sulfonic acid, camphor sulfonic acid, oxalic acid, maleic acid, succinic acid and citric acid. Pharmaceutically acceptable salts also include those in which the main compound functions as an acid and is reacted with an appropriate base to form, e.g., sodium, potassium, calcium, magnesium, ammonium, and choline salts. Those skilled in the art will further recognize that acid addition salts of the claimed compounds may be prepared by reaction of the compounds with the appropriate inorganic or organic acid via any of a number of known methods. Alternatively, alkali and alkaline earth metal salts are prepared by, for example, reacting a compound of the invention with the appropriate base via a variety of known methods.

The following are further non-limiting examples of acid salts that can be obtained by reaction with inorganic or organic acids: acetates, adipates, alginates, citrates, aspartates, benzoates, benzenesulfonates, bisulfates, butyrates, camphorates, digluconates, cyclopentanepropionates, dodecylsulfates, ethanesulfonates, glucoheptanoates, glycerophosphates, hemisulfates, heptanoates, hexanoates, fumarates, hydrobromides, hydroiodides, 2-hydroxy-ethanesulfonates, lactates, maleates, methanesulfonates, nicotinates, 2-naphthalenesulfonates, oxalates, palmoates, pectinates, persulfates, 3-phenylpropionates, picrates, pivalates, propionates, succinates, tartrates, thiocyanates, tosylates, mesylates and undecanoates.

For example, the pharmaceutically acceptable salt can be a hydrochloride, a hydrobromide, a hydroformate, or a maleate.

With regards to pharmaceutically acceptable salts, see also the list of FDA approved commercially marketed salts listed in Table I of Berge et al., Pharmaceutical Salts, J. of Phar. Sci., vol. 66, no. 1, January 1977, pp. 1-19, the disclosure of which is incorporated herein by reference.

Preferably, the salts formed are pharmaceutically acceptable for administration to mammals. However, pharmaceutically unacceptable salts of the compounds are suitable as intermediates, for example, for isolating the compound as a salt and then converting the salt back to the free base compound by treatment with an alkaline reagent. The free base can then, if desired, be converted to a pharmaceutically acceptable acid addition salt.

One of ordinary skill in the art will also recognize that some of the compounds of Formulae I-IX can exist in different polymorphic forms. As known in the art, polymorphism is an ability of a compound to crystallize as more than one distinct crystalline or “polymorphic” species. A polymorph is a solid crystalline phase of a compound with at least two different arrangements or polymorphic forms of that compound molecule in the solid state. Polymorphic forms of any given compound are defined by the same chemical formula or composition and are as distinct in chemical structure as crystalline structures of two different chemical compounds.

One of ordinary skill in the art will further recognize that compounds of Formulae I-IX can exist in different solvate forms. Solvates of the compounds of the invention may also form when solvent molecules are incorporated into the crystalline lattice structure of the compound molecule during the crystallization process.

The compounds of the invention can be administered alone or as an active ingredient of a formulation. Thus, the present invention also includes pharmaceutical compositions of compounds of Formulae I-VII, and the specific compounds listed above, containing, for example, one or more pharmaceutically acceptable carriers.

Numerous standard references are available that describe procedures for preparing various formulations suitable for administering the compounds according to the invention. Examples of potential formulations and preparations are contained, for example, in the Handbook of Pharmaceutical Excipients, American Pharmaceutical Association (current edition); Pharmaceutical Dosage Forms: Tablets (Lieberman, Lachman and Schwartz, editors) current edition, published by Marcel Dekker, Inc., as well as Remington's Pharmaceutical Sciences (Arthur Osol, editor), 1553-1593 (current edition).

In view of their high degree of PDE4 inhibition, the compounds of the present invention can be administered to anyone requiring or desiring PDE4 inhibition, and/or enhancement of cognition. Administration may be accomplished according to patient needs, for example, orally, nasally, parenterally (subcutaneously, intravenously, intramuscularly, intrasternally and by infusion), by inhalation, rectally, vaginally, topically, locally, transdermally, and by ocular administration.

Various solid oral dosage forms can be used for administering compounds of the invention including such solid forms as tablets, gelcaps, capsules, caplets, granules, lozenges and bulk powders. The compounds of the present invention can be administered alone or combined with various pharmaceutically acceptable carriers, diluents (such as sucrose, mannitol, lactose, starches) and excipients known in the art, including but not limited to suspending agents, solubilizers, buffering agents, binders, disintegrants, preservatives, colorants, flavorants, lubricants and the like. Time release capsules, tablets and gels are also advantageous in administering the compounds of the present invention.

Various liquid oral dosage forms can also be used for administering compounds of the invention, including aqueous and non-aqueous solutions, emulsions, suspensions, syrups, and elixirs. Such dosage forms can also contain suitable inert diluents known in the art such as water and suitable excipients known in the art such as preservatives, wetting agents, sweeteners, flavorants, as well as agents for emulsifying and/or suspending the compounds of the invention. The compounds of the present invention may be injected, for example, intravenously, in the form of an isotonic sterile solution. Other preparations are also possible.

Suppositories for rectal administration of the compounds of the present invention can be prepared by mixing the compound with a suitable excipient such as cocoa butter, salicylates and polyethylene glycols. Formulations for vaginal administration can be in the form of a pessary, tampon, cream, gel, paste, foam, or spray formula containing, in addition to the active ingredient, such suitable carriers as are known in the art.

For topical administration the pharmaceutical composition can be in the form of creams, ointments, liniments, lotions, emulsions, suspensions, gels, solutions, pastes, powders, sprays, and drops suitable for administration to the skin, eye, ear or nose. Topical administration may also involve transdermal administration via means such as transdermal patches.

Aerosol formulations suitable for administering via inhalation also can be made. For example, for treatment of disorders of the respiratory tract, the compounds according to the invention can be administered by inhalation in the form of a powder (e.g., micronized) or in the form of atomized solutions or suspensions. The aerosol formulation can be placed into a pressurized acceptable propellant.

The present invention further includes methods of treatment that involve inhibition of PDE4 enzymes. Thus, the present invention includes methods of selective inhibition of PDE4 enzymes in animals, e.g., mammals, especially humans, wherein such inhibition has a therapeutic effect, such as where such inhibition may relieve conditions involving neurological syndromes, such as the loss of memory, especially long-term memory. Such methods comprise administering to a patient in need thereof, especially a mammal, most especially a human, an inhibitory amount of a compound, alone or as part of a formulation, as disclosed herein.

The condition of memory impairment is manifested by impairment of the ability to learn new information and/or the inability to recall previously learned information. Memory impairment is a primary symptom of dementia and can also be a symptom associated with such diseases as Alzheimer's disease, schizophrenia, Parkinson's disease, Huntington's disease, Pick's disease, Creutzfeldt-Jakob disease, HIV, cardiovascular disease, and head trauma as well as age-related cognitive decline.

Dementias are diseases that include memory loss and additional intellectual impairment separate from memory. The present invention includes methods for treating patients suffering from memory impairment in all forms of dementia. Dementias are classified according to their cause and include: neurodegenerative dementias (e.g., Alzheimer's, Parkinson's disease, Huntington's disease, Pick's disease), vascular (e.g., infarcts, hemorrhage, cardiac disorders), mixed vascular and Alzheimer's, bacterial meningitis, Creutzfeldt-Jacob Disease, multiple sclerosis, traumatic (e.g., subdural hematoma or traumatic brain injury), infectious (e.g., HIV), genetic (down syndrome), toxic (e.g., heavy metals, alcohol, some medications), metabolic (e.g., vitamin B12 or folate deficiency), CNS hypoxia, Cushing's disease, psychiatric (e.g., depression and schizophrenia), and hydrocephalus.

The present invention includes methods for dealing with memory loss separate from dementia, including mild cognitive impairment (MCI) and age-related cognitive decline. The present invention includes methods of treatment for memory impairment as a result of disease. In another application, the invention includes methods for dealing with memory loss resulting from the use of general anesthetics, chemotherapy, radiation treatment, post-surgical trauma, and therapeutic intervention.

The compounds of the present invention may be used to treat psychiatric conditions including schizophrenia, bipolar or manic depression, major depression, and drug addiction and morphine dependence. These compounds may enhance wakefulness. PDE4 inhibitors can be used to raise cAMP levels and prevent neurons from undergoing apoptosis. PDE4 inhibitors are also known to be anti-inflammatory. The combination of anti-apoptotic and anti-inflammatory properties make these compounds useful to treat neurodegeneration resulting from any disease or injury, including stroke, spinal cord injury, Alzheimer's disease, multiple sclerosis, amylolaterosclerosis (ALS), and multiple systems atrophy (MSA).

Thus, in accordance with a preferred embodiment, the present invention includes methods of treating patients suffering from memory impairment due to, for example, Alzheimer's disease, multiple sclerosis, amylolaterosclerosis (ALS), multiple systems atrophy (MSA), schizophrenia, Parkinson's disease, Huntington's disease, Pick's disease, Creutzfeldt-Jakob disease, Rubenstein-Taybi syndrome (RSTS), depression, aging, head trauma, stroke, spinal cord injury, CNS hypoxia, cerebral senility, diabetes associated cognitive impairment, memory deficits from early exposure of anesthetic agents, multiinfarct dementia and other neurological conditions including acute neuronal diseases, as well as HIV and cardiovascular diseases, comprising administering an effective amount of a compound according to Formulae I-VII, and the specific compounds listed above, or pharmaceutically acceptable salts thereof.

The compounds of the present invention can also be used in a method of treating patients suffering from disease states characterized by decreased NMDA function, such as schizophrenia. The compounds can also be used to treat psychosis characterized by elevated levels of PDE 4, for example, various forms of depression, such as manic depression, major depression, and depression associated with psychiatric and neurological disorders.

The compounds of the present invention can also be used in methods of treating patients suffering from obesity and in treatment methods for neuronal regeneration or neurogenesis.

A subject or patient in whom administration of the therapeutic compound is an effective therapeutic regimen for a disease or disorder is preferably a human, but can be any animal, including a laboratory animal in the context of a clinical trial or screening or activity experiment. Thus, as can be readily appreciated by one of ordinary skill in the art, the methods, compounds and compositions of the present invention are particularly suited to administration to any animal, particularly a mammal, and including, but by no means limited to, humans, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., avian species, such as chickens, turkeys, songbirds, etc., i.e., for veterinary medical use.

As mentioned, the compounds of the invention also exhibit anti-inflammatory activity. As a result, the inventive compounds are useful in the treatment of a variety of allergic and inflammatory diseases, and the inflammation associated therewith, particularly disease states characterized by decreased cyclic AMP levels and/or elevated phosphodiesterase 4 levels. Thus, in accordance with a further embodiment of the invention, there is provided a method of treating allergic and inflammatory disease states, comprising administering an effective amount of a compound according to Formulae I-VII, and the specific compounds listed above, or a pharmaceutically acceptable salt thereof. Such disease states include: asthma, chronic bronchitis, chronic obstructive pulmonary disease (COPD), atopic dermatitis, urticaria, allergic rhinitis, allergic conjunctivitis, vernal conjunctivitis, esoniophilic granuloma, psoriasis, inflammatory arthritis, rheumatoid arthritis, septic shock, ulcerative colitis, Crohn's disease, reperfusion injury of the myocardium and brain, chronic glomerulonephritis, endotoxic shock, adult respiratory distress syndrome, cystic fibrosis, arterial restenosis, artherosclerosis, keratosis, rheumatoid spondylitis, osteoarthritis, pyresis, diabetes mellitus, pneumoconiosis, chronic obstructive airways disease, chronic obstructive pulmonary disease, toxic and allergic contact eczema, atopic eczema, seborrheic eczema, lichen simplex, sunburn, pruritis in the anogenital area, alopecia greata, hypertrophic scars, discoid lupus erythematosus, systemic lupus erythematosus, follicular and wide-area pyodermias, endogenous and exogenous acne, acne rosacea, Behcet's disease, anaphylactoid purpura nephritis, inflammatory bowel disease, leukemia, multiple sclerosis, gastrointestinal diseases, autoimmune diseases and the like.

PDE4 inhibitors for treating asthma, chronic bronchitis, psoriasis, allergic rhinitis, and other inflammatory diseases, and for inhibiting tumor necrosis factor are known within the art. See, e.g., WO 98/58901, JP11-18957, JP 10-072415, WO 93/25517, WO 94/14742, U.S. Pat. No. 5,814,651, and U.S. Pat. No. 5,935,978. These references also describe assays for determining PDE4 inhibition activity, and methods for synthesizing such compounds. The entire disclosures of these documents are hereby incorporated by reference.

PDE4 inhibitors may be used to prevent or ameliorate osteoporosis, as an antibiotic, for treatment of cardiovascular disease by mobilizing cholesterol from atherosclerotic lesions, to treat rheumatoid arthritis (RA), for long-term inhibition of mesenchymal-cell proliferation after transplantation, for treatment of urinary obstruction secondary to benign prostatic hyperplasia, for suppression of chemotaxis and reduction of invasion of colon cancer cells, for treatment of B cell chronic lymphocytic leukemia (B-CLL), for inhibition of uterine contractions, to attenuate pulmonary vascular ischemia-reperfusion injury (IRI), for corneal hydration, for inhibition of IL-2R expression and thereby abolishing HIV-1 DNA nuclear import into memory T cells, for augmentation of glucose-induced insulin secretion, in both the prevention and treatment of colitis, and to inhibit mast cell degranulation.

The compounds of Formulae I-VII, and the specific compounds listed above, can be administered as the sole active agent or in combination with other pharmaceutical agents such as other agents used in the treatment of cognitive impairment and/or in the treatment of psychosis, e.g., other PDE4 inhibitors, calcium channel blockers, cholinergic drugs, adenosine receptor modulators, ampakines, NMDA-R modulators, mGluR modulators, cholinesterase inhibitors (e.g., donepezil, rivastigimine, and glanthanamine), typical and atypical antipsychotics (e.g., haloperidol, risperidone, and zyprexia) and selective serotonin reuptake inhibitors (SSRIs). In such combinations, each active ingredient can be administered either in accordance with their usual dosage range or a dose below its usual dosage range.

The compounds of Formulae I-VII, and the specific compounds listed above, can be administered as the sole active agent or in combination with other pharmaceutical agents such as other agents used in the treatment of allergic and/or inflammatory conditions, e.g. respiratory conditions. Suitable examples of other pharmaceutical agents which may be used in combination with the compounds of the present invention include, but are not limited to, other PDE-4 inhibitors, 5-lipoxygenase (5-LO) inhibitors or 5-lipoxygenase activating protein (FLAP) antagonists, leukotriene antagonists (LTRAs) including antagonists of LTB₄, LTC₄, LTD₄, and LTE₄, histaminic receptor antagonists including H1 and H3 antagonists, α₁ and α₂ adrenoceptor agonist vasoconstrictor sympathomimetic agents for decongestant use (e.g., propylhexedrine, phenylephrine, phenylpropanolamine, pseudoephedrine, naphazoline hydrochloride), muscarinic receptor (M1, M2, and M3) antagonists (e.g., ipratropium salts, namely bromide, tiotropium salts, namely bromide, oxitropium salts, namely bromide, perenzepine, and telenzepine), anticholinergic agents, β₁ to β₄ (e.g. β₂) adrenoceptor agonists (e.g., isoprenaline, albuterol, salbutamol, formoterol, salmeterol), COX-1 inhibitors (NSAIDs), COX-2 selective inhibitors, nitric oxide NSAIDs, oral or inhaled glucocorticosteroids (e.g., prednisone, prednisolone, flunisolide, triamcinolone acetonide, beclomethasone diproprionate), and adenosine A2a receptor agonists. Further examples of suitable other pharmaceutical agents which may be used in combination with the compounds of the present invention are disclosed in U.S. Pat. Nos. 6,559,168 and 6,756,392, which are hereby incorporated by reference in their entireties. In such combinations, each active ingredient can be administered either in accordance with their usual dosage range or a dose below its usual dosage range.

The dosages of the compounds of the present invention depend upon a variety of factors including the particular syndrome to be treated, the severity of the symptoms, the route of administration, the frequency of the dosage interval, the particular compound utilized, the efficacy, toxicology profile, pharmacokinetic profile of the compound, and the presence of any deleterious side-effects, among other considerations.

The compounds of the invention are typically administered at dosage levels and in a manner customary for PDE4 inhibitors such as those known compounds mentioned above. For example, the compounds can be administered, in single or multiple doses, by oral administration at a dosage level of, for example, 0.001-100 mg/kg/day, preferably 0.01-70 mg/kg/day, especially 0.01-10 mg/kg/day. Unit dosage forms can contain, for example, 0.1-50 mg of active compound. For intravenous administration, the compounds can be administered, in single or multiple dosages, at a dosage level of, for example, 0.001-50 mg/kg/day, preferably 0.001-10 mg/kg/day, especially 0.01-1 mg/kg/day. Unit dosage forms can contain, for example, 0.1-10 mg of active compound.

In carrying out the procedures of the present invention it is of course to be understood that reference to particular buffers, media, reagents, cells, culture conditions and the like are not intended to be limiting, but are to be read so as to include all related materials that one of ordinary skill in the art would recognize as being of interest or value in the particular context in which that discussion is presented. For example, it is often possible to substitute one buffer system or culture medium for another and still achieve similar, if not identical, results. Those of skill in the art will have sufficient knowledge of such systems and methodologies so as to be able, without undue experimentation, to make such substitutions as will optimally serve their purposes in using the methods and procedures disclosed herein.

The present invention will now be further described by way of the following non-limiting examples. In applying the disclosure of these examples, it should be kept clearly in mind that other and different embodiments of the methods disclosed according to the present invention will no doubt suggest themselves to those of skill in the relevant art.

In the foregoing and in the following examples, all temperatures are set forth uncorrected in degrees Celsius; and, unless otherwise indicated, all parts and percentages are by weight.

The entire disclosures of all applications, patents and publications, cited above and below, are hereby incorporated by reference.

### EXPERIMENTAL EXAMPLES

### Example 1

### Synthesis of (3,4-Bis-difluoromethoxy-phenyl)-thiazol-5-ylmethyl-amine

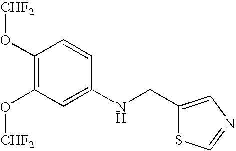

A mixture of 3,4-bis-difluoromethoxy-phenylamine (1.008 g, 4.5 mmol), thiazole-5-carbaldehyde (0.506 g, 4.5 mmol), dichloroethane (25 mL) and acetic acid (3 drops) was stirred for 1 hour at room temperature. Na(OAc)₃BH (1.90 g, 9.0 mmol) was then added, and the reaction was stirred overnight; then quenched by the addition of water and aqueous sodium hydroxide (1 M). The mixture was stirred for 30 minutes at room temperature, and the product was extracted three times using dichloromethane. The combined organic layers were dried over sodium sulfate, filtered and evaporated. The residue was purified via flash chromatography (hexanes/ethyl acetate) to 924 mg (64%) of (3,4-bis-difluoromethoxy-phenyl)-thiazol-5-ylmethylamine as a colorless oil.

The following compounds were prepared in a similar manner, but with different starting materials:
[4-methoxy-(2,2,2-trifluoroethoxy)phenyl]-3-fluorobenzyl-amine
(3,4-dimethoxyphenyl)-3-fluorobenzyl-amine
(3-ethoxy-4-methoxyphenyl)-3-fluorobenzylamine
(3-cyclobutyloxy-4-methoxyphenyl)-3-fluorobenzylamine
(3-cyclopentyloxy-4-methoxyphenyl)-3-fluorobenzylamine
(3,4-dimethoxyphenyl)-2,6-difluorobenzyl-amine
(3-ethoxy-4-methoxyphenyl)-2,6-difluorobenzyl-amine
(3-isopropoxy-4-methoxyphenyl)-2,6-difluorobenzyl-amine
(3-cyclobutyl-4-methoxyphenyl-2,6-difluorobenzyl-amine
[4-methoxy-3-[(3R)-tetrahydrofuran-3-yloxy]phenyl]-2,6-difluorobenzyl-amine
(4-difluoromethoxy-3-ethoxyphenyl)-pyridin-3-ylmethyl-amine
(3,4-Bis-difluoromethoxy-phenyl)-2,6-difluorobenzyl-amine
(3,4-Bis-difluoromethoxy-phenyl)-3-fluorobenzyl-amine
(4-difluoromethoxy-3-methoxyphenyl)-2,6-difluorobenzyl-amine
(4-difluoromethoxy-3-methoxyphenyl)-pyridin-3-ylmethyl-amine
(4-difluoromethoxy-3-methoxyphenyl)-3-fluorobenzyl-amine
(3,4-Bis-difluoromethoxy-phenyl)-2-fluorobenzyl-amine
(3,4-Bis-difluoromethoxy-phenyl)-4-fluorobenzyl-amine
[4-methoxy-3-[(3R)-tetrahydrofuran-3-yloxy]phenyl]-3-fluorobenzyl-amine
(3-cyclopentyloxy-4-methoxyphenyl)-3-cyanobenzyl-amine
(3-cyclopentyloxy-4-methoxyphenyl)-(6-oxo-1,6-dihydropyridin-3-yl)methyl-amine
(3-cyclopentyloxy-4-methoxyphenyl)-pyridin-3-ylmethyl-amine
(3-cyclopentyloxy-4-methoxyphenyl)(1H-pyrazol-4-ylmethyl)amine
(3-cyclopentyloxy-4-methoxyphenyl)-thiazol-5-ylmethyl-amine
(3-cyclopentyloxy-4-methoxyphenyl)-pyrimidin-5-ylmethyl-amine
(3-cyclopentyloxy-4-methoxyphenyl)-pyridazin-3-ylmethyl-amine
(3-cyclopentyloxy-4-methoxyphenyl)-[2-(dimethylamino)pyrimidin-5-yl]methyl-amine
(3,4-Bis-difluoromethoxy-phenyl)-pyridin-3-ylmethyl-amine
(3,4-dimethoxy-phenyl)-pyridin-3-ylmethyl-amine
[4-methoxy-3-[(3R)-tetrahydrofuran-3-yloxy]phenyl]-thiazol-5-ylmethyl-amine
(3-cyclopropylmethoxy-4-methoxy-phenyl)-thiazol-5-ylmethyl-amine
(3,4-Bis-difluoromethoxy-phenyl)-pyridazin-3-ylmethyl-amine
(3,4-Bis-difluoromethoxy-phenyl)-pyrimidin-5-ylmethyl-amine
(4-difluoromethoxy-3-ethoxyphenyl)-thiazol-5-ylmethyl-amine
(3-cyclopentyloxy-4-difluoromethoxy-phenyl)-thiazol-5-ylmethyl-amine
(4-difluoromethoxy-3-isopropoxyphenyl)-thiazol-5-ylmethyl-amine
(4-difluoromethoxy-3-isopropoxyphenyl)-pyridin-3-ylmethyl-amine
(4-difluoromethoxy-3-methoxyphenyl)-thiazol-5-ylmethyl-amine
[3,4-bis(difluoromethoxyphenyl)]-pyrazin-2-ylmethyl-amine
(3-cyclopropylmethoxy-4-difluoromethoxy-phenyl)-thiazol-5-ylmethyl-amine
(4-difluoromethoxy-3-methoxyphenyl)-pyridin-3-ylmethyl-amine
[3-(1-butyloxy)-4-difluoromethoxy-phenyl]-thiazol-5-ylmethyl-amine
[3-(1-butyloxy)-4-difluoromethoxy-phenyl]-pyridin-3-ylmethyl-amine
[3,4-bis(difluoromethoxy)phenyl]-(1-methyl-1H-pyrazol-4-yl)methyl]amine
(3-ethoxy-4-methoxyphenyl)-thiazol-5-ylmethyl-amine
(3-isopropoxy-4-methoxyphenyl)-thiazol-5-ylmethyl-amine
(3-cyclopropylmethoxy-4-methoxy-1-phenyl)-thiazol-5-ylmethyl-amine
(3-cyclopropylmethoxy-4-difluoromethoxy-phenyl)-pyridin-3-ylmethyl-amine
(3,4-dimethoxy-phenyl)-thiazol-5-ylmethyl-amine
[3,4-bis(difluoromethoxy)phenyl]-oxazol-5-ylmethyl-amine
(4-difluoromethoxy-3-ethoxyphenyl)-pyrimidin-5-ylmethyl-amine
(4-difluoromethoxy-3-ethoxyphenyl)-oxazol-5-ylmethyl-amine
(4-difluoromethoxy-3-ethoxyphenyl)-pyrazin-2-ylmethyl-amine
(3-difluoromethoxy-4-methoxyphenyl)-thiazol-5-ylmethyl-amine
(4-difluoromethoxy-3-methoxyphenyl)-pyrimidin-3-ylmethyl-amine
(4-difluoromethoxy-3-methoxyphenyl)-pyrazin-2-ylmethyl-amine
(4-difluoromethoxy-3-isopropoxyphenyl)-pyrazin-2-ylmethyl-amine
(3-cyclopropylmethoxy-4-difluoromethoxyphenyl)-pyrazin-2-ylmethyl-amine
(3-difluoromethoxy-4-methoxyphenyl)-pyrazin-2-ylmethyl-amine
(4-difluoromethoxy-3-isopropoxyphenyl)-oxazol-5-ylmethyl-amine
(3-cyclopropylmethoxy-4-difluoromethoxyphenyl)-oxazol-5-ylmethyl-amine
(3-cyclopropylmethoxy-4-difluoromethoxyphenyl)-pyrimidin-5-ylmethyl-amine
(4-difluoromethoxy-3-isopropoxyphenyl)-pyrimidin-5-ylmethyl-amine
(3,4-diethoxyphenyl)-thiazol-5-ylmethyl-amine
(3-cyclopentyloxy-4-methoxyphenyl)-(1-pyridin-3-ylethyl)amine
(3,4-dimethoxyphenyl)-(1-pyridin-3-ylethyl)amine
(4-ethoxy-3-methoxyphenyl)-thiazol-5-ylmethyl-amine
(4-difluoromethoxy-3-methoxyphenyl)-oxazol-5-ylmethyl-amine
(3-isopropyloxy-4-methoxyphenyl)-oxazol-5-ylmethyl-amine
(3-ethoxy-4-methoxyphenyl)-oxazol-5-ylmethyl-amine
(3-cyclobutyloxy-4-difluoromethoxyphenyl)-oxazol-5-ylmethyl-amine
(3-cyclopropylmethoxy-4-methoxyphenyl)-oxazol-5-ylmethyl-amine
(3-cyclobutyloxy-4-methoxyphenyl)-oxazol-5-ylmethyl-amine
[3-methoxy-4-(2,2,2-trifluoroethoxy)phenyl]-oxazol-5-ylmethyl-amine
[3-methoxy-4-(2,2,2-trifluoroethoxy)phenyl]-pyrimidin-5-ylmethyl-amine
(4-difluoromethoxy-3-ethoxyphenyl)-(5-methoxypyridin-3-yl)methyl-amine
(4-difluoromethoxy-3-ethoxyphenyl)-(4-methyloxazol-5-yl)methyl-amine
(3-cyclopropyloxy-4-methoxyphenyl)-oxazol-5-ylmethyl-amine
(3,4-diethoxyphenyl)-pyrimidin-5-ylmethyl-amine
(3-isopropoxy-4-methoxyphenyl)-pyrimidin-5-ylmethyl-amine
(3-cyclobutyloxy-4-methoxyphenyl)-pyrimidin-5-ylmethyl-amine
(3-cyclopropyloxy-4-methoxyphenyl)-pyrimidin-5-ylmethyl-amine
(3-cyclopropylmethoxy-4-methoxyphenyl)-pyrimidin-5-ylmethyl-amine
(3-ethoxy-4-methoxyphenyl)-pyrimidin-5-ylmethyl-amine
(4-ethoxy-3-isopropoxyphenyl)-pyrimidin-5-ylmethyl-amine
(3-cyclobutyloxy-4-difluoromethoxyphenyl)-pyrimidin-5-ylmethyl-amine
(3-ethoxy-4-isopropoxyphenyl)-thiazol-5-ylmethyl-amine
(4-ethoxy-3-isopropoxyphenyl)-oxazol-5-ylmethyl-amine
(4-ethoxy-3-methoxyphenyl)-pyrimidin-5-ylmethyl-amine
(3-cyclopropylmethoxy-4-difluoromethoxyphenyl)-pyrimidin-5-ylmethyl-amine
(3-cyclopropylmethoxy-4-methoxyphenyl)-pyrimidin-5-ylmethyl-amine
(3-benzyloxy-4-difluoromethoxyphenyl)-pyrimidin-5-ylmethyl-amine
(3-cyclopropylmethoxy-4-methoxyphenyl)-oxazol-5-ylmethyl-amine
(3-cyclobutyloxy-4-methoxyphenyl)-oxazol-5-ylmethyl-amine
(3-ethoxy-4-trifluoromethoxyphenyl)-oxazol-5-ylmethyl-amine
(3-cyclopropyloxy-4-methoxyphenyl)-pyrimidin-5-ylmethyl-amine
(4-ethoxy-3-methoxyphenyl)-oxazol-5-ylmethyl-amine
(3,4-diethoxyphenyl)-oxazol-5-ylmethyl-amine
(4-ethoxy-3-methoxyphenyl)-pyridin-5-ylmethyl-amine
(4-cyclopropyloxy-3-ethoxyphenyl)-pyrimidin-5-ylmethyl-amine
[4-methoxy-3-(methylthio)phenyl]-thiazol-5-ylmethyl-amino
(4-difluoromethoxy-3-ethoxyphenyl)-(2-methylthiazol-5-yl)methyl-amine
(4-difluoromethoxy-3-ethoxyphenyl)-tetrahydrofuran-3-ylmethyl-amino
(3-cyclopropyloxy-4-difluoromethoxyphenyl)-pyrimidin-5-ylmethyl-amine
(3-cyclopropyloxy-4-difluoromethoxyphenyl)-oxazol-5-ylmethyl-amine
(4-methoxy-3-trifluoromethoxyphenyl)-oxazol-5-ylmethyl-amine
(3-difluoromethoxy-4-methoxyphenyl)-oxazol-5-ylmethyl-amine
(3-difluoromethoxy-4-methoxyphenyl)-pyridin-3-ylmethyl-amine
(4-difluoromethoxy-3-ethoxyphenyl)-(1,2,3-thiadiazol-5-yl)methyl-amine.

### Example 2

### Synthesis of 4-[(3,4-Bis-difluoromethoxy-phenyl)-thiazol-5-ylmethyl-amino]-benzoic acid tert-butyl ester

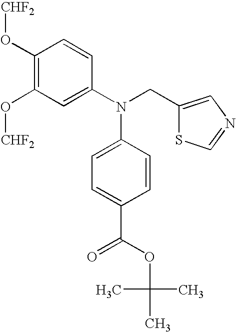

A Schlenk tube was loaded under an atmosphere of argon with (3,4-bis-difluoromethoxyphenyl)-thiazol-5-ylmethyl-amine (924 mg, 2.86 mmol), 4-bromo-benzoic acid tert-butyl ester (1.52 g, 5.91 mmol), Pd₂(dba)₃ (134 mg, 0.146 mmol) and dimethylamine (20 mL). P(t-Bu)₃ (10% in hexane, 1.5 mL, 0.573 mmol) was then added and the reaction was stirred at 80° C. under an atmosphere of argon overnight. The reaction mixture was then cooled to room temperature and filtered trough celite. The filtrate was concentrated in vacuo and the residue was purified via flash chromatography (hexane/ethyl acetate) to give 1.215 g (85%) of the crude product as an oil, which was further purified by stirring overnight at room temperature with MP-TMT (macroporous polystyrene-2,4,6-trimercaptotriazine) resin (600 mg) in dichloromethane (20 mL). The mixture was filtered through celite and the filtrate was concentrated, to afford 1.13 g (79%) of 4-[(3,4-Bis-difluoromethoxy-phenyl)-thiazol-5-ylmethyl-amino]-benzoic acid tert-butyl ester.

The following compounds were synthesized in a similar manner, but using different starting materials:
3-{(3-fluorobenzyl)[4-methoxy-3-(2,2,2-trifluoroethoxy)phenyl]amino}benzoic acid tert-butyl ester
3-[[3,4-bis(difluoromethoxy)phenyl]amino]benzoic acid, tert-butyl ester
3-[(3,4-dimethoxyphenyl)(3-fluorobenzyl)amino]benzoic acid tert-butyl ester
3-[(3-ethoxy-4-methoxyphenyl)(3-fluorobenzyl)amino]benzoic acid tert-butyl ester
3-[[3-(cyclobutyloxy)-4-methoxyphenyl](3-fluorobenzyl)amino]benzoic acid tert-butyl ester
3-[[3-(cyclopentyloxy)-4-methoxyphenyl](3-fluorobenzyl)amino]benzoic acid tert-butyl ester
3-[(2,6-difluorobenzyl)(3,4-dimethoxyphenyl)amino]benzoic acid tert-butyl ester
3-[(2,6-difluorobenzyl)(3-ethoxy-4-methoxyphenyl)amino]benzoic acid tert-butyl ester
3-[(2,6-difluorobenzyl)(3-isopropoxy-4-methoxyphenyl)amino]benzoic acid tert-butyl ester
3-[[3-(cyclobutyloxy)-4-methoxyphenyl](2,6-difluorobenzyl)amino]benzoic acid tert-butyl ester
3-((2,6-difluorobenzyl) {4-methoxy-3-[(3R)-tetrahydrofuran-3-yloxy]phenyl}amino)benzoic acid tert-butyl ester
3-[[4-(difluoromethoxy)-3-ethoxyphenyl](pyridin-3-ylmethyl)amino]benzoic acid tert-butyl ester
4-[[4-(difluoromethoxy)-3-ethoxyphenyl](pyridin-3-ylmethyl)amino]benzoic acid tert-butyl ester
3-[[3,4-bis(difluoromethoxy)phenyl](2,6-difluorobenzyl)amino]benzoic acid tert-butyl ester
3-[[3,4-bis(difluoromethoxy)phenyl](3-fluorobenzyl)amino]benzoic acid tert-butyl ester
3-{(2,6-difluorobenzyl) [4-(difluoromethoxy)-3-methoxyphenyl]amino}benzoic acid tert-butyl ester
3-[[4-(difluoromethoxy)-3-methoxyphenyl](pyridin-3-ylmethyl)amino]benzoic acid tert-butyl ester
3-[[4-(difluoromethoxy)-3-methoxyphenyl](3-fluorobenzyl)amino]benzoic acid tert-butyl ester
3-[[3,4-bis(difluoromethoxy)phenyl](2-fluorobenzyl)amino]benzoic acid tert-butyl ester
3-[[3,4-bis(difluoromethoxy)phenyl](4-fluorobenzyl)amino]benzoic acid tert-butyl ester
3-((3-fluorobenzyl) {4-methoxy-3-[(3R)-tetrahydrofuran-3-yloxy]phenyl}amino)benzonitrile
N-(3-fluorobenzyl)-4-methoxy-N-[4-(methylthio)phenyl]-3-[(3R)-tetrahydrofuran-3-yloxy]aniline
N-[4-((3-fluorobenzyl){4-methoxy-3-[(3R)-tetrahydrofuran-3-yloxy]phenyl})—N-(4-nitrophenyl)-amine
3-{(3-cyanobenzyl) [3-(cyclopentyloxy)-4-methoxyphenyl]amino}benzoic acid ethyl ester
3-{[3-(cyclopentyloxy)-4-methoxyphenyl][(6-oxo-1,6-dihydropyridin-3-yl)methyl]amino}benzoic acid ethyl ester
5-bromo-N-[3-(cyclopentyloxy)-4-methoxyphenyl]-N-(pyridin-3-ylmethyl)pyridin-2-amine
3-[[3-(cyclopentyloxy)-4-methoxyphenyl](piperidin-3-ylmethyl)amino]benzoic acid tert-butyl ester
4-{[3-(cyclopentyloxy)-4-methoxyphenyl][(6-oxo-1,6-dihydropyridin-3-yl)methyl]amino}benzoic acid ethyl ester
Ethyl {4-[[3-(cyclopentyloxy)-4-methoxyphenyl](pyridin-3-ylmethyl)amino]phenoxy}acetate
1-{4-[[3-(cyclopentyloxy)-4-methoxyphenyl](pyridin-3-ylmethyl)amino]phen-3-yloxy}propene
2-Chloropyridin-5-yl-4-(3-cyclopenyloxy-4-methoxyphenyl)-3-pyridylmethylamine
N(2)-benzyl-N(5)-[3-(cyclopentyloxy)-4-methoxyphenyl]-N(2)-methyl-N(5)-(pyridin-3-ylmethyl)pyridine-2,5-diamine
3-{{[1-(aminocarbonyl)piperidin-3-yl]methyl}[3-(cyclopentyloxy)-4-methoxyphenyl]amino}benzoic acid tert butyl ester
3-[[3-(cyclopentyloxy)-4-methoxyphenyl](1H-pyrazol-4-ylmethyl)amino]benzoic acid tert-butyl ester
3-[[3-(cyclopentyloxy)-4-methoxyphenyl](1,3-thiazol-5-ylmethyl)amino]benzoic acid tert-butyl ester
{3-[[3-(cyclopentyloxy)-4-methoxyphenyl](pyridin-3-ylmethyl)amino]phenyl}acetic acid tert-butyl ester
5-[[3-(cyclopentyloxy)-4-methoxyphenyl](pyridin-3-ylmethyl)amino]-2-ethylbenzoic acid methyl ester
3-[[3-(cyclopentyloxy)-4-methoxyphenyl](pyridin-3-ylmethyl)amino]-4-methylbenzoic acid ethyl ester
4-[[3-(cyclopentyloxy)-4-methoxyphenyl](1H-pyrazol-4-ylmethyl)amino]benzoic acid ethyl ester
4-[[3-(cyclopentyloxy)-4-methoxyphenyl](1,3-thiazol-5-ylmethyl)amino]benzoic acid ethyl ester
N-[3-(cyclopentyloxy)-4-methoxyphenyl]-N-(pyridin-3-ylmethyl)-6-pyrrolidin-1-ylpyridin-3-amine
N(5)-[3-(cyclopentyloxy)-4-methoxyphenyl]-N(2)-(2,3-dihydroxypropyl)-N(2)-methyl)-N(5)-(pyridin-3-ylmethyl)pyridine-2,5-diamine
N(5)-[3-(cyclopentyloxy)-4-methoxyphenyl]-N(2)-(2-morpholin-4-ylethyl)-N(5)-(pyridin-3-ylmethyl)pyridine-2,5-diamine
4-[[3-(cyclopentyloxy)-4-methoxyphenyl](pyrimidin-5-ylmethyl)amino]benzoic acid ethyl ester
N(2)-benzyl-N(5)-[3-(cyclopentyloxy)-4-methoxyphenyl]-N(5)-(pyridin-3-ylmethyl)pyridine-2,5-diamine
3-[[3-(cyclopentyloxy)-4-methoxyphenyl](1,3-thiazol-4-ylmethyl)amino]benzoic acid ethyl ester
3-[[3-(cyclopentyloxy)-4-methoxyphenyl](pyrimidin-5-ylmethyl)amino]benzoic acid ethyl ester
4-[[3-(cyclopentyloxy)-4-methoxyphenyl](pyridazin-3-ylmethyl)amino]benzoic acid ethyl ester
3-[[3-(cyclopentyloxy)-4-methoxyphenyl](pyridazin-3-ylmethyl)amino]benzoic acid ethyl ester
{4-[[3-(cyclopentyloxy)-4-methoxyphenyl](pyridin-3-ylmethyl)amino]phenyl}acetic acid ethyl ester
3-[[3-(cyclopentyloxy)-4-methoxyphenyl](piperidin-4-ylmethyl)amino]benzoic acid tert-butyl ester
4-([3-(cyclopentyloxy)-4-methoxyphenyl]{[2-(dimethylamino)pyrimidin-5-yl]methyl}amino)benzoic acid ethyl ester
3-([3-(cyclopentyloxy)-4-methoxyphenyl]{[2-(dimethylamino)pyrimidin-5-yl]methyl}amino)benzoic acid ethyl ester
3-{4-[[3-(cyclopentyloxy)-4-methoxyphenyl](pyridin-3-ylmethyl)amino]phenyl}propan-1-ol
5-[[3-(cyclopentyloxy)-4-methoxyphenyl](pyridin-3-ylmethyl)amino]nicotinic acid ethyl ester
3-{4-[[3-(cyclopentyloxy)-4-methoxyphenyl](pyridin-3-ylmethyl)amino]phenyl}propanoic acid ethyl ester
3-(cyclopentyloxy)-4-methoxy-N-[3-(2-methyl-1,3-thiazol-4-yl)phenyl]-N-(pyridin-3-ylmethyl)aniline
1-{3-[[3-(cyclopentyloxy)-4-methoxyphenyl](pyridin-3-ylmethyl)amino]phen-3-yloxy}propene
{3-[[3,4-bis(difluoromethoxy)phenyl](pyridin-3-ylmethyl)amino]phenyl}acetic acid ethyl ester
(5S)-5-({3-[[3-(cyclopentyloxy)-4-methoxyphenyl](pyridin-3-ylmethyl)amino]phenoxy}methyl)pyrrolidin-2-one
5-{3-[[3-(cyclopentyloxy)-4-methoxyphenyl](pyridin-3-ylmethyl)amino]phenyl}imidazolidine-2,4-dione
4-[[3,4-bis(difluoromethoxy)phenyl](1,3-thiazol-5-ylmethyl)amino]benzoic acid ethyl ester
1-{4-[[3,4-bis(difluoromethoxy)phenyl](3-pyridylmethyl)amino]phen-4-yloxy}propene 3-[[3,4-bis(difluoromethoxy)phenyl](1,3-thiazol-5-ylmethyl)amino]benzoic acid tert-butyl ester
N(5)-[3-(cyclopentyloxy)-4-methoxyphenyl]-N(2)-[2-(methylsulfonyl)ethyl]-N(5)-(pyridin-3-ylmethyl)pyridine-2,5-diamine
3,4-bis(difluoromethoxy)-N-[3-(methylthio)phenyl]-N-(pyridin-3-ylmethyl)aniline 3,4-bis(difluoromethoxy)-N-[4-(methylsulfonyl)phenyl]-N-(pyridin-3-ylmethyl)aniline
1-{4-[3-cyclopentyloxy-4-methoxy)phenyl](1,3-thiazol-5-ylmethyl)amino]phen-4-yloxy}propene
2-[{5-[[3-(cyclopentyloxy)-4-methoxyphenyl](pyridin-3-ylmethyl)amino]pyridin-2-yl}(methyl)amino]-N,N-dimethylacetamide
N(5)-[3-(cyclopentyloxy)-4-methoxyphenyl]-N(5)-(pyridin-3-ylmethyl)-N(2)-(tetrahydro-2H-pyran-4-yl)pyridine-2,5-diamine
3,4-bis(difluoromethoxy)-N-[3-(methylthio)phenyl]-N-(pyridin-3-ylmethyl)aniline
5-[[3-(cyclopentyloxy)-4-methoxyphenyl](pyridin-3-ylmethyl)amino]-N,2-dihydroxybenzamide
4-[[3-(cyclopentyloxy)-4-methoxyphenyl](pyridin-3-ylmethyl)amino]-3-methylbenzoic acid methyl ester
{4-[[3,4-bis(difluoromethoxy)phenyl](pyridin-3-ylmethyl)amino]phenyl}acetic acid ethyl ester
3-[{4-methoxy-3-[(3R)-tetrahydrofuran-3-yloxy]phenyl}(1,3-thiazol-5-ylmethyl)amino]benzoic acid tert-butyl ester
4-[{4-methoxy-3-[(3R)-tetrahydrofuran-3-yloxy]phenyl}(1,3-thiazol-5-ylmethyl)amino]benzoic acid ethyl ester
4-[[3-(cyclopropylmethoxy)-4-methoxyphenyl](1,3-thiazol-5-ylmethyl)amino]benzoic acid ethyl ester
3-[[3-(cyclopropylmethoxy)-4-methoxyphenyl](1,3-thiazol-5-ylmethyl)amino]benzoic acid tert-butyl ester
4-[[3,4-bis(difluoromethoxy)phenyl](pyridazin-3-ylmethyl)amino]benzoic acid tert-butyl ester
3-[[3,4-bis(difluoromethoxy)phenyl](pyridazin-3-ylmethyl)amino]benzoic acid tert-butyl ester
3,4-bis(difluoromethoxy)-N-[4-(ethylthio)phenyl]-N-(pyridin-3-ylmethyl)aniline
3,4-bis(difluoromethoxy)-N-[4-(methylthio)phenyl]-N-(1,3-thiazol-5-ylmethyl)aniline
3,4-bis(difluoromethoxy)-N-[4-(ethylthio)phenyl]-N-(1,3-thiazol-5-ylmethyl)aniline
3,4-bis(difluoromethoxy)-N-[3-(methylthio)phenyl]-N-(1,3-thiazol-5-ylmethyl)aniline
4-[[3,4-bis(difluoromethoxy)phenyl](pyrimidin-5-ylmethyl)amino]benzoic acid tert-butyl ester
5-[[3-(cyclopropylmethoxy)-4-methoxyphenyl](1,3-thiazol-5-ylmethyl)amino]nicotinic acid tert-butyl ester
3-[[3,4-bis(difluoromethoxy)phenyl](pyrimidin-5-ylmethyl)amino]benzoic acid tert-butyl ester
5-[[3,4-bis(difluoromethoxy)phenyl](pyridin-3-ylmethyl)amino]nicotinic acid tert butyl ester
1-{4-[[3,4-bis(difluoromethoxy)phenyl](pyridin-3-ylmethyl)amino]-2-tert-butyldimethylsilyloxyphenyl}ethanone
1-{4-[[3,4-bis(difluoromethoxy)phenyl](pyridin-3-ylmethyl)amino]phenyl}-2,2,2-trifluoroethanone
1-{4-[[3,4-bis(difluoromethoxy)phenyl](pyridin-3-ylmethyl)amino]phenyl}ethanone
1-{3-[[3,4-bis(difluoromethoxy)phenyl](pyridin-3-ylmethyl)amino]phenyl}ethanone
5-[[3,4-bis(difluoromethoxy)phenyl](pyridin-3-ylmethyl)amino]-2,3-difluoro-1-tert-butyldimethylsilyloxyphenyl
5-[[3,4-bis(difluoromethoxy)phenyl](1,3-thiazol-5-ylmethyl)amino]-2-tert-butyldimethylsilyloxybenzoic acid tert-butyl ester
4-[[3,4-bis(difluoromethoxy)phenyl](1,3-thiazol-5-ylmethyl)amino]-2-tert-butyldimethylsilyloxybenzoic acid methyl ester
3-[[3,4-bis(difluoromethoxy)phenyl](pyridin-3-ylmethyl)amino]benzaldehyde
1-{5-[[3,4-bis(difluoromethoxy)phenyl](pyridine-3-ylmethyl)amino]pyridin-3-yl}ethanone
N-[3,4-bis(difluoromethoxy)phenyl]-5-(2-methyl-1,3-dioxolan-2-yl)-N-(pyridin-3-ylmethyl)pyridin-3-amine
3,4-bis(difluoromethoxy)-N-[3-(1H-(1-triphenylmethyl)pyrazol-3-yl)phenyl]-N-(pyridin-3-ylmethyl)aniline
3-[[4-(difluoromethoxy)-3-ethoxyphenyl](1,3-thiazol-5-ylmethyl)amino]benzoic acid tert-butyl ester
5-[[3,4-bis(difluoromethoxy)phenyl](1,3-thiazol-5-ylmethyl)amino]nicotinic acid tert-butyl ester
4-[[4-(difluoromethoxy)-3-ethoxyphenyl](1,3-thiazol-5-ylmethyl)amino]benzoic acid tert-butyl ester
{4-[[3,4-bis(difluoromethoxy)phenyl](1,3-thiazol-5-ylmethyl)amino]phenyl}acetic acid ethyl ester
3[3,4-bis(difluoromethoxyphenyl)-N-(pyridin-3-ylmethyl)amino]benzonitrile
{3-[[3,4-bis(difluoromethoxy)phenyl](1,3-thiazol-5-ylmethyl)amino]phenyl}acetic acid ethyl ester
4-[[3-(cyclopentyloxy)-4-(difluoromethoxy)phenyl](1,3-thiazol-5-ylmethyl)amino]benzoic acid tert-butyl ester
4-[[4-(difluoromethoxy)-3-isopropoxyphenyl](1,3-thiazol-5-ylmethyl)amino]benzoic acid tert-butyl ester
4-[[4-(difluoromethoxy)-3-isopropoxyphenyl](pyridin-3-ylmethyl)amino]benzoic acid tert-butyl ester
3-[[4-(difluoromethoxy)-3-isopropoxyphenyl](pyridin-3-ylmethyl)amino]benzoic acid tert-butyl ester
3-[[4-(difluoromethoxy)-3-isopropoxyphenyl](1,3-thiazol-5-ylmethyl)amino]benzoic acid tert-butyl ester
3-[[3-(cyclopentyloxy)-4-(difluoromethoxy)phenyl](1,3-thiazol-5-ylmethyl)amino]benzoic acid tert-butyl ester
4-methoxy-N-[4-(methylsulfonyl)phenyl]-3-[(3R)-tetrahydrofuran-3-yloxy]-N-(1,3-thiazol-5-ylmethyl)aniline
1-{4-[[3,4-bis(difluoromethoxy)phenyl](1,3-thiazol-5-ylmethyl)amino]phenyl}ethanone
1-{3-[[3,4-bis(difluoromethoxy)phenyl](1,3-thiazol-5-ylmethyl)amino]phenoxy}-3-methoxypropan-2-ol
3,4-bis(difluoromethoxy)-N-[3-(methylthio)phenyl]-N-(pyrimidin-5-ylmethyl)aniline
3,4-bis(difluoromethoxy)-N-[4-(methylthio)phenyl]-N-(pyrimidin-5-ylmethyl)aniline
4-methoxy-N-[3-(methylsulfonyl)phenyl]-3-[(3R)-tetrahydrofuran-3-yloxy]-N-(1,3-thiazol-5-ylmethyl)aniline
3-[[4-(difluoromethoxy)-3-methoxyphenyl](1,3-thiazol-5-ylmethyl)amino]benzoic acid tert-butyl ester
1-{4-[[3,4-bis(difluoromethoxy)phenyl](pyrimidin-5-ylmethyl)amino]phenyl}ethanone
3,4-bis(difluoromethoxy)-N-[4-(methylsulfonyl)phenyl]-N-(pyrazin-2-ylmethyl)aniline
1-{4-[[3,4-bis(difluoromethoxy)phenyl](pyrazin-2-ylmethyl)amino]phenyl}ethanone
4-[[4-(difluoromethoxy)-3-methoxyphenyl](1,3-thiazol-5-ylmethyl)amino]benzoic acid tert-butyl ester
4-[[3-(cyclopropylmethoxy)-4-(difluoromethoxy)phenyl](1,3-thiazol-5-ylmethyl)amino]benzoic acid tert-butyl ester
1-{3-[[3,4-bis(difluoromethoxy)phenyl](pyridin-3-ylmethyl)amino]phenoxy}-2-methylpropan-2-ol
1-{3-[[3,4-bis(difluoromethoxy)phenyl](1,3-thiazol-5-ylmethyl)amino]phenoxy}-2-methylpropan-2-ol
4-[[4-(difluoromethoxy)-3-methoxyphenyl](pyridin-3-ylmethyl)amino]benzoic acid tert-butyl ester
3-[[3-butoxy-4-(difluoromethoxy)phenyl](1,3-thiazol-5-ylmethyl)amino]benzoic acid tert-butyl ester
3-[[3-butoxy-4-(difluoromethoxy)phenyl](pyridin-3-ylmethyl)amino]benzoic acid tert-butyl ester
3-{[3,4-bis(difluoromethoxy)phenyl][(1-methyl-1H-pyrazol-4-yl)methyl]amino}benzoic acid tert-butyl ester
4-[[3-butoxy-4-(difluoromethoxy)phenyl](pyridin-3-ylmethyl)amino]benzoic acid tert-butyl ester
4-[[3-butoxy-4-(difluoromethoxy)phenyl](1,3-thiazol-5-ylmethyl)amino]benzoic acid tert-butyl ester
4-[(3-ethoxy-4-methoxyphenyl)(1,3-thiazol-5-ylmethyl)amino]benzoic acid tert-butyl ester
4-[(3-isopropoxy-4-methoxyphenyl)(1,3-thiazol-5-ylmethyl)amino]benzoic acid tert-butyl ester
3-(cyclopropylmethoxy)-4-methoxy-N-[4-(methylsulfonyl)phenyl]-N-(1,3-thiazol-5-ylmethyl)aniline
1-{4-[[3-(cyclopropylmethoxy)-4-methoxyphenyl](1,3-thiazol-5-ylmethyl)amino]phenyl}ethanone
4-[[3-(cyclopropylmethoxy)-4-(difluoromethoxy)phenyl](pyridin-3-ylmethyl)amino]benzoic acid tert-butyl ester
3,4-bis(difluoromethoxy)-N-[4-methyl-3-(methylsulfonyl)phenyl]-N-(1,3-thiazol-5-ylmethyl)aniline
{4-[[3,4-bis(difluoromethoxy)phenyl](pyrimidin-5-ylmethyl)amino]phenyl}acetic acid ethyl ester
4-[(3,4-dimethoxyphenyl)(1,3-thiazol-5-ylmethyl)amino]benzoic acid tert-butyl ester
{3-[[3,4-bis(difluoromethoxy)phenyl](pyrimidin-5-ylmethyl)amino]phenyl}acetic acid ethyl ester
{4-[[3,4-bis(difluoromethoxy)phenyl](pyrazin-2-ylmethyl)amino]phenyl}acetic acid ethyl ester
4-[[3,4-bis(difluoromethoxy)phenyl](1,3-oxazol-5-ylmethyl)amino]benzoic acid tert-butyl ester
4-[[3,4-bis(difluoromethoxy)phenyl](pyrazin-2-ylmethyl)amino]benzoic acid tert-butyl ester
4-[[4-(difluoromethoxy)-3-ethoxyphenyl](pyrimidin-5-ylmethyl)amino]benzoic acid tert-butyl ester
4-[[4-(difluoromethoxy)-3-ethoxyphenyl](1,3-oxazol-5-ylmethyl)amino]benzoic acid tert-butyl ester
Methyl 4-[[3,4-bis(difluoromethoxy)phenyl](1,3-thiazol-5-ylmethyl)amino]-benzenesulphonate
4-[[4-(difluoromethoxy)-3-ethoxyphenyl](pyrazin-2-ylmethyl)amino]benzoic acid tert-butyl ester
3-[[3,4-bis(difluoromethoxy)phenyl](pyrazin-2-ylmethyl)amino]benzoic acid tert-butyl ester
2-[[3,4-bis(difluoromethoxy)phenyl](phenyl)amino]-1-((tert-butyldimethyl)silyloxy)ethane
3-[[3-(difluoromethoxy)-4-methoxyphenyl](1,3-thiazol-5-ylmethyl)amino]benzoic acid tert-butyl ester
4-[[3-(difluoromethoxy)-4-methoxyphenyl](1,3-thiazol-5-ylmethyl)amino]benzoic acid tert-butyl ester
4-[[4-(difluoromethoxy)-3-methoxyphenyl](pyrimidin-5-ylmethyl)amino]benzoic acid
4-[[4-(difluoromethoxy)-3-methoxyphenyl](pyrazin-2-ylmethyl)amino]benzoic acid tert-butyl ester
4-[[4-(difluoromethoxy)-3-isopropoxyphenyl](pyrazin-2-ylmethyl)amino]benzoic acid tert-butyl ester
3-[[4-(difluoromethoxy)-3-isopropoxyphenyl](pyrazin-2-ylmethyl)amino]benzoic acid tert-butyl ester
4-[[3-(cyclopropylmethoxy)-4-(difluoromethoxy)phenyl](pyrazin-2-ylmethyl)amino]benzoic acid tert-butyl ester
3-[[3-(cyclopropylmethoxy)-4-(difluoromethoxy)phenyl](pyrazin-2-ylmethyl)amino]benzoic acid tert-butyl ester
5-[[4-(difluoromethoxy)-3-isopropoxyphenyl](1,3-thiazol-5-ylmethyl)amino]nicotinic acid tert-butyl ester
4-[[3-(difluoromethoxy)-4-methoxyphenyl](pyrazin-2-ylmethyl)amino]benzoic acid tert-butyl ester
N-[3,4-bis(difluoromethoxy)phenyl]-N-(1,3-thiazol-5-ylmethyl)]-4-methyl-3-nitroaniline
N-[3,4-bis(difluoromethoxy)phenyl]-N-(1,3-thiazol-5-ylmethyl)]-3-nitroaniline
4-[[4-(difluoromethoxy)-3-isopropoxyphenyl](1,3-oxazol-5-ylmethyl)amino]benzoic acid tert-butyl ester
4-[[3-(cyclopropylmethoxy)-4-(difluoromethoxy)phenyl](1,3-oxazol-5-ylmethyl)amino]benzoic acid tert-butyl ester
4-[[3-(cyclopropylmethoxy)-4-(difluoromethoxy)phenyl](pyrimidin-5-ylmethyl)amino]benzoic acid tert-butyl ester
4-[[4-(difluoromethoxy)-3-isopropoxyphenyl](pyrimidin-5-ylmethyl)amino]benzoic acid tert-butyl ester
4-[(3,4-diethoxyphenyl)(1,3-thiazol-5-ylmethyl)amino]benzoic acid tert-butyl ester
3-[[3-(cyclopentyloxy)-4-methoxyphenyl](1-pyridin-3-ylethyl)amino]benzoic acid tert-butyl ester
3-[(3,4-dimethoxyphenyl)(1-pyridin-3-ylethyl)amino]benzoic acid tert-butyl ester
4-[(4-ethoxy-3-methoxyphenyl)(1,3-thiazol-5-ylmethyl)amino]benzoic acid tert-butyl ester
4-(difluoromethoxy)-3-ethoxy-N-[4-(methylsulfonyl)phenyl]-N-(1,3-thiazol-5-ylmethyl)aniline
{3-[[4-(difluoromethoxy)-3-ethoxyphenyl](1,3-oxazol-5-ylmethyl)amino]phenyl}acetic acid ethyl ester
3-[[3,4-bis(difluoromethoxy)phenyl](1,3-thiazol-5-ylmethyl)amino]butyric acid tert-butyl ester
{4-[[4-(difluoromethoxy)-3-ethoxyphenyl](1,3-oxazol-5-ylmethyl)amino]phenyl}acetic acid ethyl ester
{3-[[4-(difluoromethoxy)-3-ethoxyphenyl](1,3-thiazol-5-ylmethyl)amino]phenyl}acetic acid ethyl ester
4-[[4-(difluoromethoxy)-3-methoxyphenyl](1,3-oxazol-5-ylmethyl)amino]benzoic acid tert-butyl ester
4-[(3-isopropoxy-4-methoxyphenyl)(1,3-oxazol-5-ylmethyl)amino]benzoic acid tert-butyl ester
4-[(3-ethoxy-4-methoxyphenyl)(1,3-oxazol-5-ylmethyl)amino]benzoic acid tert-butyl ester
4-[[3-(cyclobutyloxy)-4-(difluoromethoxy)phenyl](1,3-oxazol-5-ylmethyl)amino]benzoic acid tert-butyl ester
4-[[3-(cyclopropylmethoxy)-4-methoxyphenyl](1,3-oxazol-5-ylmethyl)amino]benzoic acid tert-butyl ester
4-[[3-(cyclobutyloxy)-4-methoxyphenyl](1,3-oxazol-5-ylmethyl)amino]benzoic acid tert-butyl ester
4-[[3-methoxy-4-(2,2,2-trifluoroethoxy)phenyl](1,3-oxazol-5-ylmethyl)amino]benzoic acid tert-butyl ester
4-[[3-methoxy-4-(2,2,2-trifluoroethoxy)phenyl](pyrimidin-5-ylmethyl)amino]benzoic acid tert-butyl ester
4-{[4-(difluoromethoxy)-3-ethoxyphenyl][(5-methoxypyridin-3-yl)methyl]amino}benzoic acid tert-butyl ester
4-{[4-(difluoromethoxy)-3-ethoxyphenyl][(4-methyl-1,3-oxazol-5-yl)methyl]amino}benzoic acid tert-butyl ester
4-[[3-(cyclopropyloxy)-4-methoxyphenyl](1,3-oxazol-5-ylmethyl)amino]benzoic acid tert-butyl ester
4-[[4-(difluoromethoxy)-3-ethoxyphenyl](1,3-oxazol-4-ylmethyl)amino]benzoic acid methyl ester
cis-4-[[3,4-bis(difluoromethoxy)phenyl](1,3-thiazol-5-ylmethyl)amino]cyclohexanecarboxylic acid ethyl ester
4-[[4-(difluoromethoxy)-3-ethoxyphenyl](1,3-oxazol-4-ylmethyl)amino]benzoic acid tert-butyl ester
4-[(3,4-diethoxyphenyl)(pyrimidin-5-ylmethyl)amino]benzoic acid tert-butyl ester
4-[(3-isopropoxy-4-methoxyphenyl)(pyrimidin-5-ylmethyl)amino]benzoic acid tert-butyl ester
4-[[3-(cyclobutyloxy)-4-methoxyphenyl](pyrimidin-5-ylmethyl)amino]benzoic acid tert-butyl ester
4-[[3-(cyclopropyloxy)-4-methoxyphenyl](pyrimidin-5-ylmethyl)amino]benzoic acid tert-butyl ester
4-[[3-(cyclopropylmethoxy)-4-methoxyphenyl](pyrimidin-5-ylmethyl)amino]benzoic acid tert-butyl ester
4-[(3-ethoxy-4-methoxyphenyl)(pyrimidin-5-ylmethyl)amino]benzoic acid tert-butyl ester
3-[[4-(difluoromethoxy)-3-isopropoxyphenyl](1,3-oxazol-5-ylmethyl)amino]benzoic acid tert-butyl ester
3-[[3-(cyclopropylmethoxy)-4-(difluoromethoxy)phenyl](1,3-oxazol-5-ylmethyl)amino]benzoic acid tert-butyl ester
3-[[4-(difluoromethoxy)-3-ethoxyphenyl](pyrimidin-5-ylmethyl)amino]benzoic acid methyl ester
3-[(3-ethoxy-4-methoxyphenyl)(1,3-oxazol-5-ylmethyl)amino]benzoic acid tert-butyl ester
3-[[3-(cyclobutyloxy)-4-(difluoromethoxy)phenyl](1,3-oxazol-5-ylmethyl)amino]benzoic acid tert-butyl ester
4-[(4-ethoxy-3-isopropoxyphenyl)(pyrimidin-5-ylmethyl)amino]benzoic acid tert-butyl ester
3-[(3-isopropoxy-4-methoxyphenyl)(1,3-oxazol-5-ylmethyl)amino]benzoic acid tert-butyl ester
3-[[4-(difluoromethoxy)-3-ethoxyphenyl](1,3-oxazol-5-ylmethyl)amino]benzoic acid tert-butyl ester
4-[[3-(cyclobutyloxy)-4-(difluoromethoxy)phenyl](pyrimidin-5-ylmethyl)amino]benzoic acid tert-butyl ester
3-[[4-(difluoromethoxy)-3-methoxyphenyl](1,3-oxazol-5-ylmethyl)amino]benzoic acid tert-butyl ester
4-[(3-ethoxy-4-isopropoxyphenyl)(1,3-thiazol-5-ylmethyl)amino]benzoic acid tert-butyl ester
4-[(4-ethoxy-3-isopropoxyphenyl)(1,3-oxazol-5-ylmethyl)amino]benzoic acid tert-butyl ester
4-[(4-ethoxy-3-methoxyphenyl)(pyrimidin-5-ylmethyl)amino]benzoic acid tert-butyl ester
3-[(3-ethoxy-4-methoxyphenyl)(pyrimidin-5-ylmethyl)amino]benzoic acid methyl ester
3-[[3-(cyclopropylmethoxy)-4-(difluoromethoxy)phenyl](pyrimidin-5-ylmethyl)amino]benzoic acid tert-butyl ester
3-[[3-(cyclopropylmethoxy)-4-methoxyphenyl](pyrimidin-5-ylmethyl)amino]benzoic acid tert-butyl ester
3-[(3-isopropoxy-4-methoxyphenyl)(pyrimidin-5-ylmethyl)amino]benzoic acid tert-butyl ester
4-[[3-(benzyloxy)-4-(difluoromethoxy)phenyl](pyrimidin-5-ylmethyl)amino]benzoic acid tert-butyl ester
3-[[3-(cyclopropylmethoxy)-4-methoxyphenyl](1,3-oxazol-5-ylmethyl)amino]benzoic acid tert-butyl ester
3-[[3-(cyclobutyloxy)-4-methoxyphenyl](1,3-oxazol-5-ylmethyl)amino]benzoic acid tert-butyl ester
4-[[3-ethoxy-4-(trifluoromethoxy)phenyl](1,3-oxazol-5-ylmethyl)amino]benzoic acid ethyl ester
3-[[3-(cyclopropyloxy)-4-methoxyphenyl](pyrimidin-5-ylmethyl)amino]benzoic acid tert-butyl ester
4-[(4-ethoxy-3-methoxyphenyl)(1,3-oxazol-5-ylmethyl)amino]benzoic acid tert-butyl ester
4-[[4-(difluoromethoxy)-3-methoxyphenyl]amino]benzoic acid tert-butyl ester
4-[[4-(difluoromethoxy)-3-ethoxyphenyl]amino]benzoic acid tert-butyl ester
4-[[3,4-bis(difluoromethoxy)phenyl]amino]benzoic acid tert-butyl ester
4-[(3,4-diethoxyphenyl)(1,3-oxazol-5-ylmethyl)amino]benzoic acid tert-butyl ester
4-[(4-ethoxy-3-methoxyphenyl)(pyridin-3-ylmethyl)amino]benzoic acid tert-butyl ester
3-[(4-ethoxy-3-methoxyphenyl)(pyridin-3-ylmethyl)amino]benzoic acid tert-butyl ester
3-[[4-(difluoromethoxy)-3-isopropoxyphenyl](pyrimidin-5-ylmethyl)amino]benzoic acid tert-butyl ester
3-[[4-(cyclopropyloxy)-3-ethoxyphenyl](pyrimidin-5-ylmethyl)amino]benzoic acid methyl ester
3-[(4-ethoxy-3-methoxyphenyl)(1,3-oxazol-5-ylmethyl)amino]benzoic acid tert-butyl ester
4-[[4-methoxy-3-(methylthio)phenyl](1,3-thiazol-5-ylmethyl)amino]benzoic acid tert-butyl ester
4-{[4-(difluoromethoxy)-3-ethoxyphenyl][(2-methyl-1,3-thiazol-5-yl)methyl]amino}benzoic acid tert-butyl ester
3-[(4-ethoxy-3-methoxyphenyl)(1,3-thiazol-5-ylmethyl)amino]benzoic acid tert-butyl ester
3-[(3,4-diethoxyphenyl)(1,3-thiazol-5-ylmethyl)amino]benzoic acid tert-butyl ester
3-[[3-(cyclobutyloxy)-4-methoxyphenyl](pyrimidin-5-ylmethyl)amino]benzoic acid tert-butyl ester
4-[[4-(difluoromethoxy)-3-ethoxyphenyl](tetrahydrofuran-3-ylmethyl)amino]benzoic acid tert-butyl ester
4-[[3-(cyclopropyloxy)-4-(difluoromethoxy)phenyl](pyrimidin-5-ylmethyl)amino]benzoic acid tert-butyl ester
4-[[3-(cyclopropyloxy)-4-(difluoromethoxy)phenyl](1,3-oxazol-5-ylmethyl)amino]benzoic acid, tert-butyl ester
4-[[4-methoxy-3-(trifluoromethoxy)phenyl](1,3-oxazol-5-ylmethyl)amino]benzoic acid, tert-butyl ester
3-[[3-(difluoromethoxy)-4-methoxyphenyl](1,3-oxazol-5-ylmethyl)amino]benzoic acid, tert-butyl ester
4-[[3-(difluoromethoxy)-4-methoxyphenyl](pyridin-3-ylmethyl)amino]benzoic acid, tert-butyl ester
4-[[4-(difluoromethoxy)-3-ethoxyphenyl](1,2,3-thiadiazol-5-ylmethyl)amino]benzoic acid, tert-butyl ester
4-[[3-hydroxy-4-methoxyphenyl](1,3-oxazol-5-ylmethyl)amino]benzoic acid, tert-butyl ester
3-[[4-ethoxy-3-methoxyphenyl](pyrimidin-5-ylmethyl)amino]benzoic acid, tert-butyl ester
3-[[3,4-diethoxyphenyl](pyrimidin-5-ylmethyl)amino]benzoic acid, tert-butyl ester
3-[[3,4-diethoxyphenyl](1,3-oxazol-5-ylmethyl)amino]benzoic acid, tert-butyl ester
3-[[3-cyclopropyloxy-4-methoxyphenyl](1,3-oxazol-5-ylmethyl)amino]benzoic acid, tert-butyl ester
3-[[3-cyclobutyloxy-4-difluoromethoxyphenyl](pyrimidin-5-ylmethyl)amino]benzoic acid, tert-butyl ester and
3-[[3-cyclopropyloxy-4-difluoromethoxyphenyl](1,3-oxazol-5-ylmethyl)amino]benzoic acid, tert-butyl ester.

### Example 3

### Synthesis of 4-[(3,4-Bis-difluoromethoxy-phenyl)-thiazol-5-ylmethyl-amino]-benzoic acid

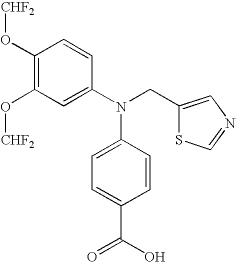

A mixture of 4-[(3,4-bis-difluoromethoxy-phenyl)-thiazol-5-ylmethyl-amino]-benzoic acid tert-butyl ester (1.13 g, 2.3 mmol) and trifluoroacetic acid (0.7 mL, 9.09 mmol) in dichloromethane (12 mL) was heated at 100° C. in a microwave oven at 75 Watts for 1 hour. The reaction mixture was then diluted with ethyl acetate and a saturated aqueous solution of sodium bicarbonate was added. The organic phase was separated, dried over sodium sulfate, filtered and evaporated. The residue was purified via flash chromatography (dichloromethane/methanol)) to afford 692 mg (68%) of the desired product as an off-white foam. Recrystallization from ethyl acetate afforded 4-[(3,4-Bis-difluoromethoxy-phenyl)-thiazol-5-ylmethyl-amino]-benzoic acid as a white crystalline solid. Mp=144.3-145.7° C.

mp 144.3-145.7° C.; ¹H NMR (DMSO-d₆) δ 12.49 (bs, 1H), 8.98 (s, 1H), 7.86 (s, 1H), 7.79 (m, 2H), 7.39-7.14 (m, 5H), 7.03-6.97 (m, 2H), 5.30 (s, 2H); MS (ESI, [M+H⁺]) m/z 443; Anal. Calcd for C₁₉H₁₄F₄N₂O₄S: C, 51.59; H, 3.19; N, 6.33. Found: C, 51.37; H, 3.07; N, 6.35.

The following compounds were prepared in a similar manner starting with the corresponding tert-butyl ester:
3-{(3-fluorobenzyl)[4-methoxy-3-(2,2,2-trifluoroethoxy)phenyl]amino}benzoic acid
3-[[3,4-bis(difluoromethoxy)phenyl]amino]benzoic acid
3-[(3,4-dimethoxyphenyl)(3-fluorobenzyl)amino]benzoic acid
3-[(3-ethoxy-4-methoxyphenyl)(3-fluorobenzyl)amino]benzoic acid
3-[[3-(cyclobutyloxy)-4-methoxyphenyl](3-fluorobenzyl)amino]benzoic acid
3-[[3-(cyclopentyloxy)-4-methoxyphenyl](3-fluorobenzyl)amino]benzoic acid
3-[(2,6-difluorobenzyl)(3,4-dimethoxyphenyl)amino]benzoic acid
3-[(2,6-difluorobenzyl)(3-ethoxy-4-methoxyphenyl)amino]benzoic acid
3-[(2,6-difluorobenzyl)(3-isopropoxy-4-methoxyphenyl)amino]benzoic acid
3-[[3-(cyclobutyloxy)-4-methoxyphenyl](2,6-difluorobenzyl)amino]benzoic acid
3-((2,6-difluorobenzyl) {4-methoxy-3-[(3R)-tetrahydrofuran-3-yloxy]phenyl}amino)benzoic
3-[[4-(difluoromethoxy)-3-ethoxyphenyl](pyridin-3-ylmethyl)amino]benzoic acid
4-[[4-(difluoromethoxy)-3-ethoxyphenyl](pyridin-3-ylmethyl)amino]benzoic acid
3-[[3,4-bis(difluoromethoxy)phenyl](2,6-difluorobenzyl)amino]benzoic acid
3-[[3,4-bis(difluoromethoxy)phenyl](3-fluorobenzyl)amino]benzoic acid
3-{(2,6-difluorobenzyl) [4-(difluoromethoxy)-3-methoxyphenyl]amino}benzoic acid
3-[[4-(difluoromethoxy)-3-methoxyphenyl](pyridin-3-ylmethyl)amino]benzoic acid
3-[[4-(difluoromethoxy)-3-methoxyphenyl](3-fluorobenzyl)amino]benzoic acid
3-[[3,4-bis(difluoromethoxy)phenyl](2-fluorobenzyl)amino]benzoic acid
3-[[3,4-bis(difluoromethoxy)phenyl](4-fluorobenzyl)amino]benzoic acid
3-{{[1-(aminocarbonyl)piperidin-3-yl]methyl}[3-(cyclopentyloxy)-4-methoxyphenyl]amino}benzoic acid
3-[[3,4-bis(difluoromethoxy)phenyl](1,3-thiazol-5-ylmethyl)amino]benzoic acid
3-[{4-methoxy-3-[(3R)-tetrahydrofuran-3-yloxy]phenyl}(1,3-thiazol-5-ylmethyl)amino]benzoic acid
3-[[4-(difluoromethoxy)-3-ethoxyphenyl](1,3-thiazol-5-ylmethyl)amino]benzoic acid
5-[[3,4-bis(difluoromethoxy)phenyl](1,3-thiazol-5-ylmethyl)amino]nicotinic acid
4-[[4-(difluoromethoxy)-3-ethoxyphenyl](1,3-thiazol-5-ylmethyl)amino]benzoic acid
4-[[3-(cyclopentyloxy)-4-(difluoromethoxy)phenyl](1,3-thiazol-5-ylmethyl)amino]benzoic acid
4-[[4-(difluoromethoxy)-3-isopropoxyphenyl](1,3-thiazol-5-ylmethyl)amino]benzoic acid
4-[[4-(difluoromethoxy)-3-isopropoxyphenyl](pyridin-3-ylmethyl)amino]benzoic acid
3-[[4-(difluoromethoxy)-3-isopropoxyphenyl](pyridin-3-ylmethyl)amino]benzoic acid
3-[[4-(difluoromethoxy)-3-isopropoxyphenyl](1,3-thiazol-5-ylmethyl)amino]benzoic acid
3-[[3-(cyclopentyloxy)-4-(difluoromethoxy)phenyl](1,3-thiazol-5-ylmethyl)amino]benzoic acid
4-[[4-(difluoromethoxy)-3-methoxyphenyl](1,3-thiazol-5-ylmethyl)amino]benzoic acid
4-[[4-(difluoromethoxy)-3-methoxyphenyl](pyridin-3-ylmethyl)amino]benzoic acid
3-[[3-butoxy-4-(difluoromethoxy)phenyl](1,3-thiazol-5-ylmethyl)amino]benzoic acid
3-[[3-butoxy-4-(difluoromethoxy)phenyl](pyridin-3-ylmethyl)amino]benzoic acid
3-{[3,4-bis(difluoromethoxy)phenyl][(1-methyl-1H-pyrazol-4-yl)methyl]amino}benzoic acid
4-[[3-butoxy-4-(difluoromethoxy)phenyl](pyridin-3-ylmethyl)amino]benzoic acid
4-[[3-butoxy-4-(difluoromethoxy)phenyl](1,3-thiazol-5-ylmethyl)amino]benzoic acid
4-[(3-ethoxy-4-methoxyphenyl)(1,3-thiazol-5-ylmethyl)amino]benzoic acid
4-[(3-isopropoxy-4-methoxyphenyl)(1,3-thiazol-5-ylmethyl)amino]benzoic acid
4-[(3,4-dimethoxyphenyl)(1,3-thiazol-5-ylmethyl)amino]benzoic acid
3-[[3-(difluoromethoxy)-4-methoxyphenyl](1,3-thiazol-5-ylmethyl)amino]benzoic acid
4-[[3-(difluoromethoxy)-4-methoxyphenyl](1,3-thiazol-5-ylmethyl)amino]benzoic acid
4-[[4-(difluoromethoxy)-3-isopropoxyphenyl](pyrazin-2-ylmethyl)amino]benzoic acid
3-[[4-(difluoromethoxy)-3-isopropoxyphenyl](pyrazin-2-ylmethyl)amino]benzoic acid
4-[[3-(cyclopropylmethoxy)-4-(difluoromethoxy)phenyl](pyrazin-2-ylmethyl)amino]benzoic acid
3-[[3-(cyclopropylmethoxy)-4-(difluoromethoxy)phenyl](pyrazin-2-ylmethyl)amino]benzoic acid
4-[(3,4-diethoxyphenyl)(1,3-thiazol-5-ylmethyl)amino]benzoic acid
3-[[3-(cyclopentyloxy)-4-methoxyphenyl](1-pyridin-3-ylethyl)amino]benzoic acid
3-[(3,4-dimethoxyphenyl)(1-pyridin-3-ylethyl)amino]benzoic acid
4-[(4-ethoxy-3-methoxyphenyl)(1,3-thiazol-5-ylmethyl)amino]benzoic acid
3-[[3,4-bis(difluoromethoxy)phenyl](1,3-thiazol-5-ylmethyl)amino]butyric acid
4-[[3-(cyclopropylmethoxy)-4-methoxyphenyl](1,3-oxazol-5-ylmethyl)amino]benzoic acid
4-{[4-(difluoromethoxy)-3-ethoxyphenyl][(5-methoxypyridin-3-yl)methyl]amino}benzoic acid
4-[[3-(cyclopropyloxy)-4-methoxyphenyl](1,3-oxazol-5-ylmethyl)amino]benzoic acid
3-[[4-(difluoromethoxy)-3-ethoxyphenyl](1,3-oxazol-5-ylmethyl)amino]benzoic acid
4-[(3-ethoxy-4-isopropoxyphenyl)(1,3-thiazol-5-ylmethyl)amino]benzoic acid
4-[(4-ethoxy-3-isopropoxyphenyl)(1,3-oxazol-5-ylmethyl)amino]benzoic acid
4-[(4-ethoxy-3-methoxyphenyl)(1,3-oxazol-5-ylmethyl)amino]benzoic acid
4-[[4-(difluoromethoxy)-3-methoxyphenyl]amino]benzoic acid
4-[[4-(difluoromethoxy)-3-ethoxyphenyl]amino]benzoic acid
4-[[3,4-bis(difluoromethoxy)phenyl]amino]benzoic acid
4-[(3,4-diethoxyphenyl)(1,3-oxazol-5-ylmethyl)amino]benzoic acid
4-[(4-ethoxy-3-methoxyphenyl)(pyridin-3-ylmethyl)amino]benzoic acid
3-[(4-ethoxy-3-methoxyphenyl)(pyridin-3-ylmethyl)amino]benzoic acid
3-[(4-ethoxy-3-methoxyphenyl)(1,3-oxazol-5-ylmethyl)amino]benzoic acid
3-[(4-ethoxy-3-methoxyphenyl)(1,3-thiazol-5-ylmethyl)amino]benzoic acid
3-[(3,4-diethoxyphenyl)(1,3-thiazol-5-ylmethyl)amino]benzoic acid
4-[[3-(cyclopropyloxy)-4-(difluoromethoxy)phenyl](1,3-oxazol-5-ylmethyl)amino]benzoic acid
4-[[4-methoxy-3-(trifluoromethoxy)phenyl](1,3-oxazol-5-ylmethyl)amino]benzoic acid
3-[[4-ethoxy-3-methoxyphenyl](pyrimidin-5-ylmethyl)amino]benzoic acid
3-[[3,4-diethoxyphenyl](pyrimidin-5-ylmethyl)amino]benzoic acid
3-[[3,4-diethoxyphenyl](1,3-oxazol-5-ylmethyl)amino]benzoic acid
3-[[3-cyclopropyloxy-4-methoxyphenyl](1,3-oxazol-5-ylmethyl)amino]benzoic acid, and
3-[[3-cyclopropyloxy-4-difluoromethoxyphenyl](1,3-oxazol-5-ylmethyl)amino]benzoic acid.

### Example 4

### Synthesis of 4-Bromo-2-ethoxy-phenol

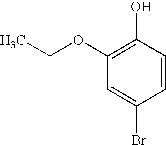

A mixture of 2-ethoxyphenol (30.0 g, 217 mmol) and TBAB [(tri-n-butylamino tribromide) ((n-Bu₄)NBr₃)](104.7 g, 217 mmol) in a 10:1 mixture of dichloromethane/methanol (385 mL) was allowed to warm from −78° C. to 0° C. over a period of 2.5 hr. The reaction was quenched by the addition of Na₂S₂O₃ (8 g in 80 mL of water) and the reaction mixture was concentrated in vacuo. The residue was extracted with diethyl ether and the organic layer was separated, washed with water and brine, dried over magnesium sulfate, filtered, and evaporated in vacuo to a volume of 400 mL. The mixture was then warmed slightly and filtered by gravity. The filtrate was concentrated, taken up in diethylether/hexanes (1:2), heated, and the mixture was) filtered while it was hot to remove the inorganic by-product. The filtrate was concentrated to afford 37.04 g of 4-bromo-2-ethoxy-phenol as a slightly colored solid. A second crop of lower purity was isolated from the filtrate, affording an additional 9.41 g of product.

### Example 5

### Synthesis of 4-Bromo-1-difluoromethoxy-2-ethoxy-benzene

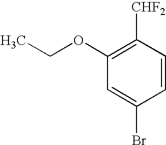

Powdered potassium hydroxide (5.17 g, 92.1 mmol) was added to a stirring solution of 4-bromo-2-ethoxy-phenol (20.0 g, 92.1 mmol) in N-methylpyrrolidinone (400 mL) at room temperature. The dark colored solution was stirred for 30 minutes, then difluorochloromethane was bubbled through the reaction mixture until saturation occurred. The reaction was stirred for an additional 30 minutes, then potassium hydroxide (10.3 g in 10 mL of water) was added dropwise maintaining the temperature below 31° C. The resulting mixture was stirred for 3 hours. Nitrogen was then bubbled through the mixture for 25 minutes, and the reaction mixture was poured into a 1:1 mixture of diethylether/water. The organic layer was separated and the aqueous layer was extracted with diethyl ether. The combined organics were washed with water and brine, then dried over sodium sulfate, filtered and evaporated. The resulting crude material was purified via flash chromatography (hexane/ethyl acetate) to afford 16.7 g of 4-bromo-1-difluoromethoxy-2-ethoxy-benzene as colorless oil.

### Example 6

### Synthesis of 4-[(Pyrimidin-5-ylmethyl)-amino]-benzoic acid tert-butyl ester

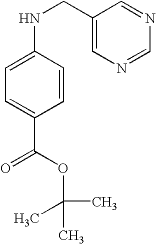

A mixture of 4-amino-benzoic acid tert-butyl ester (3.93 g, 20.4 mmol), pyrimidine-5-carboxaldehyde (2.00 g, 18.5 mmol), Na(OAc)₃BH (5.88 g, 27.8 mmol) and methanol (3 drops) in dichloroethane (75 mL) was stirred at room temperature for 16 hr. The reaction mixture was then diluted with dichloromethane, washed with an aqueous saturated solution of sodium bicarbonate and brine, dried over sodium sulfate, filtered and evaporated. The resulting crude material was purified via flash chromatography (hexane/ethyl acetate), to afford 4.02 g of 4-[(pyrimidin-5-ylmethyl)-amino]-benzoic acid tert-butyl ester.

### Example 7

### Synthesis of 4-[(4-Difluoromethoxy-3-ethoxy-phenyl)-pyrimidin-5-ylmethyl-amino]-benzoic acid tert-butyl ester

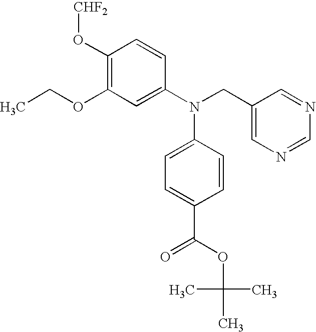

A mixture of toluene/dimethoxyethane (10 mL/3 mL) was added to a mixture of 4-[(pyrimidin-5-ylmethyl)-amino]-benzoic acid tert-butyl ester (1.42 g, 4.91 mmol), 4-bromo-1-difluoromethoxy-2-ethoxy-benzene (2.00 g, 7.49 mmol), sodium hydroxide (589 mg, 14.7 mmol), and Pd(t-Bu₃P)₂ (251 mg, 0.49 mmol) under an atmosphere of argon. The resulting mixture was stirred at room temperature for 15 minutes, then heated at 40° C. for 15 minutes, followed by heating at 60° C. for 3.5 hr. The reaction mixture was then cooled to room temperature and MP-TMT (macroporous polystyrene-2,4,6-trimercaptotriazine) resin (1.0 g) was added. The mixture was gently stirred overnight, then filtered through celite. The filtrate was evaporated in vacuo and the residue was purified via flash chromatography (hexane/ethyl acetate) to afford 2.45 g of 4-[(4-Difluoromethoxy-3-ethoxy-phenyl)-pyrimidin-5-ylmethylamino]-benzoic acid tert-butyl ester as a syrup.

### Example 8

### Synthesis of 4-[(4-Difluoromethoxy-3-ethoxy-phenyl)-pyrimidin-5-ylmethyl-amino]-benzoic acid

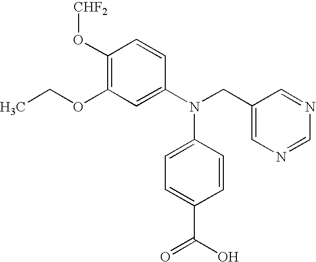

A mixture of 4-[(4-difluoromethoxy-3-ethoxy-phenyl)-pyrimidin-5-ylmethyl-amino]-benzoic acid tert-butyl ester (2.30 g, 4.88 mmol) and potassium hydroxide (2.00 g, 35.6 mmol) in ethanol (85 mL) was heated at reflux for 4 hr, then cooled to room temperature and concentrated in vacuo. The resulting residue was taken up in water and washed with dichloromethane (5 mL). Toluene (120 mL) was then added and the mixture was adjusted to pH 6.5 by the addition of 6M hydrochloric acid, then heated to 70° C. until homogeneous. The reaction mixture was allowed to cool slowly overnight during which time a light tanned solid formed. The needle-like crystals were collected by filtration and washed with toluene and water. The crystals were dried under vacuum at 50° C. for 3 days, then purified through a short silica column. The resulting solid was recrystallized from ethyl acetate to afford 1.42 g of 4-[(4-Difluoromethoxy-3-ethoxy-phenyl)-pyrimidin-5-ylmethyl-amino]-benzoic acid as fine white crystals. mp=165.3-165.9° C.

mp 165.3-165.9° C.; ¹H NMR (DMSO-d₆) δ 12.36 (bs, 1H), 9.07 (s, 1H), 8.74 (s, 2H), 7.77-7.74 (m, 2H), 7.30-6.80 (m, 6H), 5.12 (s, 2H), 4.03 (q, J=7 Hz, 2H), 1.29 (t, J=7 Hz, 3H); MS (ESI, [M+H⁺]) m/z 416; Anal. Calcd for C₂₁H₁₉F₂N₃O₄: C, 60.72; H, 4.61; N, 10.12. Found: C, 60.69; H, 4.55; N, 10.20.

The following compounds were prepared in a similar fashion starting with the corresponding tert-butyl esters:

Ester Hydrolysis with KOH
3-[[3-(cyclopentyloxy)-4-methoxyphenyl](piperidin-3-ylmethyl)amino]benzoic acid
3-[[3-(cyclopentyloxy)-4-methoxyphenyl](1H-pyrazol-4-ylmethyl)amino]benzoic acid
3-[[3-(cyclopentyloxy)-4-methoxyphenyl](1,3-thiazol-5-ylmethyl)amino]benzoic acid
4-[[3-(cyclopentyloxy)-4-methoxyphenyl](1H-pyrazol-4-ylmethyl)amino]benzoic acid
4-[[3-(cyclopentyloxy)-4-methoxyphenyl](1,3-thiazol-5-ylmethyl)amino]benzoic acid
3-[[3-(cyclopentyloxy)-4-methoxyphenyl](1,3-thiazol-4-ylmethyl)amino]benzoic acid
3-[[3-(cyclopentyloxy)-4-methoxyphenyl](pyrimidin-5-ylmethyl)amino]benzoic acid
3-[[3-(cyclopentyloxy)-4-methoxyphenyl](pyridazin-3-ylmethyl)amino]benzoic acid
3-[[3-(cyclopentyloxy)-4-methoxyphenyl](piperidin-4-ylmethyl)amino]benzoic acid
4-([3-(cyclopentyloxy)-4-methoxyphenyl]{[2-(dimethylamino)pyrimidin-5-yl]methyl}amino)benzoic acid
3-([3-(cyclopentyloxy)-4-methoxyphenyl]{[2-(dimethylamino)pyrimidin-5-yl]methyl}amino)benzoic acid
4-[[3,4-bis(difluoromethoxy)phenyl](1,3-thiazol-5-ylmethyl)amino]benzoic acid
4-[{4-methoxy-3-[(3R)-tetrahydrofuran-3-yloxy]phenyl}(1,3-thiazol-5-ylmethyl)amino]benzoic acid
4-[[3-(cyclopropylmethoxy)-4-methoxyphenyl](1,3-thiazol-5-ylmethyl)amino]benzoic acid
3-[[3-(cyclopropylmethoxy)-4-methoxyphenyl](1,3-thiazol-5-ylmethyl)amino]benzoic acid
4-[[3,4-bis(difluoromethoxy)phenyl](pyridazin-3-ylmethyl)amino]benzoic acid
3-[[3,4-bis(difluoromethoxy)phenyl](pyridazin-3-ylmethyl)amino]benzoic acid
4-[[3,4-bis(difluoromethoxy)phenyl](pyrimidin-5-ylmethyl)amino]benzoic acid
3-[[3,4-bis(difluoromethoxy)phenyl](pyrimidin-5-ylmethyl)amino]benzoic acid
4-[[4-(difluoromethoxy)-3-ethoxyphenyl](pyrimidin-5-ylmethyl)amino]benzoic acid
4-[[4-(difluoromethoxy)-3-methoxyphenyl](pyrimidin-5-ylmethyl)amino]benzoic acid
4-[[4-(difluoromethoxy)-3-methoxyphenyl](pyrazin-2-ylmethyl)amino]benzoic acid
5-[[4-(difluoromethoxy)-3-isopropoxyphenyl](1,3-thiazol-5-ylmethyl)amino]nicotinic acid
4-[[3-(cyclopropylmethoxy)-4-(difluoromethoxy)phenyl](pyrimidin-5-ylmethyl)amino]benzoic acid
4-[[4-(difluoromethoxy)-3-isopropoxyphenyl](pyrimidin-5-ylmethyl)amino]benzoic acid
4-[[3-methoxy-4-(2,2,2-trifluoroethoxy)phenyl](pyrimidin-5-ylmethyl)amino]benzoic acid
4-[(3,4-diethoxyphenyl)(pyrimidin-5-ylmethyl)amino]benzoic acid
4-[(3-isopropoxy-4-methoxyphenyl)(pyrimidin-5-ylmethyl)amino]benzoic acid
4-[[3-(cyclobutyloxy)-4-methoxyphenyl](pyrimidin-5-ylmethyl)amino]benzoic acid
4-[[3-(cyclopropyloxy)-4-methoxyphenyl](pyrimidin-5-ylmethyl)amino]benzoic acid
4-[[3-(cyclopropylmethoxy)-4-methoxyphenyl](pyrimidin-5-ylmethyl)amino]benzoic acid
4-[(3-ethoxy-4-methoxyphenyl)(pyrimidin-5-ylmethyl)amino]benzoic acid
4-[(4-ethoxy-3-isopropoxyphenyl)(pyrimidin-5-ylmethyl)amino]benzoic acid
4-[[3-(cyclobutyloxy)-4-(difluoromethoxy)phenyl](pyrimidin-5-ylmethyl)amino]benzoic acid
4-[(4-ethoxy-3-methoxyphenyl)(pyrimidin-5-ylmethyl)amino]benzoic acid
3-[[3-(cyclopropylmethoxy)-4-(difluoromethoxy)phenyl](pyrimidin-5-ylmethyl)amino]benzoic acid
4-[[3-(benzyloxy)-4-(difluoromethoxy)phenyl](pyrimidin-5-ylmethyl)amino]benzoic acid
3-[[3-(cyclopropylmethoxy)-4-methoxyphenyl](1,3-oxazol-5-ylmethyl)amino]benzoic acid, and
3-[[3-cyclobutyloxy-4-difluoromethoxyphenyl](pyrimidin-5-ylmethyl)amino]benzoic acid.

### Example 9

### Synthesis of 4-[(Pyridin-3-ylmethyl)-amino]-benzoic acid tert-butyl ester

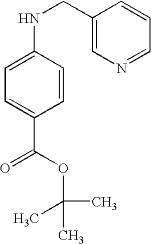

A mixture of 4-amino-benzoic acid tert-butyl ester (6.63 g, 34.3 mmol), pyridine-5-carboxaldehyde (3.34 g, 31.2 mmol) and Na(OAc)₃BH (9.91 g, 46.8 mmol) in dichloroethane (75 mL) was stirred at room temperature for 16 hr. The reaction mixture was then diluted with dichloromethane and washed with an aqueous saturated solution of sodium bicarbonate and brine, dried over sodium sulfate, filtered, and evaporated. The resulting crude material was purified via flash chromatography (hexane/ethyl acetate) to affording 8.64 g of 4-[(Pyridin-3-ylmethyl)-amino]-benzoic acid tert-butyl ester as a white solid.

### Example 10

### Synthesis of 4-[(4-Difluoromethoxy-3-ethoxy-phenyl)-pyridin-3-ylmethyl-amino]-benzoic acid tert-butyl ester

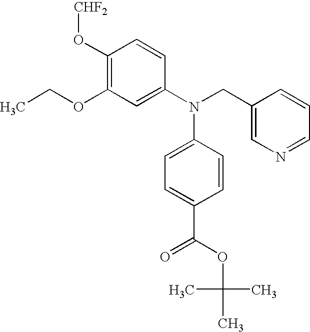

A mixture of toluene/dimethoxyethane (10 mL/3 mL) was added to a mixture of 4-[(pyridin-3-ylmethyl)-amino]-benzoic acid tert-butyl ester (1.39 g, 4.91 mmol), 4-bromo-1-difluoromethoxy-2-ethoxy-benzene (2.00 g, 7.49 mmol), sodium hydroxide (589 mg, 14.7 mmol), and Pd(t-Bu₃P)₂ (251 mg, 0.49 mmol) under an atmosphere of argon. The resulting mixture was stirred at room temperature for 15 minutes, then heated at 40° C. for 15 minutes, followed by heating to 60° C. for 3.5 hr. The reaction was then cooled to room temperature and MP-TMT (macroporous polystyrene-2,4,6-trimercaptotriazine) resin (1.0 g) was added. The mixture was gently stirred overnight, then filtered through celite. The filtrate was evaporated in vacuo and the residue was purified via flash chromatography (hexane/ethyl acetate) to afford 2.50 g of 4-[(4-Difluoromethoxy-3-ethoxy-phenyl)-pyridin-3-ylmethyl-amino]-benzoic acid tert-butyl ester as an oil.

### Example 11

### Synthesis of 4-[(4-Difluoromethoxy-3-ethoxy-phenyl)-pyridin-3-ylmethyl-amino]-benzoic acid

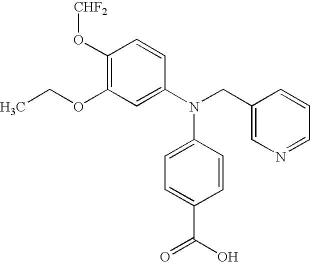

A mixture of 4-[(4-difluoromethoxy-3-ethoxy-phenyl)-pyridin-3-ylmethyl-amino]-benzoic acid tert-butyl ester (2.29 g, 4.88 mmol) and potassium hydroxide (2.00 g, 35.6 mmol) in ethanol (85 mL) was heated at reflux for 4 hr, then cooled to room temperature and concentrated in vacuo. The residue was taken up in water and washed with dichloromethane (5 mL). Toluene (120 mL) was then added and the mixture was adjusted to pH 6.5 by the addition of 6M hydrochloric acid, then heated at 70° C. until homogeneous. The mixture was cooled to (room) temperature slowly overnight, and the resulting solid was collected by filtration, washed with toluene and water, and dried under vacuum at 50° C. overnight. The solid was purified via elution through silica gel (dichloromethane/hexanes), and the resulting solid was recrystallized from ethyl acetate to give 1.43 g of 4-[(4-difluoromethoxy-3-ethoxy-phenyl)-pyridin-3-ylmethyl-amino]-benzoic acid as a white solid. mp=177.6-178.9° C.

mp 177.6-178.9° C.; ¹H NMR (DMSO-d₆) δ 12.30 (bs, 1H), 8.53 (d, J=2 Hz, 1H), 8.44 (dd, J=1.5, 4.7 Hz, 1H), 7.76-6.80 (m, 3H), 7.30-6.80 (m, 7H), 5.11 (s, 2H), 4.03 (q, J=7 Hz, 2H), 1.29 (t, J=7 Hz, 3H); MS (ESI, [M+H⁺]) m/z 415.

### Example 12

### Synthesis of 4-Bromo-1-difluoromethoxy-2-methoxy-benzene

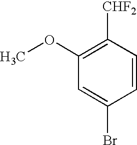

Powdered KOH (4.65 g, 82.9 mmol) was added to a stirring solution of 4-bromo-2-methoxy-phenol (18.0 g, 82.9 mmol) in NMP (1-methyl-2-pyrrolidinone) (400 mL) at room temperature under an atmosphere of nitrogen. The mixture was heated at 50° C. for 45 minutes, then cooled to room temperature. Difluorochloromethane was bubbled through the reaction mixture (approximately 20 minutes) until saturation occurred, and the mixture was then stirred for an additional 20 minutes. Potassium hydroxide (9.31 g in 5 mL of water) was then added dropwise maintaining the temperature below 33° C., and the mixture was stirred for an additional 40 minutes. Additional potassium hydroxide (4.65 g, in 5 mL of water) was then added dropwise, and the mixture was stirred for one hr. Nitrogen was then bubbled through the reaction, and water was added carefully. The pH of the resulting mixture was adjusted to 8.0 by the addition of potassium hydroxide (aqueous) and the mixture was extracted using diethylether. The combined extracts were washed with water and brine, dried over sodium sulfate, filtered, and evaporated. The crude material was purified via flash chromatography (hexanes/ethyl acetate) to afford 16.3 g of 4-bromo-1-difluoromethoxy-2-methoxy-benzene as colorless liquid.

### Example 13

### Synthesis of 4-[(4-Difluoromethoxy-3-methoxy-phenyl)-pyridin-3-ylmethyl-amino]-benzoic acid tert-butyl ester

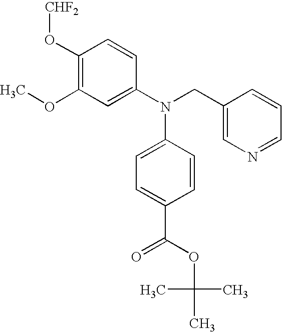

A mixture of toluene/dimethoxyethane (5/1, 18 mL) was added to a mixture of 4-[(pyridin-3-ylmethyl)-amino]-benzoic acid tert-butyl ester (2.00 g, 7.03 mmol), 4-bromo-1-difluoromethoxy-2-methoxy-benzene (2.66 g, 10.5 mmol), sodium hydroxide (0.84 g, 21.1 mmol), and Pd(t-Bu₃P)₂ (359 mg, 0.70 mmol) under an atmosphere of argon. The resulting mixture was stirred at room temperature for 15 minutes, then heated at 40° C. for 15 minutes, then heated to 60° C. for 3.5 hr. After cooling to room temperature MP-TMT (macroporous polystyrene-2,4,6-trimercaptotriazine) resin (1.0 g) was added, the mixture was gently stirred overnight, then filtered through celite. The filtrate was evaporated in vacuo and the residue was purified via flash chromatography (hexane/ethyl acetate) to afford 3.30 g of 4-[(4-difluoromethoxy-3-methoxy-phenyl)-pyridin-3-ylmethyl-amino]-benzoic acid tert-butyl ester as a syrup.

### Example 14

### Synthesis of 4-[(4-Difluoromethoxy-3-methoxy-phenyl)-pyridin-3-ylmethyl-amino]-benzoic acid

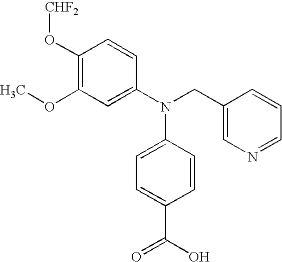

A mixture of 4-[(4-difluoromethoxy-3-methoxy-phenyl)-pyridin-3-ylmethyl-amino]-benzoic acid tert-butyl ester (3.20 g), formic acid (9 mL) and hydrochloric acid (6M, 9 mL) in toluene (10 mL) was heated at reflux for 3 hr, then cooled, and the layers were separated. The aqueous layer was carefully adjusted to pH 6.5 by the addition of an aqueous solution of sodium hydroxide, then extracted using ethyl acetate (100 mL). The combined organic extracts were washed with water and brine, dried over sodium sulfate and concentrated in vacuo. The crude residue was dissolved in hot ethyl acetate and the product was recrystallized overnight affording a white solid, which was collected by filtration and rinsed with cold ethyl acetate. The solid was dried in vacuo at 60° C. to give 913 mg of 4-[(4-Difluoromethoxy-3-methoxy-phenyl)-pyridin-3-ylmethyl-amino]-benzoic acid. The mother liquors were evaporated in vacuo and the residue purified via flash chromatography (dichloromethane/methanol). The resulting solid was recrystallized from ethyl acetate to afford a further 1.29 g of 4-[(4-Difluoromethoxy-3-methoxy-phenyl)-pyridin-3-ylmethyl-amino]-benzoic acid as white crystals. mp 183.7-184.9° C.

mp 183.7-184.9° C.; ¹H NMR (DMSO-d₆) δ 12.39 (bs, 1H), 8.55 (d, J=1.8 Hz, 1H), 8.45 (dd, J=1.6, 4.8 Hz, 1H), 7.76-7.72 (m, 3H), 7.37-6.81 (m, 7H), 5.12 (s, 2H), 3.78 (s, 3H); MS (ESI, [M+H⁺]) m/z 401; Anal. Calcd for C₂₁H₁₈F₂N₂O₄: C, 63.00; H, 4.53; N, 7.00. Found: C, 63.00; H, 4.58; N, 6.97.

The following compounds were prepared in a similar fashion starting with the corresponding tert-butyl ester:
5-[[3-(cyclopropylmethoxy)-4-methoxyphenyl](1,3-thiazol-5-ylmethyl)amino]nicotinic acid
5-[[3,4-bis(difluoromethoxy)phenyl](pyridin-3-ylmethyl)amino]nicotinic acid
3-[[4-(difluoromethoxy)-3-methoxyphenyl](1,3-thiazol-5-ylmethyl)amino]benzoic acid
4-[[3-(cyclopropylmethoxy)-4-(difluoromethoxy)phenyl](1,3-thiazol-5-ylmethyl)amino]benzoic acid
4-[[3-(cyclopropylmethoxy)-4-(difluoromethoxy)phenyl](pyridin-3-ylmethyl)amino]benzoic acid
4-[[3,4-bis(difluoromethoxy)phenyl](1,3-oxazol-5-ylmethyl)amino]benzoic acid
4-[[3,4-bis(difluoromethoxy)phenyl](pyrazin-2-ylmethyl)amino]benzoic acid
4-[[4-(difluoromethoxy)-3-ethoxyphenyl](1,3-oxazol-5-ylmethyl)amino]benzoic acid
4-[[4-(difluoromethoxy)-3-ethoxyphenyl](pyrazin-2-ylmethyl)amino]benzoic acid
3-[[3,4-bis(difluoromethoxy)phenyl](pyrazin-2-ylmethyl)amino]benzoic acid
4-[[3-(difluoromethoxy)-4-methoxyphenyl](pyrazin-2-ylmethyl)amino]benzoic acid
4-[[4-(difluoromethoxy)-3-isopropoxyphenyl](1,3-oxazol-5-ylmethyl)amino]benzoic acid
4-[[3-(cyclopropylmethoxy)-4-(difluoromethoxy)phenyl](1,3-oxazol-5-ylmethyl)amino]benzoic acid
4-[[4-(difluoromethoxy)-3-methoxyphenyl](1,3-oxazol-5-ylmethyl)amino]benzoic acid
4-[(3-isopropoxy-4-methoxyphenyl)(1,3-oxazol-5-ylmethyl)amino]benzoic acid
4-[(3-ethoxy-4-methoxyphenyl)(1,3-oxazol-5-ylmethyl)amino]benzoic acid
4-[[3-(cyclobutyloxy)-4-(difluoromethoxy)phenyl](1,3-oxazol-5-ylmethyl)amino]benzoic acid
4-[[3-(cyclobutyloxy)-4-methoxyphenyl](1,3-oxazol-5-ylmethyl)amino]benzoic acid
4-[[3-methoxy-4-(2,2,2-trifluoroethoxy)phenyl](1,3-oxazol-5-ylmethyl)amino]benzoic acid
4-{[4-(difluoromethoxy)-3-ethoxyphenyl][(4-methyl-1,3-oxazol-5-yl)methyl]amino}benzoic acid
4-[[4-(difluoromethoxy)-3-ethoxyphenyl](1,3-oxazol-4-ylmethyl)amino]benzoic acid
3-[[4-(difluoromethoxy)-3-isopropoxyphenyl](1,3-oxazol-5-ylmethyl)amino]benzoic acid
3-[[3-(cyclopropylmethoxy)-4-(difluoromethoxy)phenyl](1,3-oxazol-5-ylmethyl)amino]benzoic acid
3-[(3-ethoxy-4-methoxyphenyl)(1,3-oxazol-5-ylmethyl)amino]benzoic acid
3-[[3-(cyclobutyloxy)-4-(difluoromethoxy)phenyl](1,3-oxazol-5-ylmethyl)amino]benzoic acid
3-[(3-isopropoxy-4-methoxyphenyl)(1,3-oxazol-5-ylmethyl)amino]benzoic acid
3-[[4-(difluoromethoxy)-3-methoxyphenyl](1,3-oxazol-5-ylmethyl)amino]benzoic acid
3-[[3-(cyclobutyloxy)-4-methoxyphenyl](1,3-oxazol-5-ylmethyl)amino]benzoic acid
4-[[4-methoxy-3-(methylthio)phenyl](1,3-thiazol-5-ylmethyl)amino]benzoic acid
4-{[4-(difluoromethoxy)-3-ethoxyphenyl][(2-methyl-1,3-thiazol-5-yl)methyl]amino}benzoic acid
4-[[4-(difluoromethoxy)-3-ethoxyphenyl](tetrahydrofuran-3-ylmethyl)amino]benzoic acid
3-[[3-(difluoromethoxy)-4-methoxyphenyl](1,3-oxazol-5-ylmethyl)amino]benzoic acid
4-[[3-(difluoromethoxy)-4-methoxyphenyl](pyridin-3-ylmethyl)amino]benzoic acid
4-[[4-(difluoromethoxy)-3-ethoxyphenyl](1,2,3-thiadiazol-5-ylmethyl)amino]benzoic acid, and
4-[[3-hydroxy-4-methoxyphenyl](1,3-oxazol-5-ylmethyl)amino]benzoic acid.

### Example 15

### Synthesis of 4-[(2-Triisopropylsilanyl-oxazol-5-ylmethyl)-amino]-benzoic acid tert-butyl ester

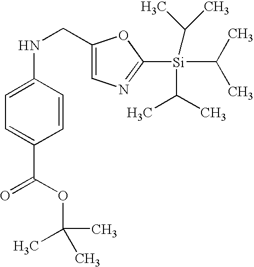

Acetic acid (10 drops) was added to a mixture of 4-amino-benzoic acid tert-butyl ester (2.99 g, 15.5 mmol) and 2-triisopropylsilanyl-oxazole-5-carbaldehyde (3.94 g, 15.5 mmol) in dichloroethane (60 mL), and the resulting mixture was stirred at room temperature for 2.5 h. Na(OAc)₃BH (6.56 g, 51.0 mmol) was then added, and the reaction was stirred at room temperature overnight, then quenched by then addition of (aqueous) sodium hydroxide (1M). The mixture was stirred for 30 minutes and the product was extracted using dichloromethane. The combined organic layers were dried over sodium sulfate, filtered and evaporated. The crude residue was purified via flash chromatography (hexane/ethyl acetate to afford 4.57 g (69%) of 4-[(2-triisopropylsilanyl-oxazol-5-ylmethyl)-amino]-benzoic acid tert-butyl ester as a white solid.

### Example 16

### Synthesis of 4-[(4-Difluoromethoxy-3-ethoxy-phenyl)-oxazolo-5-ylmethyl-amino]-benzoic acid tert-butyl ester

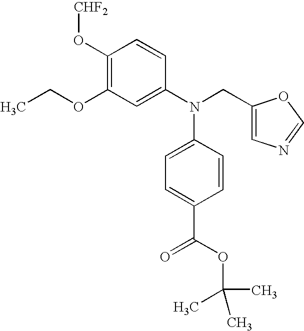

A mixture of toluene/DME (10/3, 13 mL) was added to a mixture of 4-[(2-triisopropylsilanyl-oxazol-5-ylmethyl)-amino]-benzoic acid tert-butyl ester (2.00 g, 4.23 mmol), 4-bromo-1-difluoromethoxy-2-ethoxy-benzene (1.69 g, 6.35 mmol), sodium hydroxide (508 mg, 12.7 mmol), and Pd(t-Bu₃P)₂ (216 mg, 0.42 mmol) under an atmosphere of argon. The reaction mixture was stirred at room temperature for 15 minutes, then heated to 40° C. for 15 minutes and then to 50° C. for 3.5 hr. After cooling to room temperature, MP-TMT (macroporous polystyrene-2,4,6-trimercaptotriazine) resin (1.0 g) was added. The mixture was stirred overnight, filtered through celite, and the filtrate was evaporated in vacuo. The resulting residue was purified via flash chromatography (hexane/EtOAc) to afford 2.04 g of 4-[(4-difluoromethoxy-3-ethoxy-phenyl)-oxazolo-5-ylmethyl-amino]-benzoic acid tert-butyl ester as a foamy syrup.

### Example 17

### Synthesis of 4-[(4-Difluoromethoxy-3-ethoxy-phenyl)-oxazolo-5-ylmethyl-amino]-benzoic acid

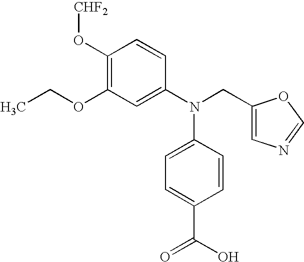

A mixture of 4-[(4-difluoromethoxy-3-ethoxy-phenyl)-oxazolo-5-ylmethyl-amino]-benzoic acid tert-butyl ester (2.35 g), formic acid (3.5 mL) and hydrochloric acid (6M, 3.5 mL) in toluene (5 mL) was heated to 60° C. for 30 minutes. The reaction mixture was then concentrated in vacuo, and the residue was taken up in water and ethyl acetate. The pH was adjusted to 6.5, the organic and aqueous layers were separated, and the aqueous layer was extracted twice with ethyl acetate. The combined organic extracts were washed with brine, dried over magnesium sulfate (,) filtered, and concentrated in vacuo. The crude residue was purified via flash chromatography (dichloromethane (/) methanol) to afford 1.62 g of the desired product (as a foam), which was recrystallized from ethyl acetate. The resulting white crystals were collected by filtration, washed with cold ethyl acetate, and dried in vacuo at 60° C. to provide 4-[(4-difluoromethoxy-3-ethoxy-phenyl)-oxazolo-5-ylmethyl-amino]-benzoic acid. Mp=111.1-113.0° C.

mp 111.1-113.0° C.; ¹H NMR (DMSO-d₆) δ 12.34 (bs, 1H), 8.30 (s, 1H), 7.77-7.74 (m, 2H), 7.31-6.82 (m, 7H), 5.06 (s, 2H), 4.03 (q, J=7 Hz, 2H), 1.31 (t, J=7 Hz, 3H); MS (ESI, [M+H⁺]) m/z 405; Anal. Calcd for C₂₀H₁₈F₂N₂O₅: C, 59.40; H, 4.49; N, 6.93. Found: C, 59.30; H, 4.37; N, 6.88.

### Example 18

### Synthesis of 4-[(2-Thiazol-5-ylmethyl)-amino]-benzoic acid tert-butyl ester

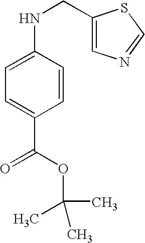

A mixture of 4-amino-benzoic acid tert-butyl ester (4.13 g, 21.4 mmol), thiazole-5-carboxaldehyde (2.20 g, 19.5 mmol) and Na(OAc)₃BH (6.18 g, 29.2 mmol) in dichloroethane (50 mL) was stirred at room temperature for 16 hr. The reaction mixture was then diluted with dichloromethane, washed with a saturated solution of sodium bicarbonate and brine, dried over sodium sulfate, filtered, and evaporated. The resulting residue was purified via flash chromatography (hexane/EtOAc) to afford 5.84 g of 4-[(2-Thiazol-5-ylmethyl)-amino]-benzoic acid tert-butyl ester as a white solid.

### Example 19

### Synthesis of 4-[(4-Difluoromethoxy-3-methoxy-phenyl)-thiazol-5-ylmethyl-amino]-benzoic acid tert-butyl ester

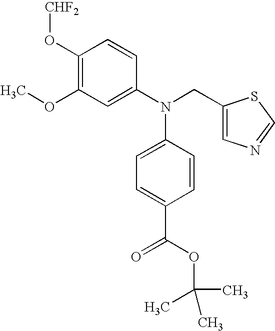

A mixture of toluene/DME (5/1, 18 mL) was added to a mixture of 4-[(2-thiazol-5ylmethyl)-amino]-benzoic acid tert-butyl ester (2.00 g, 6.89 mmol), 4-bromo-1-difluoromethoxy-2-methoxy-benzene (2.61 g, 10.3 mmol), sodium hydroxide (826 mg, 20.7 mmol), and Pd(t-Bu₃P)₂ (352 mg, 0.69 mmol) under an atmosphere of argon. The resulting mixture was stirred at room temperature for 15 minutes, then heated at 40° C. for 15 minutes, finally to 60° (60)-65° C. for 3.5 hours. After cooling to room temperature, MP-TMT (macroporous polystyrene-2,4,6-trimercaptotriazine) resin (1.0 g) was added. The mixture was stirred overnight and then filtered through celite. The filtrate was evaporated in vacuo and the residue was purified via flash chromatography (hexane/ethyl acetate) to afford 0.47 g of 4-[(4-Difluoromethoxy-3-methoxy-phenyl)-thiazol-5-ylmethyl-amino]-benzoic acid tert-butyl ester as a colorless syrup.

### Example 20

### Synthesis of 4-[(4-Difluoromethoxy-3-methoxy-phenyl)-thiazol-5-ylmethyl-amino]-benzoic acid

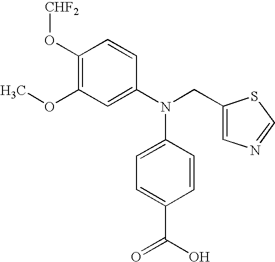

A mixture of 4-[(4-difluoromethoxy-3-methoxy-phenyl)-thiazol-5-ylmethyl-amino]-benzoic acid tert-butyl ester (2.90 g), formic acid (9 mL) and HCl (6M, 9 mL) in toluene (10 mL) was heated to reflux for 4 hr. The reaction mixture was then cooled, concentrated in vacuo, and the residue was partitioned between water (75 mL) and ethyl acetate (150 mL). The organic layer was separated and the aqueous layer was extracted twice with ethyl acetate. The combined organic extracts were washed with water (50 mL) and brine (50 mL), dried over magnesium sulfate, filtered and concentrated in vacuo. The resulting residue was purified via flash chromatography (dichloromethane/methanol) to afford the product as foam. The foam was recrystallized from ethyl acetate to give lightly colored crystals, which were (further) recrystallized from isopropanol/ethyl acetate (1/1) to give 382 mg of 4-[(4-Difluoromethoxy-3-methoxy-phenyl)-thiazol-5-ylmethyl-amino]-benzoic acid as colorless crystals. The mother liquors were concentrated and the residue recrystallized from isopropanol/ethyl acetate (1/1) to afford an additional 879 mg of 4-[(4-Difluoromethoxy-3-methoxy-phenyl)-thiazol-5-ylmethyl-amino]-benzoic acid as colorless crystals. Recrystallization of the mother liquors was then repeated, affording an additional 628 mg of crystalline 4-[(4-Difluoromethoxy-3-methoxy-phenyl)-thiazol-5-ylmethyl-amino]-benzoic acid. Mp=164.9-165.3° C.

mp 164.9-165.3° C.; ¹H NMR (DMSO-d₆) δ 12.36 (bs, 1H), 8.97 (d, J=0.8 Hz, 1H), 7.87 (s, 1H), 7.77-7.74 (m, 2H), 7.31-6.80 (m, 6H), 5.23 (s, 2H), 3.78 (s, 3H); MS (ESI, [M+H⁺]) m/z 407; Anal. Calcd for C₁₉H₁₆F₂N₂O₄S: C, 56.15; H, 3.97; N, 6.89. Found: C, 55.90; H, 3.90; N, 6.91.

### Example 21

### Ester Hydrolysis Using LiOH

The following compounds were prepared from the corresponding methyl or ethyl esters by base hydrolysis using LiOH. The general procedure involves stirring 1 equivalent of ester in a 1:1 mixture of THF and H₂O (20 mL/mmol) with 12 equivalents of LiOH for about 16 hours while heating to reflux. The mixture is cooled to room temperature, acidified with dilute HCl and extracted with EtOAc. The organic fraction is washed with brine, dried (Na₂SO₄) and concentrated. Purification by chromatography over SiO₂ using 4% MeOH and 0.5% AcOH in CH₂Cl₂ provided the desired acids below.
3-{(3-cyanobenzyl) [3-(cyclopentyloxy)-4-methoxyphenyl]amino}benzoic acid
3-{[3-(cyclopentyloxy)-4-methoxyphenyl][(6-oxo-1,6-dihydropyridin-3-yl)methyl]amino}benzoic acid
4-{[3-(cyclopentyloxy)-4-methoxyphenyl][(6-oxo-1,6-dihydropyridin-3-yl)methyl]amino}benzoic acid
{3-[[3-(cyclopentyloxy)-4-methoxyphenyl](pyridin-3-ylmethyl)amino]phenyl}acetic acid
5-[[3-(cyclopentyloxy)-4-methoxyphenyl](pyridin-3-ylmethyl)amino]-2-ethylbenzoic acid
3-[[3-(cyclopentyloxy)-4-methoxyphenyl](pyridin-3-ylmethyl)amino]-4-methylbenzoic acid
4-[[3-(cyclopentyloxy)-4-methoxyphenyl](pyrimidin-5-ylmethyl)amino]benzoic acid
4-[[3-(cyclopentyloxy)-4-methoxyphenyl](pyridazin-3-ylmethyl)amino]benzoic acid
{4-[[3-(cyclopentyloxy)-4-methoxyphenyl](pyridin-3-ylmethyl)amino]phenyl}acetic acid
5-[[3-(cyclopentyloxy)-4-methoxyphenyl](pyridin-3-ylmethyl)amino]nicotinic acid
3-{4-[[3-(cyclopentyloxy)-4-methoxyphenyl](pyridin-3-ylmethyl)amino]phenyl}propanoic acid
{3-[[3,4-bis(difluoromethoxy)phenyl](pyridin-3-ylmethyl)amino]phenyl}acetic acid
4-[[3-(cyclopentyloxy)-4-methoxyphenyl](pyridin-3-ylmethyl)amino]-3-methylbenzoic acid
{4-[[3,4-bis(difluoromethoxy)phenyl](pyridin-3-ylmethyl)amino]phenyl}acetic acid
5-[[3,4-bis(difluoromethoxy)phenyl](1,3-thiazol-5-ylmethyl)amino]-2-hydroxybenzoic acid
4-[[3,4-bis(difluoromethoxy)phenyl](1,3-thiazol-5-ylmethyl)amino]-2-hydroxybenzoic acid
{4-[[3,4-bis(difluoromethoxy)phenyl](1,3-thiazol-5-ylmethyl)amino]phenyl}acetic acid
{3-[[3,4-bis(difluoromethoxy)phenyl](1,3-thiazol-5-ylmethyl)amino]phenyl}acetic acid
{4-[[3,4-bis(difluoromethoxy)phenyl](pyrimidin-5-ylmethyl)amino]phenyl}acetic acid
{3-[[3,4-bis(difluoromethoxy)phenyl](pyrimidin-5-ylmethyl)amino]phenyl}acetic acid
{4-[[3,4-bis(difluoromethoxy)phenyl](pyrazin-2-ylmethyl)amino]phenyl}acetic acid
{3-[[4-(difluoromethoxy)-3-ethoxyphenyl](1,3-oxazol-5-ylmethyl)amino]phenyl}acetic acid
{4-[[4-(difluoromethoxy)-3-ethoxyphenyl](1,3-oxazol-5-ylmethyl)amino]phenyl}acetic acid
{3-[[4-(difluoromethoxy)-3-ethoxyphenyl](1,3-thiazol-5-ylmethyl)amino]phenyl}acetic acid
4-[[4-(difluoromethoxy)-3-ethoxyphenyl](1,3-oxazol-4-ylmethyl)amino]benzoic acid
cis-4-[[3,4-bis(difluoromethoxy)phenyl](1,3-thiazol-5-ylmethyl)amino]cyclohexanecarboxylic acid, and
4-[[3-ethoxy-4-(trifluoromethoxy)phenyl](1,3-oxazol-5-ylmethyl)amino]benzoic acid.

The following compounds were prepared in a similar fashion as describe above, but the NaOH was used instead of LiOH:
3-[[4-(difluoromethoxy)-3-ethoxyphenyl](pyrimidin-5-ylmethyl)amino]benzoic acid
3-[(3-ethoxy-4-methoxyphenyl)(pyrimidin-5-ylmethyl)amino]benzoic acid
3-[[3-(cyclopropylmethoxy)-4-methoxyphenyl](pyrimidin-5-ylmethyl)amino]benzoic acid
3-[(3-isopropoxy-4-methoxyphenyl)(pyrimidin-5-ylmethyl)amino]benzoic acid
3-[[3-(cyclopropyloxy)-4-methoxyphenyl](pyrimidin-5-ylmethyl)amino]benzoic acid
3-[[4-(difluoromethoxy)-3-isopropoxyphenyl](pyrimidin-5-ylmethyl)amino]benzoic acid
3-[[4-(cyclopropyloxy)-3-ethoxyphenyl](pyrimidin-5-ylmethyl)amino]benzoic acid
3-[[3-(cyclobutyloxy)-4-methoxyphenyl](pyrimidin-5-ylmethyl)amino]benzoic acid, and
4-[[3-(cyclopropyloxy)-4-(difluoromethoxy)phenyl](pyrimidin-5-ylmethyl)amino]benzoic acid.

### Example 22

### Synthesis of 4-[[3,4-bis(difluoromethoxy)phenyl](pyridin-3-ylmethyl)amino]-N-(methylsulfonyl)benzamide

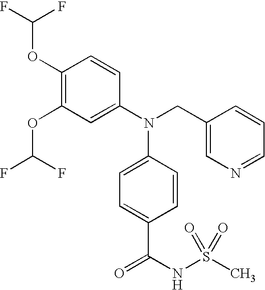

4-[[3,4-bis(difluoromethoxy)phenyl](pyridin-3-ylmethyl)amino]benzoic acid (50 mg, 0.11 mmol), methanesulfonamide (22 mg, 0.23 mmol), EDCI (44 mg), DMAP (17 mg) and DMF were combined and stirred for 16 hours at room temperature and then at 100° C. for 1 hour. The mixture was cooled, diluted with 40 mL of EtOAc and washed with 10 mL of 1M HCl, 10 mL of H₂O, 10 mL of brine, dried (Na₂SO₄) and concentrated. The residue was purified by HPLC over a reverse phase column using a gradient elution going from 20 to 80% CH₃CN in H₂O with 0.5% formic acid over 6 minutes with a flow rate of 45 mL/minute to provide 2.3 mg of 4-[[3,4-bis(difluoromethoxy)phenyl](pyridin-3-ylmethyl)amino]-N-(methylsulfonyl)benzamide.

The following compound was prepared in a similar fashion but starting with ethanesulfonaminde rather than methansulfonamide: 4-[[3,4-bis(difluoromethoxy)phenyl]-(pyridin-3-ylmethyl)amino]-N-(ethylsulfonyl)benzamide.

### Example 23

### Preparation of 4-[[3-(cyclopentyloxy)-4-methoxyphenyl](pyridin-3-ylmethyl)amino]phenol

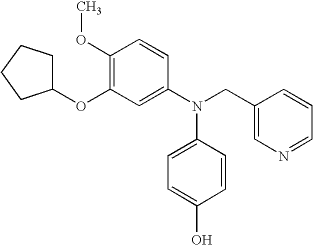

### 1. Preparation of (3-cyclopentyloxy-4-methoxy-phenyl)-[4-[((E and Z)-propenyl)oxy]-phenyl]-pyridin-3-ylmethyl-amine

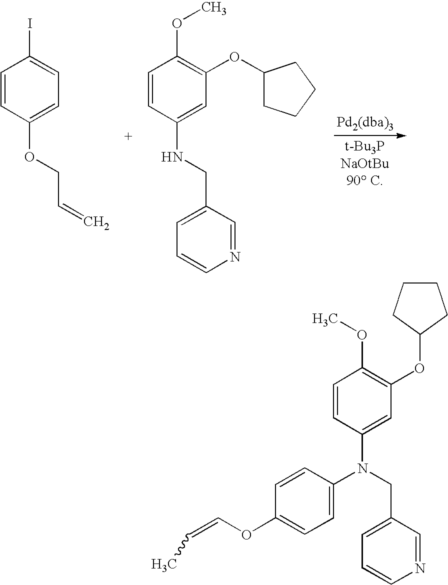

To a mixture of 400 mg (1.53 mmol) of 4-allyloxy-iodobenzene and 371 mg (1.0 mmol) 3-cyclopentyloxy-4-methoxy-phenyl)-pyridin-3-ylmethyl-amine in toluene 5 mL were added 70 mg of Pd₂(dba)₃, tri-t-butylphosphine (40 μL) and sodium t-butoxide (500 mg). The reaction mixture was heated for 18 h at 90° C., after which time the disappearance of the starting amine was observed by TLC. The reaction mixture was cooled and passed through a 10 g column of silica gel, eluting with methylene chloride, then 5% acetone/methylene chloride. The crude material (300 mg), whose MS was consistent with product, was isolated and repurified by flash chromatography, using 20% acetone/hexane as eluent to give 250 mg of material. An analytical sample was obtained by dissolving 100 mg of the material in methylene chloride and stirring with MP-TMT (macroporous polystyrene-2,4,6-trimercaptotriazine) overnight. The resin was removed by filtration and washed with methylene chloride. The filtrate was concentrated in vacuo to provide the desired intermediate.

### 2. Preparation of 4-[(3-cyclopentyloxy-4-methoxy-phenyl)-pyridin-3-ylmethyl-amino]-phenol

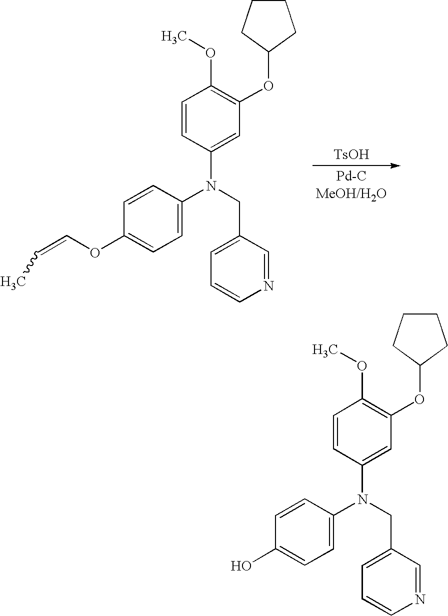

The product from Step 1 (150 mg), TsOH (200 mg) and Pd—C (70 mg) were heated overnight in of 5:1 MeOH/H₂O (1 mL). The cooled reaction mixture was then filtered through Celite® and the filtrate concentrated in vacuo. The residue was diluted with ethyl acetate and basified with NaHCO₃ (aq). The organic layer was separated, washed with brine, dried over Na₂SO₄ (anhyd) and concentrated in vacuo. The residue was purified by flash chromatography, eluting with 20% acetone/methylene chloride, to obtain 90 mg of product, confirmed by NMR spectroscopy.

The following analogs were made in a similar fashion:
3-[[3-(cyclopentyloxy)-4-methoxyphenyl](pyridin-3-ylmethyl)amino]phenol
4-[[3,4-bis(difluoromethoxy)phenyl](3-pyridylmethyl)amino]phenol, and
4-[3-cyclopentyloxy-4-methoxy)phenyl](1,3-thiazol-5-ylmethyl)amino]phenol

### Example 24

### Synthesis of ethyl {4-[[3-(cyclopentyloxy)-4-methoxyphenyl](pyridin-3-ylmethyl)amino]phenoxy}acetate

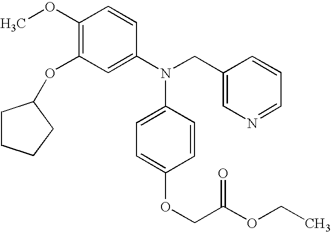

To a solution of 4-[[3-(cyclopentyloxy)-4-methoxyphenyl](pyridin-3-ylmethyl)amino]phenol: (270 mg, 0.69 mmol) in DMF was added NaH (60% oil dispersion; 80 mg, 2.0 mmol) and the mixture stirred for 15 minutes. Ethyl bromoacetate (117 mg, 0.69 mmol) was added and stirring continued for an additional 30 minutes. Saturated aq. NH₄Cl was added and the mixture was extracted with ethyl acetate. The organic fraction was isolated and washed with brine, dried (Na₂SO₄) and concentrated. Purification by flash chromatography using 20% acetone in hexanes to provide ethyl {4-[[3-(cyclopentyloxy)-4-methoxyphenyl](pyridin-3-ylmethyl)amino]phenoxy}acetate. Elemental Analysis Calcd for C₂₈H₃₂N₂O₃: Theory C, 70.56; H, 6.77; N, 5.88. Found C, 70.34; H, 6.55; N, 5.93.

### Example 25

### Synthesis of 2-{4-[[3-(cyclopentyloxy)-4-methoxyphenyl](pyridin-3-ylmethyl)amino]phenoxy}ethanol

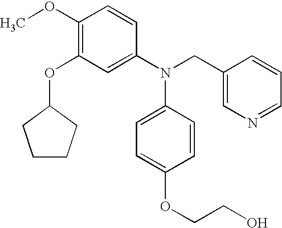

To an ice-cold solution of ethyl {4-[[3-(cyclopentyloxy)-4-methoxyphenyl](pyridin-3-ylmethyl)amino]phenoxy}acetate (70 mg, 0.15 mmol) in THF was added a 0.5 M solution of LAH in THF and the solution stirred for 10 minutes. Saturated aq. NH₄Cl was added followed by EtOAc. The precipitates were removed by filtration and the combined filtrate was dried over Na₂SO₄ and purified by flash chromatography to provide 2-{4-[[3-(cyclopentyloxy)-4-methoxyphenyl](pyridin-3-ylmethyl)amino]phenoxy}ethanol. Elemental analysis calcd for C₂₆H₃₀N₂O₄+0.5H₂O: Theory C, 70.39; H, 6.99; N,6.31. Found C, 70.86; H, 6.81; N, 6.41.

### Example 26

### Synthesis of 3,4-bis(difluoromethoxy)-N-[3-(methylsulfonyl)phenyl]-N-(pyridin-3-ylmethyl)aniline

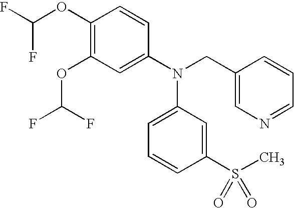

To a solution of 3,4-bis(difluoromethoxy)-N-[3-(methylthio)phenyl]-N-(pyridin-3-ylmethyl)aniline (130 mg, 0.30 mmol) in CH₂Cl₂ (4 mL) with stirring at 0° C. was added MCPBA (213 mg, 0.74 mmol) in two portions. The mixture was stirred at 0° C. for 20 minutes and then quenched by the addition of 20% aq. Na₂SO₃ and saturated aq NaHCO₃ solution. The mixture was then extracted with EtOAc and the organic fraction was washed with H₂O, brine, dried (MgSO₄) and concentrated to give a residue. Purification by chromatography over SiO₂ using 50% EtOAc in hexanes as eluant provided 98 mg of 3,4-bis(difluoromethoxy)-N-[3-(methylsulfonyl)phenyl]-N-(pyridin-3-ylmethyl)aniline as a clear oil.

The following compounds were made in a similar fashion:
N-(3-fluorobenzyl)-4-methoxy-N-[4-(methylsulfonyl)phenyl]-3-[(3R)-tetrahydrofuran-3-yloxy]aniline
3,4-bis(difluoromethoxy)-N-[4-(methylsulfonyl)phenyl]-N-(1,3-thiazol-5-ylmethyl)aniline
3,4-bis(difluoromethoxy)-N-[4-(ethylsulfonyl)phenyl]-N-(1,3-thiazol-5-ylmethyl)aniline
3,4-bis(difluoromethoxy)-N-[3-(methylsulfonyl)phenyl]-N-(1,3-thiazol-5-ylmethyl)aniline, and
3,4-bis(difluoromethoxy)-N-[3-(methylsulfonyl)phenyl]-N-(pyrimidin-5-ylmethyl)aniline.

### Example 27

### Synthesis of 1-{4-[[34-bis(difluoromethoxy)phenyl](pyridin-3-ylmethyl)amino]-2-hydroxyphenyl}ethanone

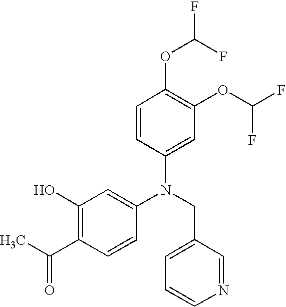

To a solution of 1-{4-[[3,4-bis(difluoromethoxy)phenyl](pyridin-3-ylmethyl)amino]-2-tert-butyldimethylsilyloxyphenyl}ethanone (1 mmol) in toluene was added 100 mg of pTsOH and the mixture was warmed to 80° C. for 30 minutes with stirring. The mixture was cooled to room temperature, diluted with EtOAc and washed with saturated aq. NaHCO₃, H₂O, brine, dried (Na₂SO₄) and concentrated. Purification by chromatography over SiO₂ using 10% acetone in CH₂Cl₂ provided 1-{4-[[3,4-bis(difluoromethoxy)phenyl](pyridin-3-ylmethyl)amino]-2-hydroxyphenyl}ethanone. Elemental analysis calculated for C₂₂H₁₈F₄N₂O₄. Theory C, 58.67; H, 4.03; N, 6.22. Found C, 58.71; H, 3.97; N, 6.15.

The following compounds were prepared in a similar fashion:
2-[[3,4-bis(difluoromethoxy)phenyl](phenyl)amino]ethanol.

### Example 28

### Synthesis of 5-[[34-bis(difluoromethoxy)phenyl](pyridin-3-ylmethyl)amino]-2,3-difluoro-1-hydroxyphenyl

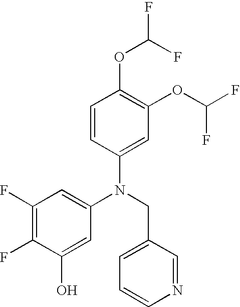

5-[[3,4-bis(difluoromethoxy)phenyl](pyridin-3-ylmethyl)amino]-2,3-difluoro-1-(tert-butyldimethylsilyl)oxyphenyl and 0.5M NaOMe in MeOH (5 mL) were combined and stirred for 30 minutes. The mixture was diluted with EtOAc and washed with dilute HCl, brine, dried (Na₂SO₄) and concentrated. Purification by chromatography over SiO₂ using 10% acetone in CH₂Cl₂ provided 50 mg of 5-[[3,4-bis(difluoromethoxy)phenyl](pyridin-3-ylmethyl)amino]-2,3-difluoro-1-hydroxyphenyl.

### Example 29

### Synthesis of 5-[3-[[3,4-bis(difluoromethoxy)phenyl](pyridin-3-ylmethyl)amino]phenyl]-13=oxazolidin-2-one

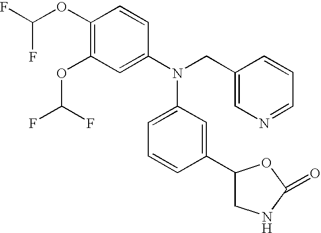

### Step 1. Synthesis of 3-[[3,4-bis(difluoromethoxy)phenyl](pyridin-3-ylmethyl)amino]phenyl}-oxirane

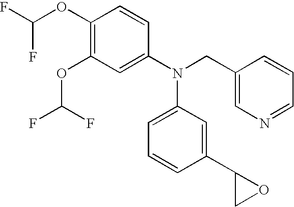

To a solution of sodium hydride (60% in mineral oil, 225 mg, 5.63 mmol, washed with hexanes twice) in DMSO (10mL) was added trimethylsulfoxonium iodide (1.19 g, 5.4 mmol). The suspension was stirred at room temperature for 45 minutes. A solution of 3-[[3,4-bis(difluoromethoxy)phenyl](pyridin-3-ylmethyl)amino]phenyl}carboxaldehyde (2.03 g, 4.8 mmol) in DMSO (5 mL) was added to the reaction mixture and stirred at room temperature for 2 hours. The reaction was diluted with water and extracted with ethyl acetate 3 times. The organic extracts were dried with magnesium sulfate, concentrated, and chromatographed (20% ethyl acetate/hexane gradient to 50% ethyl acetate/hexane) to give 409 mg of a tan oil (20%).

### Step 2. Synthesis of 1-{3-[[3,4-bis(difluoromethoxy)phenyl](pyridin-3-ylmethyl)amino]phenyl}-2-azidoethan-1-ol and 2-{3-[[3,4-bis(difluoromethoxy)phenyl](pyridin-3-ylmethyl)amino]phenyl}-2-azidoethan-1-ol

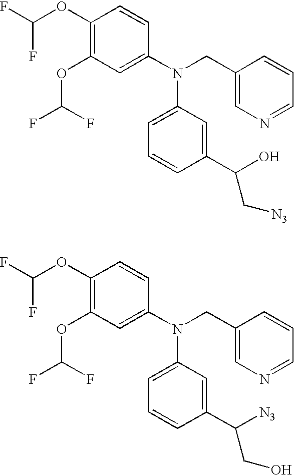

Sodium azide (75 mg, 1.154 mmol) was added to a solution of 3-[[3,4-bis(difluoromethoxy)phenyl](pyridin-3-ylmethyl)amino]phenyl}-oxirane (248 mg, 0.571 mmol) and lithium perchlorate (141 mg, 1.325 mmol) in acetonitrile-acetone-water (7:1:1, 9 mL). The reaction was stirred overnight at 40° C., concentrated, and chromatographed (20% ethyl acetate/hexane, gradient to 35%, then 45%) to give 24 mg (9%) of secondary alcohol 1-{3-[[3,4-bis(difluoromethoxy)phenyl](pyridin-3-ylmethyl)amino]phenyl}-2-azidoethan-1-ol and 163 mg (60%) of primary alcohol 2-{3-[[3,4-bis(difluoromethoxy)phenyl](pyridin-3-ylmethyl)amino]phenyl}-2-azidoethan-1-ol. Rf (1:1 hexane/ethyl acetate 0.2 secondary alcohol, 0.1 primary alcohol).

### Step 3. 1-{3-[[3,4-bis(difluoromethoxy)phenyl](pyridin-3-ylmethyl)amino]phenyl}-2-aminoethan-1-ol

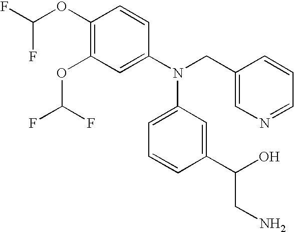

A solution of azide-alcohol 1-{3-[[3,4-bis(difluoromethoxy)phenyl](pyridin-3-ylmethyl)amino]phenyl}-2-azidoethan-1-ol (105 mg, 0.220 mmol) and tin chloride dihydrate (104 mg, 0.461 mmol) in methanol (4 mL) was stirred overnight at room temperature. The reaction was diluted with ethyl acetate and 1N NaOH was added and stirred for 1 hour. The reaction mixture was extracted with ethyl acetate 3 times and the organic extracts were dried with sodium sulfate. The crude residue was concentrated and chromatographed (10% MeOH (w/5% NH₄OH)/CH₂Cl₂) to give 42 mg of a white solid (42%).

### Step 4. 1-{3-[[3,4-bis(difluoromethoxy)phenyl](pyridin-3-ylmethyl)amino]phenyl}-2-(tert-butyloxycarbonylamino)ethan-1-ol

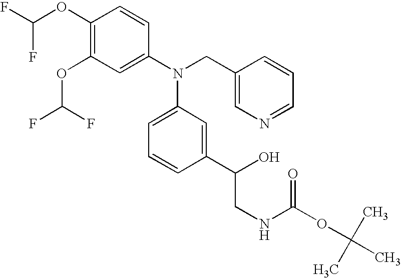

A solution of amino alcohol 1-{3-[[3,4-bis(difluoromethoxy)phenyl](pyridin-3-ylmethyl)amino]phenyl}-2-aminoethan-1-ol (42 mg, 0.093 mmol), di-tert-butyl dicarbonate (23 mg, 0.105 mmol), potassium carbonate (32 mg, 0.232 mmol) in water-THF (1:1, 4 mL) was stirred at room temperature for 3 hours. The reaction mixture was concentrated and chromatographed (50% Ethyl acetate/hexane) to give 1-{3-[[3,4-bis(difluoromethoxy)phenyl](pyridin-3-ylmethyl)amino]phenyl}-2-(tert-butyloxycarbonylamino)ethan-1-ol as a colorless oil (29 mg, 56%).

### Step 5. 5-{3-[[3,4-bis(difluoromethoxy)phenyl](pyridin-3-ylmethyl)amino]phenyl}-1,3-oxazolidin-2-one

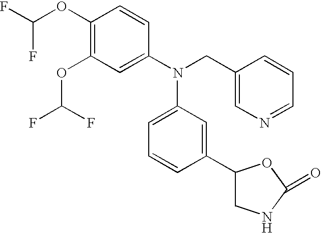

To a solution of 1-{3-[[3,4-bis(difluoromethoxy)phenyl](pyridin-3-ylmethyl)amino]phenyl}-2-(tert-butyloxycarbonylamino)ethan-1-ol (29 mg, 0.053 mmol) in THF (3 mL), was added sodium hydride (60% in mineral oil, 3 mg, 0.075 mmol). The slurry was stirred overnight at 65°. The crude reaction was concentrated and chromatographed (3% MeOH (w/5% NH₄OH)/CH₂Cl₂) to give oxazolidinone 101 as a colorless oil (11 mg, 44%).

### Example 30

### Synthesis of 4-{3-[[3,4-bis(difluoromethoxy)phenyl](pyridin-3-ylmethyl)amino]phenyl}-1,3-oxazolidin-2-one

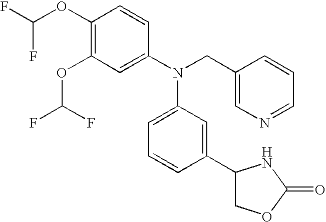

### Step 1. 2-{3-[[3,4-bis(difluoromethoxy)phenyl](pyridin-3-ylmethyl)amino]phenyl}-2-aminoethan-1-ol

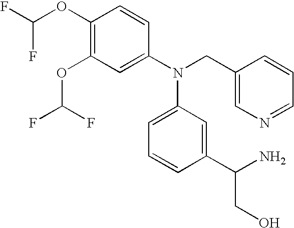

A solution of 2-{3-[[3,4-bis(difluoromethoxy)phenyl](pyridin-3-ylmethyl)amino]phenyl}-2-azidoethan-1-ol (267 mg, 0.560 mmol) and tin chloride dihydrate (263 mg, 1.166 mmol) in methanol (8 mL) was stirred overnight at room temperature. The reaction was concentrated, diluted with ethyl acetate and 1N NaOH was added and stirred for 1 hour. The reaction mixture was extracted with ethyl acetate 3 times and the organic extracts were dried with sodium sulfate. The crude residue was concentrated and chromatographed (10% MeOH (w/5% NH₄OH)/CH₂Cl₂) to give 60 mg of 2-{3-[[3,4-bis(difluoromethoxy)phenyl](pyridin-3-ylmethyl)amino]phenyl}-2-aminoethan-1-ol as a colorless oil (24%)

### Step 2. 2-{3-[[3,4-bis(difluoromethoxy)phenyl](pyridin-3-ylmethyl)amino]phenyl}-2-(tert-butyloxycarbonylamino)ethan-1-ol

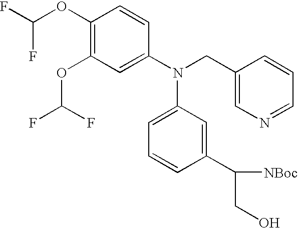

A solution of 2-{3-[[3,4-bis(difluoromethoxy)phenyl](pyridin-3-ylmethyl)amino]phenyl}-2-aminoethan-1-ol (60 mg, 0.133 mmol), di-tert-butyl dicarbonate (34 mg, 0.156 mmol), potassium carbonate (37 mg, 0.268 mmol) in water-THF (1:1, 4 mL) was stirred at room temperature for 3 hours. The reaction mixture was concentrated and chromatographed (50% Ethyl acetate/hexane) to give 2-{3-[[3,4-bis(difluoromethoxy)phenyl](pyridin-3-ylmethyl)amino]phenyl}-2-(tert-butyloxycarbonylamino)ethan-1-ol as a colorless oil (54 mg, 74%).

### Step 3. 4-{3-[[3,4-bis(difluoromethoxy)phenyl](pyridin-3-ylmethyl)amino]phenyl}-1,3-oxazolidin-2-one

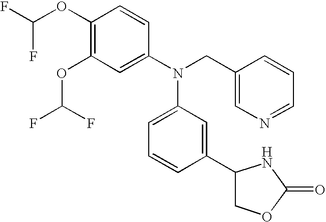

To a solution of 2-{3-[[3,4-bis(difluoromethoxy)phenyl](pyridin-3-ylmethyl)amino]phenyl}-2-(tert-butyloxycarbonylamino)ethan-1-ol (54 mg, 0.098 mmol) in THF (5 mL), was added sodium hydride (60% in mineral oil, 5 mg 0.125 mmol). The slurry was stirred for 3 hours at 65°. The crude reaction was concentrated and chromatographed (3% MeOH (w/5% NH₄OH)/CH₂Cl₂) to give 4-{3-[[3,4-bis(difluoromethoxy)phenyl](pyridin-3-ylmethyl)amino]phenyl}-1,3-oxazolidin-2-one as a colorless oil (12 mg, 26%).

### Example 31

### Synthesis of 3,4-bis(difluoromethoxy)-N-[3-(1H)-pyrazol-3-yl)phenyl]-N-(pyridin-3-ylmethyl)aniline

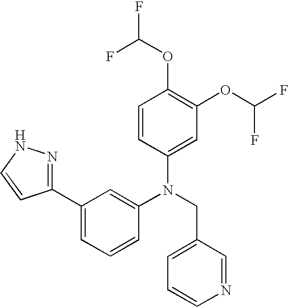

A solution of 3,4-bis(difluoromethoxy)-N-[3-(1H)-1-triphenylmethyl-pyrazol-3-yl)phenyl]-N-(pyridin-3-ylmethyl)aniline (0.3 g, 0.42 mmol), CF₃CO₂H (1 mL) and CH₂Cl₂ (3 mL) was stirred for 30 minutes and then diluted with H₂O and EtOAc. The solution was basified with NaHCO₃ and the organic fraction was separated and washed with H₂O, brine, dried (Na₂SO₄), and concentrated. Purification by chromatography over SiO₂ using 3% MeOH in CH₂Cl₂ gave 100 mg of 3,4-bis(difluoromethoxy)-N-[3-(1H)-pyrazol-3-yl)phenyl]-N-(pyridin-3-ylmethyl)aniline as a glassy solid.

### Example 32

### Synthesis of 3,4-bis(difluoromethoxy)-N-(pyridin-3-ylmethyl)-N-[3-(1H-1,2,4-triazol-5-yl)phenyl]aniline

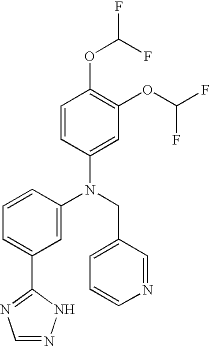

A solution of 3,4-bis(difluoromethoxy)-N-(3-cyanophenyl)-N-(pyridin-3-ylmethyl)aniline (75 mg,) in MeOH was treated with 60% NaH (20 mg) and formic hydrazide (50 mg). The mixture was stirred for 30 minutes at room temperature and then heated to reflux for 16 hours. The mixture was cooled, concentrated and purified by chromatography over SiO2 using 3.5% MeOH in CH2Cl2 as eluant to provide 15 mg of 3,4-bis(difluoromethoxy)-N-(pyridin-3-ylmethyl)-N-[3-(1H-1,2,4-triazol-5-yl)phenyl]aniline.

### Example 33

### Synthesis of 4-[[3,4-bis(difluoromethoxy)phenyl](1,3-thiazol-5-ylmethyl)amino]-benzenesulphonic acid

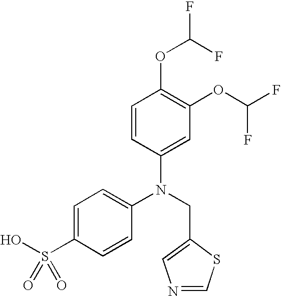

A mixture of isobutyl 4-[[3,4-bis(difluoromethoxy)phenyl](1,3-thiazol-5-ylmethyl)amino]-benzenesulphonate (100 mg), acetone (2 mL) and NaI (100 mg) was heated to reflux for 16 hours. The mixture was cooled, concentrated and used as such in the next step.

### Example 34

### Synthesis of 4-[[3,4-bis(difluoromethoxy)phenyl](1,3-thiazol-5-ylmethyl)amino]-N,N-dimethylbenzenesulfonamide

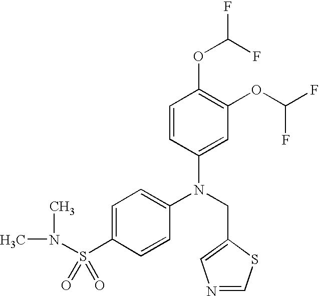

4-[[3,4-bis(difluoromethoxy)phenyl](1,3-thiazol-5-ylmethyl)amino]-benzenesulphonic acid (from above) was taken up in 3 mL of CH2Cl2, and 2 drops of DMF and 100 μL of oxalyl chloride were added and the mixture stirred for 30 minutes. All volatiles were removed under reduced pressure and the residue was taken up in THF (2 mL) and 40% aq. Me2NH was added. The reaction mixture stirred for 30 minutes, was diluted with EtOAc and washed with H2O, brine, dried (Na2SO4) and concentrated. Purification by chromatography over SiO2 using 8% acetone in hexanes as eluant to give 20 mg of 4-[[3,4-bis(difluoromethoxy)phenyl](1,3-thiazol-5-ylmethyl)amino]-N,N-dimethylbenzenesulfonamide.

The following compounds were prepared in a similar fashion:
4-[[3,4-bis(difluoromethoxy)phenyl](1,3-thiazol-5-ylmethyl)amino]benzenesulfonamide
4-[[3,4-bis(difluoromethoxy)phenyl](1,3-thiazol-5-ylmethyl)amino]-N-methylbenzenesulfonamide.

### Example 35

### Synthesis of 4-[[34-bis(difluoromethoxy)phenyl](1,3-thiazol-5-ylmethyl)amino]-N-methylbenzamide

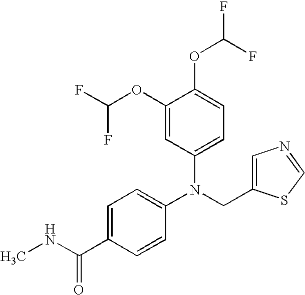

Oxalyl chloride (43 μL, 0.5 mmol) was added to a solution of 4-[[3,4-bis(difluoromethoxy)phenyl](1,3-thiazol-5-ylmethyl)amino]-benzoic acid (109 mg, 0.25 mmol), DMF (1 drop) and CH2Cl2 (2 mL). The mixture stirred at 38° C. for 1.5 hours and was concentrated under reduced pressure. The residue was diluted with THF (2 mL) followed by the addition of 2.0 M methylamine in THF (600 μL, 1.2 mmol). The mixture stirred for 1 hour at room temperature and then filtered through celite and concentrated. Purification by chromatography over SiO2 using 5% MeOH, 1% TEA, 94% CH2Cl2 as eluant provided 64 mg of 4-[[3,4-bis(difluoromethoxy)phenyl](1,3-thiazol-5-ylmethyl)amino]-N-methylbenzamide.

The following compounds were prepared in a similar fashion:
4-[[3,4-bis(difluoromethoxy)phenyl](1,3-thiazol-5-ylmethyl)amino]benzamide
4-[[3,4-bis(difluoromethoxy)phenyl](1,3-thiazol-5-ylmethyl)amino]-N,N-dimethylbenzamide.

### Example 36

### Synthesis of N-{5-[[3,4-bis(difluoromethoxy)phenyl](1,3-thiazol-5-ylmethyl)amino]-2-methylphenyl}methanesulfonamide

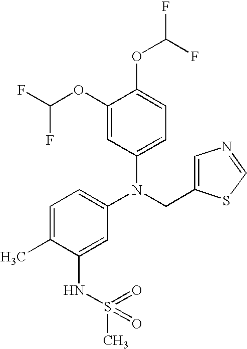

To a mixture of N-(3-amino-4-methylphenyl)-N-[[3,4-bis(difluoromethoxy)phenyl](1,3-thiazol-5-ylmethyl)amine (65 mg, 0.15 mmol) and Amberlite IRA67 (54 mg, 0.30 mmol) in CH₂Cl₂ in a vial was added methanesulfonylchloride (17.8 μL, 0.23 mmol). The vial was sealed and stirred for several hours and then DMAP was added. This mixture stirred for 3 days and then diluted with CH₂Cl₂ and PS-trisamine and (macroporous polystyrene-2,4,6-trimercaptotriazine) TMT were added. This mixture stirred for 16 hours and was then filtered and concentrated. Purification by preparative TLC using 2.5% MeOH in CH₂Cl₂ gave 18 mg of N-{5-[[3,4-bis(difluoromethoxy)phenyl](1,3-thiazol-5-ylmethyl)amino]-2-methylphenyl}methanesulfonamide.

The follow compounds were prepared in a similar manner:
N-{3-[[3,4-bis(difluoromethoxy)phenyl](1,3-thiazol-5-ylmethyl)amino]phenyl}-methanesulfonamide
N-{3-[[3,4-bis(difluoromethoxy)phenyl](1,3-thiazol-5-ylmethyl)amino]phenyl}-ethanesulfonamide.

### Example 37

### Synthesis of 1-[[3,4-bis(difluoromethoxy)phenyl](3-chlorophenyl)amino]-2-methylpropan-2-ol

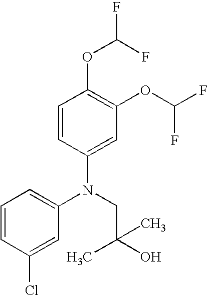

1-[[3,4-bis(difluoromethoxy)phenyl](3-chlorophenyl)amine (100 mg, 0.33 mmol), 2,2-dimethyloxirane (200 μL), CaCO₃ (40 mg, 0.4 mmol) and DMF were combined and warmed to 90° C. for 3 days. The mixture was diluted with H₂O and extracted with EtOAc. The organic fractions were combined and washed with H₂O, brine, dried (Na₂SO₄) and concentrated. Purification by chromatography over SiO₂ using 20% CH₂Cl₂ in hexanes to elute unreacted starting material and 10% acetone in hexanes to elute the product provided 24 mg of 1-[[3,4-bis(difluoromethoxy)phenyl](3-chlorophenyl)amino]-2-methylpropan-2-ol as an oil.

The following compounds were prepared in a similar fashion, but with different starting materials:
1-[[3,4-bis(difluoromethoxy)phenyl](3-chlorophenyl)amino]-propan-2,3-oxirane
3-[[3,4-bis(difluoromethoxy)phenyl](3-chlorophenyl)amino]propan-1-ol.

### Example 38

### Synthesis of 1-[[34-bis(difluoromethoxy)phenyl](3-chlorophenyl)amino]propan-2-ol

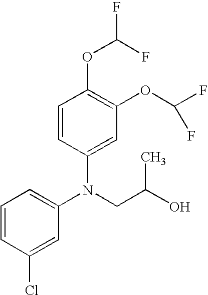

A solution of 1-[[3,4-bis(difluoromethoxy)phenyl](3-chlorophenyl)amino]propan-2,3-oxirane (40 mg, 0.1 mmol) in THF (3 mL) was treated with a 1M solution of LiAlH₄ in THF (0.5 mL, 0.5 mmol). The mixture was stirred at room temperature for 10 minutes and quenched by the addition of NH₄Cl. EtOAc was added and the mixture was filtered through a pad of celite, dried (Na₂SO₄) and concentrated. Purification by chromatography over SiO₂ using 25% EtOAc in hexanes as eluant provided 25 mg of 1-[[3,4-bis(difluoromethoxy)phenyl](3-chlorophenyl)amino]propan-2-ol as an oil.

### Example 39

### Preparation of 4-[(3-cyclopentyloxy-4-methoxy-phenyl)-pyridin-3-ylmethyl-amino]-phenol

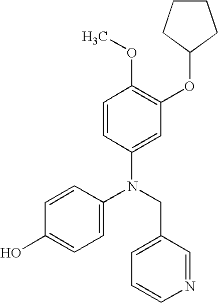

### 1. Preparation of (3-cyclopentyloxy-4-methoxy-phenyl)-[4-[((E and Z)-propenyl)oxy]-phenyl]-pyridin-3-ylmethyl-amine

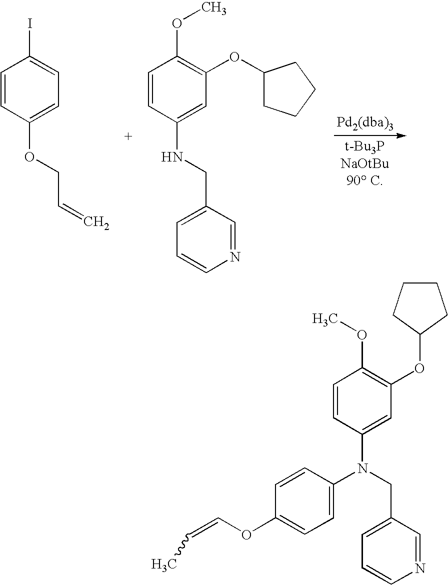

To a mixture of 400 mg (1.53 mmol) of 4-allyloxy-iodobenzene and 371 mg (1.0 mmol) 3-cyclopentyloxy-4-methoxy-phenyl)-pyridin-3-ylmethyl-amine in toluene (mL) were added 70 mg of Pd₂(dba)₃, tri-t-butylphosphine (40 μL) and sodium t-butoxide (500 mg). The reaction mixture was heated for 18 h at 90° C., after which time the disappearance of the starting amine was observed by TLC. The reaction mixture was cooled and passed through a 10 g column of silica gel, eluting with methylene chloride, then 5% acetone/methylene chloride. The crude material (300 mg), whose MS was consistent with product, was isolated and repurified by flash chromatography, using 20% acetone/hexane as eluent to give 250 mg of material. An analytical sample was obtained by dissolving 100 mg of the material in methylene chloride and stirring with MP-TMT overnight. The resin was removed by filtration and washed with methylene chloride. The filtrate was concentrated in vacuo to provide the desired intermediate.

### 2. Preparation of 4-[(3-cyclopentyloxy-4-methoxy-phenyl)-pyridin-3-ylmethyl-amino]-phenol

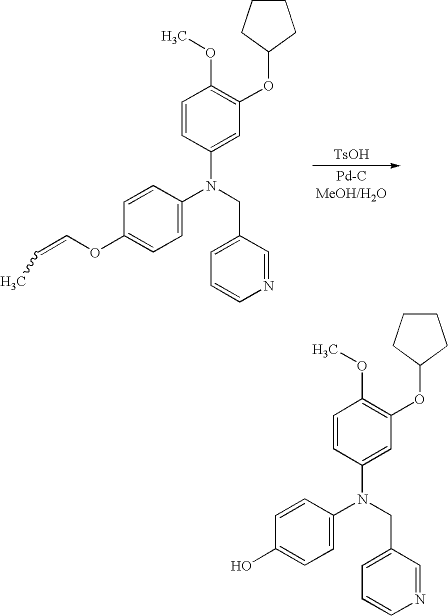

The product from Step 1 (150 mg), TsOH (200 mg) and Pd—C (70 mg) were heated overnight in of 5:1 MeOH/H₂O (1 mL). The cooled reaction mixture was then filtered through Celite® and the filtrate concentrated in vacuo. The residue was diluted with ethyl acetate and basified with NaHCO₃ (aq). The organic layer was separated, washed with brine, dried over Na₂SO₄ (anhyd) and concentrated in vacuo. The residue was purified by flash chromatography, eluting with 20% acetone/methylene chloride, to obtain 90 mg of product, confirmed by NMR spectroscopy.

### Example 40

### Synthesis of 3-(cyclopentyloxy)-N-(4-{[(4S)-2,2-dimethyl-1,3-dioxolan-4-yl]methoxy}phenyl)-4-methoxy-N-(pyridin-3-ylmethyl)aniline

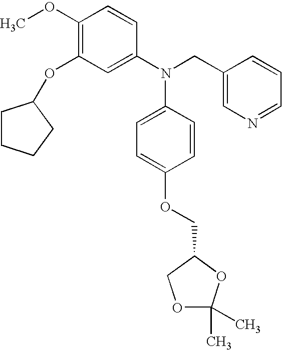

To a solution of 4-[(3-cyclopentyloxy-4-methoxy-phenyl)-pyridin-3-ylmethyl-amino]-phenol (50 mg, 0.13 mmol) in DMF (2 mL) was added NaH (60% dispersion; 10 mg, 0.25 mmol) and the mixture stirred at room temperature for 15 minutes. (R)-2,2-Dimethyl-1,3-dioxolane-4-ylmethyl p-toluenesulfonate (100 mg, 0.35 mmol) was added and the mixture was stirred at 60° C. for 15 minutes. The mixture was cooled to room temperature and diluted with H₂O and extracted with EtOAc. The organic fraction was collected and washed with H₂O, brine, dried (Na₂SO₄) and concentrated. Purification by chromatography over SiO₂ using 20% acetone in hexanes as eluant provided about 40 mg of 3-(cyclopentyloxy)-N-(4-{[(4S)-2,2-dimethyl-1,3-dioxolan-4-yl]methoxy}phenyl)-4-methoxy-N-(pyridin-3-ylmethyl)aniline, which was used as such in the next step.

### Example 41

### Synthesis of (2R)-3-{4-[[3-(cyclopentyloxy)-4-methoxyphenyl](pyridin-3-ylmethyl)amino]phenoxy}propane-1,2-diol

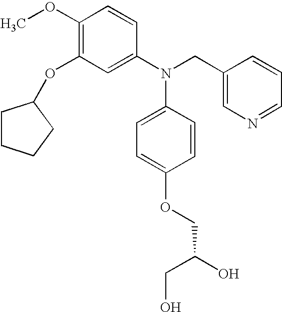

3-(Cyclopentyloxy)-N-(4-{[(4S)-2,2-dimethyl-1,3-dioxolan-4-yl]methoxy}phenyl)-4-methoxy-N-(pyridin-3-ylmethyl)aniline (50 mg, 0.1 mmol) was dissolved in 1 mL of 20% aqueous acetone and 200 μL of 6N HCl was added. The mixture stirred for 20 minutes, was basified by addition of saturated aq. NaHCO₃ and then extracted with EtOAc. The organic fraction was washed with water, brine, dried (Na₂SO₄) and concentrated. Purification by chromatography over SiO₂ using 3% MeOH in CH₂Cl₂ provided 17 mg of (2R)-3-{4-[[3-(cyclopentyloxy)-4-methoxyphenyl](pyridin-3-ylmethyl)amino]phenoxy}propane-1,2-diol as a gum.

The following compounds were prepared in a similar fashion:
2R)-3-{3-[[3-(cyclopentyloxy)-4-methoxyphenyl](pyridin-3-ylmethyl)amino]phenoxy}propane-1,2-diol
(2R)-3-{4-[[3-(cyclopentyloxy)-4-methoxyphenyl](1,3-thiazol-5-ylmethyl)amino]phenoxy}propane-1,2-diol
(2R)-3-{4-[(3,4-dimethoxyphenyl)(pyridin-3-ylmethyl)amino]phenoxy}propane-1,2-diol
5-[[3,4-bis(difluoromethoxy)phenyl](pyridin-3-ylmethyl)amino]-2-{[(2R)-2,3-dihydroxypropyl]oxy}benzoic acid
(2R)-3-{3-[[3,4-bis(difluoromethoxy)phenyl](1,3-thiazol-5-ylmethyl)amino]phenoxy}propane-1,2-diol
(2R)-3-{4-[[3,4-bis(difluoromethoxy)phenyl](1,3-thiazol-5-ylmethyl)amino]phenoxy}propane-1,2-diol
(2R)-3-{3-[[3,4-bis(difluoromethoxy)phenyl](1,3-thiazol-5-ylmethyl)amino]phenoxy}propane-1,2-diol.

### Example 42

### Synthesis of 5-[[3-(cyclopentyloxy)-4-methoxyphenyl](pyridin-3-ylmethyl)amino]-1,2-benzisoxazol-3(2H)-one

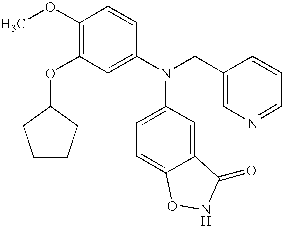

Step 1. Methyl 5-[[3-(cyclopentyloxy)-4-methoxyphenyl](pyridin-3-ylmethyl)amino]-2-(tert-butyldimethylsilyloxybenzoate (140 mg, 0.25 mmol), 50% hydroxylamine (2.5 mL) and MeOH (5 mL) were combined and stirred for 1 hour followed by the addition of 0.5M NaOMe (1.0 mL). The mixture was concentrated and the remaining aqueous fraction was washed with hexanes (3 times) and acidified with 1N HCl carefully to adjust the pH to 6.5 and a precipitate formed. The aqueous phase was decanted and the remaining precipitate was washed with water and dried in a vacuum oven to give 103 mg of 5-[[3-(cyclopentyloxy)-4-methoxyphenyl](pyridin-3-ylmethyl)amino]-N,2-dihydroxybenzamide as an off white solid that turned dark upon storage.

Step 2. 5-[[3-(Cyclopentyloxy)-4-methoxyphenyl](pyridin-3-ylmethyl)amino]-N,2-dihydroxybenzamide (53 mg, 0.12 mmol), triphenylphosphine (37 mg, 0.14 mmol) and THF were combined and then DEAD (22 μl, 0.14 mmol) was added drop-wise with stirring. The mixture stirred for 24 hours and was diluted with CH₂Cl₂ and washed with water, brine, dried (Na₂SO₄) and concentrated. Purification by chromatography over SiO₂ using EtOAc as eluant gave crude material in need of further purification. Additional chromatography over SiO₂ using a gradient elution going from 2% to 5% MeOH in CH₂Cl₂ gave 15 mg of 5-[[3-(cyclopentyloxy)-4-methoxyphenyl](pyridine-3-ylmethyl)amino]-1,2-benzisoxazol-3(2H)-one as a purple foam.

The following compounds were prepared in a similar fashion:
5-[[3,4-bis(difluoromethoxy)phenyl](1,3-thiazol-5-ylmethyl)amino]-1,2-benzisoxazol-3(2H)-one
6-[[3,4-bis(difluoromethoxy)phenyl](1,3-thiazol-5-ylmethyl)amino]-1,2-benzisoxazol-3(2H)-one.

### Example 43

### Synthesis of 5-{4-[[3-(cyclopentyloxy)-4-methoxyphenyl](pyridin-3-ylmethyl)amino]phenyl}-imidazolidine-2,4-dione

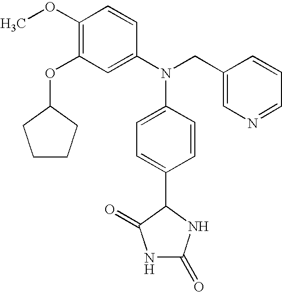

4-[[3-(cyclopentyloxy)-4-methoxyphenyl](pyridin-3-ylmethyl)amino]benzaldehyde (380 mg, 0.94 mmol), ammonium carbonate (907 mg, 9.44 mmol), KCN (86 mg, 1.32 mmol), MeOH (15 mL) and H₂O (15 mL) were combined and heated to 55° C. for 3 hours. The volatiles were removed in vacuo and the residue was taken up in EtOAc. The organic layer was washed with H₂O, brine, dried (Na₂SO₄) and concentrated. Purification by chromatography over SiO₂ using 5% MeOH in CH₂Cl₂ provided 140 mg of 5-{4-[[3-(cyclopentyloxy)-4-methoxyphenyl](pyridin-3-ylmethyl)amino]phenyl}imidazolidine-2,4-dione.

The following compounds were prepared in a similar fashion:
5-{3-[[3,4-bis(difluoromethoxy)phenyl](pyridine-3-ylmethyl)amino]phenyl}imidazolidine-2,4-dione
5-{3-[[3,4-bis(difluoromethoxy)phenyl](1,3-thiazol-5-ylmethyl)amino]phenyl}-1,3,5-trimethylimidazolidine-2,4-dione
5-{3-[[3,4-bis(difluoromethoxy)phenyl](1,3-thiazol-5-ylmethyl)amino]phenyl}imidazolidine-2,4-dione.

### Example 44

### Synthesis of 4-{3-[[3,4-bis(difluoromethoxy)phenyl](pyridin-3-ylmethyl)amino]phenyl}-imidazolidin-2-one

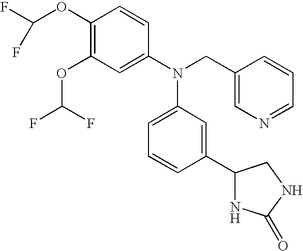

To a solution of 5-{3-[[3,4-bis(difluoromethoxy)phenyl](pyridine-3-ylmethyl)amino]phenyl}imidazolidine-2,4-dione (80 mg, 0.16 mmol) in THF (10 mL) at 0° C. with stirring was added a 1.0 M solution of LAH in THF (490 μL, 0.49 mmol) drop-wise. The mixture was then warmed to reflux for 5 h and then maintained at 50° C. for 16 h. The mixture was cooled to 0° C. and quenched by the addition of MeOH and water. The precipitates were removed by filtration and the crude product was filtered through SiO₂ using 5% MeOH in CH₂Cl₂ as eluant. Further purification by SiO₂ chromatography using a gradient elution going form 0 to 10% MeOH in EtOAc provided 4-{3-[[3,4-bis(difluoromethoxy)phenyl](pyridin-3-ylmethyl)amino]phenyl}imidazolidin-2-one as an amorphous solid.

### Example 45

### Synthesis of 2-{3-[[3,4-bis(difluoromethoxy)phenyl](pyridin-3-ylmethyl)amino]phenyl}propan-2-ol

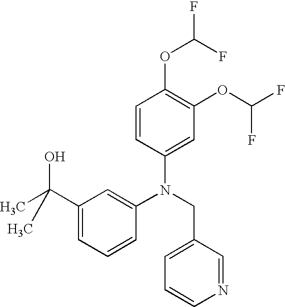

To a solution of 1-{3-[[3,4-bis(difluoromethoxy)phenyl](pyridin-3-ylmethyl)amino]phenyl}ethanone (80 mg, 0.18 mmol) in THF was added 3M MeMgCl (100 μL, 0.30 mmol) and the mixture stirred for 10 minutes. Saturated NH₄Cl and EtOAc were added and the mixture was filtered through celite, dried (Na₂SO₄) and concentrated. The residue was purified by chromatography over SiO₂ using 10% acetone in CH₂Cl₂ as eluant to provide 50 mg of 2-{3-[[3,4-bis(difluoromethoxy)phenyl](pyridin-3-ylmethyl)amino]phenyl}propan-2-ol as an oil.

### Example 46

### Synthesis of 4-{3-[[3-(cyclopentyloxy)-4-methoxyphenyl](pyridin-3-ylmethyl)amino]phenyl}-1-methyl-1H-pyrazol-5-ol

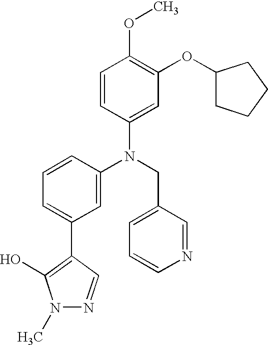

A solution of 3-{[3-(cyclopentyloxy-4-methoxy)phenyl(pyridin-3-ylmethyl)amino]phenyl}acetic acid (100 mg, 0.23 mmol) and 200 mL of bis(dimethylamino)-tert-butoxymethane (Bredereck's reagent) in THF was heated to reflux for 30 minutes. The mixture was cooled, concentrated and the residue was dissolved in MeOH and 100 mL of methyl hydrazine was added and the mixture was again warmed to reflux for 30 minutes. The mixture was cooled to room temperature, diluted with EtOAc and washed with water, brine, dried (Na₂SO₄) and concentrated. Purification by chromatography over SiO₂ using 8% MeOH in CH₂Cl₂ gave 20 mg of 4-{3-[[3-(cyclopentyloxy)-4-methoxyphenyl](pyridin-3-ylmethyl)amino]phenyl}-1-methyl-1H-pyrazol-5-ol.

The following compound was made in a similar fashion: 4-{3-[[3,4-bis(difluoromethoxy)phenyl](pyridin-3-ylmethyl)amino]phenyl}-2-methyl-1,2-dihydro-3H-pyrazol-3-one.

| | | LC/MS RT |
|---|---|---|
| | | (min) |
| | | observed mass |
| | | (m/z) |
| Cmpd | | mp |
| No | Structure | (° C.) |
| 1 | 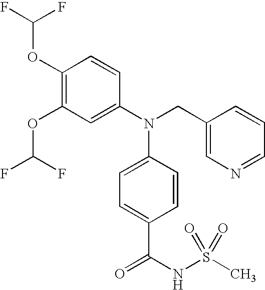 | (M + H)+ = 514.0 |
| 2 | 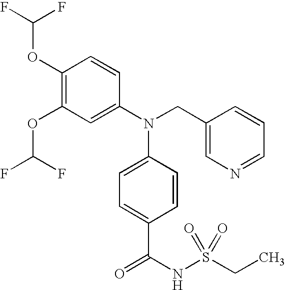 | (M + H)+ = 528.1 |
| 3 | 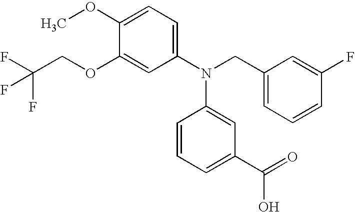 | — |
| 4 | 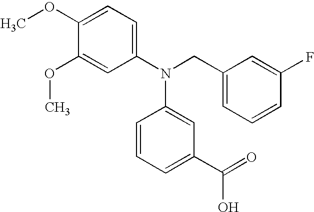 | — |
| 5 | 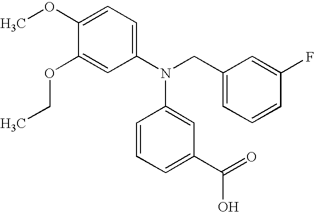 | — |
| 6 | 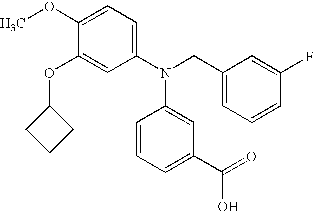 | — |
| 7 | 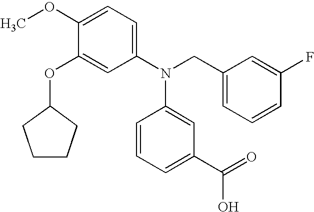 | — |
| 8 | 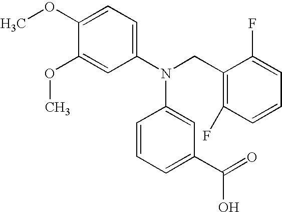 | — |
| 9 | 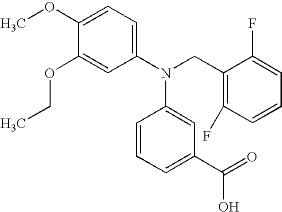 | — |
| 10 | 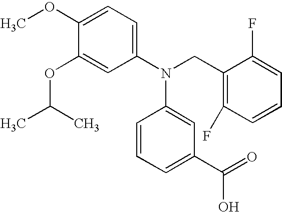 | — |
| 11 | 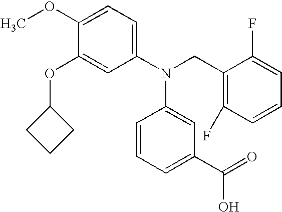 | — |
| 12 | 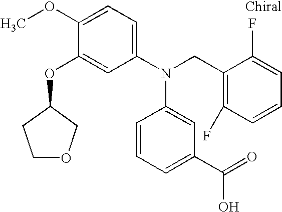 | — |
| 13 | 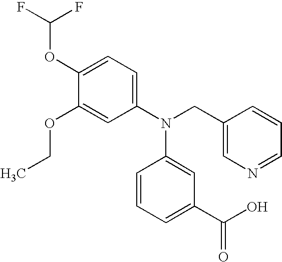 | [M + H] = 415.6 |
| 14 | 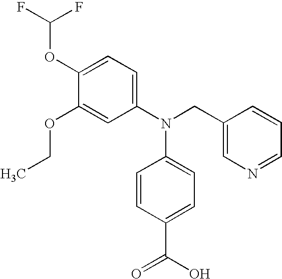 | [M + H] = 415.6 |
| 15 | 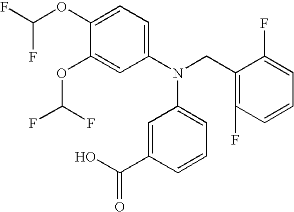 | [M − H] = 470.4 |
| 16 | 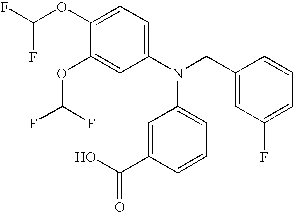 | [M − H] = 452.3 |
| 17 | 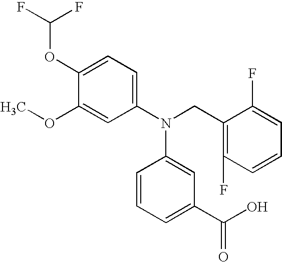 | [M − H] = 434.4 |
| 18 | 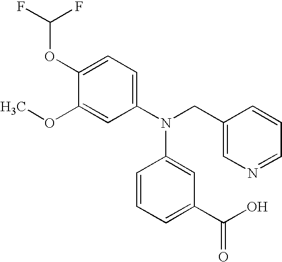 | [M − H] = 399.3 |
| 19 | 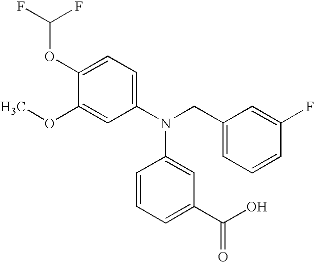 | [M − H] = 416.4 |
| 20 | 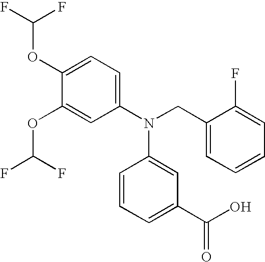 | [M − H] = 452.3 |
| 21 | 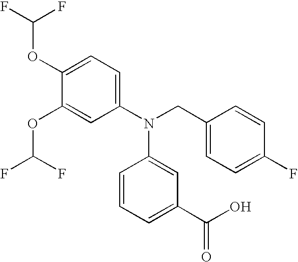 | [M − H] = 452.3 |
| 22 | 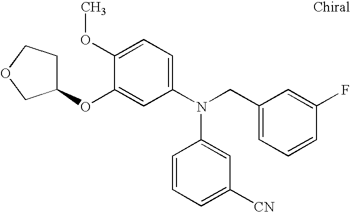 | [M + H] = 419.4 |
| 23 | 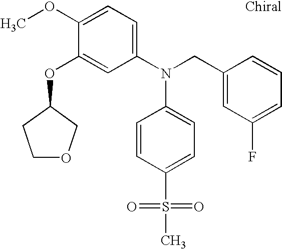 | [M + H] = 473.0 |
| 24 | 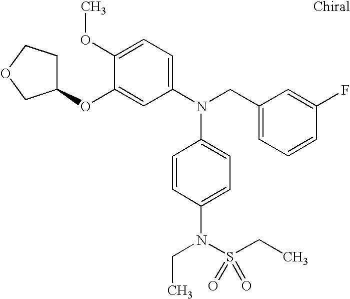 | [M + H] = 529.6 |
| 25 | 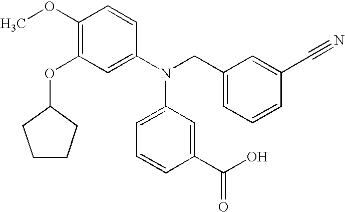 | [M + H] = 435 mp = 186.9-188.5° C. |
| 26 | 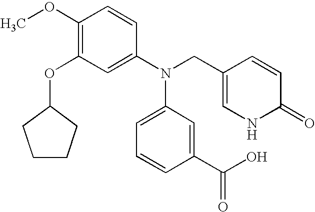 | [M + H] = 433 C = 72.07, H = 6.50, N = 6.40 |
| 27 | 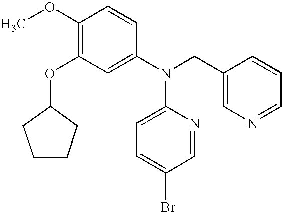 | [M + H] = 454 |
| 28 | 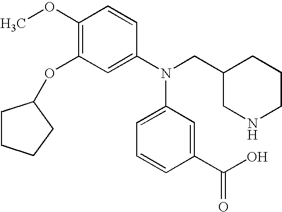 | [M + H]+ = 425 |
| 29 | 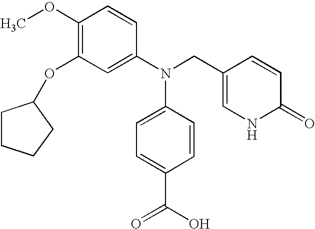 | [M + H] = 435 mp = 143.7-145.9° C. |
| 30 | 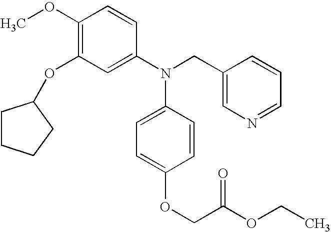 | [M + H] = 476 C 70.34, H 6.55, N 5.93, calculated C 70.56, H 6.77, N 5.88. |
| 31 | 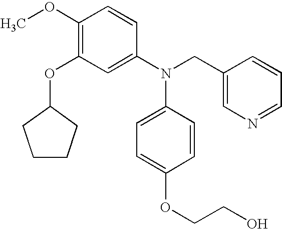 | [M + H] = 435; C 70.86, H 6.81, N 6.41, calculated C 70.39, H 6.99, N 6.31. |
| 32 | 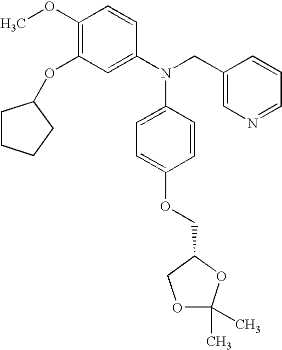 | [M + H] = 505; C 70.99, H 7.03, N 5.63, calculated C 70.93, H 7.15, N 5.5. |
| 33 | 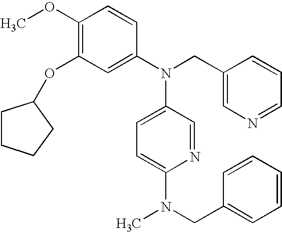 | [M + H] = 495 |
| 34 | 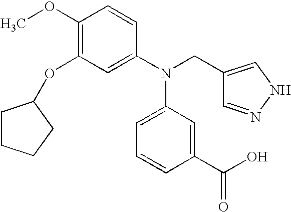 | [M + H]+ = 408 |
| 35 | 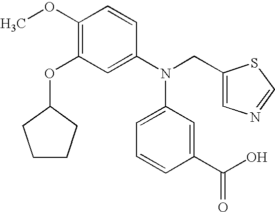 | mp = 65.6-70.6° C.; [M + H]+ = 425 |
| 36 | 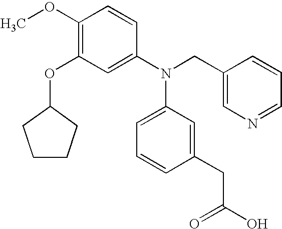 | [M + H]+ = 432 C 72.05, H 6.36, N 6.57, calculated C 72.20, H 6.53, N 6.48 |
| 37 | 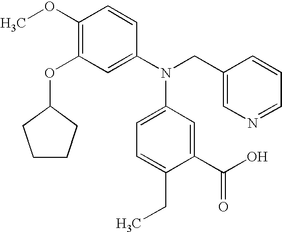 | [M + H]+ = 446 C 70.49, H 6.67, N 5.85, calculated C 70.39, H 6.90, N 6.09 |
| 38 | 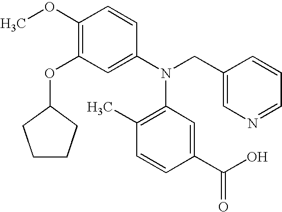 | [M + H]+ = 433 |
| 39 | 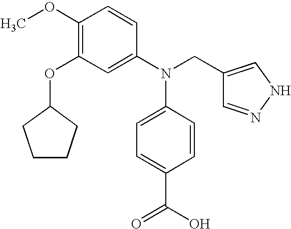 | mp = 108.9-112.3° C.; [M + H]+ = 408 |
| 40 | 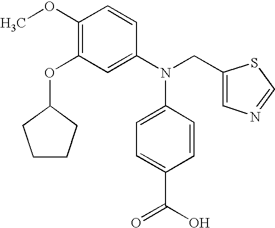 | mp = 88.0-90.3° C.; [M + H]+ = 425 |
| 41 | 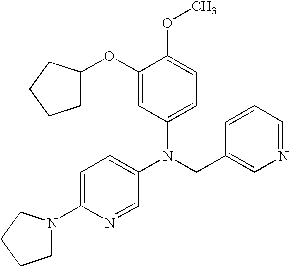 | [M + H] = 445 |
| 42 | 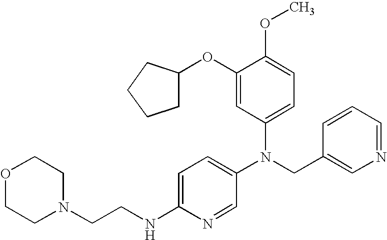 | [M + H] = 504 |
| 43 | 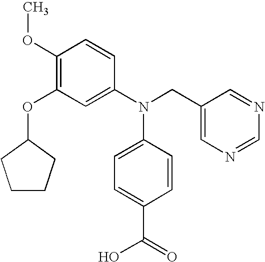 | [M + H] = 420 mp = 156.6-159.8° C. |
| 44 | 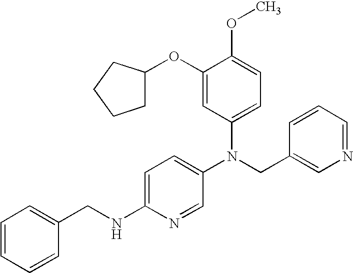 | [M + H] = 481 |
| 45 | 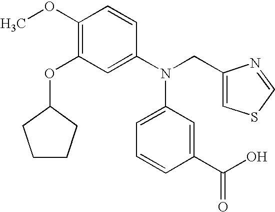 | mp = 75.5-77.7° C.; [M + H]+ = 425 |
| 46 | 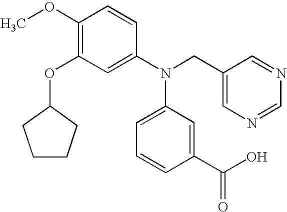 | [M + H] = 420 |
| 47 | 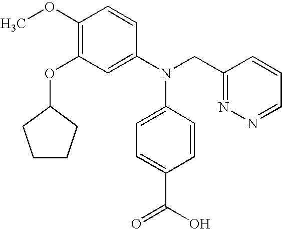 | [M + H] = 420 mp = 151.8-152.6° C. |
| 48 | 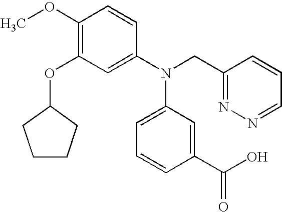 | [M + H] = 420 mp = 178.9-184.3° C. |
| 49 | 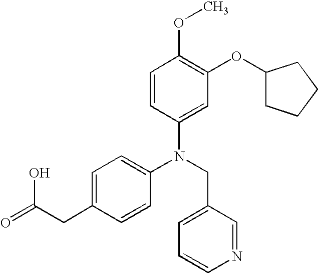 | [M + H]+ = 433 |
| 50 | 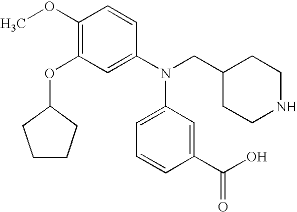 | mp = 228.8-232.1° C.; [M + H]+ = 425 |
| 51 | 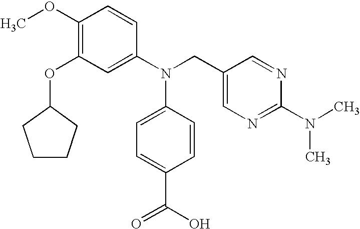 | mp = 95.9-99.5° C.; [M + H]+ = 463 |
| 52 | 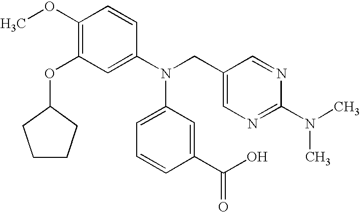 | mp = 82.0-84.7° C.; [M + H]+ = 463 |
| 53 | 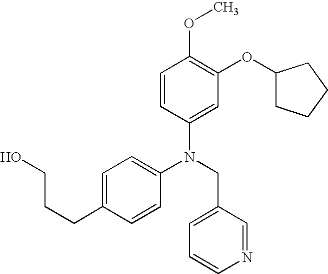 | [M + H] = 433 C 72.84, H 7.26, N 6.33, calculated C 72.67, H 7.26, N 6.23 |
| 54 | 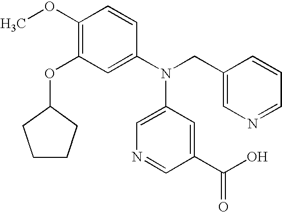 | [M + H] = 420 |
| 55 | 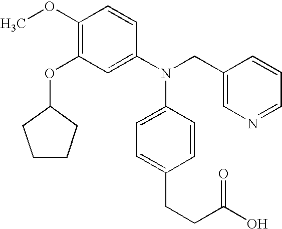 | [M + H]+ = 447 C 69.76, H 6.79, N 5.40, calculated C 72.62, H 6.77, N 6.27 |
| 56 | 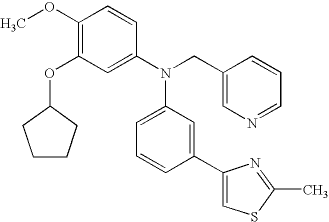 | [M + H] = 472 |
| 57 | 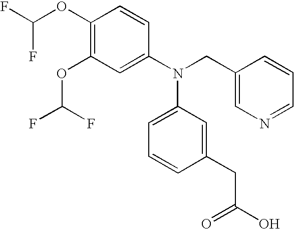 | [M + H]+ = 451 C 58.22, H 3.93, N 6.20, calculated C 58.67, H 4.03, N 6.22 |
| 58 | 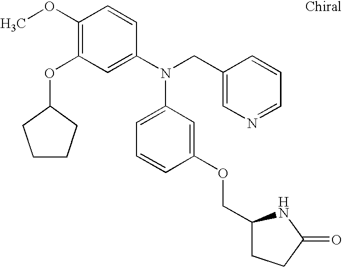 | [M + H] = 488 C 68.88, H 6.34, N 8.22, calculated C 68.59, H 6.60, N 8.19 |
| 59 | 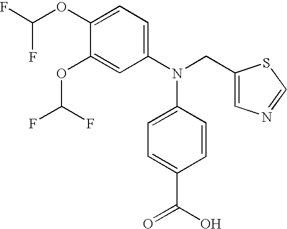 | Mp = 144.3°-145.7° C. |
| 60 | 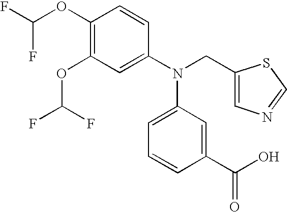 | EA: calc'd C: 51.59, H: 3.19, N: 6.33; found C: 51.28, H: 3.22, N: 6.25; [M + H]+ = 433 |
| 61 | 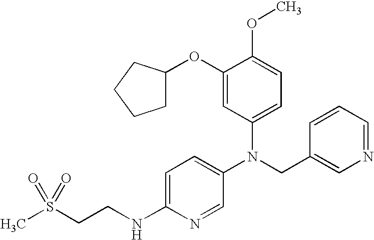 | [M + H] = 497 |
| 62 | 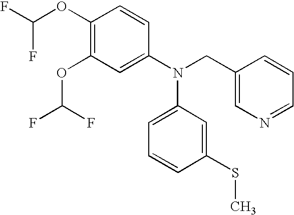 | [M + H] = 439 |
| 63 | 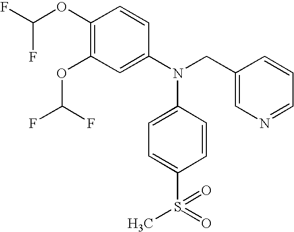 | [M + H] = 471 |
| 64 | 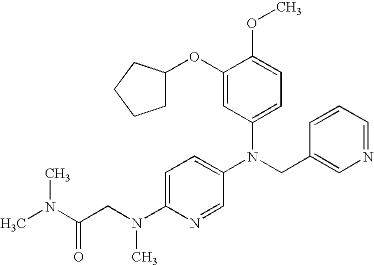 | [M + H] = 490 |
| 65 | 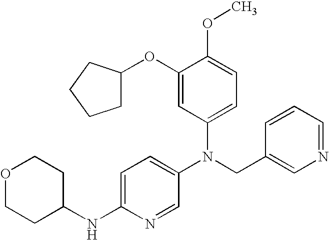 | [M + H] = 475 |
| 66 | 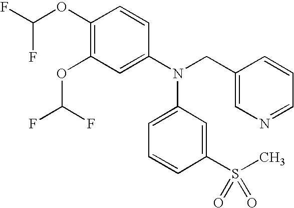 | [M + H] = 471 |
| 67 | 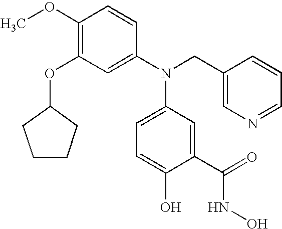 | [M + H] = 432 |
| 68 | 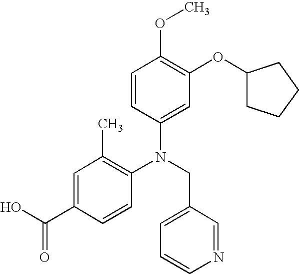 | [M + H]+ = 433 C 72.12, H 6.44, N 6.54, calculated C 72.2, H 6.53, N 6.48 |
| 69 | 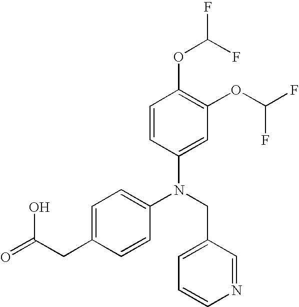 | [M + H]+ = 451 Mp = 107.4-108.1 |
| 70 | 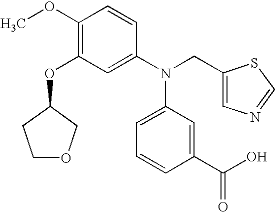 | mp = 60.3-61.9° C.; [M + H]+ = 427 |
| 71 | 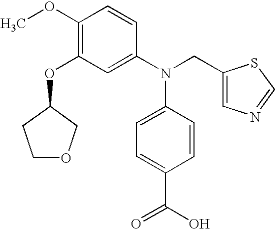 | mp = 60.3-60.8° C.; [M + H]+ = 427 |
| 72 | 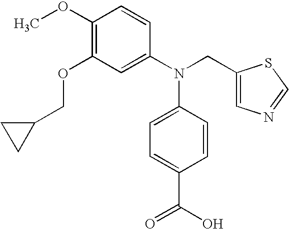 | mp = 128.7-129.4° C.; [M + H]+ = 411 |
| 73 | 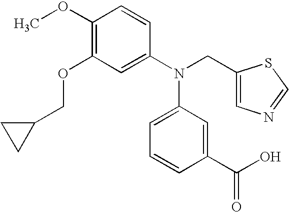 | mp = 137.4-139.4° C.; [M + H]+ = 411 |
| 74 | 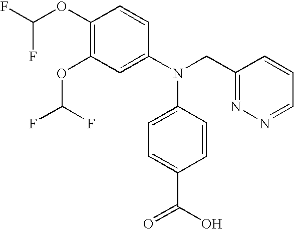 | [M + H] = 438 mp = 162.6-164.8° C. |
| 75 | 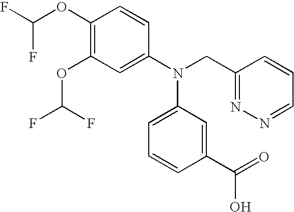 | [M + H] = 438 |
| 76 | 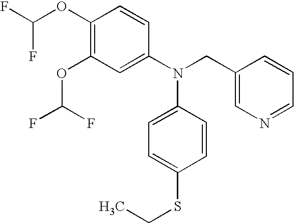 | [M + H] = 453 |
| 77 | 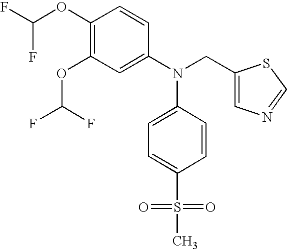 | [M + H] = 477 |
| 78 | 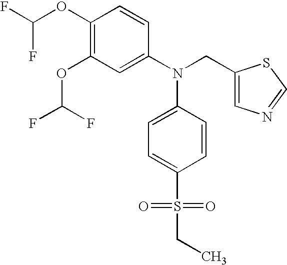 | [M + H]+ = 491 |
| 79 | 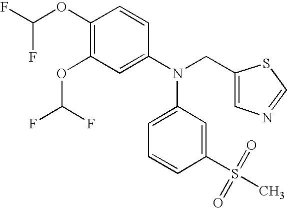 | [M + H]+ = 477 |
| 80 | 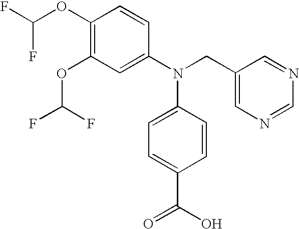 | [M + H] = 438 mp = 186.7-187.4° C. |
| 81 | 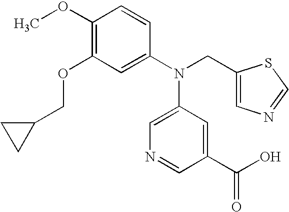 | [M + H] = 412 |
| 82 | 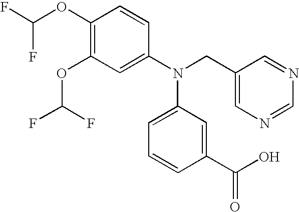 | [M + H] = 438 |
| 83 | 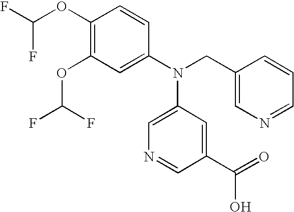 | [M + H] = 438 |
| 84 | 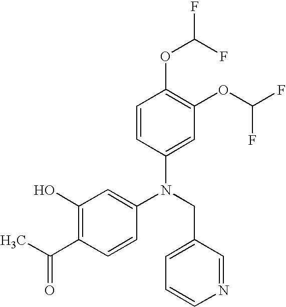 | [M + H] = 451 C 58, 71, H 3.97, N 6.15 calculated C 58.67, H 4.03, N 6.22. |
| 85 | 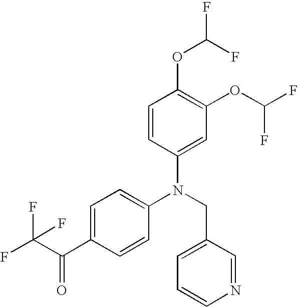 | [M + H]+ = 507 C 53.87, H 3.01, N 5.76 calculated C 54.11, H 3.10, N 5.74 |
| 86 | 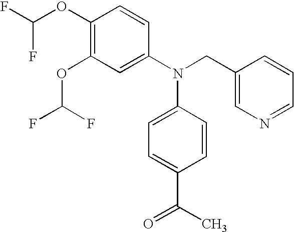 | [M + H] = 435 |
| 87 | 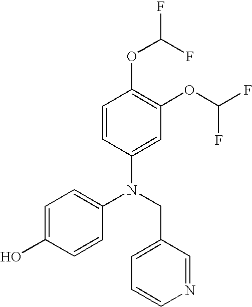 | [M + H] = 409 C 58.86, H 3.84, N 6.89 calculated C 58.83, H 3.95, N 6.86 |
| 88 | 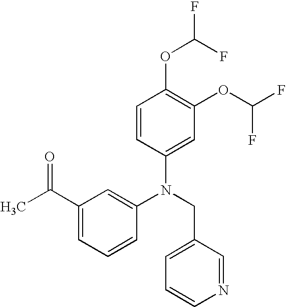 | [M + H]+ = 435 C 59.67, H 4.29, N 6.32 calculated with o.5 mol water C 59.67, H 4.15, N 6.54. |
| 89 | 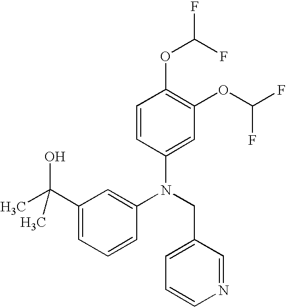 | [M + H]+ = 451 C 60.59, H 4.78, N 6.31 calculated with 0.3 mol water C 60.6, H 5.00, N 6.15. |
| 90 | 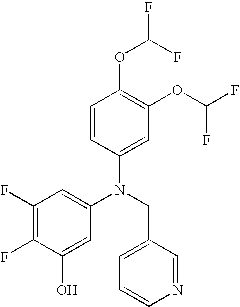 | [M + H] = 445 C 53.65, H 3.30, N 6.11 calculated with 0.2 mol water C 53.62, H 3.24, N 6.26. |
| 91 | 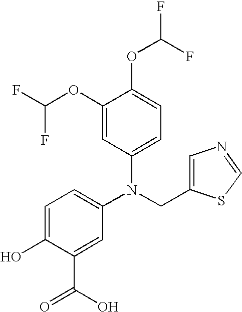 | [M + H] = 459 |
| 92 | 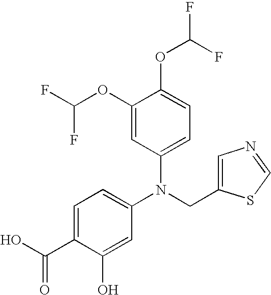 | [M + H] = 459 |
| 93 | 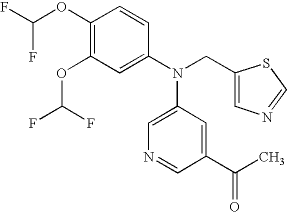 | |
| 94 | 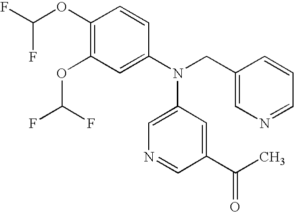 | [M + H] = 436 |
| 95 | 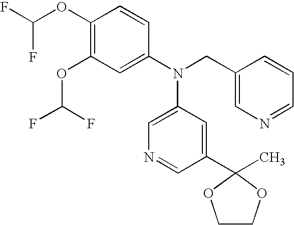 | [M + H] = 480 |
| 96 | 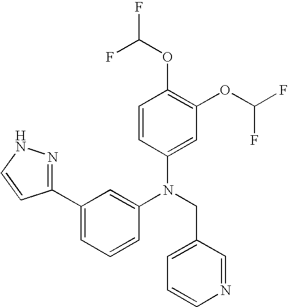 | [M + H]+ = 459 C: 60.26, H: 3.96, N: 12.22, found C: 60.06, H: 3.86, N: 12.13 |
| 97 | 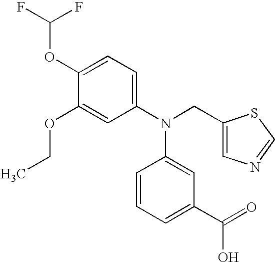 | EA: calc'd C: 57.13, H: 4.32, N: 6.66, found C: 56.76, H: 4.13, N: 6.73; [M + H]+ = 421 |
| 98 | 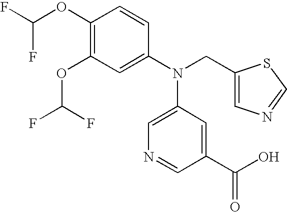 | [M + H] = 444 |
| 99 | 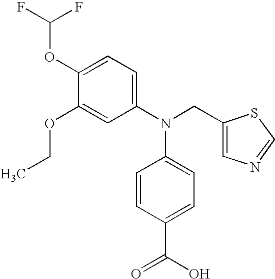 | EA: calc'd C: 57.13, H: 4.32, N: 6.66; found C: 56.78, H: 4.26, N: 6.69; mp = 67.0-69.3; [M + H]+ = 421 |
| 100 | 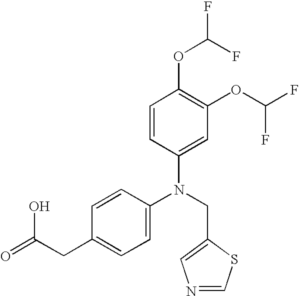 | [M + H]+ = 457 |
| 101 | 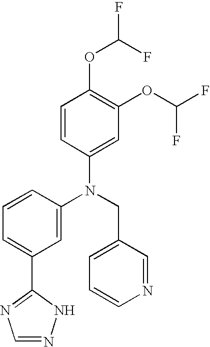 | [M + H]+ = 460 |
| 102 | 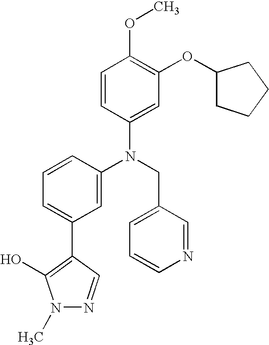 | [M + H]+ = 471 |
| 103 | 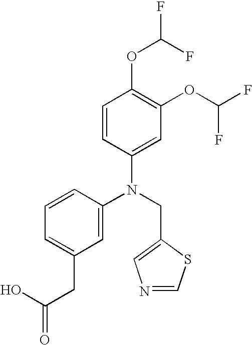 | [M + H]+ = 457 |
| 104 | 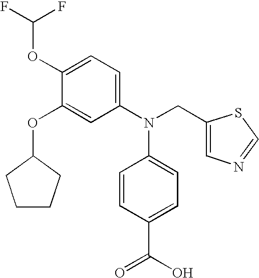 | mp = 67.3-69.7° C.; [M + H]+ = 461 |
| 105 | 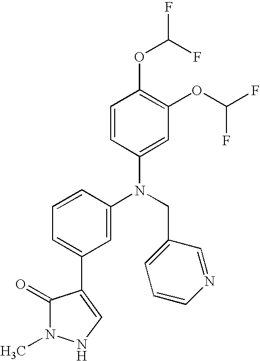 | [M + H]+ = 489 |
| 106 | 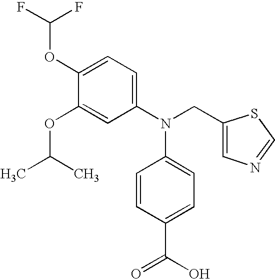 | mp = 158.0-158.5° C.; [M + H]+ = 435 |
| 107 | 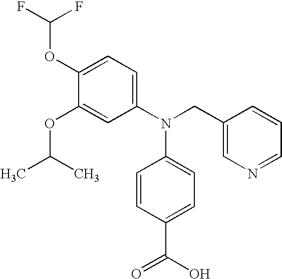 | mp = 66.5-67.9; [M + H]+ = 429 |
| 108 | 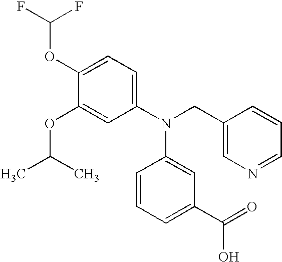 | mp = 57.3-58.9; [M + H]+ = 429 |
| 109 | 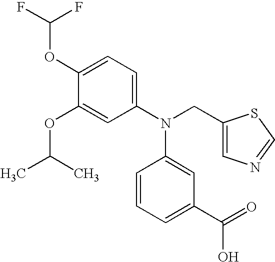 | mp = 58.6-60.0; [M + H]+ = 435 |
| 110 | 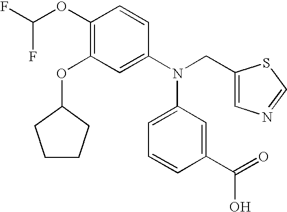 | mp = 56.3-58.0; [M + H]+ = 461 |
| 111 | 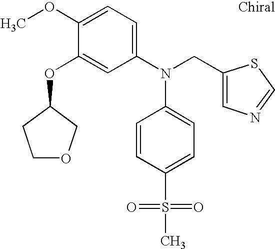 | [M + H]+ = 461 |
| 112 | 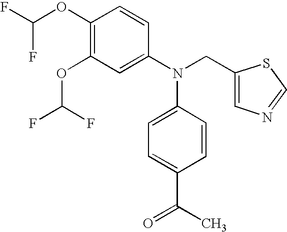 | [M + H]+ = 441 |
| 113 | 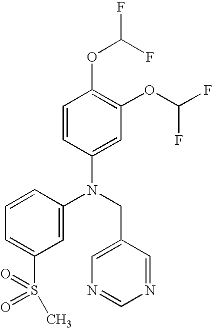 | [M + H]+ = 472 |
| 114 | 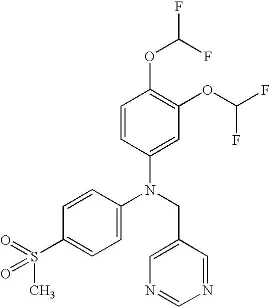 | [M + H]+ = 472 |
| 115 | 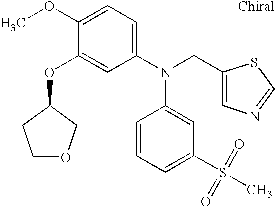 | [M + H]+ = 461 |
| 116 | 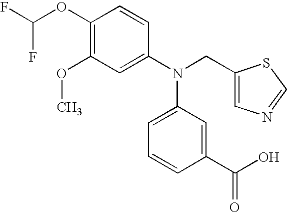 | [M + H] = 407 |
| 117 | 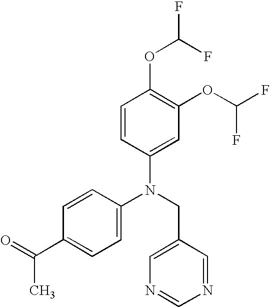 | [M + H]+ = 437 |
| 118 | 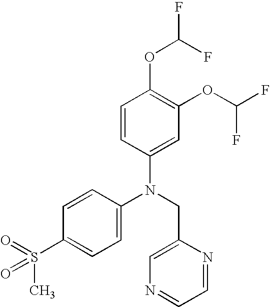 | [M + H]+ = 472 |
| 119 | 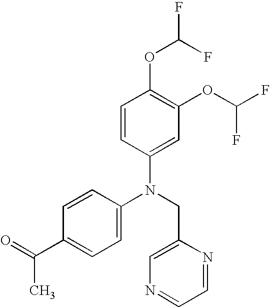 | [M + H]+ = 436 |
| 120 | 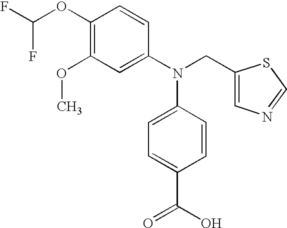 | [M + H] = 407 Mp = 164.9°-165.3° C. |
| 121 | 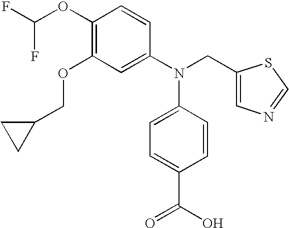 | [M + H] = 447 |
| 122 | 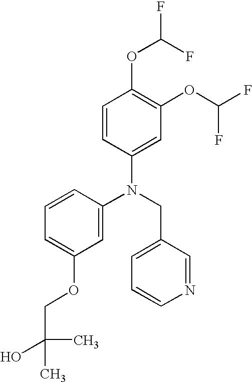 | [M + H]+ = 481 C 59.86, H, 5.20, N 5.73 calculated C 60.00, H 5.03, N 5.83 |
| 123 | 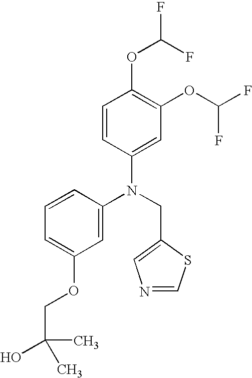 | [M + H]+ = 487 C 54.29, H, 4.43, N 5.78 calculated C 54.32, H 4.56, N 5.76 |
| 124 | 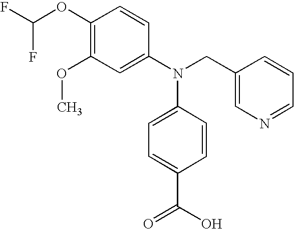 | [M + H] = 401 Mp = 182.4°-183.4° C. |
| 125 | 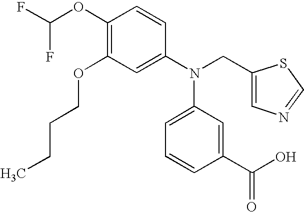 | EA: calcd C: 58.92, H: 4.94, N: 6.25, found C: 58.66, H: 4.86, N: 6.28; [M + H]+ = 449 |
| 126 | 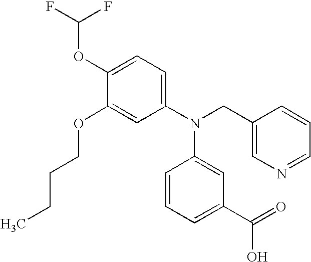 | EA: calcd C: 65.15, H: 5.47, N: 6.33, found C: 65.07, H: 5.43, N: 6.44; [M + H]+ = 443 |
| 127 | 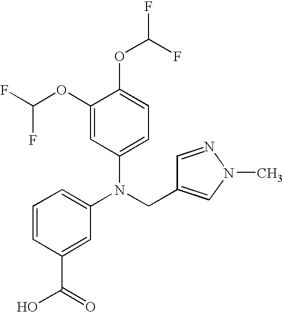 | [M + H]+ = 440 |
| 128 | 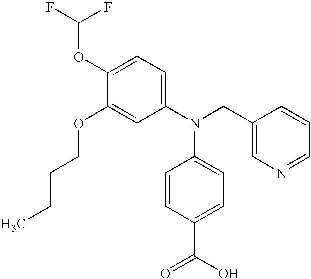 | mp = 133.7-135.1; [M + H]+ = 443 |
| 129 | 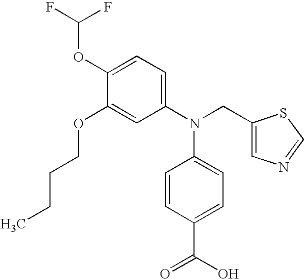 | mp = 158.7-159.5; [M + H]+ = 449 |
| 130 | 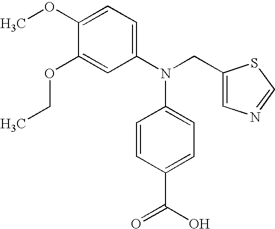 | mp = 73.1-74.5; [M + H]+ = 385 |
| 131 | 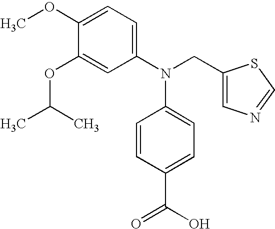 | mp = 67.8-69.1; [M + H]+ = 399 |
| 132 | 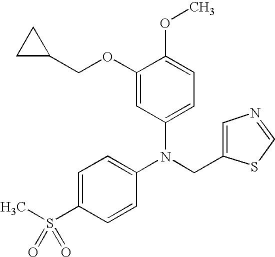 | [M + H] = 445 |
| 133 | 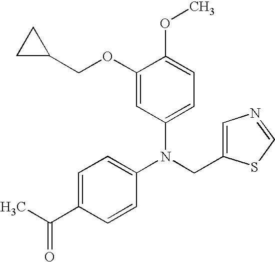 | [M + H] = 409 |
| 134 | 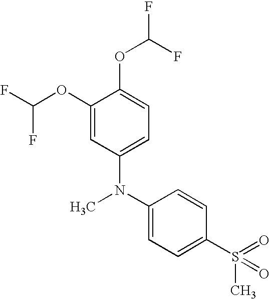 | [M + H] = 394 |
| 135 | 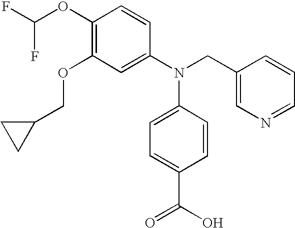 | [M + H] = 441 |
| 136 | 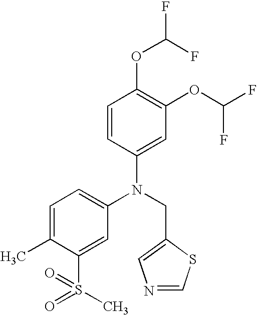 | [M + H]+ = 491 C 48.55, H, 3.62, N 5.57 calculated C 48.97, H 3.70, N 5.71 |
| 137 | 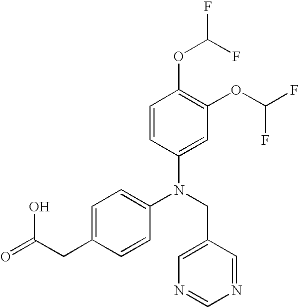 | [M + H]+ = 452 |
| 138 | 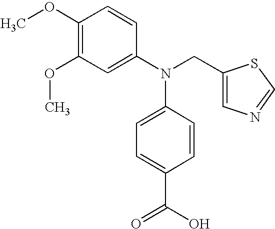 | mp = 86.4-88.3; [M + H]+ = 371 |
| 139 | 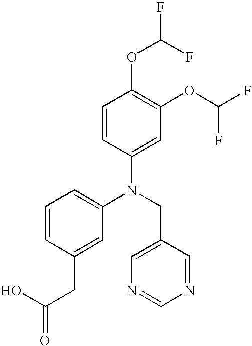 | [M + H]+ = 452 |
| 140 | 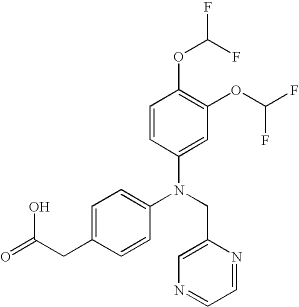 | [M + H]+ = 452 |
| 141 | 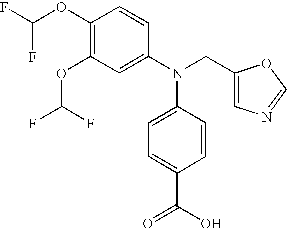 | [M + H] = 427 mp = 132.4-135.0 |
| 142 | 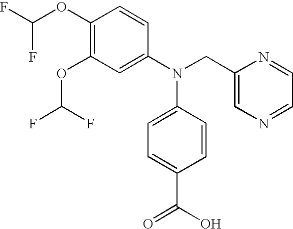 | [M + H] = 438 mp 158.0-161.1 |
| 143 | 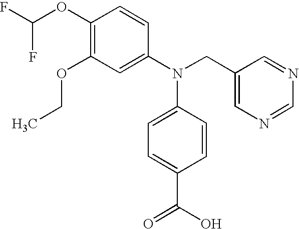 | [M + H] = 416 Mp = 165.3°-165.9° C. |
| 144 | 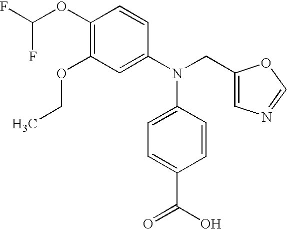 | [M + H] = 405 Mp = 111.1°-113.0° C. |
| 145 | 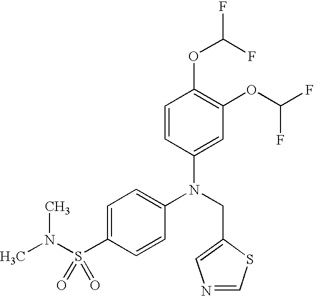 | [M + H]+ = 506 C 47.27, H 3.86, N 8.15 calculated C 47.52, H 3.79, N 8.31 |
| 146 | 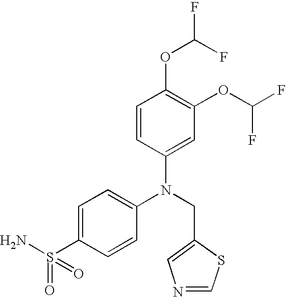 | [M + H]+ = 478 C 44.89, H 3.39, N 8.91 calculated C 45.28, H 3.17, N 8.80 |
| 147 | 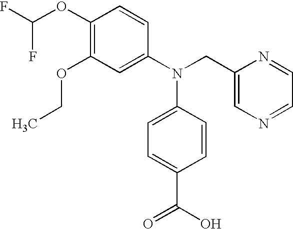 | [M + H] = 416 |
| 148 | 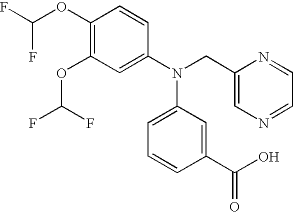 | [M + H] = 438 |
| 149 | 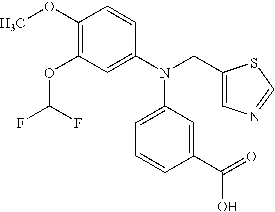 | mp = 86.4-88.3; [M + H]+ = 407 |
| 150 | 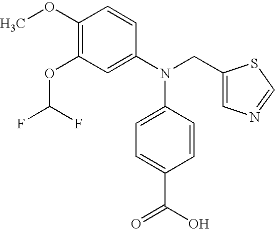 | mp = 86.4-88.3; [M + H]+ = 407 |
| 151 | 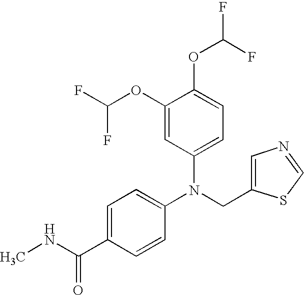 | [M + H] = 456 |
| 152 | 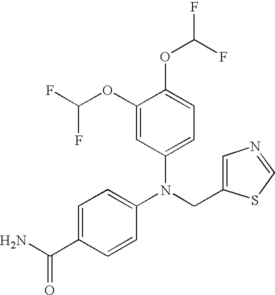 | [M + H] = 442 |
| 153 | 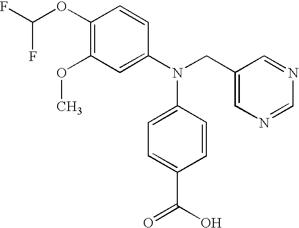 | [M + H] = 402 mp = 191.3-191.5 |
| 154 | 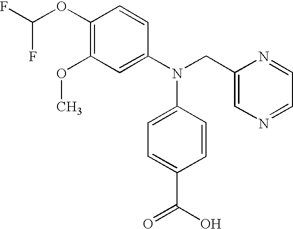 | [M + H] = 402 mp = 157.2-157.7 |
| 155 | 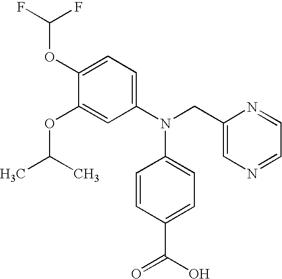 | mp = 132.3-135.7; [M + H]+ = 430 |
| 156 | 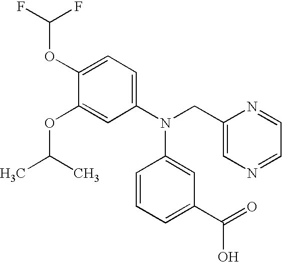 | mp = 81.2-82.0; [M + H]+ = 430 |
| 157 | 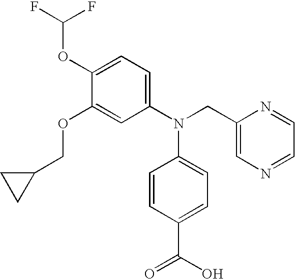 | mp = 81.2-82.3; [M + H]+ = 442 |
| 158 | 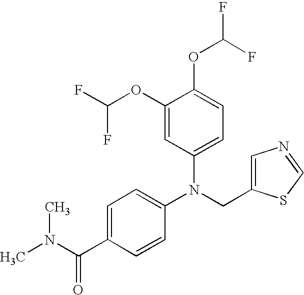 | [M + H] = 470 |
| 159 | 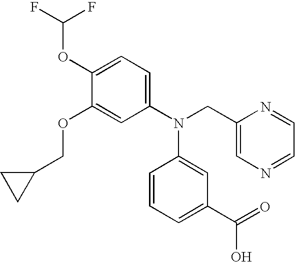 | mp = 72.7-73.1; [M + H]+ = 442 |
| 160 | 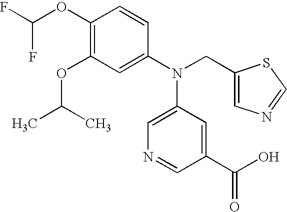 | [M + H] = 436 mp = 121.7-123.1 |
| 161 | 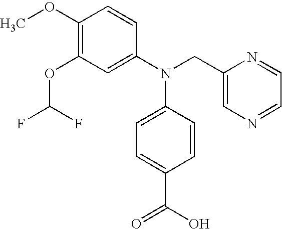 | [M + H] = 402 |
| 162 | 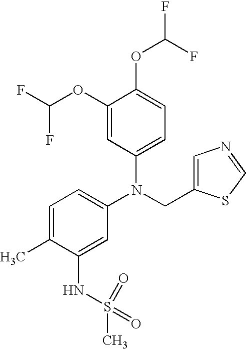 | [M + H] = 506 |
| 163 | 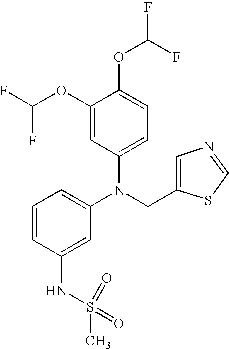 | [M + H] = 492 |
| 164 | 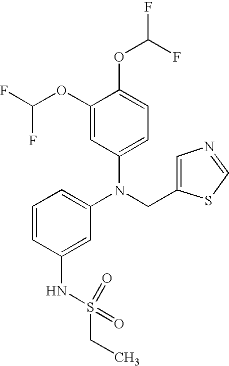 | [M + H] = 506 |
| 165 | 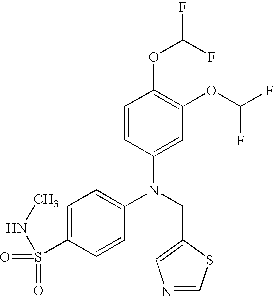 | [M + H]+ = 492 mp = 105.5-106.4 |
| 166 | 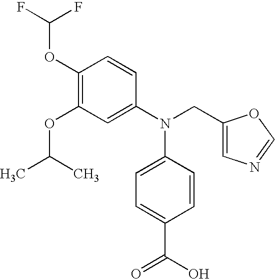 | [M + H] = 419 |
| 167 | 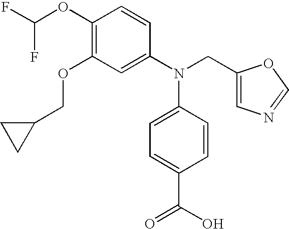 | [M + H] = 431 |
| 168 | 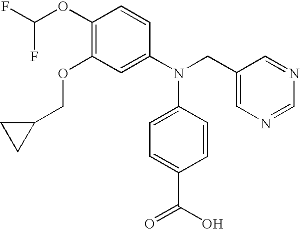 | [M + H] = 442 mp = 265.1-266.6 |
| 169 | 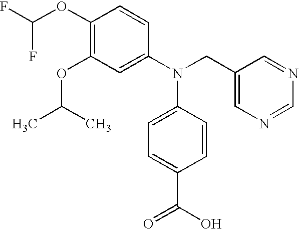 | [M + H] = 430 |
| 170 | 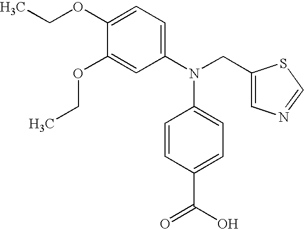 | mp = 77.0-79.0; [M + H] = 399 |
| 171 | 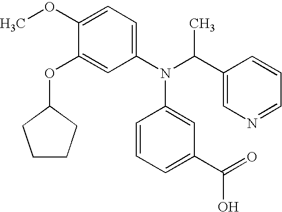 | [M + H]+ = 433 |
| 172 | 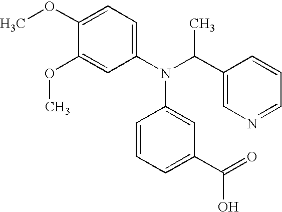 | [M + H]+ = 379 |
| 173 | 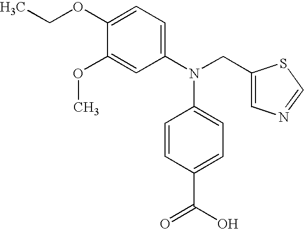 | mp = 69.3-70.3; [M + H]+ = 395 |
| 174 | 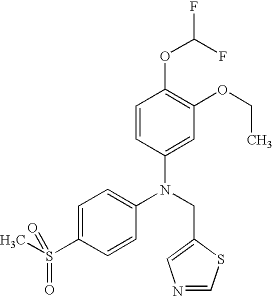 | [M + H]+ = 455 C 51.52, H 4.39, N 5.99 calculated C 51.46, H 4.36, N 5.99 |
| 175 | 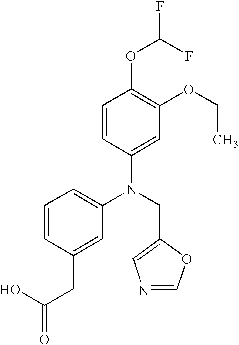 | [M + H]+ = 419 |
| 176 | 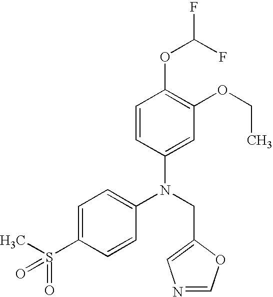 | [M + H]+ = 439 C 54.55, H 4.61, N 6.37 calculated C 54.79, H 4.60, N 6.39 |
| 177 | 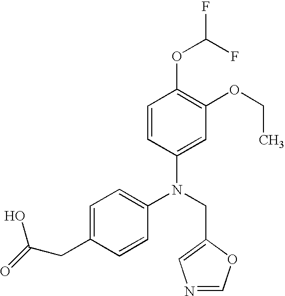 | [M + H]+ = 419 |
| 178 | 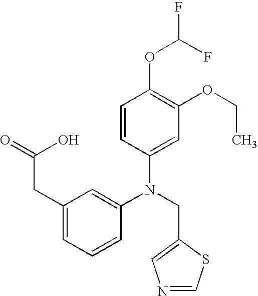 | [M + H]+ = 435 C 55.59, H 4.49, N 5.87 calculated with 0.3 mol of DCM C 55.62, H 4.51, N 6.09 |
| 179 | 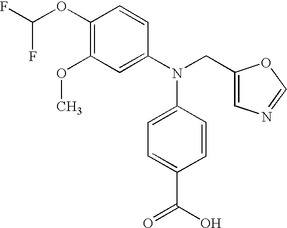 | [M + H] = 391 |
| 180 | 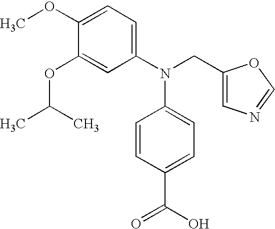 | mp = 60.3-61.0; [M + H]+ = 383 |
| 181 | 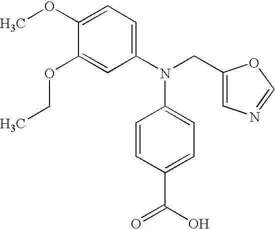 | mp = 175.6-176.9; [M + H]+ = 369 |
| 182 | 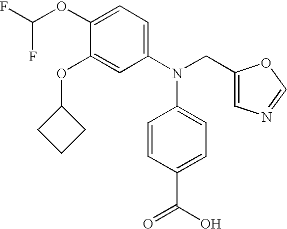 | [M + H]+ = 431 |
| 183 | 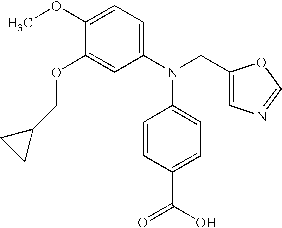 | mp = 160.0-161.7; [M + H]+ = 395 |
| 184 | 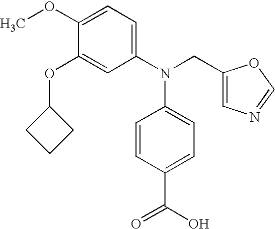 | mp = 148.0-150.3; [M + H]+ = 395 |
| 185 | 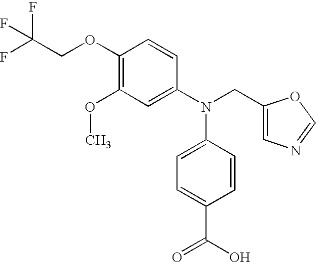 | [M + H] = 423 |
| 186 | 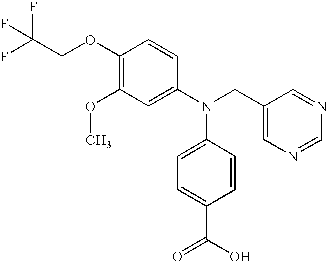 | [M + H] = 434 mp = 208.7-209.9 |
| 187 | 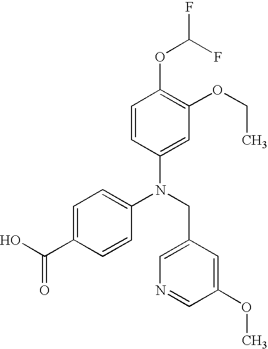 | [M + H]+ = 445 |
| 188 | 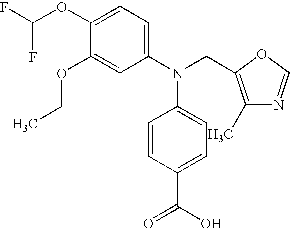 | [M + H]+ = 419, [M − H]− = 417 |
| 189 | 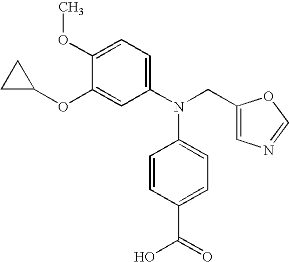 | [M + H]+ = 381 |
| 190 | 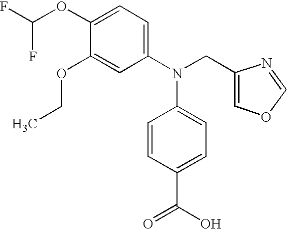 | [M + H]+ = 405, [M − H]− = 403 |
| 191 | 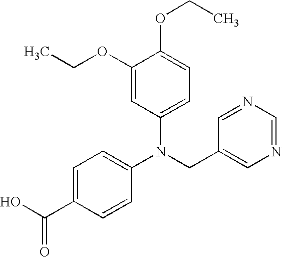 | [M + H] = 394 |
| 192 | 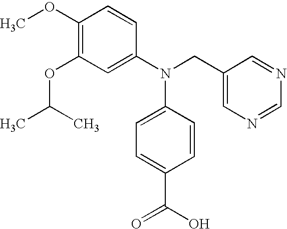 | [M + H] = 394 mp = 181.7-183.6 |
| 193 | 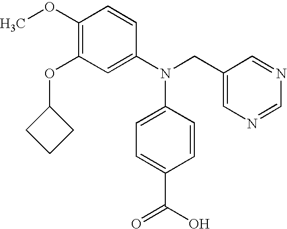 | [M + H] = 406 mp = 174.0-177.9 |
| 194 | 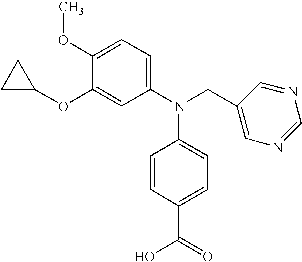 | [M + H] = 392 C 65.83, H 5.31, N 10.4 calculated C 65.94, H 5.21, N 10.34 |
| 195 | 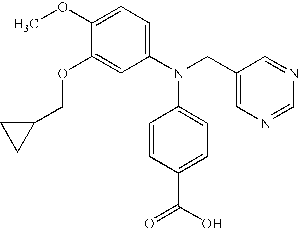 | mp = 185.6-187.2; [M + H]+ = 406 |
| 196 | 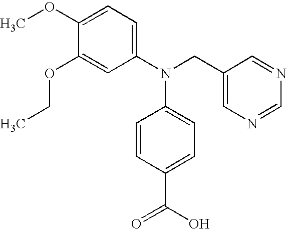 | mp = 190.9-191.9; [M + H]+ = 380 |
| 197 | 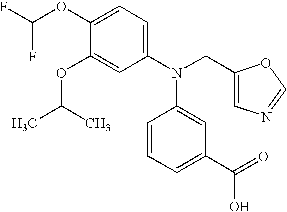 | [M + H]+ = 419 |
| 198 | 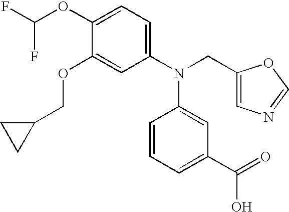 | mp = 60.2-61.0; [M + H]+ = 431 |
| 199 | 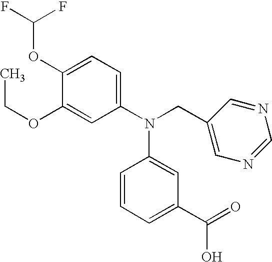 | [M + H]+ = 416 C 60.19 H 4.39, N 9.69 calculated C 60.72, H 4.61, N 10.12 |
| 200 | 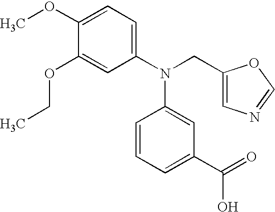 | mp = 158.0-159.8; [M + H]+ = 369 |
| 201 | 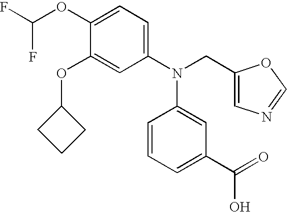 | [M + H]+ = 431, [M − H]− = 429 |
| 202 | 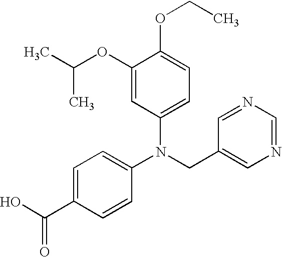 | [M + H] = 408 |
| 203 | 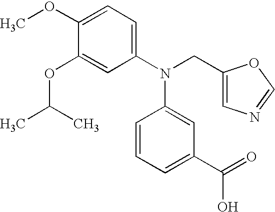 | [M + H]+ = 383, [M − H]− = 381 |
| 204 | 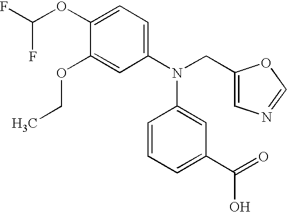 | [M + H]+ = 405, [M − H]− = 403 |
| 205 | 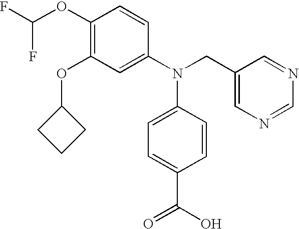 | [M + H] = 442 mp = 135.1-136.3 |
| 206 | 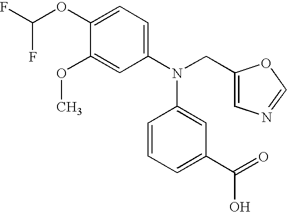 | [M + H]+ = 391, [M − H]− = 389 |
| 207 | 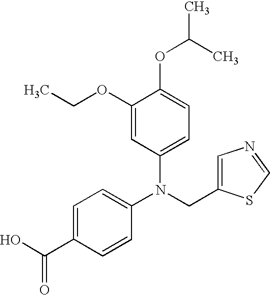 | [M + H]+ = 413 |
| 208 | 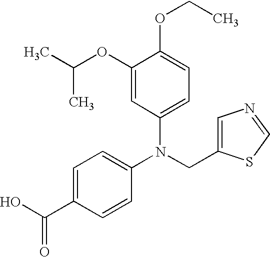 | [M + H]+ = 396 |
| 209 | 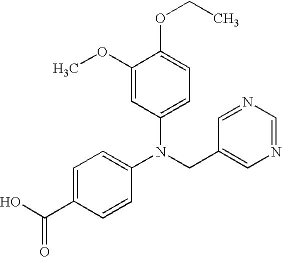 | [M + H]+ = 380 |
| 210 | 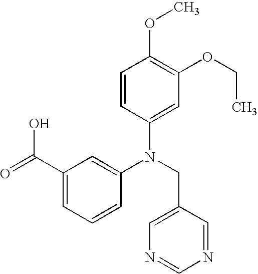 | [M + H]+ = 380 C 64.59, H 5.60, N 10.11 calculated C 66.48, H 5.58, N 11.07 |
| 211 | 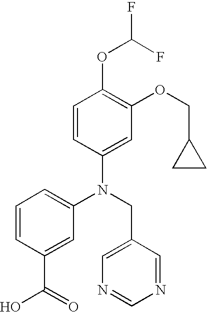 | [M + H]+ = 442 C 61.34, H 4.76, N 9.25 calculated with 0.5 mol of water C 61.33, H 4.92, N 9.33 |
| 212 | 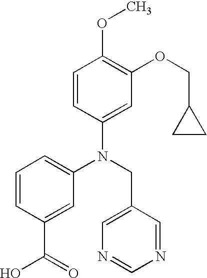 | [M + H]+ = 406 C 66.85, H 5.66, N 10.01 calculated with 0.45 mol of water C 66.80, H 5.83, N 10.16 |
| 213 | 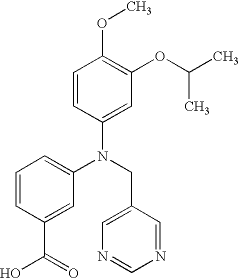 | [M + H]+ = 394 C 65.54, H 5.82, N 10.26 calculated with 0.15 mol of DCM C 65.5, H 5.78, N 10.35 |
| 214 | 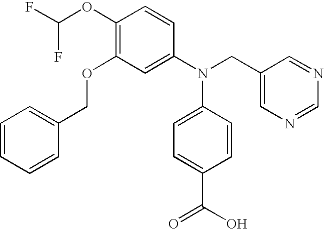 | [M + H] = 478 mp = 157.4-158.2 |
| 215 | 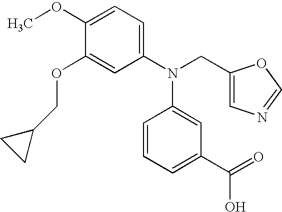 | [M + H]+ = 395 |
| 216 | 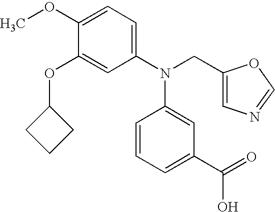 | [M + H]+ = 395, [M − H]− = 393 |
| 217 | 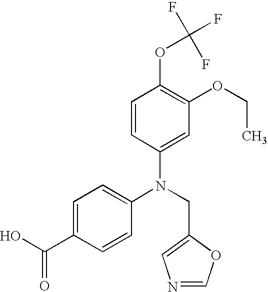 | [M + H] = 423 |
| 218 | 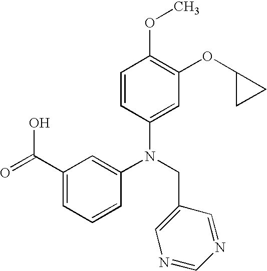 | [M = H] = 392 |
| 219 | 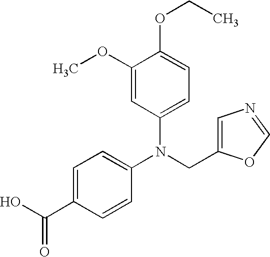 | M + H = 369 |
| 220 | 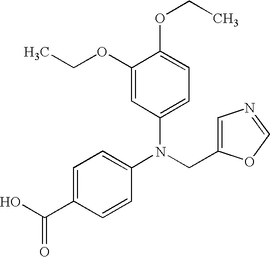 | M + H = 383 |
| 221 | 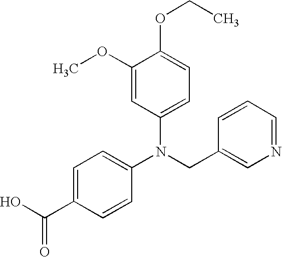 | M + H = 379 |
| 222 | 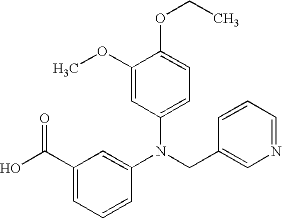 | M + H = 379 |
| 223 | 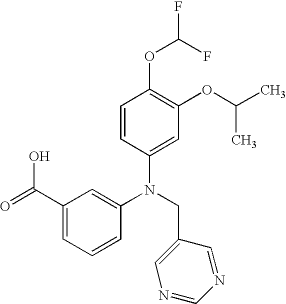 | [M + H]+ = 430 |
| 224 | 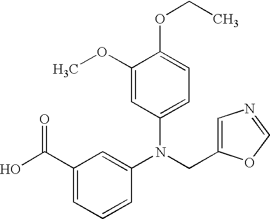 | M + H = 369 |
| 225 | 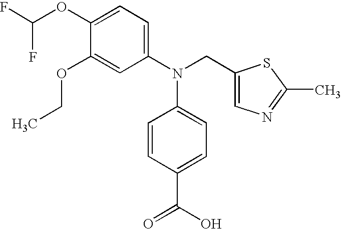 | mp = 73.1-74.1; [M − H]− = 433 |
| 226 | 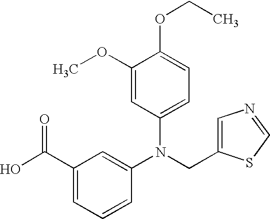 | [M + H]+ = 385 |
| 227 | 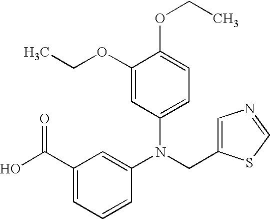 | [M + H]+ = 399 |
| 228 | 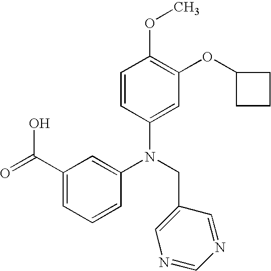 | [M + H]+ = 406 |
| 229 | 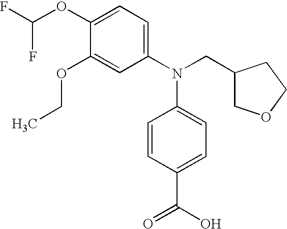 | [M + H]+ = 408 |
| 230 | 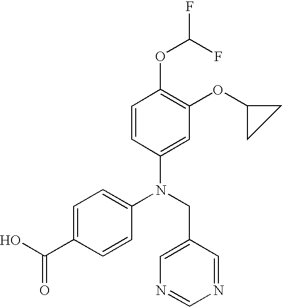 | [M + H]+ = 428 |
| 231 | 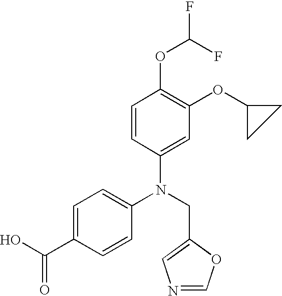 | [M + H]+ = 417 |
| 232 | 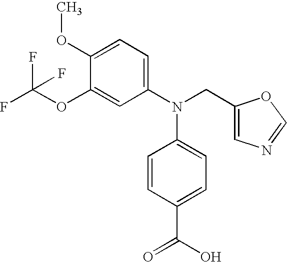 | [M + H]+ = 409 |
| 233 | 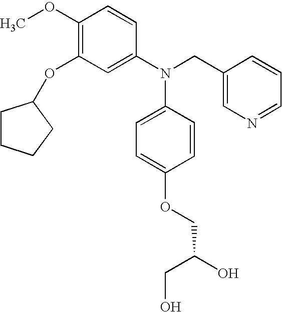 | [M + H] = 465 |
| 234 | 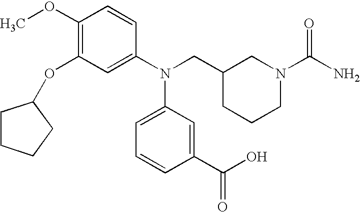 | [M + H]+ = 468 |
| 235 | 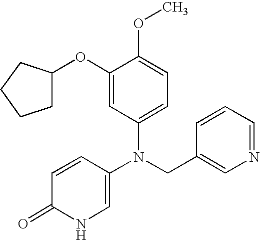 | M + H = 320 |
| 236 | 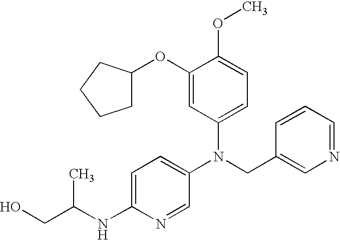 | [M + H] = 449 |
| 237 | 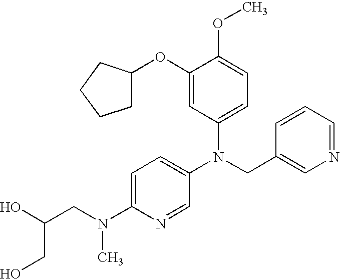 | [M + H] = 479 |
| 238 | 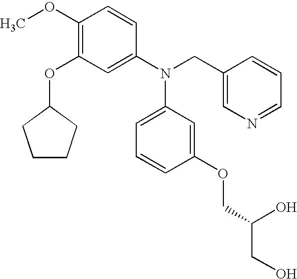 | [M + H] = 465 |
| 239 | 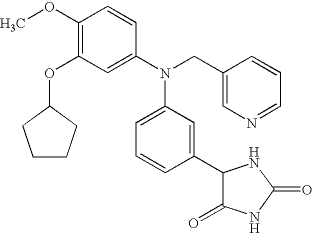 | [M + H] = 473 mp = 99.7-105.0 |
| 240 | 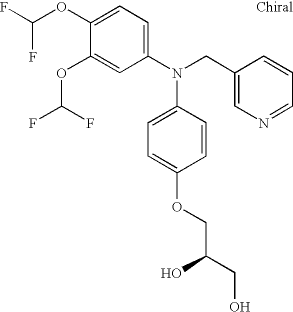 | [M + H] = 483 C 57.14, H 4.56, N 5.84, calculated C 57.26, H 4.6, N 5.8 |
| 241 | 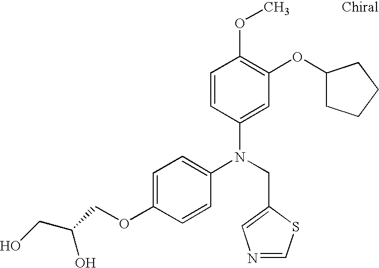 | [M + H] = 471 C 63.56, H 6.35, N 6.00, calculated C 63.81, H 6.43, N 5.95 |
| 242 | 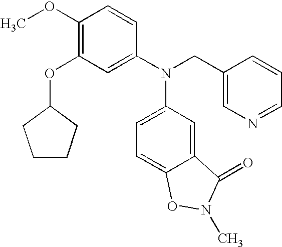 | [M + H] = 446 |
| 243 | 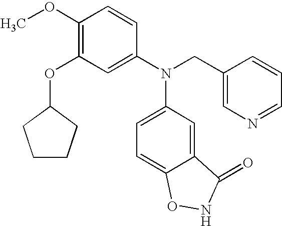 | [M + H] = 432 |
| 244 | 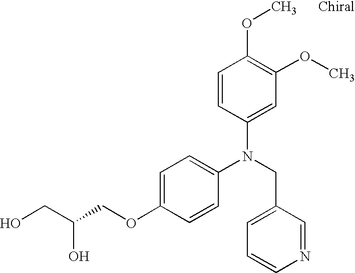 | [M + H] = 411 C 64.36, H 6.17, N 6.58, calculated C 64.69, H 6.19, N 6.49 |
| 245 | 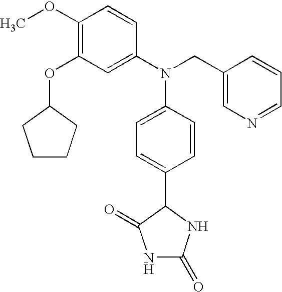 | [M + H] = 473 mp = 108.0-112.5 |
| 246 | 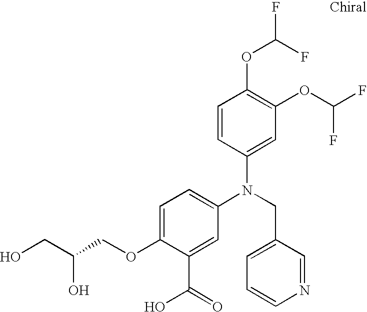 | [M + H]+ = 527 C 52.69, H 4.43, N 4.95, calculated C 52.90, H 4.40, N 5.14 |
| 247 | 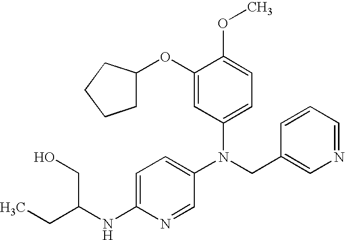 | [M + H] = 463 |
| 248 | 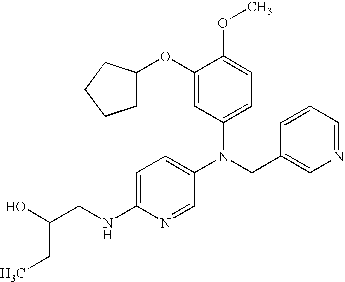 | [M + H] = 463 |
| 249 | 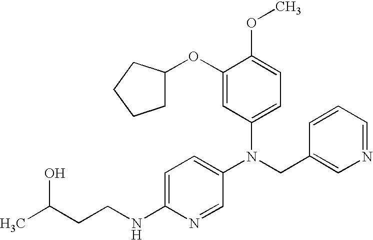 | [M + H] = 463 |
| 250 | 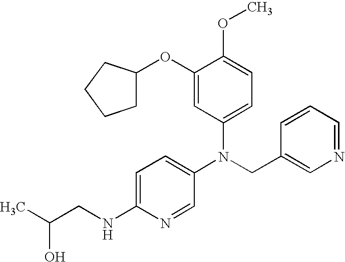 | [M + H] = 449 |
| 250 | 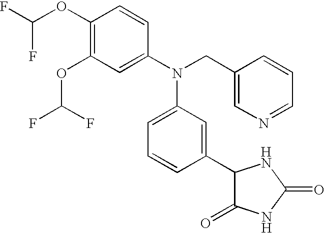 | [M + H] = 491 mp = 70.0-90.7 |
| 252 | 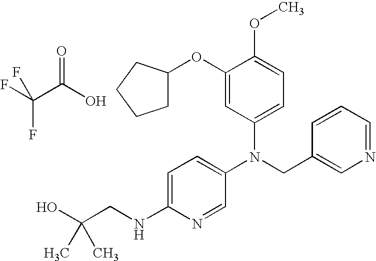 | [M + H] = 463 |
| 253 | 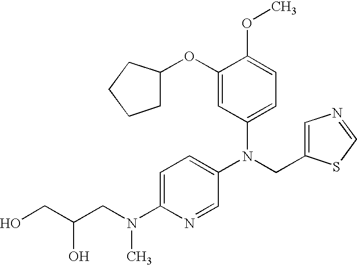 | [M + H] = 485 |
| 254 | 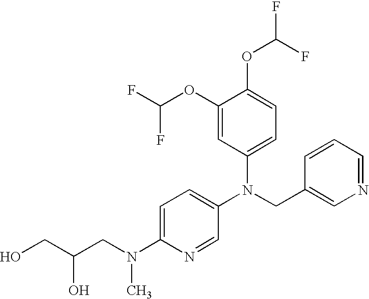 | [M + H] = 497 |
| 255 | 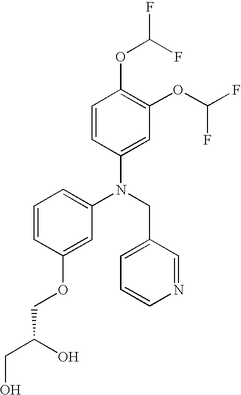 | [M + H] = 483 C 56.95, H 4.57, N 5.87, calculated C 57.26, H 4.6, N 5.81 |
| 256 | 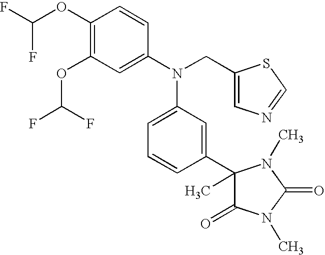 | [M + H] = 540 |
| 257 | 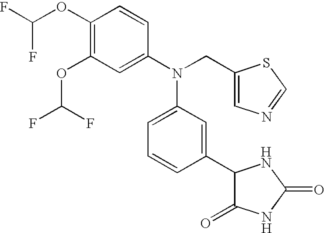 | [M + H] = 497 mp = 63.1-82.3° C. |
| 258 | 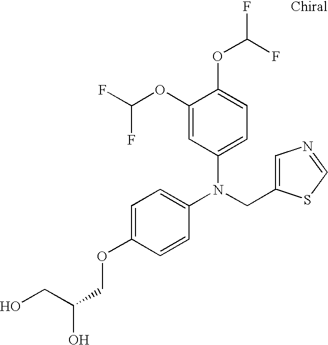 | [M + H] = 489 |
| 259 | 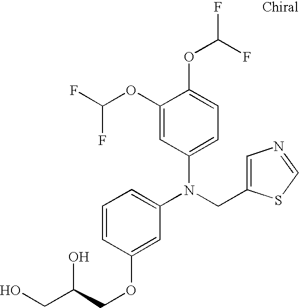 | [M + H] = 489 |
| 260 | 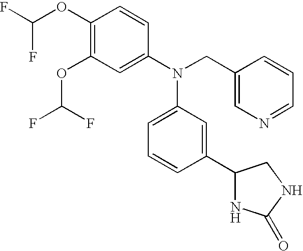 | [M + H] = 477 |
| 261 | 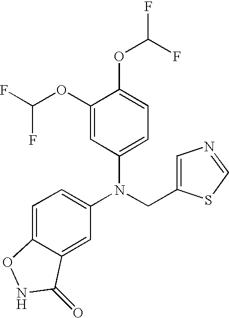 | [M + H] = 456 |
| 262 | 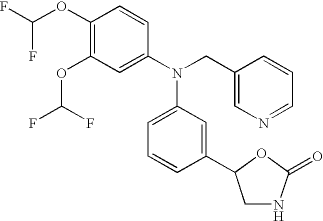 | [M + H]+ = 478 |
| 263 | 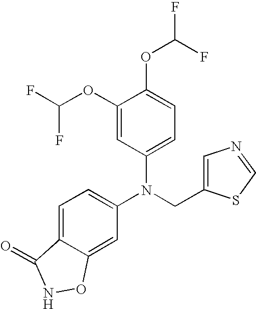 | [M + H] = 456 |
| 264 | 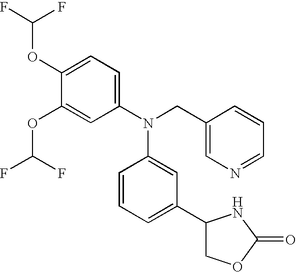 | [M + H]+ = 478 |
| 265 | 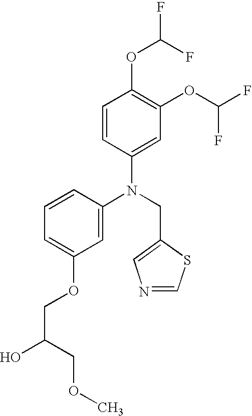 | [M + H]+ = 503 |
| 266 | 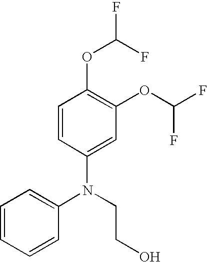 | [M + H] = 346 |
| 267 | 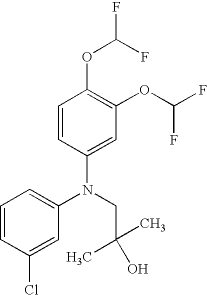 | [M + H] = 408 |
| 268 | 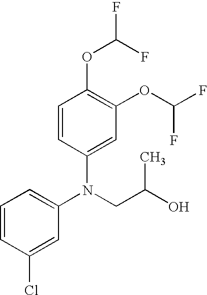 | [M + H] = 394 |
| 269 | 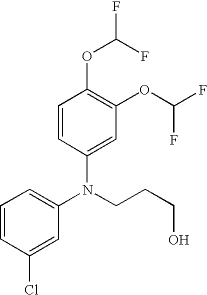 | [M + H] = 394 |
| 270 | 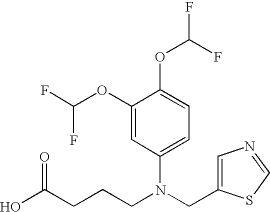 | [M + H]+ = 409 |
| 271 | 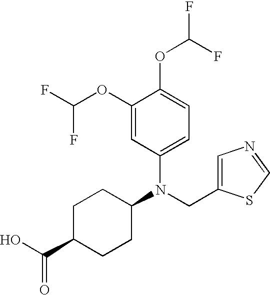 | [M + H] = 459 |
| 272 | 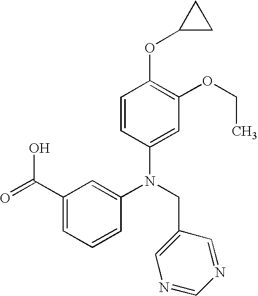 | [M + H]+ = 406 |
| 273 | 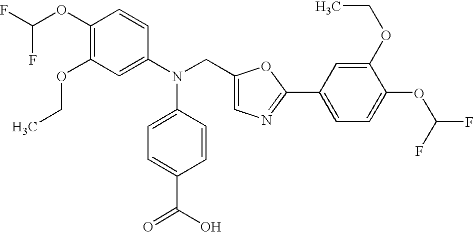 | [M + H] = 591 mp = 178.4-178.9° C. |
| 279 | 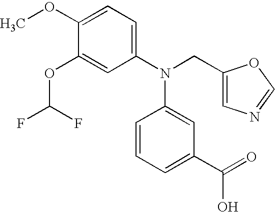 | [M + H]+ = 389 |
| 280 | 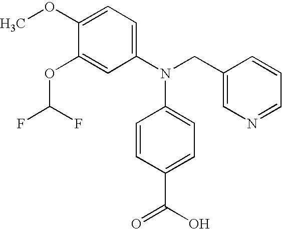 | [M + H]+ = 401 |
| 281 | 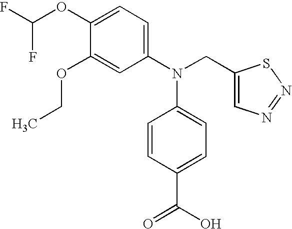 | [M + H]+ = 422 |
| 282 | 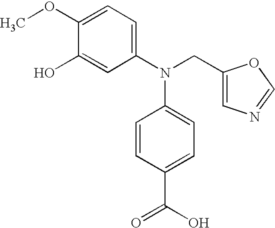 | [M + H]+ = 341 |
| 283 | 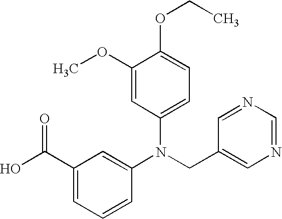 | [M + H] = 380 |
| 284 | 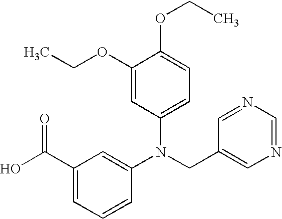 | [M + H]+ = 394 |
| 285 | 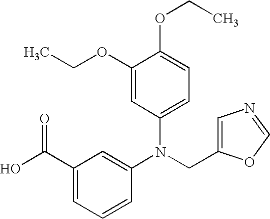 | [M + H] = 383; C: 65.64; H: 5.76: N: 7.40. calcd: C: 65.96; H: 5.80; N: 7.33 |
| 286 | 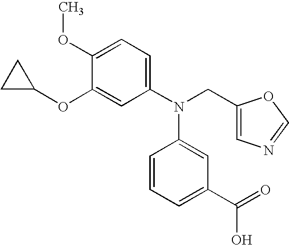 | [M = H] = 381 |
| 287 | 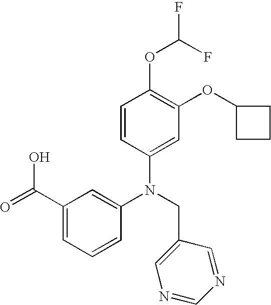 | [M + H] = 442 |
| 288 | 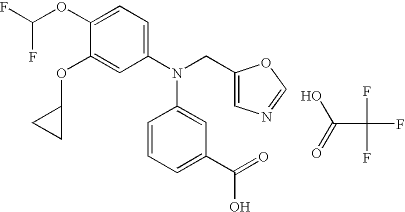 | [M = H] = 417 |
| 289 | 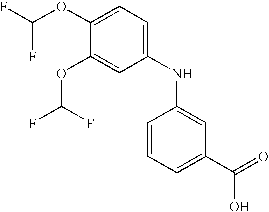 | — |
| 290 | 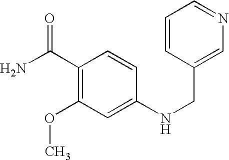 | [M + H] 258 |
| 291 | 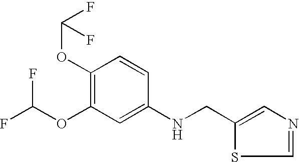 | [M = 1] = 323 |
| 292 | 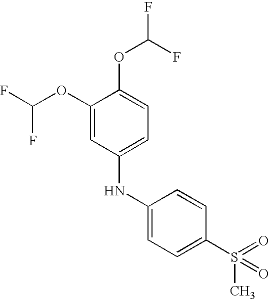 | [M + H] = 379 |
| 293 | 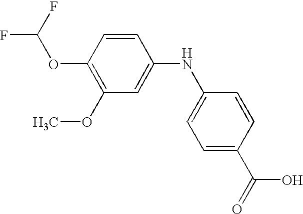 | M + H = 310 |
| 294 | 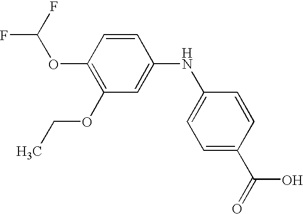 | M + H = 324 |
| 295 | 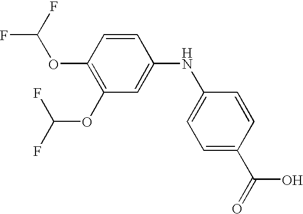 | M + H = 346 |

### Example 47

### In Vitro Measurement of Type 4 Phosphodiesterase Inhibition Activity

Human PDE4 was obtained from baculovirus-infected Sf9 cells that expressed the recombinant enzyme. The cDNA encoding hPDE-4D6 was subcloned into a baculovirus vector.

Insect cells (Sf9) were infected with the baculovirus and cells were cultured until protein was expressed. The baculovirus-infected cells were lysed and the lysate was used as source of hPDE-4D6 enzyme. The enzyme was partially purified using a DEAE ion exchange chromatography. This procedure can be repeated using cDNA encoding other PDE-4 enzymes.

### Assay:

Type 4 phosphodiesterases convert cyclic adenosine monophosphate (cAMP) to 5′-adenosine monophosphate (5′-AMP). Nucleotidase converts 5′-AMP to adenosine. Therefore the combined activity of PDE4 and nucleotidase converts cAMP to adenosine. Adenosine is readily separated from cAMP by neutral alumina columns. Phosphodiesterase inhibitors block the conversion of cAMP to adenosine in this assay; consequently, PDE4 inhibitors cause a decrease in adenosine.

Cell lysates (40 ul) expressing hPDE-4D6 were combined with 50 ul of assay mix and 10 μl of inhibitors and incubated for 12 min at room temperature. Final concentrations of assay components were: 0.4 ug enzyme, 10 mM Tris-HCl (pH 7.5), 1 mM MgCl₂, 3 uM cAMP, 0.002 U 5′-nucleotidase, and 3×10⁴ cpm of [3H]cAMP. The reaction was stopped by adding 100 μl of boiling 5mN HCl. An aliquot of 75 μl of reaction mixture was transferred from each well to alumina columns (Multiplate; Millipore). Labeled adenosine was eluted into an OptiPlate by spinning at 2000 rpm for 2 min; 150 μl per well of scintillation fluid was added to the OptiPlate. The plate was sealed, shaken for about 30 min, and cpm of [³H]adenosine was determined using a Wallac Triflux®.

All test compounds are dissolved in 100% DMSO and diluted into the assay such that the final concentration of DMSO is 0.1%. DMSO does not affect enzyme activity at this concentration.

A decrease in adenosine concentration is indicative of inhibition of PDE activity. IC₅₀ values were determined by screening 6 to 12 concentrations of compound ranging from 0.1 nM to 10,000 nM and then plotting drug concentration versus ³H-adenosine concentration. Nonlinear regression software (Assay Explorer@) was used to estimate pIC₅₀ values.

IC₅₀ values for the preferred compounds of the invention are typically less than 300 nM, or even more preferred, less than 10.0 nM. A representative list of compounds with bioassay results appears in the table below.

| Com- | | PDE4D6 |
|---|---|---|
| pound | | IC₅₀ |
| No | MOLSTRUCTURE | (nM) |
| 14 | 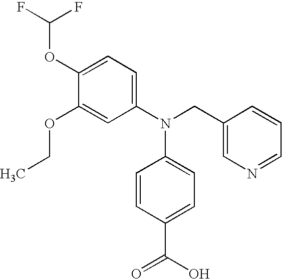 | 2.54 |
| 47 | 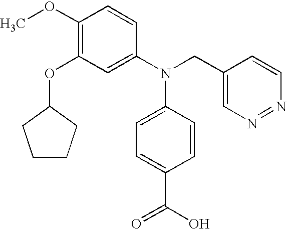 | 127.50 |
| 59 | 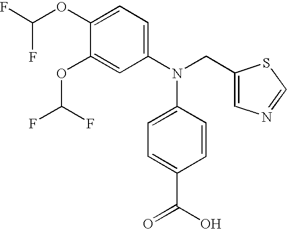 | 5.69 |
| 72 | 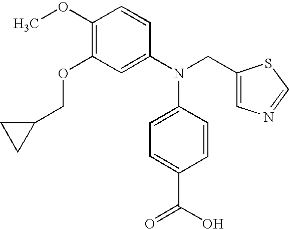 | 25.06 |
| 80 | 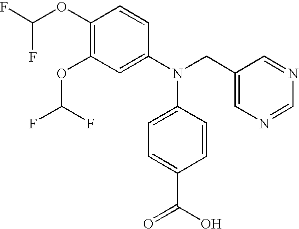 | 6.17 |
| 111 | 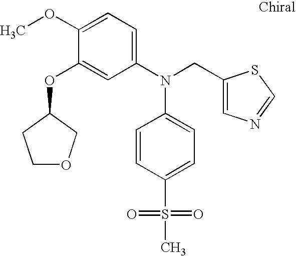 | 29.00 |
| 116 | 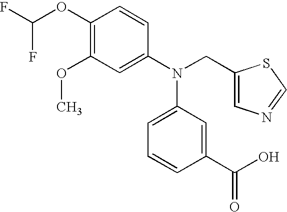 | 7.00 |
| 124 | 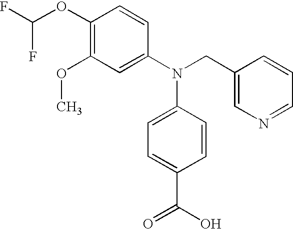 | 11.20 |
| 130 | 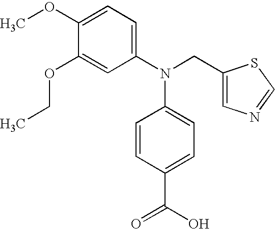 | 8.60 |
| 131 | 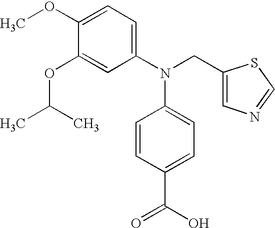 | 17.30 |
| 137 | 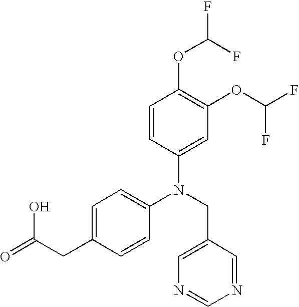 | 139.00 |
| 138 | 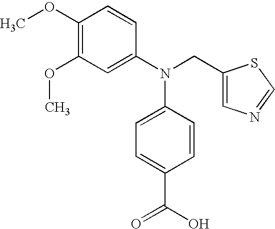 | 61.00 |
| 139 | 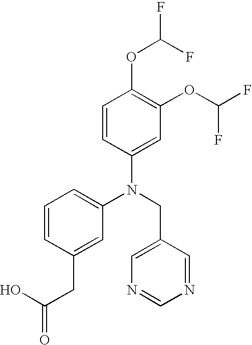 | 53.00 |
| 141 | 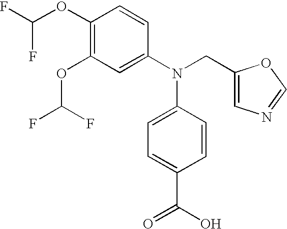 | 27.00 |
| 142 | 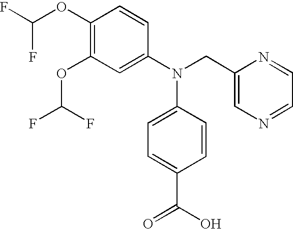 | 289.00 |
| 143 | 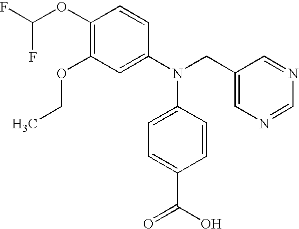 | 7.50 |
| 182 | 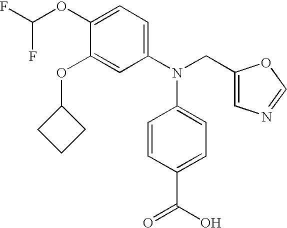 | 3.80 |
| 189 | 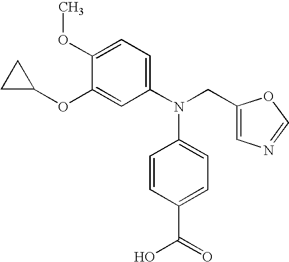 | 2.30 |
| 190 | 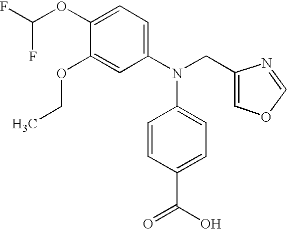 | 20.00 |
| 217 | 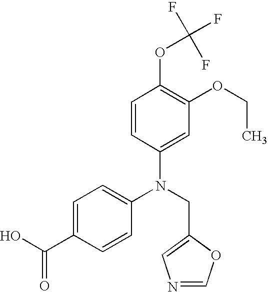 | 12.00 |
| 218 | 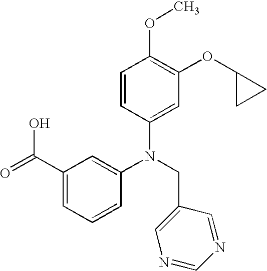 | 1.08 |
| 294 | 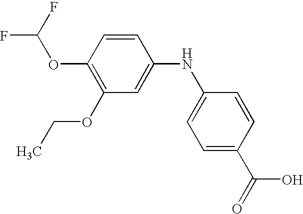 | 34.90 |
| 295 | 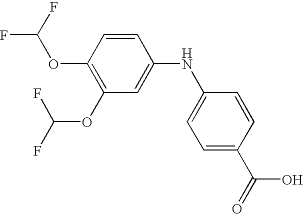 | 108.00 |
| 225 | 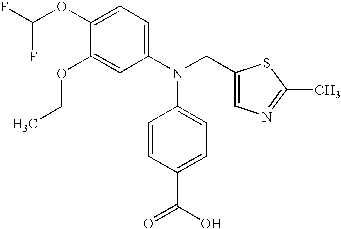 | 4.38 |
| 232 | 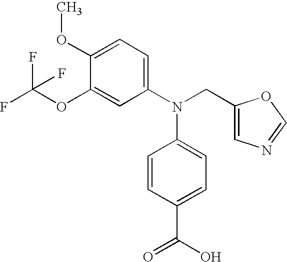 | 6.94 |
| 281 | 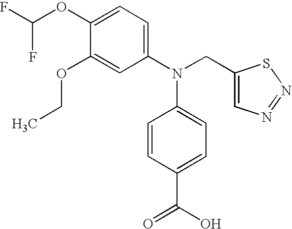 | 0.16 |

### Example 48

### Method A

### Passive Avoidance in Rats, an In Vivo Test for Learning and Memory

The test was performed as previously described (Zhang, H.-T., Crissman, A. M., Dorairaj, N. R., Chandler, L. J., and O'Donnell, J. M., Neuropsychopharmacology, 2000, 23, 198-204.). The apparatus (Model E10-16SC, Coulboum Instruments, Allentown, Pa.) consisted of a two-compartment chamber with an illuminated compartment connected to a darkened compartment by a guillotine door. The floor of the darkened compartment consisted of stainless steel rods through which an electric foot-shock could be delivered from a constant current source. All experimental groups were first habituated to the apparatus the day before the start of the experiment. During the training, the rat (Male Sprague-Dawley (Harlan) weighing 250 to 350 g) was placed in the illuminated compartment facing away from the closed guillotine door for 1 minute before the door was raised. The latency for entering the darkened compartment was recorded. After the rat entered the darkened compartment, the door was closed and a 0.5 mA electric shock was administered for 3 seconds. Twenty-four hours later, the rat was administered 0.1 mg/kg MK-801 or saline, 30 minutes prior to the injection of saline or test compound (dosed from 0.1 to 2.5 mg/kg, i.p.), which was 30 minutes before the retention test started. The rat was again placed in the illuminated compartment with the guillotine door open. The latency for entering the darkened compartment was recorded for up to 180 seconds, at which time the trial was terminated.

All data were analyzed by analyses of variance (ANOVA); individual comparisons were made using Kewman-Keuls tests. Naïve rats required less than 30 seconds, on average, to cross from the illuminated compartment to the darkened compartment. However, 24 hours after the electric shock exposure, most rats pretreated with vehicle did not re-enter the darkened compartment; the average latency was increased up to 175 seconds (p<0.001). Pretreatment with MK-801 (0.1 mg/kg) markedly reduced this latency when compared to the vehicle (p<0.001).

### Example 49

### Method B

### Radial Arm Maze Task in Rats, an In Vivo Test for Learning and Memory

The test was performed as previously described (Zhang, H.-T., Crissman, A. M., Dorairaj, N. R., Chandler, L. J., and O'Donnell, J. M., Neuropsychopharmacology, 2000, 23, 198-204.). Five days after initial housing, rats (male Sprague-Dawley (Harlan) weighing 250 to 350 g) were placed in the eight-arm radial maze (each arm was 60×10×12 cm high; the maze was elevated 70 cm above the floor) for acclimation for two days. Rats were then placed individually in the center of the maze for 5 minutes with food pellets placed close to the food wells, and then, the next day, in the wells at the end of the arms; 2 sessions a day were conducted. Next, four randomly selected arms were then baited with one pellet of food each. The rat was restricted to the center platform (26 cm in diameter) for 15 seconds and then allowed to move freely throughout the maze until it collected all pellets of food or 10 minutes passed, whichever came first. Four parameters were recorded: 1) working memory errors, i.e., entries into baited arms that had already been visited during the same trial; 2) reference memory errors, i.e., entries into unbaited arms; 3) total arm entries; and 4) the test duration (seconds), i.e., the time spent in the collection of all the pellets in the maze. If the working memory error was zero and the average reference memory error was less than one in five successive trials, the rats began the drug tests. MK-801 or saline was injected 15 minutes prior to vehicle or test agent, which was given 45 minutes before the test. Experiments were performed in a lighted room, which contained several extra-maze visual cues.

All data were analyzed by analyses of variance (ANOVA); individual comparisons were made using Kewman-Keuls tests. Compared to control, MK-801 (0.1 mg/kg, i.p.) increased the frequencies of both working and reference memory errors (p<0.01). This amnesic effect of MK-801 on working memory is reversed in a statistically significant manner by the administration of actual test compounds in a dose-dependent fashion.

The preceding examples can be repeated with similar success by substituting the generically or specifically described reactants and/or operating conditions of this invention for those used in the preceding examples.

While the invention has been illustrated with respect to the production and of particular compounds, it is apparent that variations and modifications of the invention can be made without departing from the spirit or scope of the invention.

1. A compound according to Formula I: 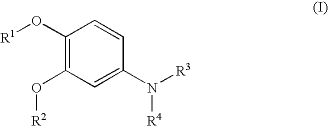
wherein
R¹ is C₁₋₄-alkyl, which is branched or unbranched, and which is unsubstituted or substituted one or more times by halogen, or
C₃₋₁₀-cycloalkyl which is unsubstituted or substituted one or more times by halogen, hydroxy, oxo, cyano, C₁₋₄-alkyl, C₁₋₄-alkoxy, or combinations thereof;
R² is H,
C₁₋₁₂-alkyl, which is branched or unbranched, and which is unsubstituted or substituted one or more times by halogen, hydroxy, cyano, C₁₋₄-alkoxy, oxo or combinations thereof, and wherein one or more —CH₂CH₂— groups are each optionally replaced in each case by —CH═CH— or —C≡C—,
C₃₋₁₀-cycloalkyl which is unsubstituted or substituted one or more times by halogen, hydroxy, oxo, cyano, C₁₋₄-alkyl, C₁₋₄-alkoxy, or combinations thereof,
C₄₋₁₆-cycloalkylalkyl which is unsubstituted or substituted in the cycloalkyl portion and/or the alkyl portion one or more times by halogen, oxo, cyano, hydroxy, C₁₋₄-alkyl, C₁₋₄-alkoxy or combinations thereof,
C₆₋₁₄-aryl which is unsubstituted or substituted one or more times by halogen, CF₃, OCF₃, C₁₋₈-alkyl, hydroxy, C₁₋₈-alkoxy, nitro, methylenedioxy, ethylenedioxy, cyano, or combinations thereof,
C₆₋₁₄-aryl-C₁₋₅-alkyl, in which the alkyl portion is branched or unbranched, which is unsubstituted or is substituted in the aryl portion one or more times by halogen, CF₃, OCF₃, C₁₋₈-alkyl, hydroxy, C₁₋₈-alkoxy, nitro, cyano, methylenedioxy, ethylenedioxy, or combinations thereof, and wherein in the alkyl portion one or more —CH₂CH₂— groups are each optionally replaced by —CH═CH— or —C≡C—, and one or more —CH₂— groups are each optionally replaced by —O— or —NH— and/or the alkyl portion is optionally substituted by halogen, oxo, hydroxy, cyano, or combinations thereof,
a partially unsaturated C₅₋₁₄-carbocyclic group, which is unsubstituted or substituted one or more times by halogen, C₁₋₈-alkyl, C₁₋₈-alkoxy, hydroxy, nitro, cyano, oxo, or combinations thereof,
a heterocyclic group, which is saturated, partially saturated or unsaturated, having 5 to 10 ring atoms in which at least 1 ring atom is a N, O or S atom, which is unsubstituted or substituted one or more times by halogen, hydroxy, C₆₋₁₄-aryl, C₁₋₈-alkyl, C₁₋₈-alkoxy, cyano, trifluoromethyl, nitro, oxo, or combinations thereof, or
a heterocycle-alkyl group, wherein the heterocyclic portion is saturated, partially saturated or unsaturated, and has 5 to 10 ring atoms in which at least 1 ring atom is a N, O or S atom, and the alkyl portion is branched or unbranched, the heterocycle-alkyl group is unsubstituted or substituted one or more times in the heterocyclic portion by halogen, OCF₃, hydroxy, C₆₋₁₄-aryl, C₁₋₈-alkyl, C₁₋₈-alkoxy, cyano, trifluoromethyl, nitro, oxo, or combinations thereof, wherein in the alkyl portion one or more —CH₂CH₂— groups are each optionally replaced by —CH═CH— or —C≡C—, and one or more —CH₂— groups are each optionally replaced by —O— or —NH— and/or the alkyl portion is optionally substituted by halogen, oxo, hydroxy, cyano, or combinations thereof;
R³ is H,
C₁₋₈-alkyl which is branched or unbranched, and which is unsubstituted or substituted one or more times with halogen, hydroxyl, cyano, C₁₋₄-alkoxy, or combinations thereof,
a partially unsaturated C₅₋₁₄-carbocycle-C₁₋₅-alkyl group wherein the alkyl portion which is branched or unbranched, and which is unsubstituted or substituted in the carbocyclic portion one or more times by halogen, C₁₋₈-alkyl, C₁₋₈-alkoxy, nitro, cyano, oxo, or combinations thereof, and the alkyl portion is optionally substituted by halogen, C₁₋₄-alkoxy, cyano or combinations thereof,
C₇₋₁₉-arylalkyl having 7 to 19 carbon atoms, wherein the aryl portion has 6 to 14 carbon atoms and the alkyl portion, which is branched or unbranched, has 1 to 5 carbon atoms, and which is unsubstituted or substituted, in the aryl portion, one or more times by halogen, trifluoromethyl, CF₃O, nitro, amino, C₁₋₈-alkyl, C₁₋₈-alkoxy, C₁₋₈-alkylamino, di-(C₁₋₈-alkyl)amino, and/or substituted in the alkyl portion by halogen, cyano, or methyl, or
a heterocycle-C₁₋₅-alkyl group, wherein the heterocyclic portion is saturated, partially saturated or unsaturated, wherein the heterocyclic portion has 5 to 10 ring atoms in which at least 1 ring atom is a N, O or S atom, and the alkyl portion is branched or unbranched, the heterocycle-C₁₋₅-alkyl group is unsubstituted or substituted one or more times in the heterocyclic portion by halogen, C₁₋₈-alkyl, C₁₋₈-alkoxy, cyano, trifluoromethyl, CF₃O, nitro, oxo, amino, C₁₋₈-alkylamino, di-(C₁₋₈-alkyl)amino, aminocarbonyl, unsubstituted C₆₋₁₄-aryl, or C₆₋₁₄-aryl substituted one or more times by C₁₋₄-alkyl, C₁₋₄-alkoxy, halogenated C₁₋₄-alkyl, and/or halogenated C₁₋₄-alkoxy, or combinations thereof, and/or substituted in the alkyl portion by halogen, cyano, or methyl or combinations thereof;
R⁴ is H,
C₁₋₈-alkyl which is branched or unbranched and which is unsubstituted or substituted one or more times with halogen, carboxy, cyano, C₁₋₄-alkoxy, or combinations thereof,
C₃₋₁₀-cycloalkyl which is unsubstituted or substituted one or more times by halogen, hydroxy, oxo, carboxy, cyano, C₁₋₄-alkyl, C₁₋₄-alkoxy, or combinations thereof,
C₆₋₁₄-aryl and which is unsubstituted or substituted one or more times by halogen,
C₁₋₈-alkyl, C₂₋₈-alkenyl, C₂₋₈-alkynyl, hydroxy, C₁₋₈-alkoxy, C₁₋₈-alkoxy-C₁₋₈-alkoxy, nitro, methylenedioxy, ethylenedioxy, trifluoromethyl, OCF₃, amino, amino-C₁₋₈-alkyl, amino-C₂₋₈-alkoxy, C₁₋₈-alkylamino, di-(C₁₋₈-alkyl)amino, C₁₋₈-hydroxyalkyl, —C(O)—NHOH, pyrrolyl, tetrazole-5-yl, 2(-heterocycle)tetrazole-5-yl, C₁₋₈-hydroxyalkoxy, C₁₋₈-dihydroxyalkoxy, carboxy, C₁₋₈-alkoxy-carbonyl, cyano, acyl, C₁₋₈-alkylthio, C₁₋₈-alkylsulfinyl, C₁₋₈-alkylsulfonyl, phenoxy, tri-C₁₋₈-alkylsilyloxy, R⁵-L-, or combinations thereof, or
a heterocyclic group, which is fully saturated, partially saturated or unsaturated, having 5 to 10 ring atoms in which at least 1 ring atom is a heteroatom, which is unsubstituted or substituted one or more times by halogen, C₁₋₈-alkyl, hydroxy, C₁₋₈-alkoxyl, oxo, C₁₋₈-alkoxy-C₁₋₈-alkoxy, nitro, methylenedioxy, ethylenedioxy, trifluoromethyl, amino, amino-C₁₋₈-alkyl, C₁₋₈-alkylamino, amino-C₂₋₈-alkoxy, di-C₁₋₈-alkylamino, C₁₋₈-hydroxyalkyl, —C(O)—NHOH, tetrazole-5-yl, C₁₋₈-hydroxyalkoxy, carboxy, C₁₋₈-alkoxy-carbonyl, cyano, acyl, C₁₋₈-alkylthio, C₁₋₈-alkylsulfinyl, C₁₋₈-alkylsulfonyl, phenoxy, tri-C₁₋₈-alkylsilyloxy, R⁵-L-, or combinations thereof;
R⁵ is H,
C₁₋₈-alkyl which is unsubstituted or substituted one or more times with halogen,
C₁₋₄-alkyl, C₁₋₄-alkoxy, oxo, hydroxy, or combinations thereof,
C₁₋₈-alkylamino or di-C₁₋₈-alkylamino,
a partially unsaturated carbocycle-alkyl group wherein the carbocyclic portion has 5 to 14 carbon atoms and the alkyl portion has 1 to 5 carbon atoms, which is unsubstituted or substituted one or more times by halogen, alkyl, alkoxy, nitro, cyano, oxo, or combinations thereof,
C₃₋₁₀-cycloalkyl which is unsubstituted or substituted one or more times by halogen, hydroxy, oxo, cyano, C₁₋₄-alkoxy, C₁₋₄-alkyl, or combinations thereof,
C₄₋₁₆-cycloalkylalkyl which is unsubstituted or substituted in the cycloalkyl portion and/or the alkyl portion one or more times by halogen, oxo, cyano, hydroxy, alkyl, alkoxy or combinations thereof,
C₆₋₁₄-aryl which is unsubstituted or substituted one or more times by halogen, C₁₋₈-alkyl, hydroxy, C₁₋₈-alkoxy, C₁₋₈-alkoxy-C₁₋₈-alkoxy, nitro, methylenedioxy, ethylenedioxy, trifluoromethyl, amino, amino-C₁₋₈-alkyl, amino-C₂₋₈-alkoxy, C₁₋₈-alkylamino, di-C₁₋₈-alkylamino, C₁₋₈-hydroxyalkyl, —C(O)—NHOH, tetrazole-5-yl, C₁₋₈-hydroxyalkoxy, carboxy, C₁₋₈-alkoxy-carbonyl, cyano, acyl, C₁₋₈-alkylthio, C₁₋₈-alkylsulfinyl, C₁₋₈-alkylsulfonyl, phenoxy, C₃₋₁₀-cycloalkyl, C₆₋₁₄-aryl, heteroaryl or combinations thereof,
C₇₋₁₉-arylalkyl wherein the aryl portion has 6 to 14 carbon atoms and the alkyl portion, which is branched or unbranched, has 1 to 5 carbon atoms, and which is unsubstituted or substituted, in the aryl portion, one or more times by halogen, trifluoromethyl, CF₃O, nitro, amino, C₁₋₈-alkyl, C₁₋₈-alkoxy, amino, C₁₋₈-alkylamino, or di-C₁₋₈-alkylamino, and/or substituted in the alkyl portion by halogen, cyano, or methyl,
a heterocyclic group, which is saturated, partially saturated or unsaturated, having 5 to 10 ring atoms in which at least 1 ring atom is a N, O or S atom, which is unsubstituted or substituted one or more times by halogen, C₁₋₈-alkyl, hydroxy, C₁₋₈-alkoxy, C₁₋₈-alkoxy-C₁₋₈-alkoxy, oxo, nitro, methylenedioxy, ethylenedioxy, trifluoromethyl, amino, amino-C₁₋₈-alkyl, amino-C₂₋₈-alkoxy, C₁₋₈-alkylamino, di-C₁₋₈-alkylamino, C₁₋₈-hydroxyalkyl, —C(O)—NHOH, tetrazole-5-yl, C₁₋₈-hydroxyalkoxy, carboxy, C₁₋₈-alkoxy-carbonyl, cyano, acyl, C₁₋₈-alkylthio, C₁₋₈-alkylsulfinyl, C₁₋₈-alkylsulfonyl, phenoxy, C₃₋₁₀-cycloalkyl, C₆₋₁₄-aryl, heteroaryl or combinations thereof, or
a heterocycle-alkyl group, wherein the heterocyclic portion is saturated, partially saturated or unsaturated, and has 5 to 10 ring atoms in which at least 1 ring atom is a N, O or S atom, and the alkyl portion which is branched or unbranched and has 1 to 5 carbon atoms, the heterocycle-alkyl group is unsubstituted or substituted one or more times in the heterocyclic portion by halogen, C₁₋₈-alkyl, C₁₋₈-alkoxy, cyano, trifluoromethyl, CF₃O, nitro, oxo, amino, C₁₋₈-alkylamino, di-C₁₋₈-alkylamino, or combinations thereof and/or substituted in the alkyl portion by halogen, cyano, or methyl or combinations thereof;
L is a single bond or a divalent aliphatic radical having 1 to 8 carbon atoms wherein one or more —CH₂— groups are each optionally replaced by —O—, —S—, —SO—, —SO₂—, —NR⁶—, —SO₂NH—, —NHSO₂—, —SO₂NR⁶—, —NR⁶SO₂—, —CHOH—, —CO—, —NR⁶CO—, —CONR⁶—, —NHCONH—, —OCONH, —NHCOO—, —SCONH—, —SCSNH—, or —NHCSNH—; and
R⁶ is H,
C₁₋₈-alkyl which is branched or unbranched and which is unsubstituted or substituted one or more times with halogen, C₁₋₄-alkyl, C₁₋₄-alkoxy, oxo, or combinations thereof;
C₇₋₁₉-arylalkyl wherein the aryl portion has 6 to 14 carbon atoms and the alkyl portion, which is branched or unbranched, has 1 to 5 carbon atoms, and which is unsubstituted or substituted, in the aryl portion, one or more times by halogen, trifluoromethyl, CF₃O, nitro, amino, C₁₋₈-alkyl, C₁₋₈-alkoxy, C₁₋₈-alkylamino, or di-C₁₋₈-alkylamino, and/or substituted in the alkyl portion by halogen, cyano, or methyl;
C₆₋₁₄-aryl which is unsubstituted or substituted one or more times by halogen, C₁₋₈-alkyl, hydroxy, C₁₋₈-alkoxy, C₁₋₈-alkoxy-C₁₋₈-alkoxy, nitro, methylenedioxy, ethylenedioxy, trifluoromethyl, amino, amino-C₁₋₈-alkyl, amino-C₂₋₈-alkoxy, C₁₋₈-alkylamino, di-C₁₋₈-alkylamino, C₁₋₈-hydroxyalkyl, —C(O)—NHOH, tetrazole-5-yl, C₁₋₈-hydroxyalkoxy, carboxy, C₁₋₈-alkoxy-carbonyl, cyano, acyl, C₁₋₈-alkylthio, C₁₋₈-alkylsulfinyl, or C₁₋₈-alkylsulfonyl; or
a pharmaceutically acceptable salt or solvate or N-oxide thereof, or solvate of pharmaceutically acceptable salt or N-oxide thereof;
with the proviso that at least one of R³ and R⁴ is other than H; and
with the further proviso that:
R¹ is cycloalkyl having 3 to 10 which is unsubstituted or substituted one or more times by halogen, hydroxy, oxo, cyano, C₁₋₄-alkyl, C₁₋₄-alkoxy, or combinations thereof; and/or
R³ is alkyl having 1 to 8 carbon atoms, which is branched or unbranched and which is substituted one or more times with hydroxyl, or
a heterocycle-alkyl group, wherein the heterocyclic portion is saturated, partially saturated or unsaturated and has 5 to 10 ring atoms in which at least 1 ring atom is a N, O or S atom, and the alkyl portion, which is branched or unbranched, has 1 to 5 carbon atoms, the heterocycle-alkyl group is substituted one or more times in the heterocycle portion by at least aminocarbonyl, unsubstituted aryl having 6 to 14 carbon atoms, aryl having 6 to 14 carbon atoms which is substituted one or more times by C₁₋₄-alkyl, C₁₋₄-alkoxy, halogenated C₁₋₄-alkyl, and/or halogenated C₁₋₄-alkoxy, or combinations thereof, and is optionally substituted in the alkyl portion by halogen, cyano, or methyl or combinations thereof; and/or
R⁴ is alkyl having 1 to 8 carbon atoms, which is branched or unbranched and which is unsubstituted or substituted one or more times with halogen, carboxy, cyano, C₁₋₄-alkoxy, or combinations thereof, or
cycloalkyl having 3 to 10 carbon atoms, which is substituted one or more times by carboxy,
aryl having 6 to 14 carbon atoms and which is substituted one or more times by at least substituted-C₁₋₈-alkoxy-C₁₋₈-alkoxy wherein the substituted-C₁₋₈-alkoxy is substituted one or more times by halogen, hydroxy, carboxy, and/or cyano, or
a heterocyclic group, which is fully saturated or partially saturated, having 5 to 10 ring atoms in which at least 1 ring atom is a heteroatom, which is unsubstituted or substituted one or more times by halogen, C₁₋₈-alkyl, hydroxy, C₁₋₈-alkoxy, oxo, C₁₋₈-alkoxy-C₁₋₈-alkoxy, nitro, methylenedioxy, ethylenedioxy, trifluoromethyl, amino, amino-C₁₋₈-alkyl, amino-C₂₋₈-alkoxy, C₁₋₈-alkylamino, di-C₁₋₈-alkylamino, (substituted-C₁₋₈-alkyl)amino wherein the alkyl group is substituted one or more times by halogen, hydroxy, carboxy and/or cyano, C₁₋₈-alkyl(substituted-C₁₋₈-alkyl)amino wherein the substituted-C₁₋₈-alkyl is substituted one or more times by halogen, hydroxy, carboxy and/or cyano, C₁₋₈-hydroxyalkyl, —C(O)—NHOH, tetrazole-5-yl, C₁₋₈-hydroxyalkoxy, carboxy, C₁₋₈-alkoxy-carbonyl, cyano, acyl, C₁₋₈-alkylthio, C₁₋₈-alkylsulfinyl, C₁₋₈-alkylsulfonyl, phenoxy, tri-C₁₋₈-alkylsilyloxy, R⁵-L-, or combinations thereof, or
a heterocyclic group, which is unsaturated and has 5 to 10 ring atoms in which at least 1 ring atom is a heteroatom, which is substituted one or more times by at least oxo, (substituted-C₁₋₈-alkyl)amino wherein the substituted-C₁₋₈-alkyl is substituted one or more times by halogen, hydroxy, carboxy and/or cyano, or C₁₋₈-alkyl(substituted-C₁₋₈-alkyl)amino wherein the substituted-C₁₋₈-alkyl is substituted one or more times by halogen, hydroxy, carboxy and/or cyano, or combinations thereof; and/or
R⁵ is a heterocyclic group, which is saturated, partially saturated or unsaturated, having 5 to 10 ring atoms in which at least 1 ring atom is a N, O or S atom, which is substituted one or more times by at least oxo.

2. A compound according to claim 1, wherein optionally substituted radicals have 1 to 3 substituents.

3. A compound according to claim 1, wherein R¹ is C₁₋₄-alkyl, halogenated C₁₋₄-alkyl, or optionally substituted C₃₋₁₀-cycloalkyl.

4. A compound according to claim 1, wherein R¹ is methyl, ethyl, isopropyl, difluoromethyl, trifluoroethyl, trifluoromethyl, or cyclopropyl.

5. A compound according to claim 1, wherein R² is (i) C₁₋₈-alkyl or halogenated C₁₋₈-alkyl, (ii) C₃₋₈-cycloalkyl which is unsubstituted or substituted, (iii) C₄₋₁₂-cycloalkylalkyl which is unsubstituted or substituted, (iv) arylalkyl which is unsubstituted or substituted, or (v) tetrahydrofuranyl.

6. A compound according to claim 5, wherein R² is methyl, ethyl, isopropyl, butyl, difluoromethyl, trifluoroethyl, trifluoromethyl, cyclopropyl, cyclobutyl, cyclopentyl, cyclopropylmethyl, benzyl, or tetrahydrofuranyl.

7. A compound according to claim 1, wherein R³ is
(i) C₁₋₄-alkyl which is branched or unbranched and which is unsubstituted or substituted one or more times by hydroxyl and/or carboxy;
(ii) C₇₋₁₉-arylalkyl which is optionally substituted in the alkyl and/or the aryl portions;
(iii) a heterocyclic-alkyl which is optionally substituted in the alkyl and/or the heteroaryl portions, and wherein the heterocyclic portion is unsaturated or partially unsaturated; or
(iv) a heterocyclic-alkyl group which is optionally substituted, wherein the heterocyclic portion is fully saturated.

8. A compound according to claim 7, wherein R³ is benzyl, pyrazolylmethyl, pyridinylmethyl, oxazolylmethyl, thiazolylmethyl, pyrimidinylmethyl, pyridizinylmethyl, pyrazinylmethyl, and thiadiazolylmethyl, which in each case is unsubstituted or substituted.

9. A compound according to claim 1, wherein R⁴ is aryl or heteroaryl, which in each case is unsubstituted or is substituted one or more times by substituents selected OH, F, Cl, CF₃, methyl, ethyl, oxo, methoxy, ethoxy, hydroxyalkoxy, (dihydroxy)alkoxy, NHCOCH₃, (substituted-alkyl)amino, alkyl(substituted-alkyl)amino, CN, CONHOH, CONHCH₂CH₂OH, COOH, CH₂COOH, (CH₂)₂COOH, and COOalkyl.

10. A compound according to claim 1, wherein R¹ is cycloalkyl having 3 to 10 which is unsubstituted or substituted one or more times by halogen, hydroxy, oxo, cyano, C₁₋₄-alkyl, C₁₋₄-alkoxy, or combinations thereof.

11. A compound according to claim 10, wherein said compound is 3-[[4-(cyclopropyloxy)-3-ethoxyphenyl](pyrimidin-5-ylmethyl)amino]benzoic acid, or a pharmaceutically acceptable salt.

12. A compound according to claim 1, wherein R³ is
alkyl having 1 to 8 carbon atoms, which is branched or unbranched and which is substituted one or more times with hydroxyl, or
a heterocycle-alkyl group, wherein the heterocyclic portion is saturated, partially saturated or unsaturated and has 5 to 10 ring atoms in which at least 1 ring atom is a N, O or S atom, and the alkyl portion, which is branched or unbranched, has 1 to 5 carbon atoms, the heterocycle-alkyl group is substituted one or more times in the heterocycle portion by at least aminocarbonyl, unsubstituted aryl having 6 to 14 carbon atoms, or aryl having 6 to 14 carbon atoms and which is substituted one or more times by C₁₋₄-alkyl, C₁₋₄-alkoxy, halogenated C₁₋₄-alkyl, and/or halogenated C₁₋₄-alkoxy, or combinations thereof, and is optionally substituted in the alkyl portion by halogen, cyano, or methyl or combinations thereof.

13. A compound according to claim 12, wherein said compound is selected from 2-[[3,4bis(difluoromethoxy)phenyl](phenyl)amino]-ethanol, 1-[[3,4-bis(difluoromethoxy)phenyl](3-chlorophenyl)amino]-2-methylpropan-2-ol, 1-[[3,4-bis(difluoromethoxy)phenyl](3-chlorophenyl)amino]propan-2-ol, 3-[[3,4-bis(difluoromethoxy)phenyl](3-chlorophenyl)amino]propan-1-ol, and pharmaceutically acceptable salts thereof.

14. A compound according to claim 1, wherein R⁴ is
alkyl having 1 to 8 carbon atoms, which is branched or unbranched and which is unsubstituted or substituted one or more times with halogen, carboxy, cyano, C₁₋₄-alkoxy, or combinations thereof,
cycloalkyl having 3 to 10, preferably 3 to 8 carbon atoms, which is substituted one or more times by carboxy,
aryl having 6 to 14 carbon atoms and which is substituted one or more times by at least (C₁₋₈-alkoxy substituted one or more times by halogen, hydroxy, carboxy, and/or cyano)-C₁₋₈-alkoxy, dihydroxyalkoxy, or combinations thereof,
a heterocyclic group, which is fully saturated or partially saturated, having 5 to 10 ring atoms in which at least 1 ring atom is a heteroatom, which is unsubstituted or substituted one or more times by halogen, C₁₋₈-alkyl, hydroxy, C₁₋₈-alkoxy, oxo, C₁₋₈-alkoxy-C₁₋₈-alkoxy, nitro, methylenedioxy, ethylenedioxy, trifluoromethyl, amino, amino-C₁₋₈-alkyl, amino-C₂₋₈-alkoxy, C₁₋₈-alkylamino, di-C₁₋₈-alkylamino, (C₁₋₈-alkyl substituted one or more times by halogen, hydroxy, carboxy and/or cyano)amino, C₁₋₈-alkyl(C₁₋₈-alkyl substituted one or more times by halogen, hydroxy, carboxy and/or cyano)amino, C₁₋₈-hydroxyalkyl, —C(O)—NHOH, tetrazole-5-yl, C₁₋₈-hydroxyalkoxy, carboxy, C₁₋₈-alkoxy-carbonyl, cyano, acyl, C₁₋₈-alkylthio, C₁₋₈-alkylsulfinyl, C₁₋₈-alkylsulfonyl, phenoxy, tri-C₁₋₈-alkylsilyloxy, R⁵-L-, or combinations thereof, or
a heterocyclic group, which is unsaturated having 5 to 10 ring atoms in which at least 1 ring atom is a heteroatom, which is substituted one or more times by at least oxo, (C₁₋₈-alkyl substituted one or more times by halogen, hydroxy, carboxy and/or cyano, or C₁₋₈-alkyl(C₁₋₈-alkyl substituted one or more times by halogen, hydroxy, carboxy and/or cyano), or combinations thereof.

15. A compound according to claim 14, wherein said compound is 4-[[3,4-bis(difluoromethoxy)phenyl](1,3-thiazol-5-ylmethyl)amino]butanoic acid, or a pharmaceutically acceptable salt thereof.

16. A compound according to claim 14, wherein said compound is cis-4-[[3,4-bis(difluoromethoxy)phenyl](1,3-thiazol-5-ylmethyl)amino]cyclohexane-carboxylic acid, or a pharmaceutically acceptable salt thereof.

17. A compound according to claim 14, wherein said compound is selected from (2R)-3-{4-[[3-(cyclopentyloxy)-4-methoxyphenyl](pyridin-3-ylmethyl)amino]-phenoxy}propane-1,2-diol,
(2R)-3-{3-[[3-(cyclopentyloxy)-4-methoxyphenyl](pyridin-3-ylmethyl)amino]-phenoxy}propane-1,2-diol,
(2R)-3-{4-[[3,4-bis(difluoromethoxy)phenyl](pyridin-3-ylmethyl)amino]-phenoxy}propane-1,2-diol,
(2R)-3-{4-[[3-(cyclopentyloxy)-4-methoxyphenyl](1,3-thiazol-5-ylmethyl)amino]-phenoxy}propane-1,2-diol,
(2R)-3-{4-[(3,4-dimethoxyphenyl)(pyridin-3-ylmethyl)amino]phenoxy}propane-1,2-diol,
5-[[3,4-bis(difluoromethoxy)phenyl](pyridin-3-ylmethyl)amino]-2-{[(2R)-2,3-dihydroxypropyl]oxy}benzoic acid,
(2R)-3-{3-[[3,4-bis(difluoromethoxy)phenyl](pyridin-3-ylmethyl)amino]-phenoxy}propane-1,2-diol,
(2R)-3-{4-[[3,4-bis(difluoromethoxy)phenyl](1,3-thiazol-5-ylmethyl)amino]-phenoxy}propane-1,2-diol,
(2R)-3-{3-[[3,4-bis(difluoromethoxy)phenyl](1,3-thiazol-5-ylmethyl)amino]-phenoxy}propane-1,2-diol,
1-{3-[[3,4-bis(difluoromethoxy)phenyl](1,3-thiazol-5-ylmethyl)amino]phenoxy}-3-methoxypropan-2-ol,
and pharmaceutically acceptable salts thereof.

18. A compound according to claim 14, wherein said compound is selected from
5-[[3-(cyclopentyloxy)-4-methoxyphenyl](pyridin-3-ylmethyl)amino]pyridin-2(1H)-one,
5-[[3-(cyclopentyloxy)-4-methoxyphenyl](pyridin-3-ylmethyl)amino]-2-methyl-1,2-benzisoxazol-3(2H)-one,
5-[[3-(cyclopentyloxy)-4-methoxyphenyl](pyridin-3-ylmethyl)amino]-1,2-benzisoxazol-3(2H)-one,
5-[[3,4-bis(difluoromethoxy)phenyl](1,3-thiazol-5-ylmethyl)amino]-1,2-benzisoxazol-3(2H)-one,
6-[[3,4-bis(difluoromethoxy)phenyl](1,3-thiazol-5-ylmethyl)amino]-1,2-benzisoxazol-3(2H)-one,
and pharmaceutically acceptable salts thereof.

19. A compound according to claim 14, wherein said compound is selected from
5-[[3-(cyclopentyloxy)-4-methoxyphenyl](pyridin-3-ylmethyl)amino]pyridin-2(1H)-one,
2-({5-[[3-(cyclopentyloxy)-4-methoxyphenyl](pyridin-3-ylmethyl)amino]pyridin-2-yl}amino)propan-1-ol,
3-[{5-[[3-(cyclopentyloxy)-4-methoxyphenyl](pyridin-3-ylmethyl)amino]pyridin-2-yl}(methyl)amino]propane-1,2-diol,
2-({5-[[3-(cyclopentyloxy)-4-methoxyphenyl](pyridin-3-ylmethyl)amino]pyridin-2-yl}amino)butan-1-ol,
1-({5-[[3-(cyclopentyloxy)-4-methoxyphenyl](pyridin-3-ylmethyl)amino]pyridin-2-yl}amino)butan-2-ol
4-({5-[[3-(cyclopentyloxy)-4-methoxyphenyl](pyridin-3-ylmethyl)amino]pyridin-2-yl}amino)butan-2-ol,
1-({5-[[3-(cyclopentyloxy)-4-methoxyphenyl](pyridin-3-ylmethyl)amino]pyridin-2-yl}amino)-2-methylpropan-2-ol trifluoroacetic acid salt,
3-[{5-[[3-(cyclopentyloxy)-4-methoxyphenyl](1,3-thiazol-5-ylmethyl)amino]pyridin-2-yl}(methyl)amino]propane-1,2-diol,
3-[{5-[[3,4-bis(difluoromethoxy)phenyl](pyridin-3-ylmethyl)amino]pyridin-2-yl}(methyl)amino]propane-1,2-diol,
and pharmaceutically acceptable salts thereof.

20. A compound according to claim 1, wherein R⁵ is a heterocyclic group, which is saturated, partially saturated or unsaturated, having 5 to 10 ring atoms in which at least 1 ring atom is a N, O or S atom, which is substituted one or more times by at least oxo.

21. A compound according to claim 20, wherein said compound is selected from
(5S)-5-({3-[[3-(cyclopentyloxy)-4-methoxyphenyl](pyridin-3-ylmethyl)amino]phenoxy}-methyl)pyrrolidin-2-one,
4-{3-[[3,4-bis(difluoromethoxy)phenyl]-(pyridin-3-ylmethyl)amino]phenyl}-2-methyl-1,2-dihydro-3H-pyrazol-3-one,
5-{3-[[3-(cyclopentyloxy)-4-methoxyphenyl](pyridin-3-ylmethyl)amino]phenyl}imidazolidine-2,4-dione,
5-{4-[[3-(cyclopentyloxy)-4-methoxyphenyl](pyridin-3-ylmethyl)amino]phenyl}-imidazolidine-2,4-dione,
1-({5-[[3-(cyclopentyloxy)-4-methoxyphenyl](pyridin-3-ylmethyl)amino]pyridin-2-yl}amino)propan-2-ol,
5-{3-[[3,4-bis(difluoromethoxy)phenyl](1,3-thiazol-5-ylmethyl)amino]phenyl}-1,3,5-trimethylimidazolidine-2,4-dione,
5-{3-[[3,4-bis(difluoromethoxy)phenyl](1,3-thiazol-5-ylmethyl)amino]phenyl}imidazolidine-2,4-dione,
4-{3-[[3,4-bis(difluoromethoxy)phenyl](pyridin-3-ylmethyl)amino]phenyl}imidazolidin-2-one,
5-{3-[[3,4-bis(difluoromethoxy)phenyl](pyridin-3-ylmethyl)amino]phenyl}-1,3-oxazolidin-2-one,
4-{3-[[3,4-bis(difluoromethoxy)phenyl](pyridin-3-ylmethyl)amino]phenyl}-1,3-oxazolidin-2-one,
and pharmaceutically acceptable salts thereof.

22. A compound according to claim 1, wherein said compound is of subformulae II or III: 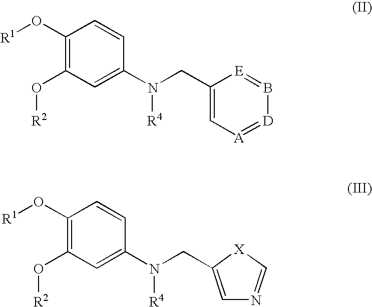
wherein R¹, R², and R⁴ are as defined in Formula I, one or two of A, B, D and E are N, and the others are CH, and X is O or S; or
a pharmaceutically acceptable salt or solvate or N-oxide thereof, or solvate of pharmaceutically acceptable salt or N-oxide thereof.

23. A compound according to claim 22, wherein
A is N, and B, D and E are CH;
A and B are both N, and D and E are CH;
A and E are both N, and D and B are CH; or
A and D are both N, and B and E are CH.

24. A compound according to claim 22, wherein R⁴ is aryl which is substituted or unsubstituted.

25. A compound according to claim 22, wherein R¹ is halogenated alkyl, and R² is alkyl or halogenated alkyl.

26. A compound according to claim 25, wherein R¹ is CHF₂, R² is methyl, ethyl, or CHF₂, A is N, B is CH or N, D is CH, X is O or S, and R⁴ is substituted phenyl.

27. A compound according to claim 1, wherein said compound is of subformula IV: 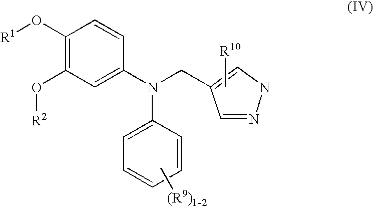
wherein
R¹ is C₁₋₄-alkyl or halogenated C₁₋₄-alkyl,
R² is C₁₋₄-alkyl, halogenated C₁₋₄-alkyl, or C-₃₋₁₀-cycloalkyl,
R⁹ is halogen, C₁₋₄-alkyl, hydroxy, C₁₋₄-alkoxy, C₁₋₄-alkoxy-C₁₋₈-alkoxy, nitro, trifluoromethyl, OCF₃, amino, C₁₋₄-alkylamino, di-(C₁₋₄-alkyl)amino, C₁₋₈-hydroxyalkyl, C₁₋₄-hydroxyalkoxy, C₁₋₄-dihydroxyalkoxy, carboxy, carboxy-C₁₋₄-alkyl, C₁₋₄-alkoxy-carbonyl, C₁₋₄-alkylsulfonyl, aminosulfonyl, aminocarbonyl, C₁₋₄-alkylaminocarbonyl, di-C₁₋₄-alkylaminocarbonyl, C₁₋₄-alkyl-SO₂—NH—, C₁₋₄-alkyl-NH—SO₂—, or cyano, and
R¹⁰ is H or C₁₋₄-alkyl, or
a pharmaceutically acceptable salt or solvate or N-oxide thereof, or solvate of pharmaceutically acceptable salt or N-oxide thereof.

28. A compound according to claim 1, wherein said compound is of subformulae Va or Vb: 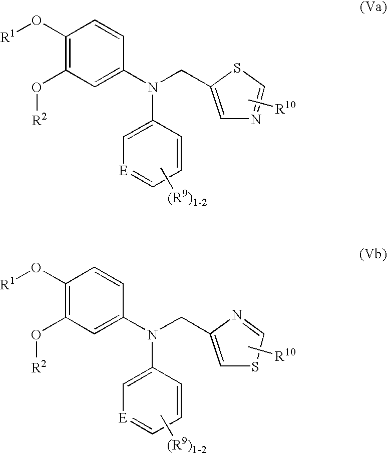
wherein
E is N or CH,
R¹ is C₁₋₄-alkyl or halogenated C₁₋₄-alkyl,
R² is C₁₋₄-alkyl, halogenated C₁₋₄-alkyl, C₁₋₃-₁₀-cycloalkyl, C₄₋₁₂-cycloalkylalkyl, or tetrahydrofuranyl,
R⁹ is halogen, C₁₋₄-alkyl, hydroxy, C₁₋₄-alkoxy, C₁₋₄-alkoxy-C₁₋₈-alkoxy, nitro, trifluoromethyl, OCF₃, amino, C₁₋₄-alkylamino, di-(C₁₋₄-alkyl)amino, C₁₋₈-hydroxyalkyl, C₁₋₄-hydroxyalkoxy, C₁₋₄-dihydroxyalkoxy, C₁₋₄-alkoxy-C₁₋₄-hydroxyalkoxy, carboxy, carboxy-C₁₋₄-alkyl, C₁₋₄-alkoxy-carbonyl, C₁₋₄-alkylsulfonyl, aminosulfonyl, aminocarbonyl, C₁₋₄-alkylaminocarbonyl, di-C₁₋₄-alkylaminocarbonyl, C₁₋₄-alkyl-SO₂—NH—, C₁₋₄-alkyl-NH—SO₂—, cyano, trimethylimidazolidinedione, or imidazolidinedione, and
R¹⁰ is H or C₁₋₄-alkyl, or
a pharmaceutically acceptable salt or solvate or N-oxide thereof, or solvate of pharmaceutically acceptable salt or N-oxide thereof.

29. A compound according to claim 1, wherein said compound is of subformula VI: 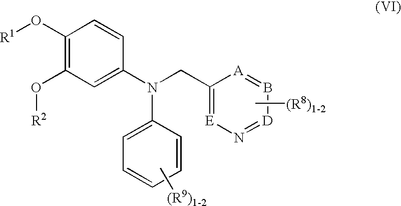
wherein
one of A, B, D and E is N and the others are each CH or CR⁸ if the carbon atom carries the R⁸ substituent,
R¹ is C₁₋₄-alkyl or halogenated C₁₋₄-alkyl,
R² is C₁₋₄-alkyl, halogenated C₁₋₄-alkyl, C-₃₋₁₀-cycloalkyl, or C₄₋₁₂-cycloalkylalkyl,
R⁸ is amino, C₁₋₄-alkylamino, or di-(C₁₋₄-alkyl)amino, and
R⁹ is halogen, C₁₋₄-alkyl, hydroxy, C₁₋₄-alkoxy, C₁₋₄-alkoxy-C₁₋₈-alkoxy, nitro, trifluoromethyl, OCF₃, amino, C₁₋₄-alkylamino, di-(C₁₋₄-alkyl)amino, C₁₋₈-hydroxyalkyl, C₁₋₄-hydroxyalkoxy, C₁₋₄-dihydroxyalkoxy, carboxy, carboxy-C₁₋₄-alkyl (HOOC—CH₂—), C₁₋₄-alkyl-carbonyl, C₁₋₄-alkoxy-carbonyl, C₁₋₄-alkylsulfonyl, aminosulfonyl, aminocarbonyl, C₁₋₄-alkylaminocarbonyl, di-C₁₋₄-alkylaminocarbonyl, C₁₋₄-alkyl-SO₂—NH—, C₁₋₄-alkyl-NH—SO₂— or cyano, or
a pharmaceutically acceptable salt or solvate or N-oxide thereof, or solvate of pharmaceutically acceptable salt or N-oxide thereof.

30. A compound according to claim 29, wherein A is N.

31. A compound according to claim 29, wherein B is N.

32. A compound according to claim 29, wherein D is N.

33. A compound according to claim 29, wherein E is N.

34. A compound according to claim 1, wherein said compound is of subformula VII: 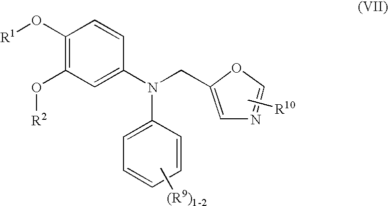
wherein
R¹ is C₁₋₄-alkyl or halogenated C₁₋₄-alkyl,
R² is H, C₁₋₄-alkyl, halogenated C₁₋₄-alkyl, C-₃₋₁₀-cycloalkyl, or C-₄₋₁₂-cycloalkylalkyl,
R⁹ is halogen, C₁₋₄-alkyl, hydroxy, C₁₋₄-alkoxy, C₁₋₄-alkoxy-C₁₋₈-alkoxy, nitro, trifluoromethyl, OCF₃, amino, C₁₋₄-alkylamino, di-(C₁₋₄-alkyl)amino, C₁₋₈-hydroxyalkyl, C₁₋₄-hydroxyalkoxy, C₁₋₄-dihydroxyalkoxy, carboxy, carboxy-C₁₋₄-alkyl, C₁₋₄-alkoxy-carbonyl, C₁₋₄-alkylsulfonyl, aminosulfonyl, aminocarbonyl, C₁₋₄-alkylaminocarbonyl, di-C₁₋₄-alkylaminocarbonyl, C₁₋₄-alkyl-SO₂—NH—, C₁₋₄-alkyl-NH—SO₂—, or cyano, and
R¹⁰ is H or C₁₋₄-alkyl, or
a pharmaceutically acceptable salt or solvate or N-oxide thereof, or solvate of pharmaceutically acceptable salt or N-oxide thereof.

35. A compound selected from:
1) 4-[[3,4-bis(difluoromethoxy)phenyl](pyridin-3-ylmethyl)amino]-N-(methylsulfonyl)benzamide,
2) 4-[[3,4-bis(difluoromethoxy)phenyl](pyridin-3-ylmethyl)amino]-N-(ethylsulfonyl)benzamide,
3) 3-{(3-fluorobenzyl) [4-methoxy-3-(2,2,2-trifluoroethoxy)phenyl]amino}benzoic acid,
21 MEMORY-0058
4) 3-[(3,4-dimethoxyphenyl)(3-fluorobenzyl)amino]benzoic acid,
5) 3-[(3-ethoxy-4-methoxyphenyl)(3-fluorobenzyl)amino]benzoic acid,
6) 3-[[3-(cyclobutyloxy)-4-methoxyphenyl](3-fluorobenzyl)amino]benzoic acid,
7) 3-[[3-(cyclopentyloxy)-4-methoxyphenyl](3-fluorobenzyl)amino]benzoic acid,
8) 3-[(2,6-difluorobenzyl)(3,4-dimethoxyphenyl)amino]benzoic acid,
9) 3-[(2,6-difluorobenzyl)(3-ethoxy-4-methoxyphenyl)amino]benzoic acid,
10) 3-[(2,6-difluorobenzyl)(3-isopropoxy-4-methoxyphenyl)amino]benzoic acid,
11) 3-[[3-(cyclobutyloxy)-4-methoxyphenyl](2,6-difluorobenzyl)amino]benzoic acid,
12) 3-((2,6-difluorobenzyl) {4-methoxy-3-[(3R)-tetrahydrofuran-3-yloxy]phenyl}amino)benzoic acid,
13) 3-[[4-(difluoromethoxy)-3-ethoxyphenyl](pyridin-3-ylmethyl)amino]benzoic acid,
14) 4-[[4-(difluoromethoxy)-3-ethoxyphenyl](pyridin-3-ylmethyl)amino]benzoic acid,
15) 3-[[3,4-bis(difluoromethoxy)phenyl](2,6-difluorobenzyl)amino]benzoic acid,
16) 3-[[3,4-bis(difluoromethoxy)phenyl](3-fluorobenzyl)amino]benzoic acid,
17) 3-{(2,6-difluorobenzyl)[4-(difluoromethoxy)-3-methoxyphenyl]amino}benzoic acid,
18) 3-[[4-(difluoromethoxy)-3-methoxyphenyl](pyridin-3-ylmethyl)amino]benzoic acid,
19) 3-[[4-(difluoromethoxy)-3-methoxyphenyl](3-fluorobenzyl)amino]benzoic acid,
20) 3-[[3,4-bis(difluoromethoxy)phenyl](2-fluorobenzyl)amino]benzoic acid,
21) 3-[[3,4-bis(difluoromethoxy)phenyl](4-fluorobenzyl)amino]benzoic acid,
22) 3-((3-fluorobenzyl) {4-methoxy-3-[(3R)-tetrahydrofuran-3-yloxy]phenyl}amino)benzonitrile,
23) N-(3-fluorobenzyl)-4-methoxy-N-[4-(methylsulfonyl)phenyl]-3-[(3R)-tetrahydrofuran-3-yloxy]aniline,
24) N-ethyl-N-[4-((3-fluorobenzyl) {4-methoxy-3-[(3R)-tetrahydrofuran-3-yloxy]phenyl}amino)phenyl]ethanesulfonamide,
25) 3-{(3-cyanobenzyl)[3-(cyclopentyloxy)-4-methoxyphenyl]amino}benzoic acid,
26) 3-{[3-(cyclopentyloxy)-4-methoxyphenyl][(6-oxo-1,6-dihydropyridin-3-yl)methyl]amino}benzoic acid,
27) 5-bromo-N-[3-(cyclopentyloxy)-4-methoxyphenyl]-N-(pyridin-3-ylmethyl)pyridin-2-amine,
28) 3-[[3-(cyclopentyloxy)-4-methoxyphenyl](piperidin-3-ylmethyl)amino]benzoic acid,
29) 4-{[3-(cyclopentyloxy)-4-methoxyphenyl][(6-oxo-1,6-dihydropyridin-3-yl)methyl]amino}benzoic acid,
30) Ethyl {4-[[3-(cyclopentyloxy)-4-methoxyphenyl](pyridin-3-ylmethyl)amino]phenoxy}acetate,
31) 2-{4-[[3-(cyclopentyloxy)-4-methoxyphenyl](pyridin-3-ylmethyl)amino]phenoxy}ethanol,
32) 3-(cyclopentyloxy)-N-(4-{[(4S)-2,2-dimethyl-1,3-dioxolan-4-yl]methoxy}phenyl)-4-methoxy-N-(pyridin-3-ylmethyl)aniline,
33) N(2)-benzyl-N(5)-[3-(cyclopentyloxy)-4-methoxyphenyl]-N(2)-methyl-N(5)-(pyridin-3-ylmethyl)pyridine-2,5-diamine,
34) 3-[[3-(cyclopentyloxy)-4-methoxyphenyl](1H-pyrazol-4-ylmethyl)amino]benzoic acid,
35) 3-[[3-(cyclopentyloxy)-4-methoxyphenyl](1,3-thiazol-5-ylmethyl)amino]benzoic acid,
36) {3-[[3-(cyclopentyloxy)-4-methoxyphenyl](pyridin-3-ylmethyl)amino]phenyl}acetic acid,
37) 5-[[3-(cyclopentyloxy)-4-methoxyphenyl](pyridin-3-ylmethyl)amino]-2-ethylbenzoic acid,
38) 3-[[3-(cyclopentyloxy)-4-methoxyphenyl](pyridin-3-ylmethyl)amino]-4-methylbenzoic acid,
39) 4-[[3-(cyclopentyloxy)-4-methoxyphenyl](1H-pyrazol-4-ylmethyl)amino]benzoic acid,
40) 4-[[3-(cyclopentyloxy)-4-methoxyphenyl](1,3-thiazol-5-ylmethyl)amino]benzoic acid,
41) N-[3-(cyclopentyloxy)-4-methoxyphenyl]-N-(pyridin-3-ylmethyl)-6-pyrrolidin-1-ylpyridin-3-amine,
42) N(5)-[3-(cyclopentyloxy)-4-methoxyphenyl]-N(2)-(2-morpholin-4-ylethyl)-N(5)-(pyridin-3-ylmethyl)pyridine-2,5-diamine,
43) 4-[[3-(cyclopentyloxy)-4-methoxyphenyl](pyrimidin-5-ylmethyl)amino]benzoic acid,
44) N(2)-benzyl-N(5)-[3-(cyclopentyloxy)-4-methoxyphenyl]-N(5)-(pyridin-3-ylmethyl)pyridine-2,5-diamine,
45) 3-[[3-(cyclopentyloxy)-4-methoxyphenyl](1,3-thiazol-4-ylmethyl)amino]benzoic acid,
46) 3-[[3-(cyclopentyloxy)-4-methoxyphenyl](pyrimidin-5-ylmethyl)amino]benzoic acid,
47) 4-[[3-(cyclopentyloxy)-4-methoxyphenyl](pyridazin-3-ylmethyl)amino]benzoic acid,
48) 3-[[3-(cyclopentyloxy)-4-methoxyphenyl](pyridazin-3-ylmethyl)amino]benzoic acid,
49) {4-[[3-(cyclopentyloxy)-4-methoxyphenyl](pyridin-3-ylmethyl)amino]phenyl}acetic acid,
50) 3-[[3-(cyclopentyloxy)-4-methoxyphenyl](piperidin-4-ylmethyl)amino]benzoic acid,
51) 4-([3-(cyclopentyloxy)-4-methoxyphenyl]{[2-(dimethylamino)pyrimidin-5-yl]methyl}amino)benzoic acid,
52) 3-([3-(cyclopentyloxy)-4-methoxyphenyl]{[2-(dimethylamino)pyrimidin-5-yl]methyl}amino)benzoic acid,
53) 3-{4-[[3-(cyclopentyloxy)-4-methoxyphenyl](pyridin-3-ylmethyl)amino]phenyl}propan-1-ol,
54) 5-[[3-(cyclopentyloxy)-4-methoxyphenyl](pyridin-3-ylmethyl)amino]nicotinic acid,
55) 3-{4-[[3-(cyclopentyloxy)-4-methoxyphenyl](pyridin-3-ylmethyl)amino]phenyl}propanoic acid,
56) 3-(cyclopentyloxy)-4-methoxy-N-[3-(2-methyl-1,3-thiazol-4-yl)phenyl]-N-(pyridin-3-ylmethyl)aniline,
57) {3-[[3,4-bis(difluoromethoxy)phenyl](pyridin-3-ylmethyl)amino]phenyl}acetic acid,
58) (5S)-5-({3-[[3-(cyclopentyloxy)-4-methoxyphenyl](pyridin-3-ylmethyl)amino]phenoxy}methyl)pyrrolidin-2-one,
59) 4-[[3,4-bis(difluoromethoxy)phenyl](1,3-thiazol-5-ylmethyl)amino]benzoic acid,
60) 3-[[3,4-bis(difluoromethoxy)phenyl](1,3-thiazol-5-ylmethyl)amino]benzoic acid,
61) N(5)-[3-(cyclopentyloxy)-4-methoxyphenyl]-N(2)-[2-(methylsulfonyl)ethyl]-N(5)-(pyridin-3-ylmethyl)pyridine-2,5-diamine,
62) 3,4-bis(difluoromethoxy)-N-[3-(methylthio)phenyl]-N-(pyridin-3-ylmethyl)aniline,
63) 3,4-bis(difluoromethoxy)-N-[4-(methylsulfonyl)phenyl]-N-(pyridin-3-ylmethyl)aniline,
64) 2-[{5-[[3-(cyclopentyloxy)-4-methoxyphenyl](pyridin-3-ylmethyl)amino]pyridin-2-yl}(methyl)amino]-N,N-dimethylacetamide,
65) N(5)-[3-(cyclopentyloxy)-4-methoxyphenyl]-N(5)-(pyridin-3-ylmethyl)-N(2)-(tetrahydro-2H-pyran-4-yl)pyridine-2,5-diamine,
66) 3,4-bis(difluoromethoxy)-N-[3-(methylsulfonyl)phenyl]-N-(pyridin-3-ylmethyl)aniline,
67) 5-[[3-(cyclopentyloxy)-4-methoxyphenyl](pyridin-3-ylmethyl)amino]-N,2-dihydroxybenzamide,
68) 4-[[3-(cyclopentyloxy)-4-methoxyphenyl](pyridin-3-ylmethyl)amino]-3-methylbenzoic acid,
69) {4-[[3,4-bis(difluoromethoxy)phenyl](pyridin-3-ylmethyl)amino]phenyl}acetic acid,
70) 3-[{4-methoxy-3-[(3R)-tetrahydrofuran-3-yloxy]phenyl}(1,3-thiazol-5-ylmethyl)amino]benzoic acid,
71) 4-[{4-methoxy-3-[(3R)-tetrahydrofuran-3-yloxy]phenyl}(1,3-thiazol-5-ylmethyl)amino]benzoic acid,
72) 4-[[3-(cyclopropylmethoxy)-4-methoxyphenyl](1,3-thiazol-5-ylmethyl)amino]benzoic acid,
73) 3-[[3-(cyclopropylmethoxy)-4-methoxyphenyl](1,3-thiazol-5-ylmethyl)amino]benzoic acid,
74) 4-[[3,4-bis(difluoromethoxy)phenyl](pyridazin-3-ylmethyl)amino]benzoic acid,
75) 3-[[3,4-bis(difluoromethoxy)phenyl](pyridazin-3-ylmethyl)amino]benzoic acid,
76) 3,4-bis(difluoromethoxy)-N-[4-(ethylthio)phenyl]-N-(pyridin-3-ylmethyl)aniline,
77) 3,4-bis(difluoromethoxy)-N-[4-(methylsulfonyl)phenyl]-N-(1,3-thiazol-5-ylmethyl)aniline,
78) 3,4-bis(difluoromethoxy)-N-[4-(ethylsulfonyl)phenyl]-N-(1,3-thiazol-5-ylmethyl)aniline,
79) 3,4-bis(difluoromethoxy)-N-[3-(methylsulfonyl)phenyl]-N-(1,3-thiazol-5-ylmethyl)aniline,
80) 4-[[3,4-bis(difluoromethoxy)phenyl](pyrimidin-5-ylmethyl)amino]benzoic acid,
81) 5-[[3-(cyclopropylmethoxy)-4-methoxyphenyl](1,3-thiazol-5-ylmethyl)amino]nicotinic acid,
82) 3-[[3,4-bis(difluoromethoxy)phenyl](pyrimidin-5-ylmethyl)amino]benzoic acid,
83) 5-[[3,4-bis(difluoromethoxy)phenyl](pyridin-3-ylmethyl)amino]nicotinic acid,
84) 1-{4-[[3,4-bis(difluoromethoxy)phenyl](pyridin-3-ylmethyl)amino]-2-hydroxyphenyl}ethanone,
85) 1-{4-[[3,4-bis(difluoromethoxy)phenyl](pyridin-3-ylmethyl)amino]phenyl}-2,2,2-trifluoroethanone,
86) 1-{4-[[3,4-bis(difluoromethoxy)phenyl](pyridin-3-ylmethyl)amino]phenyl}ethanone,
87) 4-[[3,4-bis(difluoromethoxy)phenyl](pyridin-3-ylmethyl)amino]phenol,
88) 1-{3-[[3,4-bis(difluoromethoxy)phenyl](pyridin-3-ylmethyl)amino]phenyl}ethanone,
89) 2-{3-[[3,4-bis(difluoromethoxy)phenyl](pyridin-3-ylmethyl)amino]phenyl}propan-2-ol,
90) 5-[[3,4-bis(difluoromethoxy)phenyl](pyridin-3-ylmethyl)amino]-2,3-difluorophenol,
91) 5-[[3,4-bis(difluoromethoxy)phenyl](1,3-thiazol-5-ylmethyl)amino]-2-hydroxybenzoic acid,
92) 4-[[3,4-bis(difluoromethoxy)phenyl](1,3-thiazol-5-ylmethyl)amino]-2-hydroxybenzoic acid,
93) 1-{5-[[3,4-bis(difluoromethoxy)phenyl](1,3-thiazol-5-ylmethyl)amino]pyridin-3-yl}ethanone,
94) 1-{5-[[3,4-bis(difluoromethoxy)phenyl](pyridin-3-ylmethyl)amino]pyridin-3-yl}ethanone,
95) N-[3,4-bis(difluoromethoxy)phenyl]-5-(2-methyl-1,3-dioxolan-2-yl)-N-(pyridin-3-ylmethyl)pyridin-3-amine,
96) 3,4-bis(difluoromethoxy)-N-[3-(1H-pyrazol-3-yl)phenyl]-N-(pyridin-3-ylmethyl)aniline,
97) 3-[[4-(difluoromethoxy)-3-ethoxyphenyl](1,3-thiazol-5-ylmethyl)amino]benzoic acid,
98) 5-[[3,4-bis(difluoromethoxy)phenyl](1,3-thiazol-5-ylmethyl)amino]nicotinic acid,
99) 4-[[4-(difluoromethoxy)-3-ethoxyphenyl](1,3-thiazol-5-ylmethyl)amino]benzoic acid,
100) {4-[[3,4-bis(difluoromethoxy)phenyl](1,3-thiazol-5-ylmethyl)amino]phenyl}acetic acid,
101) 3,4-bis(difluoromethoxy)-N-(pyridin-3-ylmethyl)-N-[3-(1H-1,2,4-triazol-5-yl)phenyl]aniline,
102) 4-{3-[[3-(cyclopentyloxy)-4-methoxyphenyl](pyridin-3-ylmethyl)amino]phenyl}-1-methyl-1H-pyrazol-5-ol,
103) {3-[[3,4-bis(difluoromethoxy)phenyl](1,3-thiazol-5-ylmethyl)amino]phenyl}acetic acid,
104) 4-[[3-(cyclopentyloxy)-4-(difluoromethoxy)phenyl](1,3-thiazol-5-ylmethyl)amino]benzoic acid,
105) 4-{3-[[3,4-bis(difluoromethoxy)phenyl](pyridin-3-ylmethyl)amino]phenyl}-2-methyl-1,2-dihydro-3H-pyrazol-3-one,
106) 4-[[4-(difluoromethoxy)-3-isopropoxyphenyl](1,3-thiazol-5-ylmethyl)amino]benzoic acid,
107) 4-[[4-(difluoromethoxy)-3-isopropoxyphenyl](pyridin-3-ylmethyl)amino]benzoic acid,
108) 3-[[4-(difluoromethoxy)-3-isopropoxyphenyl](pyridin-3-ylmethyl)amino]benzoic acid,
109) 3-[[4-(difluoromethoxy)-3-isopropoxyphenyl](1,3-thiazol-5-ylmethyl)amino]benzoic acid,
110) 3-[[3-(cyclopentyloxy)-4-(difluoromethoxy)phenyl](1,3-thiazol-5-ylmethyl)amino]benzoic acid,
111) 4-methoxy-N-[4-(methylsulfonyl)phenyl]-3-[(3R)-tetrahydrofuran-3-yloxy]-N-(1,3-thiazol-5-ylmethyl)aniline,
112) 1-{4-[[3,4-bis(difluoromethoxy)phenyl](1,3-thiazol-5-ylmethyl)amino]phenyl}ethanone,
113) 3,4-bis(difluoromethoxy)-N-[3-(methylsulfonyl)phenyl]-N-(pyrimidin-5-ylmethyl)aniline,
114) 3,4-bis(difluoromethoxy)-N-[4-(methylsulfonyl)phenyl]-N-(pyrimidin-5-ylmethyl)aniline,
115) 4-methoxy-N-[3-(methylsulfonyl)phenyl]-3-[(3R)-tetrahydrofuran-3-yloxy]-N-(1,3-thiazol-5-ylmethyl)aniline,
116) 3-[[4-(difluoromethoxy)-3-methoxyphenyl](1,3-thiazol-5-ylmethyl)amino]benzoic acid,
117) 1-{4-[[3,4-bis(difluoromethoxy)phenyl](pyrimidin-5-ylmethyl)amino]phenyl}ethanone,
118) 3,4-bis(difluoromethoxy)-N-[4-(methylsulfonyl)phenyl]-N-(pyrazin-2-ylmethyl)aniline,
119) 1-{4-[[3,4-bis(difluoromethoxy)phenyl](pyrazin-2-ylmethyl)amino]phenyl}ethanone,
120) 4-[[4-(difluoromethoxy)-3-methoxyphenyl](1,3-thiazol-5-ylmethyl)amino]benzoic acid,
121) 4-[[3-(cyclopropylmethoxy)-4-(difluoromethoxy)phenyl](1,3-thiazol-5-ylmethyl)amino]benzoic acid,
122) 1-{3-[[3,4-bis(difluoromethoxy)phenyl](pyridin-3-ylmethyl)amino]phenoxy}-2-methylpropan-2-ol,
123) 1-{3-[[3,4-bis(difluoromethoxy)phenyl](1,3-thiazol-5-ylmethyl)amino]phenoxy}-2-methylpropan-2-ol,
124) 4-[[4-(difluoromethoxy)-3-methoxyphenyl](pyridin-3-ylmethyl)amino]benzoic acid,
125) 3-[[3-butoxy-4-(difluoromethoxy)phenyl](1,3-thiazol-5-ylmethyl)amino]benzoic acid,
126) 3-[[3-butoxy-4-(difluoromethoxy)phenyl](pyridin-3-ylmethyl)amino]benzoic acid,
127) 3-{[3,4-bis(difluoromethoxy)phenyl][(1-methyl-1H-pyrazol-4-yl)methyl]amino}benzoic acid,
128) 4-[[3-butoxy-4-(difluoromethoxy)phenyl](pyridin-3-ylmethyl)amino]benzoic acid,
129) 4-[[3-butoxy-4-(difluoromethoxy)phenyl](1,3-thiazol-5-ylmethyl)amino]benzoic acid,
130) 4-[(3-ethoxy-4-methoxyphenyl)(1,3-thiazol-5-ylmethyl)amino]benzoic acid,
131) 4-[(3-isopropoxy-4-methoxyphenyl)(1,3-thiazol-5-ylmethyl)amino]benzoic acid,
132) 3-(cyclopropylmethoxy)-4-methoxy-N-[4-(methylsulfonyl)phenyl]-N-(1,3-thiazol-5-ylmethyl)aniline,
133) 1-{4-[[3-(cyclopropylmethoxy)-4-methoxyphenyl](1,3-thiazol-5-ylmethyl)amino]phenyl}ethanone,
134) 3,4-bis(difluoromethoxy)-N-methyl-N-[4-(methylsulfonyl)phenyl]aniline,
135) 4-[[3-(cyclopropylmethoxy)-4-(difluoromethoxy)phenyl](pyridin-3-ylmethyl)amino]benzoic acid,
136) 3,4-bis(difluoromethoxy)-N-[4-methyl-3-(methylsulfonyl)phenyl]-N-(1,3-thiazol-5-ylmethyl)aniline,
137) {4-[[3,4-bis(difluoromethoxy)phenyl](pyrimidin-5-ylmethyl)amino]phenyl}acetic acid,
138) 4-[(3,4-dimethoxyphenyl)(1,3-thiazol-5-ylmethyl)amino]benzoic acid,
139) {3-[[3,4-bis(difluoromethoxy)phenyl](pyrimidin-5-ylmethyl)amino]phenyl}acetic acid,
140) {4-[[3,4-bis(difluoromethoxy)phenyl](pyrazin-2-ylmethyl)amino]phenyl}acetic acid,
141) 4-[[3,4-bis(difluoromethoxy)phenyl](1,3-oxazol-5-ylmethyl)amino]benzoic acid,
142) 4-[[3,4-bis(difluoromethoxy)phenyl](pyrazin-2-ylmethyl)amino]benzoic acid,
143) 4-[[4-(difluoromethoxy)-3-ethoxyphenyl](pyrimidin-5-ylmethyl)amino]benzoic acid,
144) 4-[[4-(difluoromethoxy)-3-ethoxyphenyl](1,3-oxazol-5-ylmethyl)amino]benzoic acid,
145) 4-[[3,4-bis(difluoromethoxy)phenyl](1,3-thiazol-5-ylmethyl)amino]-N,N-dimethylbenzenesulfonamide,
146) 4-[[3,4-bis(difluoromethoxy)phenyl](1,3-thiazol-5-ylmethyl)amino]benzenesulfonamide,
147) 4-[[4-(difluoromethoxy)-3-ethoxyphenyl](pyrazin-2-ylmethyl)amino]benzoic acid,
148) 3-[[3,4-bis(difluoromethoxy)phenyl](pyrazin-2-ylmethyl)amino]benzoic acid,
149) 3-[[3-(difluoromethoxy)-4-methoxyphenyl](1,3-thiazol-5-ylmethyl)amino]benzoic acid,
150) 4-[[3-(difluoromethoxy)-4-methoxyphenyl](1,3-thiazol-5-ylmethyl)amino]benzoic acid,
151) 4-[[3,4-bis(difluoromethoxy)phenyl](1,3-thiazol-5-ylmethyl)amino]-N-methylbenzamide,
152) 4-[[3,4-bis(difluoromethoxy)phenyl](1,3-thiazol-5-ylmethyl)amino]benzamide,
153) 4-[[4-(difluoromethoxy)-3-methoxyphenyl](pyrimidin-5-ylmethyl)amino]benzoic acid,
154) 4-[[4-(difluoromethoxy)-3-methoxyphenyl](pyrazin-2-ylmethyl)amino]benzoic acid,
155) 4-[[4-(difluoromethoxy)-3-isopropoxyphenyl](pyrazin-2-ylmethyl)amino]benzoic acid,
156) 3-[[4-(difluoromethoxy)-3-isopropoxyphenyl](pyrazin-2-ylmethyl)amino]benzoic acid,
157) 4-[[3-(cyclopropylmethoxy)-4-(difluoromethoxy)phenyl](pyrazin-2-ylmethyl)amino]benzoic acid,
158) 4-[[3,4-bis(difluoromethoxy)phenyl](1,3-thiazol-5-ylmethyl)amino]-N,N-dimethylbenzamide,
159) 3-[[3-(cyclopropylmethoxy)-4-(difluoromethoxy)phenyl](pyrazin-2-ylmethyl)amino]benzoic acid,
160) 5-[[4-(difluoromethoxy)-3-isopropoxyphenyl](1,3-thiazol-5-ylmethyl)amino]nicotinic acid,
161) 4-[[3-(difluoromethoxy)-4-methoxyphenyl](pyrazin-2-ylmethyl)amino]benzoic acid,
162) N-{5-[[3,4-bis(difluoromethoxy)phenyl](1,3-thiazol-5-ylmethyl)amino]-2-methylphenyl}methane sulfonamide,
163) N-{3-[[3,4-bis(difluoromethoxy)phenyl](1,3-thiazol-5-ylmethyl)amino]phenyl}methanesulfonamide,
164) N-{3-[[3,4-bis(difluoromethoxy)phenyl](1,3-thiazol-5-ylmethyl)amino]phenyl}ethanesulfonamide,
165) 4-[[3,4-bis(difluoromethoxy)phenyl](1,3-thiazol-5-ylmethyl)amino]-N-methylbenzenesulfonamide,
166) 4-[[4-(difluoromethoxy)-3-isopropoxyphenyl](1,3-oxazol-5-ylmethyl)amino]benzoic acid,
167) 4-[[3-(cyclopropylmethoxy)-4-(difluoromethoxy)phenyl](1,3-oxazol-5-ylmethyl)amino]benzoic acid,
168) 4-[[3-(cyclopropylmethoxy)-4-(difluoromethoxy)phenyl](pyrimidin-5-ylmethyl)amino]benzoic acid,
169) 4-[[4-(difluoromethoxy)-3-isopropoxyphenyl](pyrimidin-5-ylmethyl)amino]benzoic acid,
170) 4-[(3,4-diethoxyphenyl)(1,3-thiazol-5-ylmethyl)amino]benzoic acid,
171) 3-[[3-(cyclopentyloxy)-4-methoxyphenyl](1-pyridin-3-ylethyl)amino]benzoic acid,
172) 3-[(3,4-dimethoxyphenyl)(1-pyridin-3-ylethyl)amino]benzoic acid,
173) 4-[(4-ethoxy-3-methoxyphenyl)(1,3-thiazol-5-ylmethyl)amino]benzoic acid,
174) 4-(difluoromethoxy)-3-ethoxy-N-[4-(methylsulfonyl)phenyl]-N-(1,3-thiazol-5-ylmethyl)aniline,
175) {3-[[4-(difluoromethoxy)-3-ethoxyphenyl](1,3-oxazol-5-ylmethyl)amino]phenyl}acetic acid,
176) 4-(difluoromethoxy)-3-ethoxy-N-[4-(methylsulfonyl)phenyl]-N-(1,3-oxazol-5-ylmethyl)aniline,
177) {4-[[4-(difluoromethoxy)-3-ethoxyphenyl](1,3-oxazol-5-ylmethyl)amino]phenyl}acetic acid,
178) {3-[[4-(difluoromethoxy)-3-ethoxyphenyl](1,3-thiazol-5-ylmethyl)amino]phenyl}acetic acid,
179) 4-[[4-(difluoromethoxy)-3-methoxyphenyl](1,3-oxazol-5-ylmethyl)amino]benzoic acid,
180) 4-[(3-isopropoxy-4-methoxyphenyl)(1,3-oxazol-5-ylmethyl)amino]benzoic acid,
181) 4-[(3-ethoxy-4-methoxyphenyl)(1,3-oxazol-5-ylmethyl)amino]benzoic acid,
182) 4-[[3-(cyclobutyloxy)-4-(difluoromethoxy)phenyl](1,3-oxazol-5-ylmethyl)amino]benzoic acid,
183) 4-[[3-(cyclopropylmethoxy)-4-methoxyphenyl](1,3-oxazol-5-ylmethyl)amino]benzoic acid,
184) 4-[[3-(cyclobutyloxy)-4-methoxyphenyl](1,3-oxazol-5-ylmethyl)amino]benzoic acid,
185) 4-[[3-methoxy-4-(2,2,2-trifluoroethoxy)phenyl](1,3-oxazol-5-ylmethyl)amino]benzoic acid,
186) 4-[[3-methoxy-4-(2,2,2-trifluoroethoxy)phenyl](pyrimidin-5-ylmethyl)amino]benzoic acid,
187) 4-{[4-(difluoromethoxy)-3-ethoxyphenyl][(5-methoxypyridin-3-yl)methyl]amino}benzoic acid,
188) 4-{[4-(difluoromethoxy)-3-ethoxyphenyl][(4-methyl-1,3-oxazol-5-yl)methyl]amino}benzoic acid,
189) 4-[[3-(cyclopropyloxy)-4-methoxyphenyl](1,3-oxazol-5-ylmethyl)amino]benzoic acid,
190) 4-[[4-(difluoromethoxy)-3-ethoxyphenyl](1,3-oxazol-4-ylmethyl)amino]benzoic acid,
191) 4-[(3,4-diethoxyphenyl)(pyrimidin-5-ylmethyl)amino]benzoic acid,
192) 4-[(3-isopropoxy-4-methoxyphenyl)(pyrimidin-5-ylmethyl)amino]benzoic acid,
193) 4-[[3-(cyclobutyloxy)-4-methoxyphenyl](pyrimidin-5-ylmethyl)amino]benzoic acid,
194) 4-[[3-(cyclopropyloxy)-4-methoxyphenyl](pyrimidin-5-ylmethyl)amino]benzoic acid,
195) 4-[[3-(cyclopropylmethoxy)-4-methoxyphenyl](pyrimidin-5-ylmethyl)amino]benzoic acid,
196) 4-[(3-ethoxy-4-methoxyphenyl)(pyrimidin-5-ylmethyl)amino]benzoic acid,
197) 3-[[4-(difluoromethoxy)-3-isopropoxyphenyl](1,3-oxazol-5-ylmethyl)amino]benzoic acid,
198) 3-[[3-(cyclopropylmethoxy)-4-(difluoromethoxy)phenyl](1,3-oxazol-5-ylmethyl)amino]benzoic acid,
199) 3-[[4-(difluoromethoxy)-3-ethoxyphenyl](pyrimidin-5-ylmethyl)amino]benzoic acid,
200) 3-[(3-ethoxy-4-methoxyphenyl)(1,3-oxazol-5-ylmethyl)amino]benzoic acid,
201) 3-[[3-(cyclobutyloxy)-4-(difluoromethoxy)phenyl](1,3-oxazol-5-ylmethyl)amino]benzoic acid,
202) 4-[(4-ethoxy-3-isopropoxyphenyl)(pyrimidin-5-ylmethyl)amino]benzoic acid,
203) 3-[(3-isopropoxy-4-methoxyphenyl)(1,3-oxazol-5-ylmethyl)amino]benzoic acid,
204) 3-[[4-(difluoromethoxy)-3-ethoxyphenyl](1,3-oxazol-5-ylmethyl)amino]benzoic acid,
205) 4-[[3-(cyclobutyloxy)-4-(difluoromethoxy)phenyl](pyrimidin-5-ylmethyl)amino]benzoic acid,
206) 3-[[4-(difluoromethoxy)-3-methoxyphenyl](1,3-oxazol-5-ylmethyl)amino]benzoic acid,
207) 4-[(3-ethoxy-4-isopropoxyphenyl)(1,3-thiazol-5-ylmethyl)amino]benzoic acid,
208) 4-[(4-ethoxy-3-isopropoxyphenyl)(1,3-oxazol-5-ylmethyl)amino]benzoic acid,
209) 4-[(4-ethoxy-3-methoxyphenyl)(pyrimidin-5-ylmethyl)amino]benzoic acid,
210) 3-[(3-ethoxy-4-methoxyphenyl)(pyrimidin-5-ylmethyl)amino]benzoic acid,
211) 3-[[3-(cyclopropylmethoxy)-4-(difluoromethoxy)phenyl](pyrimidin-5-ylmethyl)amino]benzoic acid,
212) 3-[[3-(cyclopropylmethoxy)-4-methoxyphenyl](pyrimidin-5-ylmethyl)amino]benzoic acid,
213) 3-[(3-isopropoxy-4-methoxyphenyl)(pyrimidin-5-ylmethyl)amino]benzoic acid,
214) 4-[[3-(benzyloxy)-4-(difluoromethoxy)phenyl](pyrimidin-5-ylmethyl)amino]benzoic acid,
215) 3-[[3-(cyclopropylmethoxy)-4-methoxyphenyl](1,3-oxazol-5-ylmethyl)amino]benzoic acid,
216) 3-[[3-(cyclobutyloxy)-4-methoxyphenyl](1,3-oxazol-5-ylmethyl)amino]benzoic acid,
217) 4-[[3-ethoxy-4-(trifluoromethoxy)phenyl](1,3-oxazol-5-ylmethyl)amino]benzoic acid,
218) 3-[[3-(cyclopropyloxy)-4-methoxyphenyl](pyrimidin-5-ylmethyl)amino]benzoic acid,
219) 4-[(4-ethoxy-3-methoxyphenyl)(1,3-oxazol-5-ylmethyl)amino]benzoic acid,
220) 4-[(3,4-diethoxyphenyl)(1,3-oxazol-5-ylmethyl)amino]benzoic acid,
221) 4-[(4-ethoxy-3-methoxyphenyl)(pyridin-3-ylmethyl)amino]benzoic acid,
222) 3-[(4-ethoxy-3-methoxyphenyl)(pyridin-3-ylmethyl)amino]benzoic acid,
223) 3-[[4-(difluoromethoxy)-3-isopropoxyphenyl](pyrimidin-5-ylmethyl)amino]benzoic acid,
224) 3-[(4-ethoxy-3-methoxyphenyl)(1,3-oxazol-5-ylmethyl)amino]benzoic acid,
225) 4-{[4-(difluoromethoxy)-3-ethoxyphenyl][(2-methyl-1,3-thiazol-5-yl)methyl]amino}benzoic acid,
226) 3-[(4-ethoxy-3-methoxyphenyl)(1,3-thiazol-5-ylmethyl)amino]benzoic acid,
227) 3-[(3,4-diethoxyphenyl)(1,3-thiazol-5-ylmethyl)amino]benzoic acid,
228) 3-[[3-(cyclobutyloxy)-4-methoxyphenyl](pyrimidin-5-ylmethyl)amino]benzoic acid,
229) 4-[[4-(difluoromethoxy)-3-ethoxyphenyl](tetrahydrofuran-3-ylmethyl)amino]benzoic acid,
230) 4-[[3-(cyclopropyloxy)-4-(difluoromethoxy)phenyl](pyrimidin-5-ylmethyl)amino]benzoic acid,
231) 4-[[3-(cyclopropyloxy)-4-(difluoromethoxy)phenyl](1,3-oxazol-5-ylmethyl)amino]benzoic acid,
232) 4-[[4-methoxy-3-(trifluoromethoxy)phenyl](1,3-oxazol-5-ylmethyl)amino]benzoic acid,
233) (2R)-3-{4-[[3-(cyclopentyloxy)-4-methoxyphenyl](pyridin-3-ylmethyl)amino]phenoxy}propane-1,2-diol,
234) 3-{{[1-(aminocarbonyl)piperidin-3-yl]methyl}[3-(cyclopentyloxy)-4-methoxyphenyl]amino}benzoic acid,
235) 5-[[3-(cyclopentyloxy)-4-methoxyphenyl](pyridin-3-ylmethyl)amino]pyridin-2(1H)-one,
236) 2-({5-[[3-(cyclopentyloxy)-4-methoxyphenyl](pyridin-3-ylmethyl)amino]pyridin-2-yl}amino)propan-1-ol,
237) 3-[{5-[[3-(cyclopentyloxy)-4-methoxyphenyl](pyridin-3-ylmethyl)amino]pyridin-2-yl}(methyl)amino]propane-1,2-diol,
238) (2R)-3-{3-[[3-(cyclopentyloxy)-4-methoxyphenyl](pyridin-3-ylmethyl)amino]phenoxy}propane-1,2-diol,
239) 5-{3-[[3-(cyclopentyloxy)-4-methoxyphenyl](pyridin-3-ylmethyl)amino]phenyl}imidazolidine-2,4-dione,
240) (2R)-3-{4-[[3,4-bis(difluoromethoxy)phenyl](pyridin-3-ylmethyl)amino]phenoxy}propane-1,2-diol,
241) (2R)-3-{4-[[3-(cyclopentyloxy)-4-methoxyphenyl](1,3-thiazol-5-ylmethyl)amino]phenoxy}propane-1,2-diol,
242) 5-[[3-(cyclopentyloxy)-4-methoxyphenyl](pyridin-3-ylmethyl)amino]-2-methyl-1,2-benzisoxazol-3(2H)-one,
243) 5-[[3-(cyclopentyloxy)-4-methoxyphenyl](pyridin-3-ylmethyl)amino]-1,2-benzisoxazol-3(2H)-one,
244) (2R)-3-{4-[(3,4-dimethoxyphenyl)(pyridin-3-ylmethyl)amino]phenoxy}propane-1,2-diol,
245) 5-{4-[[3-(cyclopentyloxy)-4-methoxyphenyl](pyridin-3-ylmethyl)amino]phenyl}imidazolidine-2,4-dione,
246) 5-[[3,4-bis(difluoromethoxy)phenyl](pyridin-3-ylmethyl)amino]-2-{[(2R)-2,3-dihydroxypropyl]oxy}benzoic acid,
247) 2-({5-[[3-(cyclopentyloxy)-4-methoxyphenyl](pyridin-3-ylmethyl)amino]pyridin-2-yl}amino)butan-1-ol,
248) 1-({5-[[3-(cyclopentyloxy)-4-methoxyphenyl](pyridin-3-ylmethyl)amino]pyridin-2-yl}amino)butan-2-ol,
249) 4-({5-[[3-(cyclopentyloxy)-4-methoxyphenyl](pyridin-3-ylmethyl)amino]pyridin-2-yl}amino)butan-2-ol,
250) 1-({5-[[3-(cyclopentyloxy)-4-methoxyphenyl](pyridin-3-ylmethyl)amino]pyridin-2-yl}amino)propan-2-ol,
251) 5-{3-[[3,4-bis(difluoromethoxy)phenyl](pyridin-3-ylmethyl)amino]phenyl}imidazolidine-2,4-dione,
252) 1-({5-[[3-(cyclopentyloxy)-4-methoxyphenyl](pyridin-3-ylmethyl)amino]pyridin-2-yl}amino)-2-methylpropan-2-ol trifluoroacetic acid salt,
253) 3-[{5-[[3-(cyclopentyloxy)-4-methoxyphenyl](1,3-thiazol-5-ylmethyl)amino]pyridin-2-yl}(methyl)amino]propane-1,2-diol,
254) 3-[{5-[[3,4-bis(difluoromethoxy)phenyl](pyridin-3-ylmethyl)amino]pyridin-2-yl}(methyl)amino]propane-1,2-diol,
255) (2R)-3-{3-[[3,4-bis(difluoromethoxy)phenyl](pyridin-3-ylmethyl)amino]phenoxy}propane-1,2-diol,
256) 5-{3-[[3,4-bis(difluoromethoxy)phenyl](1,3-thiazol-5-ylmethyl)amino]phenyl}-1,3,5-trimethylimidazolidine-2,4-dione,
257) 5-{3-[[3,4-bis(difluoromethoxy)phenyl](1,3-thiazol-5-ylmethyl)amino]phenyl}imidazolidine-2,4-dione,
258) (2R)-3-{4-[[3,4-bis(difluoromethoxy)phenyl](1,3-thiazol-5-ylmethyl)amino]phenoxy}propane-1,2-diol,
259) (2R)-3-{3-[[3,4-bis(difluoromethoxy)phenyl](1,3-thiazol-5-ylmethyl)amino]phenoxy}propane-1,2-diol,
260) 4-{3-[[3,4-bis(difluoromethoxy)phenyl](pyridin-3-ylmethyl)amino]phenyl}imidazolidin-2-one,
261) 5-[[3,4-bis(difluoromethoxy)phenyl](1,3-thiazol-5-ylmethyl)amino]-1,2-benzisoxazol-3(2H)-one,
262) 5-{3-[[3,4-bis(difluoromethoxy)phenyl](pyridin-3-ylmethyl)amino]phenyl}-1,3-oxazolidin-2-one,
263) 6-[[3,4-bis(difluoromethoxy)phenyl](1,3-thiazol-5-ylmethyl)amino]-1,2-benzisoxazol-3(2H)-one,
264) 4-{3-[[3,4-bis(difluoromethoxy)phenyl](pyridin-3-ylmethyl)amino]phenyl}-1,3-oxazolidin-2-one,
265) 1-{3-[[3,4-bis(difluoromethoxy)phenyl](1,3-thiazol-5-ylmethyl)amino]phenoxy}-3-methoxypropan-2-ol,
266) 2-[[3,4-bis(difluoromethoxy)phenyl](phenyl)amino]ethanol,
267) 1-[[3,4-bis(difluoromethoxy)phenyl](3-chlorophenyl)amino]-2-methylpropan-2-ol,
268) 1-[[3,4-bis(difluoromethoxy)phenyl](3-chlorophenyl)amino]propan-2-ol,
269) 3-[[3,4-bis(difluoromethoxy)phenyl](3-chlorophenyl)amino]propan-1-o,
270) 4-[[3,4-bis(difluoromethoxy)phenyl](1,3-thiazol-5-ylmethyl)amino]butanoic acid,
271) cis-4-[[3,4-bis(difluoromethoxy)phenyl](1,3-thiazol-5-ylmethyl)amino]cyclohexanecarboxylic acid,
272) 3-[[4-(cyclopropyloxy)-3-ethoxyphenyl](pyrimidin-5-ylmethyl)amino]benzoic acid,
273) 4-[[4-(difluoromethoxy)-3-ethoxyphenyl]({2-[4-(difluoromethoxy)-3-ethoxyphenyl]-1,3-oxazol-5-yl}methyl)amino]benzoic acid,
279) 3-[[3-(difluoromethoxy)-4-methoxyphenyl](1,3-oxazol-5-ylmethyl)amino]benzoic acid,
280) 4-[[3-(difluoromethoxy)-4-methoxyphenyl](pyridin-3-ylmethyl)amino]benzoic acid,
281) 4-[[4-(difluoromethoxy)-3-ethoxyphenyl](1,2,3-thiadiazol-5-ylmethyl)amino]benzoic acid,
282) 4-[[3-hydroxy-4-methoxyphenyl](1,3-oxazol-5-ylmethyl)amino]benzoic acid,
283) 3-[[4-ethoxy-3-methoxyphenyl](pyrimidin-5-ylmethyl)amino]benzoic acid,
284) 3-[[3,4-diethoxyphenyl](pyrimidin-5-ylmethyl)amino]benzoic acid,
285) 3-[[3,4-diethoxyphenyl](1,3-oxazol-5-ylmethyl)amino]benzoic acid,
286) 3-[[3-cyclopropyloxy-4-methoxyphenyl](1,3-oxazol-5-ylmethyl)amino]benzoic acid,
287) 3-[[3-cyclobutyloxy-4-difluoromethoxyphenyl](pyrimidin-5-ylmethyl)amino]benzoic acid, and
288) 3-[[3-cyclopropyloxy-4-difluoromethoxyphenyl](1,3-oxazol-5-ylmethyl)amino]benzoic acid,
wherein a compound which is listed above in either a free base form or in the form of a pharmaceutically acceptable salt can also be in the form of a solvate,
wherein a compound which is listed above in either a free base form or in the form of a pharmaceutically acceptable salt can also be in the form of an N-oxide,
wherein a compound which is listed above in a free base form or solvate or N-oxide thereof, or in the form of a pharmaceutically acceptable salt or solvate thereof can also be in the form of a polymorph, and
wherein if the compound listed above exhibits chirality it can be in the form of a mixture of enantiomers such as a racemate or a mixture of diastereomers, or can be in the form of a single enantiomer or a single diastereomer.

36. A compound according to claim 35, wherein said compound is selected from:
1) 4-[[3,4-bis(difluoromethoxy)phenyl](pyridin-3-ylmethyl)amino]-N-(methylsulfonyl)benzamide,
2) 4-[[3,4-bis(difluoromethoxy)phenyl](pyridin-3-ylmethyl)amino]-N-(ethylsulfonyl)benzamide,
3) 3-{(3-fluorobenzyl) [4-methoxy-3-(2,2,2-trifluoroethoxy)phenyl]amino}benzoic acid,
4) 3-[(3,4-dimethoxyphenyl)(3-fluorobenzyl)amino]benzoic acid,
5) 3-[(3-ethoxy-4-methoxyphenyl)(3-fluorobenzyl)amino]benzoic acid,
6) 3-[[3-(cyclobutyloxy)-4-methoxyphenyl](3-fluorobenzyl)amino]benzoic acid,
7) 3-[[3-(cyclopentyloxy)-4-methoxyphenyl](3-fluorobenzyl)amino]benzoic acid,
8) 3-[(2,6-difluorobenzyl)(3,4-dimethoxyphenyl)amino]benzoic acid,
9) 3-[(2,6-difluorobenzyl)(3-ethoxy-4-methoxyphenyl)amino]benzoic acid, and
10) 3-[(2,6-difluorobenzyl)(3-isopropoxy-4-methoxyphenyl)amino]benzoic acid,
11) 3-[[3-(cyclobutyloxy)-4-methoxyphenyl](2,6-difluorobenzyl)amino]benzoic acid,
12) 3-((2,6-difluorobenzyl) {4-methoxy-3-[(3R)-tetrahydrofuran-3-yloxy]phenyl}amino)benzoic acid,
13) 3-[[4-(difluoromethoxy)-3-ethoxyphenyl](pyridin-3-ylmethyl)amino]benzoic acid,
14) 4-[[4-(difluoromethoxy)-3-ethoxyphenyl](pyridin-3-ylmethyl)amino]benzoic acid,
15) 3-[[3,4-bis(difluoromethoxy)phenyl](2,6-difluorobenzyl)amino]benzoic acid,
wherein a compound which is listed above in either a free base form or in the form of a pharmaceutically acceptable salt can also be in the form of a solvate,
wherein a compound which is listed above in either a free base form or in the form of a pharmaceutically acceptable salt can also be in the form of an N-oxide,
wherein a compound which is listed above in a free base form or solvate or N-oxide thereof, or in the form of a pharmaceutically acceptable salt or solvate thereof can also be in the form of a polymorph, and
wherein if the compound listed above exhibits chirality it can be in the form of a mixture of enantiomers such as a racemate or a mixture of diastereomers, or can be in the form of a single enantiomer or a single diastereomer.

37. A compound according to claim 35, wherein said compound is selected from:
16) 3-[[3,4-bis(difluoromethoxy)phenyl](3-fluorobenzyl)amino]benzoic acid,
17) 3-{(2,6-difluorobenzyl)[4-(difluoromethoxy)-3-methoxyphenyl]amino}benzoic acid,
18) 3-[[4-(difluoromethoxy)-3-methoxyphenyl](pyridin-3-ylmethyl)amino]benzoic acid,
19) 3-[[4-(difluoromethoxy)-3-methoxyphenyl](3-fluorobenzyl)amino]benzoic acid,
20) 3-[[3,4-bis(difluoromethoxy)phenyl](2-fluorobenzyl)amino]benzoic acid,
21) 3-[[3,4-bis(difluoromethoxy)phenyl](4-fluorobenzyl)amino]benzoic acid,
22) 3-((3-fluorobenzyl) {4-methoxy-3-[(3R)-tetrahydrofuran-3-yloxy]phenyl}amino)benzonitrile,
23) N-(3-fluorobenzyl)-4-methoxy-N-[4-(methylsulfonyl)phenyl]-3-[(3R)-tetrahydrofuran-3-yloxy]aniline,
24) N-ethyl-N-[4-((3-fluorobenzyl) {4-methoxy-3-[(3R)-tetrahydrofuran-3-yloxy]phenyl}amino)phenyl]ethanesulfonamide,
25) 3-{(3-cyanobenzyl)[3-(cyclopentyloxy)-4-methoxyphenyl]amino}benzoic acid,
26) 3-{[3-(cyclopentyloxy)-4-methoxyphenyl][(6-oxo-1,6-dihydropyridin-3-yl)methyl]amino}benzoic acid,
27) 5-bromo-N-[3-(cyclopentyloxy)-4-methoxyphenyl]-N-(pyridin-3-ylmethyl)pyridin-2-amine,
28) 3-[[3-(cyclopentyloxy)-4-methoxyphenyl](piperidin-3-ylmethyl)amino]benzoic acid,
29) 4-{[3-(cyclopentyloxy)-4-methoxyphenyl][(6-oxo-1,6-dihydropyridin-3-yl)methyl]amino}benzoic acid,
30) Ethyl {4-[[3-(cyclopentyloxy)-4-methoxyphenyl](pyridin-3-ylmethyl)amino]phenoxy}acetate,
wherein a compound which is listed above in either a free base form or in the form of a pharmaceutically acceptable salt can also be in the form of a solvate,
wherein a compound which is listed above in either a free base form or in the form of a pharmaceutically acceptable salt can also be in the form of an N-oxide,
wherein a compound which is listed above in a free base form or solvate or N-oxide thereof, or in the form of a pharmaceutically acceptable salt or solvate thereof can also be in the form of a polymorph, and
wherein if the compound listed above exhibits chirality it can be in the form of a mixture of enantiomers such as a racemate or a mixture of diastereomers, or can be in the form of a single enantiomer or a single diastereomer.

38. A compound according to claim 35, wherein said compound is selected from:
31) 2-{4-[[3-(cyclopentyloxy)-4-methoxyphenyl](pyridin-3-ylmethyl)amino]phenoxy}ethanol,
32) 3-(cyclopentyloxy)-N-(4-{[(4S)-2,2-dimethyl-1,3-dioxolan-4-yl]methoxy}phenyl)-4-methoxy-N-(pyridin-3-ylmethyl)aniline,
33) N(2)-benzyl-N(5)-[3-(cyclopentyloxy)-4-methoxyphenyl]-N(2)-methyl-N(5)-(pyridin-3-ylmethyl)pyridine-2,5-diamine,
34) 3-[[3-(cyclopentyloxy)-4-methoxyphenyl](1H-pyrazol-4-ylmethyl)amino]benzoic acid,
35) 3-[[3-(cyclopentyloxy)-4-methoxyphenyl](1,3-thiazol-5-ylmethyl)amino]benzoic acid,
36) {3-[[3-(cyclopentyloxy)-4-methoxyphenyl](pyridin-3-ylmethyl)amino]phenyl}acetic acid,
37) 5-[[3-(cyclopentyloxy)-4-methoxyphenyl](pyridin-3-ylmethyl)amino]-2-ethylbenzoic acid,
38) 3-[[3-(cyclopentyloxy)-4-methoxyphenyl](pyridin-3-ylmethyl)amino]-4-methylbenzoic acid,
39) 4-[[3-(cyclopentyloxy)-4-methoxyphenyl](1H-pyrazol-4-ylmethyl)amino]benzoic acid,
40) 4-[[3-(cyclopentyloxy)-4-methoxyphenyl](1,3-thiazol-5-ylmethyl)amino]benzoic acid,
41) N-[3-(cyclopentyloxy)-4-methoxyphenyl]-N-(pyridin-3-ylmethyl)-6-pyrrolidin-1-ylpyridin-3-amine,
42) N(5)-[3-(cyclopentyloxy)-4-methoxyphenyl]-N(2)-(2-morpholin-4-ylethyl)-N(5)-(pyridin-3-ylmethyl)pyridine-2,5-diamine,
43) 4-[[3-(cyclopentyloxy)-4-methoxyphenyl](pyrimidin-5-ylmethyl)amino]benzoic acid,
44) N(2)-benzyl-N(5)-[3-(cyclopentyloxy)-4-methoxyphenyl]-N(5)-(pyridin-3-ylmethyl)pyridine-2,5-diamine,
45) 3-[[3-(cyclopentyloxy)-4-methoxyphenyl](1,3-thiazol-4-ylmethyl)amino]benzoic acid,
wherein a compound which is listed above in either a free base form or in the form of a pharmaceutically acceptable salt can also be in the form of a solvate,
wherein a compound which is listed above in either a free base form or in the form of a pharmaceutically acceptable salt can also be in the form of an N-oxide,
wherein a compound which is listed above in a free base form or solvate or N-oxide thereof, or in the form of a pharmaceutically acceptable salt or solvate thereof can also be in the form of a polymorph, and
wherein if the compound listed above exhibits chirality it can be in the form of a mixture of enantiomers such as a racemate or a mixture of diastereomers, or can be in the form of a single enantiomer or a single diastereomer.

39. A compound according to claim 35, wherein said compound is selected from:
46) 3-[[3-(cyclopentyloxy)-4-methoxyphenyl](pyrimidin-5-ylmethyl)amino]benzoic acid,
47) 4-[[3-(cyclopentyloxy)-4-methoxyphenyl](pyridazin-3-ylmethyl)amino]benzoic acid,
48) 3-[[3-(cyclopentyloxy)-4-methoxyphenyl](pyridazin-3-ylmethyl)amino]benzoic acid,
49) {4-[[3-(cyclopentyloxy)-4-methoxyphenyl](pyridin-3-ylmethyl)amino]phenyl}acetic acid,
50) 3-[[3-(cyclopentyloxy)-4-methoxyphenyl](piperidin-4-ylmethyl)amino]benzoic acid,
51) 4-([3-(cyclopentyloxy)-4-methoxyphenyl]{[2-(dimethylamino)pyrimidin-5-yl]methyl}amino)benzoic acid,
52) 3-([3-(cyclopentyloxy)-4-methoxyphenyl]{[2-(dimethylamino)pyrimidin-5-yl]methyl}amino)benzoic acid,
53) 3-{4-[[3-(cyclopentyloxy)-4-methoxyphenyl](pyridin-3-ylmethyl)amino]phenyl}propan-1-ol,
54) 5-[[3-(cyclopentyloxy)-4-methoxyphenyl](pyridin-3-ylmethyl)amino]nicotinic acid,
55) 3-{4-[[3-(cyclopentyloxy)-4-methoxyphenyl](pyridin-3-ylmethyl)amino]phenyl}propanoic acid,
56) 3-(cyclopentyloxy)-4-methoxy-N-[3-(2-methyl-1,3-thiazol-4-yl)phenyl]-N-(pyridin-3-ylmethyl)aniline,
57) {3-[[3,4-bis(difluoromethoxy)phenyl](pyridin-3-ylmethyl)amino]phenyl}acetic acid,
58) (5S)-5-({3-[[3-(cyclopentyloxy)-4-methoxyphenyl](pyridin-3-ylmethyl)amino]phenoxy}methyl)pyrrolidin-2-one,
59) 4-[[3,4-bis(difluoromethoxy)phenyl](1,3-thiazol-5-ylmethyl)amino]benzoic acid,
60) 3-[[3,4-bis(difluoromethoxy)phenyl](1,3-thiazol-5-ylmethyl)amino]benzoic acid,
wherein a compound which is listed above in either a free base form or in the form of a 41 MEMORY-0058 pharmaceutically acceptable salt can also be in the form of a solvate,
wherein a compound which is listed above in either a free base form or in the form of a pharmaceutically acceptable salt can also be in the form of an N-oxide,
wherein a compound which is listed above in a free base form or solvate or N-oxide thereof, or in the form of a pharmaceutically acceptable salt or solvate thereof can also be in the form of a polymorph, and
wherein if the compound listed above exhibits chirality it can be in the form of a mixture of enantiomers such as a racemate or a mixture of diastereomers, or can be in the form of a single enantiomer or a single diastereomer.

40. A compound according to claim 35, wherein said compound is selected from:
61) N(5)-[3-(cyclopentyloxy)-4-methoxyphenyl]-N(2)-[2-(methylsulfonyl)ethyl]-N(5)-(pyridin-3-ylmethyl)pyridine-2,5-diamine,
62) 3,4-bis(difluoromethoxy)-N-[3-(methylthio)phenyl]-N-(pyridin-3-ylmethyl)aniline,
63) 3,4-bis(difluoromethoxy)-N-[4-(methylsulfonyl)phenyl]-N-(pyridin-3-ylmethyl)aniline,
64) 2-[{5-[[3-(cyclopentyloxy)-4-methoxyphenyl](pyridin-3-ylmethyl)amino]pyridin-2-yl}(methyl)amino]-N,N-dimethylacetamide,
65) N(5)-[3-(cyclopentyloxy)-4-methoxyphenyl]-N(5)-(pyridin-3-ylmethyl)-N(2)-(tetrahydro-2H-pyran-4-yl)pyridine-2,5-diamine,
66) 3,4-bis(difluoromethoxy)-N-[3-(methylsulfonyl)phenyl]-N-(pyridin-3-ylmethyl)aniline,
67) 5-[[3-(cyclopentyloxy)-4-methoxyphenyl](pyridin-3-ylmethyl)amino]-N,2-dihydroxybenzamide,
68) 4-[[3-(cyclopentyloxy)-4-methoxyphenyl](pyridin-3-ylmethyl)amino]-3-methylbenzoic acid,
69) {4-[[3,4-bis(difluoromethoxy)phenyl](pyridin-3-ylmethyl)amino]phenyl}acetic acid,
70) 3-[{4-methoxy-3-[(3R)-tetrahydrofuran-3-yloxy]phenyl}(1,3-thiazol-5-ylmethyl)amino]benzoic acid,
71) 4-[{4-methoxy-3-[(3R)-tetrahydrofuran-3-yloxy]phenyl}(1,3-thiazol-5-ylmethyl)amino]benzoic acid,
72) 4-[[3-(cyclopropylmethoxy)-4-methoxyphenyl](1,3-thiazol-5-ylmethyl)amino]benzoic acid,
73) 3-[[3-(cyclopropylmethoxy)-4-methoxyphenyl](1,3-thiazol-5-ylmethyl)amino]benzoic acid,
74) 4-[[3,4-bis(difluoromethoxy)phenyl](pyridazin-3-ylmethyl)amino]benzoic acid,
75) 3-[[3,4-bis(difluoromethoxy)phenyl](pyridazin-3-ylmethyl)amino]benzoic acid,
wherein a compound which is listed above in either a free base form or in the form of a pharmaceutically acceptable salt can also be in the form of a solvate,
wherein a compound which is listed above in either a free base form or in the form of a pharmaceutically acceptable salt can also be in the form of an N-oxide,
wherein a compound which is listed above in a free base form or solvate or N-oxide thereof, or in the form of a pharmaceutically acceptable salt or solvate thereof can also be in the form of a polymorph, and
wherein if the compound listed above exhibits chirality it can be in the form of a mixture of enantiomers such as a racemate or a mixture of diastereomers, or can be in the form of a single enantiomer or a single diastereomer.

41. A compound according to claim 35, wherein said compound is selected from:
76) 3,4-bis(difluoromethoxy)-N-[4-(ethylthio)phenyl]-N-(pyridin-3-ylmethyl)aniline,
77) 3,4-bis(difluoromethoxy)-N-[4-(methylsulfonyl)phenyl]-N-(1,3-thiazol-5-ylmethyl)aniline,
78) 3,4-bis(difluoromethoxy)-N-[4-(ethylsulfonyl)phenyl]-N-(1,3-thiazol-5-ylmethyl)aniline,
79) 3,4-bis(difluoromethoxy)-N-[3-(methylsulfonyl)phenyl]-N-(1,3-thiazol-5-ylmethyl)aniline,
80) 4-[[3,4-bis(difluoromethoxy)phenyl](pyrimidin-5-ylmethyl)amino]benzoic acid,
81) 5-[[3-(cyclopropylmethoxy)-4-methoxyphenyl](1,3-thiazol-5-ylmethyl)amino]nicotinic acid,
82) 3-[[3,4-bis(difluoromethoxy)phenyl](pyrimidin-5-ylmethyl)amino]benzoic acid,
83) 5-[[3,4-bis(difluoromethoxy)phenyl](pyridin-3-ylmethyl)amino]nicotinic acid,
84) 1-{4-[[3,4-bis(difluoromethoxy)phenyl](pyridin-3-ylmethyl)amino]-2-hydroxyphenyl}ethanone,
85) 1-{4-[[3,4-bis(difluoromethoxy)phenyl](pyridin-3-ylmethyl)amino]phenyl}-2,2,2-trifluoroethanone,
86) 1-{4-[[3,4-bis(difluoromethoxy)phenyl](pyridin-3-ylmethyl)amino]phenyl}ethanone,
87) 4-[[3,4-bis(difluoromethoxy)phenyl](pyridin-3-ylmethyl)amino]phenol,
88) 1-{3-[[3,4-bis(difluoromethoxy)phenyl](pyridin-3-ylmethyl)amino]phenyl}ethanone,
89) 2-{3-[[3,4-bis(difluoromethoxy)phenyl](pyridin-3-ylmethyl)amino]phenyl}propan-2-ol,
90) 5-[[3,4-bis(difluoromethoxy)phenyl](pyridin-3-ylmethyl)amino]-2,3-difluorophenol,
wherein a compound which is listed above in either a free base form or in the form of a pharmaceutically acceptable salt can also be in the form of a solvate,
wherein a compound which is listed above in either a free base form or in the form of a pharmaceutically acceptable salt can also be in the form of an N-oxide,
wherein a compound which is listed above in a free base form or solvate or N-oxide thereof, or in the form of a pharmaceutically acceptable salt or solvate thereof can also be in the form of a polymorph, and
wherein if the compound listed above exhibits chirality it can be in the form of a mixture of enantiomers such as a racemate or a mixture of diastereomers, or can be in the form of a single enantiomer or a single diastereomer.

42. A compound according to claim 35, wherein said compound is selected from:
91) 5-[[3,4-bis(difluoromethoxy)phenyl](1,3-thiazol-5-ylmethyl)amino]-2-hydroxybenzoic acid,
92) 4-[[3,4-bis(difluoromethoxy)phenyl](1,3-thiazol-5-ylmethyl)amino]-2-hydroxybenzoic acid,
93) 1-{5-[[3,4-bis(difluoromethoxy)phenyl](1,3-thiazol-5-ylmethyl)amino]pyridin-3-yl}ethanone,
94) 1-{5-[[3,4-bis(difluoromethoxy)phenyl](pyridin-3-ylmethyl)amino]pyridin-3-yl}ethanone,
95) N-[3,4-bis(difluoromethoxy)phenyl]-5-(2-methyl-1,3-dioxolan-2-yl)-N-(pyridin-3-ylmethyl)pyridin-3-amine,
96) 3,4-bis(difluoromethoxy)-N-[3-(1H-pyrazol-3-yl)phenyl]-N-(pyridin-3-ylmethyl)aniline,
97) 3-[[4-(difluoromethoxy)-3-ethoxyphenyl](1,3-thiazol-5-ylmethyl)amino]benzoic acid,
98) 5-[[3,4-bis(difluoromethoxy)phenyl](1,3-thiazol-5-ylmethyl)amino]nicotinic acid,
99) 4-[[4-(difluoromethoxy)-3-ethoxyphenyl](1,3-thiazol-5-ylmethyl)amino]benzoic acid,
100) {4-[[3,4-bis(difluoromethoxy)phenyl](1,3-thiazol-5-ylmethyl)amino]phenyl}acetic acid,
101) 3,4-bis(difluoromethoxy)-N-(pyridin-3-ylmethyl)-N-[3-(1H-1,2,4-triazol-5-yl)phenyl]aniline,
102) 4-{3-[[3-(cyclopentyloxy)-4-methoxyphenyl](pyridin-3-ylmethyl)amino]phenyl}-1-methyl-1H-pyrazol-5-ol,
103) {3-[[3,4-bis(difluoromethoxy)phenyl](1,3-thiazol-5-ylmethyl)amino]phenyl}acetic acid,
104) 4-[[3-(cyclopentyloxy)-4-(difluoromethoxy)phenyl](1,3-thiazol-5-ylmethyl)amino]benzoic acid,
105) 4-{3-[[3,4-bis(difluoromethoxy)phenyl](pyridin-3-ylmethyl)amino]phenyl}-2-methyl-1,2-dihydro-3H-pyrazol-3-one,
wherein a compound which is listed above in either a free base form or in the form of a pharmaceutically acceptable salt can also be in the form of a solvate,
wherein a compound which is listed above in either a free base form or in the form of a pharmaceutically acceptable salt can also be in the form of an N-oxide,
wherein a compound which is listed above in a free base form or solvate or N-oxide thereof, or in the form of a pharmaceutically acceptable salt or solvate thereof can also be in the form of a polymorph, and
wherein if the compound listed above exhibits chirality it can be in the form of a mixture of enantiomers such as a racemate or a mixture of diastereomers, or can be in the form of a single enantiomer or a single diastereomer.

43. A compound according to claim 35, wherein said compound is selected from:
106) 4-[[4-(difluoromethoxy)-3-isopropoxyphenyl](1,3-thiazol-5-ylmethyl)amino]benzoic acid,
107) 4-[[4-(difluoromethoxy)-3-isopropoxyphenyl](pyridin-3-ylmethyl)amino]benzoic acid,
108) 3-[[4-(difluoromethoxy)-3-isopropoxyphenyl](pyridin-3-ylmethyl)amino]benzoic acid,
109) 3-[[4-(difluoromethoxy)-3-isopropoxyphenyl](1,3-thiazol-5-ylmethyl)amino]benzoic acid,
110) 3-[[3-(cyclopentyloxy)-4-(difluoromethoxy)phenyl](1,3-thiazol-5-ylmethyl)amino]benzoic acid,
111) 4-methoxy-N-[4-(methylsulfonyl)phenyl]-3-[(3R)-tetrahydrofuran-3-yloxy]-N-(1,3-thiazol-5-ylmethyl)aniline,
112) 1-{4-[[3,4-bis(difluoromethoxy)phenyl](1,3-thiazol-5-ylmethyl)amino]phenyl}ethanone,
113) 3,4-bis(difluoromethoxy)-N-[3-(methylsulfonyl)phenyl]-N-(pyrimidin-5-ylmethyl)aniline,
114) 3,4-bis(difluoromethoxy)-N-[4-(methylsulfonyl)phenyl]-N-(pyrimidin-5-ylmethyl)aniline,
115) 4-methoxy-N-[3-(methylsulfonyl)phenyl]-3-[(3R)-tetrahydrofuran-3-yloxy]-N-(1,3-thiazol-5-ylmethyl)aniline,
116) 3-[[4-(difluoromethoxy)-3-methoxyphenyl](1,3-thiazol-5-ylmethyl)amino]benzoic acid,
117) 1-{4-[[3,4-bis(difluoromethoxy)phenyl](pyrimidin-5-ylmethyl)amino]phenyl}ethanone,
118) 3,4-bis(difluoromethoxy)-N-[4-(methylsulfonyl)phenyl]-N-(pyrazin-2-ylmethyl)aniline,
119) 1-{4-[[3,4-bis(difluoromethoxy)phenyl](pyrazin-2-ylmethyl)amino]phenyl}ethanone,
120) 4-[[4-(difluoromethoxy)-3-methoxyphenyl](1,3-thiazol-5-ylmethyl)amino]benzoic acid,
wherein a compound which is listed above in either a free base form or in the form of a pharmaceutically acceptable salt can also be in the form of a solvate,
wherein a compound which is listed above in either a free base form or in the form of a pharmaceutically acceptable salt can also be in the form of an N-oxide,
wherein a compound which is listed above in a free base form or solvate or N-oxide thereof, or in the form of a pharmaceutically acceptable salt or solvate thereof can also be in the form of a polymorph, and
wherein if the compound listed above exhibits chirality it can be in the form of a mixture of enantiomers such as a racemate or a mixture of diastereomers, or can be in the form of a single enantiomer or a single diastereomer.

44. A compound according to claim 35, wherein said compound is selected from:
121) 4-[[3-(cyclopropylmethoxy)-4-(difluoromethoxy)phenyl](1,3-thiazol-5-ylmethyl)amino]benzoic acid,
122) 1-{3-[[3,4-bis(difluoromethoxy)phenyl](pyridin-3-ylmethyl)amino]phenoxy}-2-methylpropan-2-ol,
123) 1-{3-[[3,4-bis(difluoromethoxy)phenyl](1,3-thiazol-5-ylmethyl)amino]phenoxy}-2-methylpropan-2-ol,
124) 4-[[4-(difluoromethoxy)-3-methoxyphenyl](pyridin-3-ylmethyl)amino]benzoic acid,
125) 3-[[3-butoxy-4-(difluoromethoxy)phenyl](1,3-thiazol-5-ylmethyl)amino]benzoic acid,
126) 3-[[3-butoxy-4-(difluoromethoxy)phenyl](pyridin-3-ylmethyl)amino]benzoic acid,
127) 3-{[3,4-bis(difluoromethoxy)phenyl][(1-methyl-1H-pyrazol-4-yl)methyl]amino}benzoic acid,
128) 4-[[3-butoxy-4-(difluoromethoxy)phenyl](pyridin-3-ylmethyl)amino]benzoic acid,
129) 4-[[3-butoxy-4-(difluoromethoxy)phenyl](1,3-thiazol-5-ylmethyl)amino]benzoic acid,
130) 4-[(3-ethoxy-4-methoxyphenyl)(1,3-thiazol-5-ylmethyl)amino]benzoic acid,
131) 4-[(3-isopropoxy-4-methoxyphenyl)(1,3-thiazol-5-ylmethyl)amino]benzoic acid,
132) 3-(cyclopropylmethoxy)-4-methoxy-N-[4-(methylsulfonyl)phenyl]-N-(1,3-thiazol-5-ylmethyl)aniline,
133) 1-{4-[[3-(cyclopropylmethoxy)-4-methoxyphenyl](1,3-thiazol-5-ylmethyl)amino]phenyl}ethanone,
134) 3,4-bis(difluoromethoxy)-N-methyl-N-[4-(methylsulfonyl)phenyl]aniline,
135) 4-[[3-(cyclopropylmethoxy)-4-(difluoromethoxy)phenyl](pyridin-3-ylmethyl)amino]benzoic acid,
wherein a compound which is listed above in either a free base form or in the form of a pharmaceutically acceptable salt can also be in the form of a solvate,
wherein a compound which is listed above in either a free base form or in the form of a pharmaceutically acceptable salt can also be in the form of an N-oxide,
wherein a compound which is listed above in a free base form or solvate or N-oxide thereof, or in the form of a pharmaceutically acceptable salt or solvate thereof can also be in the form of a polymorph, and
wherein if the compound listed above exhibits chirality it can be in the form of a mixture of enantiomers such as a racemate or a mixture of diastereomers, or can be in the form of a single enantiomer or a single diastereomer.

45. A compound according to claim 35, wherein said compound is selected from:
136) 3,4-bis(difluoromethoxy)-N-[4-methyl-3-(methylsulfonyl)phenyl]-N-(1,3-thiazol-5-ylmethyl)aniline,
137) {4-[[3,4-bis(difluoromethoxy)phenyl](pyrimidin-5-ylmethyl)amino]phenyl}acetic acid,
138) 4-[(3,4-dimethoxyphenyl)(1,3-thiazol-5-ylmethyl)amino]benzoic acid,
139) {3-[[3,4-bis(difluoromethoxy)phenyl](pyrimidin-5-ylmethyl)amino]phenyl}acetic acid,
140) {4-[[3,4-bis(difluoromethoxy)phenyl](pyrazin-2-ylmethyl)amino]phenyl}acetic acid,
141) 4-[[3,4-bis(difluoromethoxy)phenyl](1,3-oxazol-5-ylmethyl)amino]benzoic acid,
142) 4-[[3,4-bis(difluoromethoxy)phenyl](pyrazin-2-ylmethyl)amino]benzoic acid,
143) 4-[[4-(difluoromethoxy)-3-ethoxyphenyl](pyrimidin-5-ylmethyl)amino]benzoic acid,
144) 4-[[4-(difluoromethoxy)-3-ethoxyphenyl](1,3-oxazol-5-ylmethyl)amino]benzoic acid,
145) 4-[[3,4-bis(difluoromethoxy)phenyl](1,3-thiazol-5-ylmethyl)amino]-N,N-dimethylbenzenesulfonamide,
146) 4-[[3,4-bis(difluoromethoxy)phenyl](1,3-thiazol-5-ylmethyl)amino]benzenesulfonamide,
147) 4-[[4-(difluoromethoxy)-3-ethoxyphenyl](pyrazin-2-ylmethyl)amino]benzoic acid,
148) 3-[[3,4-bis(difluoromethoxy)phenyl](pyrazin-2-ylmethyl)amino]benzoic acid,
149) 3-[[3-(difluoromethoxy)-4-methoxyphenyl](1,3-thiazol-5-ylmethyl)amino]benzoic acid,
150) 4-[[3-(difluoromethoxy)-4-methoxyphenyl](1,3-thiazol-5-ylmethyl)amino]benzoic acid,
wherein a compound which is listed above in either a free base form or in the form of a pharmaceutically acceptable salt can also be in the form of a solvate,
wherein a compound which is listed above in either a free base form or in the form of a pharmaceutically acceptable salt can also be in the form of an N-oxide,
wherein a compound which is listed above in a free base form or solvate or N-oxide thereof, or in the form of a pharmaceutically acceptable salt or solvate thereof can also be in the form of a polymorph, and
wherein if the compound listed above exhibits chirality it can be in the form of a mixture 49 MEMORY-0058 of enantiomers such as a racemate or a mixture of diastereomers, or can be in the form of a single enantiomer or a single diastereomer.

46. A compound according to claim 35, wherein said compound is selected from:
151) 4-[[3,4-bis(difluoromethoxy)phenyl](1,3-thiazol-5-ylmethyl)amino]-N-methylbenzamide,
152) 4-[[3,4-bis(difluoromethoxy)phenyl](1,3-thiazol-5-ylmethyl)amino]benzamide,
153) 4-[[4-(difluoromethoxy)-3-methoxyphenyl](pyrimidin-5-ylmethyl)amino]benzoic acid,
154) 4-[[4-(difluoromethoxy)-3-methoxyphenyl](pyrazin-2-ylmethyl)amino]benzoic acid,
155) 4-[[4-(difluoromethoxy)-3-isopropoxyphenyl](pyrazin-2-ylmethyl)amino]benzoic acid,
156) 3-[[4-(difluoromethoxy)-3-isopropoxyphenyl](pyrazin-2-ylmethyl)amino]benzoic acid,
157) 4-[[3-(cyclopropylmethoxy)-4-(difluoromethoxy)phenyl](pyrazin-2-ylmethyl)amino]benzoic acid,
158) 4-[[3,4-bis(difluoromethoxy)phenyl](1,3-thiazol-5-ylmethyl)amino]-N,N-dimethylbenzamide,
159) 3-[[3-(cyclopropylmethoxy)-4-(difluoromethoxy)phenyl](pyrazin-2-ylmethyl)amino]benzoic acid,
160) 5-[[4-(difluoromethoxy)-3-isopropoxyphenyl](1,3-thiazol-5-ylmethyl)amino]nicotinic acid,
161) 4-[[3-(difluoromethoxy)-4-methoxyphenyl](pyrazin-2-ylmethyl)amino]benzoic acid,
162) N-{5-[[3,4-bis(difluoromethoxy)phenyl](1,3-thiazol-5-ylmethyl)amino]-2-methylphenyl}methane sulfonamide,
163) N-{3-[[3,4-bis(difluoromethoxy)phenyl](1,3-thiazol-5-ylmethyl)amino]phenyl}methanesulfonamide,
164) N-{3-[[3,4-bis(difluoromethoxy)phenyl](1,3-thiazol-5-ylmethyl)amino]phenyl}ethanesulfonamide,
165) 4-[[3,4-bis(difluoromethoxy)phenyl](1,3-thiazol-5-ylmethyl)amino]-N-methylbenzenesulfonamide,
wherein a compound which is listed above in either a free base form or in the form of a pharmaceutically acceptable salt can also be in the form of a solvate,
wherein a compound which is listed above in either a free base form or in the form of a pharmaceutically acceptable salt can also be in the form of an N-oxide,
wherein a compound which is listed above in a free base form or solvate or N-oxide thereof, or in the form of a pharmaceutically acceptable salt or solvate thereof can also be in the form of a polymorph, and
wherein if the compound listed above exhibits chirality it can be in the form of a mixture of enantiomers such as a racemate or a mixture of diastereomers, or can be in the form of a single enantiomer or a single diastereomer.

47. A compound according to claim 35, wherein said compound is selected from:
166) 4-[[4-(difluoromethoxy)-3-isopropoxyphenyl](1,3-oxazol-5-ylmethyl)amino]benzoic acid,
167) 4-[[3-(cyclopropylmethoxy)-4-(difluoromethoxy)phenyl](1,3-oxazol-5-ylmethyl)amino]benzoic acid,
168) 4-[[3-(cyclopropylmethoxy)-4-(difluoromethoxy)phenyl](pyrimidin-5-ylmethyl)amino]benzoic acid,
169) 4-[[4-(difluoromethoxy)-3-isopropoxyphenyl](pyrimidin-5-ylmethyl)amino]benzoic acid,
170) 4-[(3,4-diethoxyphenyl)(1,3-thiazol-5-ylmethyl)amino]benzoic acid,
171) 3-[[3-(cyclopentyloxy)-4-methoxyphenyl](1-pyridin-3-ylethyl)amino]benzoic acid,
172) 3-[(3,4-dimethoxyphenyl)(1-pyridin-3-ylethyl)amino]benzoic acid,
173) 4-[(4-ethoxy-3-methoxyphenyl)(1,3-thiazol-5-ylmethyl)amino]benzoic acid,
174) 4-(difluoromethoxy)-3-ethoxy-N-[4-(methylsulfonyl)phenyl]-N-(1,3-thiazol-5-ylmethyl)aniline,
175) {3-[[4-(difluoromethoxy)-3-ethoxyphenyl](1,3-oxazol-5-ylmethyl)amino]phenyl}acetic acid,
176) 4-(difluoromethoxy)-3-ethoxy-N-[4-(methylsulfonyl)phenyl]-N-(1,3-oxazol-5-ylmethyl)aniline,
177) {4-[[4-(difluoromethoxy)-3-ethoxyphenyl](1,3-oxazol-5-ylmethyl)amino]phenyl}acetic acid,
178) {3-[[4-(difluoromethoxy)-3-ethoxyphenyl](1,3-thiazol-5-ylmethyl)amino]phenyl}acetic acid,
179) 4-[[4-(difluoromethoxy)-3-methoxyphenyl](1,3-oxazol-5-ylmethyl)amino]benzoic acid,
180) 4-[(3-isopropoxy-4-methoxyphenyl)(1,3-oxazol-5-ylmethyl)amino]benzoic acid,
wherein a compound which is listed above in either a free base form or in the form of a pharmaceutically acceptable salt can also be in the form of a solvate,
wherein a compound which is listed above in either a free base form or in the form of a pharmaceutically acceptable salt can also be in the form of an N-oxide,
wherein a compound which is listed above in a free base form or solvate or N-oxide thereof, or in the form of a pharmaceutically acceptable salt or solvate thereof can also be in the form of a polymorph, and
wherein if the compound listed above exhibits chirality it can be in the form of a mixture of enantiomers such as a racemate or a mixture of diastereomers, or can be in the form of a single enantiomer or a single diastereomer.

48. A compound according to claim 35, wherein said compound is selected from:
181) 4-[(3-ethoxy-4-methoxyphenyl)(1,3-oxazol-5-ylmethyl)amino]benzoic acid,
182) 4-[[3-(cyclobutyloxy)-4-(difluoromethoxy)phenyl](1,3-oxazol-5-ylmethyl)amino]benzoic acid,
183) 4-[[3-(cyclopropylmethoxy)-4-methoxyphenyl](1,3-oxazol-5-ylmethyl)amino]benzoic acid,
184) 4-[[3-(cyclobutyloxy)-4-methoxyphenyl](1,3-oxazol-5-ylmethyl)amino]benzoic acid,
185) 4-[[3-methoxy-4-(2,2,2-trifluoroethoxy)phenyl](1,3-oxazol-5-ylmethyl)amino]benzoic acid,
186) 4-[[3-methoxy-4-(2,2,2-trifluoroethoxy)phenyl](pyrimidin-5-ylmethyl)amino]benzoic acid,
187) 4-{[4-(difluoromethoxy)-3-ethoxyphenyl][(5-methoxypyridin-3-yl)methyl]amino}benzoic acid,
188) 4-{[4-(difluoromethoxy)-3-ethoxyphenyl][(4-methyl-1,3-oxazol-5-yl)methyl]amino}benzoic acid,
189) 4-[[3-(cyclopropyloxy)-4-methoxyphenyl](1,3-oxazol-5-ylmethyl)amino]benzoic acid,
190) 4-[[4-(difluoromethoxy)-3-ethoxyphenyl](1,3-oxazol-4-ylmethyl)amino]benzoic acid,
191) 4-[(3,4-diethoxyphenyl) (pyrimidin-5-ylmethyl)amino]benzoic acid,
192) 4-[(3-isopropoxy-4-methoxyphenyl)(pyrimidin-5-ylmethyl)amino]benzoic acid,
193) 4-[[3-(cyclobutyloxy)-4-methoxyphenyl](pyrimidin-5-ylmethyl)amino]benzoic acid,
194) 4-[[3-(cyclopropyloxy)-4-methoxyphenyl](pyrimidin-5-ylmethyl)amino]benzoic acid,
195) 4-[[3-(cyclopropylmethoxy)-4-methoxyphenyl](pyrimidin-5-ylmethyl)amino]benzoic acid,
wherein a compound which is listed above in either a free base form or in the form of a pharmaceutically acceptable salt can also be in the form of a solvate,
wherein a compound which is listed above in either a free base form or in the form of a pharmaceutically acceptable salt can also be in the form of an N-oxide,
wherein a compound which is listed above in a free base form or solvate or N-oxide thereof, or in the form of a pharmaceutically acceptable salt or solvate thereof can also be in the form of a polymorph, and
wherein if the compound listed above exhibits chirality it can be in the form of a mixture of enantiomers such as a racemate or a mixture of diastereomers, or can be in the form of a single enantiomer or a single diastereomer.

49. A compound according to claim 35, wherein said compound is selected from:
196) 4-[(3-ethoxy-4-methoxyphenyl)(pyrimidin-5-ylmethyl)amino]benzoic acid,
197) 3-[[4-(difluoromethoxy)-3-isopropoxyphenyl](1,3-oxazol-5-ylmethyl)amino]benzoic acid,
198) 3-[[3-(cyclopropylmethoxy)-4-(difluoromethoxy)phenyl](1,3-oxazol-5-ylmethyl)amino]benzoic acid,
199) 3-[[4-(difluoromethoxy)-3-ethoxyphenyl](pyrimidin-5-ylmethyl)amino]benzoic acid,
200) 3-[(3-ethoxy-4-methoxyphenyl)(1,3-oxazol-5-ylmethyl)amino]benzoic acid,
201) 3-[[3-(cyclobutyloxy)-4-(difluoromethoxy)phenyl](1,3-oxazol-5-ylmethyl)amino]benzoic acid,
202) 4-[(4-ethoxy-3-isopropoxyphenyl)(pyrimidin-5-ylmethyl)amino]benzoic acid,
203) 3-[(3-isopropoxy-4-methoxyphenyl)(1,3-oxazol-5-ylmethyl)amino]benzoic acid,
204) 3-[[4-(difluoromethoxy)-3-ethoxyphenyl](1,3-oxazol-5-ylmethyl)amino]benzoic acid,
205) 4-[[3-(cyclobutyloxy)-4-(difluoromethoxy)phenyl](pyrimidin-5-ylmethyl)amino]benzoic acid,
206) 3-[[4-(difluoromethoxy)-3-methoxyphenyl](1,3-oxazol-5-ylmethyl)amino]benzoic acid,
207) 4-[(3-ethoxy-4-isopropoxyphenyl)(1,3-thiazol-5-ylmethyl)amino]benzoic acid,
208) 4-[(4-ethoxy-3-isopropoxyphenyl)(1,3-oxazol-5-ylmethyl)amino]benzoic acid,
209) 4-[(4-ethoxy-3-methoxyphenyl)(pyrimidin-5-ylmethyl)amino]benzoic acid,
210) 3-[(3-ethoxy-4-methoxyphenyl)(pyrimidin-5-ylmethyl)amino]benzoic acid,
wherein a compound which is listed above in either a free base form or in the form of a pharmaceutically acceptable salt can also be in the form of a solvate,
wherein a compound which is listed above in either a free base form or in the form of a pharmaceutically acceptable salt can also be in the form of an N-oxide,
wherein a compound which is listed above in a free base form or solvate or N-oxide thereof, or in the form of a pharmaceutically acceptable salt or solvate thereof can also be in the form of a polymorph, and
wherein if the compound listed above exhibits chirality it can be in the form of a mixture of enantiomers such as a racemate or a mixture of diastereomers, or can be in the form of a single enantiomer or a single diastereomer.

50. A compound according to claim 35, wherein said compound is selected from:
211) 3-[[3-(cyclopropylmethoxy)-4-(difluoromethoxy)phenyl](pyrimidin-5-ylmethyl)amino]benzoic acid,
212) 3-[[3-(cyclopropylmethoxy)-4-methoxyphenyl](pyrimidin-5-ylmethyl)amino]benzoic acid,
213) 3-[(3-isopropoxy-4-methoxyphenyl)(pyrimidin-5-ylmethyl)amino]benzoic acid,
214) 4-[[3-(benzyloxy)-4-(difluoromethoxy)phenyl](pyrimidin-5-ylmethyl)amino]benzoic acid,
215) 3-[[3-(cyclopropylmethoxy)-4-methoxyphenyl](1,3-oxazol-5-ylmethyl)amino]benzoic acid,
216) 3-[[3-(cyclobutyloxy)-4-methoxyphenyl](1,3-oxazol-5-ylmethyl)amino]benzoic acid,
217) 4-[[3-ethoxy-4-(trifluoromethoxy)phenyl](1,3-oxazol-5-ylmethyl)amino]benzoic acid,
218) 3-[[3-(cyclopropyloxy)-4-methoxyphenyl](pyrimidin-5-ylmethyl)amino]benzoic acid,
219) 4-[(4-ethoxy-3-methoxyphenyl)(1,3-oxazol-5-ylmethyl)amino]benzoic acid,
220) 4-[(3,4-diethoxyphenyl)(1,3-oxazol-5-ylmethyl)amino]benzoic acid,
221) 4-[(4-ethoxy-3-methoxyphenyl)(pyridin-3-ylmethyl)amino]benzoic acid,
222) 3-[(4-ethoxy-3-methoxyphenyl)(pyridin-3-ylmethyl)amino]benzoic acid,
223) 3-[[4-(difluoromethoxy)-3-isopropoxyphenyl](pyrimidin-5-ylmethyl)amino]benzoic acid,
224) 3-[(4-ethoxy-3-methoxyphenyl)(1,3-oxazol-5-ylmethyl)amino]benzoic acid,
225) 4-{[4-(difluoromethoxy)-3-ethoxyphenyl][(2-methyl-1,3-thiazol-5-yl)methyl]amino}benzoic acid,
wherein a compound which is listed above in either a free base form or in the form of a pharmaceutically acceptable salt can also be in the form of a solvate,
wherein a compound which is listed above in either a free base form or in the form of a pharmaceutically acceptable salt can also be in the form of an N-oxide,
wherein a compound which is listed above in a free base form or solvate or N-oxide thereof, or in the form of a pharmaceutically acceptable salt or solvate thereof can also be in the form of a polymorph, and
wherein if the compound listed above exhibits chirality it can be in the form of a mixture of enantiomers such as a racemate or a mixture of diastereomers, or can be in the form of a single enantiomer or a single diastereomer.

51. A compound according to claim 35, wherein said compound is selected from:
226) 3-[(4-ethoxy-3-methoxyphenyl)(1,3-thiazol-5-ylmethyl)amino]benzoic acid,
227) 3-[(3,4-diethoxyphenyl)(1,3-thiazol-5-ylmethyl)amino]benzoic acid,
228) 3-[[3-(cyclobutyloxy)-4-methoxyphenyl](pyrimidin-5-ylmethyl)amino]benzoic acid,
229) 4-[[4-(difluoromethoxy)-3-ethoxyphenyl](tetrahydrofuran-3-ylmethyl)amino]benzoic acid,
230) 4-[[3-(cyclopropyloxy)-4-(difluoromethoxy)phenyl](pyrimidin-5-ylmethyl)amino]benzoic acid,
231) 4-[[3-(cyclopropyloxy)-4-(difluoromethoxy)phenyl](1,3-oxazol-5-ylmethyl)amino]benzoic acid,
232) 4-[[4-methoxy-3-(trifluoromethoxy)phenyl](1,3-oxazol-5-ylmethyl)amino]benzoic acid,
233) (2R)-3-{4-[[3-(cyclopentyloxy)-4-methoxyphenyl](pyridin-3-ylmethyl)amino]phenoxy}propane-1,2-diol,
234) 3-{{[1-(aminocarbonyl)piperidin-3-yl]methyl}[3-(cyclopentyloxy)-4-methoxyphenyl]amino}benzoic acid,
235) 5-[[3-(cyclopentyloxy)-4-methoxyphenyl](pyridin-3-ylmethyl)amino]pyridin-2(1H)-one,
236) 2-({5-[[3-(cyclopentyloxy)-4-methoxyphenyl](pyridin-3-ylmethyl)amino]pyridin-2-yl}amino)propan-1-ol,
237) 3-[{5-[[3-(cyclopentyloxy)-4-methoxyphenyl](pyridin-3-ylmethyl)amino]pyridin-2-yl}(methyl)amino]propane-1,2-diol,
238) (2R)-3-{3-[[3-(cyclopentyloxy)-4-methoxyphenyl](pyridin-3-ylmethyl)amino]phenoxy}propane-1,2-diol,
239) 5-{3-[[3-(cyclopentyloxy)-4-methoxyphenyl](pyridin-3-ylmethyl)amino]phenyl}imidazolidine-2,4-dione,
240) (2R)-3-{4-[[3,4-bis(difluoromethoxy)phenyl](pyridin-3-ylmethyl)amino]phenoxy}propane-1,2-diol,
wherein a compound which is listed above in either a free base form or in the form of a pharmaceutically acceptable salt can also be in the form of a solvate,
wherein a compound which is listed above in either a free base form or in the form of a pharmaceutically acceptable salt can also be in the form of an N-oxide,
wherein a compound which is listed above in a free base form or solvate or N-oxide thereof, or in the form of a pharmaceutically acceptable salt or solvate thereof can also be in the form of a polymorph, and
wherein if the compound listed above exhibits chirality it can be in the form of a mixture of enantiomers such as a racemate or a mixture of diastereomers, or can be in the form of a single enantiomer or a single diastereomer.

52. A compound according to claim 35, wherein said compound is selected from:
241) (2R)-3-{4-[[3-(cyclopentyloxy)-4-methoxyphenyl](1,3-thiazol-5-ylmethyl)amino]phenoxy}propane-1,2-diol,
242) 5-[[3-(cyclopentyloxy)-4-methoxyphenyl](pyridin-3-ylmethyl)amino]-2-methyl-1,2-benzisoxazol-3(2H)-one,
243) 5-[[3-(cyclopentyloxy)-4-methoxyphenyl](pyridin-3-ylmethyl)amino]-1,2-benzisoxazol-3(2H)-one,
244) (2R)-3-{4-[(3,4-dimethoxyphenyl)(pyridin-3-ylmethyl)amino]phenoxy}propane-1,2-diol,
245) 5-{4-[[3-(cyclopentyloxy)-4-methoxyphenyl](pyridin-3-ylmethyl)amino]phenyl}imidazolidine-2,4-dione,
246) 5-[[3,4-bis(difluoromethoxy)phenyl](pyridin-3-ylmethyl)amino]-2-{[(2R)-2,3-dihydroxypropyl]oxy}benzoic acid,
247) 2-({5-[[3-(cyclopentyloxy)-4-methoxyphenyl](pyridin-3-ylmethyl)amino]pyridin-2-yl}amino)butan-1-ol,
248) 1-({5-[[3-(cyclopentyloxy)-4-methoxyphenyl](pyridin-3-ylmethyl)amino]pyridin-2-yl}amino)butan-2-ol,
249) 4-({5-[[3-(cyclopentyloxy)-4-methoxyphenyl](pyridin-3-ylmethyl)amino]pyridin-2-yl}amino)butan-2-ol,
250) 1-({5-[[3-(cyclopentyloxy)-4-methoxyphenyl](pyridin-3-ylmethyl)amino]pyridin-2-yl}amino)propan-2-ol,
251) 5-{3-[[3,4-bis(difluoromethoxy)phenyl](pyridin-3-ylmethyl)amino]phenyl}imidazolidine-2,4-dione,
252) 1-({5-[[3-(cyclopentyloxy)-4-methoxyphenyl](pyridin-3-ylmethyl)amino]pyridin-2-yl}amino)-2-methylpropan-2-ol trifluoroacetic acid salt,
253) 3-[{5-[[3-(cyclopentyloxy)-4-methoxyphenyl](1,3-thiazol-5-ylmethyl)amino]pyridin-2-yl}(methyl)amino]propane-1,2-diol,
254) 3-[{5-[[3,4-bis(difluoromethoxy)phenyl](pyridin-3-ylmethyl)amino]pyridin-2-yl}(methyl)amino]propane-1,2-diol,
255) (2R)-3-{3-[[3,4-bis(difluoromethoxy)phenyl](pyridin-3-ylmethyl)amino]phenoxy}propane-1,2-diol,
wherein a compound which is listed above in either a free base form or in the form of a pharmaceutically acceptable salt can also be in the form of a solvate,
wherein a compound which is listed above in either a free base form or in the form of a pharmaceutically acceptable salt can also be in the form of an N-oxide,
wherein a compound which is listed above in a free base form or solvate or N-oxide thereof, or in the form of a pharmaceutically acceptable salt or solvate thereof can also be in the form of a polymorph, and
wherein if the compound listed above exhibits chirality it can be in the form of a mixture of enantiomers such as a racemate or a mixture of diastereomers, or can be in the form of a single enantiomer or a single diastereomer.

53. A compound according to claim 35, wherein said compound is selected from:
256) 5-{3-[[3,4-bis(difluoromethoxy)phenyl](1,3-thiazol-5-ylmethyl)amino]phenyl}-1,3,5-trimethylimidazolidine-2,4-dione,
257) 5-{3-[[3,4-bis(difluoromethoxy)phenyl](1,3-thiazol-5-ylmethyl)amino]phenyl}imidazolidine-2,4-dione,
258) (2R)-3-{4-[[3,4-bis(difluoromethoxy)phenyl](1,3-thiazol-5-ylmethyl)amino]phenoxy}propane-1,2-diol,
259) (2R)-3-{3-[[3,4-bis(difluoromethoxy)phenyl](1,3-thiazol-5-ylmethyl)amino]phenoxy}propane-1,2-diol,
260) 4-{3-[[3,4-bis(difluoromethoxy)phenyl](pyridin-3-ylmethyl)amino]phenyl}imidazolidin-2-one,
261) 5-[[3,4-bis(difluoromethoxy)phenyl](1,3-thiazol-5-ylmethyl)amino]-1,2-benzisoxazol-3(2H)-one,
262) 5-{3-[[3,4-bis(difluoromethoxy)phenyl](pyridin-3-ylmethyl)amino]phenyl}-1,3-oxazolidin-2-one,
263) 6-[[3,4-bis(difluoromethoxy)phenyl](1,3-thiazol-5-ylmethyl)amino]-1,2-benzisoxazol-3(2H)-one,
264) 4-{3-[[3,4-bis(difluoromethoxy)phenyl](pyridin-3-ylmethyl)amino]phenyl}-1,3-oxazolidin-2-one,
265) 1-{3-[[3,4-bis(difluoromethoxy)phenyl](1,3-thiazol-5-ylmethyl)amino]phenoxy}-3-methoxypropan-2-ol,
266) 2-[[3,4-bis(difluoromethoxy)phenyl](phenyl)amino]ethanol,
267) 1-[[3,4-bis(difluoromethoxy)phenyl](3-chlorophenyl)amino]-2-methylpropan-2-ol,
268) 1-[[3,4-bis(difluoromethoxy)phenyl](3-chlorophenyl)amino]propan-2-ol,
269) 3-[[3,4-bis(difluoromethoxy)phenyl](3-chlorophenyl)amino]propan-1-ol,
270) 4-[[3,4-bis(difluoromethoxy)phenyl](1,3-thiazol-5-ylmethyl)amino]butanoic acid,
wherein a compound which is listed above in either a free base form or in the form of a pharmaceutically acceptable salt can also be in the form of a solvate,
wherein a compound which is listed above in either a free base form or in the form of a pharmaceutically acceptable salt can also be in the form of an N-oxide,
wherein a compound which is listed above in a free base form or solvate or N-oxide thereof, or in the form of a pharmaceutically acceptable salt or solvate thereof can also be in the form of a polymorph, and
wherein if the compound listed above exhibits chirality it can be in the form of a mixture of enantiomers such as a racemate or a mixture of diastereomers, or can be in the form of a single enantiomer or a single diastereomer.

54. A compound according to claim 35, wherein said compound is selected from:
271) cis-4-[[3,4-bis(difluoromethoxy)phenyl](1,3-thiazol-5-ylmethyl)amino]cyclohexanecarboxylic acid,
272) 3-[[4-(cyclopropyloxy)-3-ethoxyphenyl](pyrimidin-5-ylmethyl)amino]benzoic acid,
273) 4-[[4-(difluoromethoxy)-3-ethoxyphenyl]({2-[4-(difluoromethoxy)-3-ethoxyphenyl]-1,3-oxazol-5-yl}methyl)amino]benzoic acid,
279) 3-[[3-(difluoromethoxy)-4-methoxyphenyl](1,3-oxazol-5-ylmethyl)amino]benzoic acid,
280) 4-[[3-(difluoromethoxy)-4-methoxyphenyl](pyridin-3-ylmethyl)amino]benzoic acid,
281) 4-[[4-(difluoromethoxy)-3-ethoxyphenyl](1,2,3-thiadiazol-5-ylmethyl)amino]benzoic acid,
282) 4-[[3-hydroxy-4-methoxyphenyl](1,3-oxazol-5-ylmethyl)amino]benzoic acid,
283) 3-[[4-ethoxy-3-methoxyphenyl](pyrimidin-5-ylmethyl)amino]benzoic acid,
284) 3-[[3,4-diethoxyphenyl](pyrimidin-5-ylmethyl)amino]benzoic acid,
285) 3-[[3,4-diethoxyphenyl](1,3-oxazol-5-ylmethyl)amino]benzoic acid,
286) 3-[[3-cyclopropyloxy-4-methoxyphenyl](1,3-oxazol-5-ylmethyl)amino]benzoic acid,
287) 3-[[3-cyclobutyloxy-4-difluoromethoxyphenyl](pyrimidin-5-ylmethyl)amino]benzoic acid, and
288) 3-[[3-cyclopropyloxy-4-difluoromethoxyphenyl](1,3-oxazol-5-ylmethyl)amino]benzoic acid,
wherein a compound which is listed above in either a free base form or in the form of a pharmaceutically acceptable salt can also be in the form of a solvate,
wherein a compound which is listed above in either a free base form or in the form of a pharmaceutically acceptable salt can also be in the form of an N-oxide,
wherein a compound which is listed above in a free base form or solvate or N-oxide thereof, or in the form of a pharmaceutically acceptable salt or solvate thereof can also be in the form of a polymorph, and
wherein if the compound listed above exhibits chirality it can be in the form of a mixture of enantiomers such as a racemate or a mixture of diastereomers, or can be in the form of a single enantiomer or a single diastereomer.

55-62. (canceled)

63. A compound to claim 1, wherein said compound is of subformula VIII or IX: 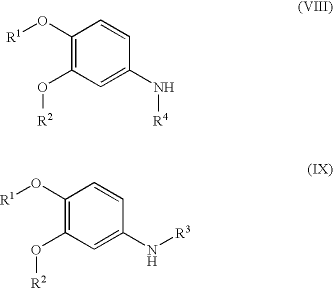
wherein R¹, R², R³, and R⁴ are as defined in claim 1.

64. A compound according to claim 63, wherein said compound is selected from:
289) 3-{[3,4-bis(difluoromethoxy)phenyl]amino}benzoic acid,
290) 2-methoxy-4-[(pyridin-3-ylmethyl)amino]benzamide,
291) 3,4-bis(difluoromethoxy)-N-(1,3-thiazol-5-ylmethyl)aniline,
292) 3,4-bis(difluoromethoxy)-N-[4-(methylsulfonyl)phenyl]aniline,
293) 4-{[4-(difluoromethoxy)-3-methoxyphenyl]amino}benzoic acid,
294) 4-{[4-(difluoromethoxy)-3-ethoxyphenyl]amino}benzoic acid, and
295) 4-{[3,4-bis(difluoromethoxy)phenyl]amino}benzoic acid.

65. A pharmaceutical composition comprising a compound according to claim 1, and a pharmaceutically acceptable carrier.

66. (canceled)

67. A composition according to claim 65, wherein said composition further comprises one or more additional pharmaceutical agent or agents selected from calcium channel blockers, cholinergic drugs, adenosine receptor modulators, ampakines, NMDA-R modulators, mGluR modulators, cholinesterase inhibitors, selective serotonin reuptake inhibitors, and combinations thereof.

68. A composition according to claim 67, wherein said additional pharmaceutical agent is donepezil.

69. A method for enhancing cognition in a patient in whom such enhancement is desired comprising administering to said patient an effective amount of a compound according to claim 1.

70. (canceled)

71. (canceled)

72. A method of treating a patient suffering from cognition impairment or decline comprising administering to said patient an effective amount of a compound according to claim 1.

73. A method according to claim 72, wherein said patient is a human.

74. A method according to claim 73, wherein said patient is suffering from memory impairment.

75. (canceled)

76. A method according to claim 74, wherein said patient is suffering from memory impairment due to Alzheimer's disease, multiple sclerosis, amylolaterosclerosis, multiple systems atrophy, schizophrenia, Parkinson's disease, Huntington's disease, Pick's disease, Creutzfeldt-Jakob disease, Rubenstein-Taybi syndrome, depression, aging, head trauma, stroke, spinal cord injury, CNS hypoxia, cerebral senility, diabetes associated cognitive impairment, memory deficits from early exposure of anesthetic agents, multiinfarct dementia, HIV or cardiovascular disease.

77-80. (canceled)

81. A method of treating a patient suffering from an allergic or inflammatory disease comprising administering to said patient an effective amount of a compound according to claim 1.

82-116. (canceled)
